(19) United States
(12) Patent Application Publication
(10) Pub. No.: US 2024/0174665 A1
(43) Pub. Date: May 30, 2024

(54) CDK2 INHIBITORS AND USES THEREOF

(71) Applicant: Kymera Therapeutics, Inc., Watertown, MA (US)

(72) Inventors: Philip COLLIER, Watertown, MA (US); Xiaozhang ZHENG, Watertown, MA (US); Xiao ZHU, Watertown, MA (US); Melissa FORD, Watertown, MA (US); Matthew M. WEISS, Watertown, MA (US); Robert AVERSA, Watertown, MA (US); Kiran Vijayakumari MAHASENAN, Watertown, MA (US); Yi ZHANG, Watertown, MA (US)

(21) Appl. No.: 18/453,163

(22) Filed: Aug. 21, 2023

Related U.S. Application Data
(60) Provisional application No. 63/373,014, filed on Aug. 19, 2022.
(60) Provisional application No. 63/383,042, filed on Nov. 9, 2022.

(51) Int. Cl. C07D 471/04; C07D 215/227; C07D 231/40; C07D 239/42; C07D 239/84; C07D 401/04; C07D 401/12; C07D 401/14; C07D 403/04; C07D 403/12; C07D 403/14; C07D 405/14; C07D 413/12; C07D 473/18; C07D 487/04; C07D 491/107; C07D 491/20; C07D 495/04; C07D 513/04; C07D 519/00

(52) U.S. Cl. CPC C07D 471/04; C07D 215/227; C07D 231/40; C07D 239/42; C07D 239/84; C07D 401/04; C07D 401/12; C07D 401/14; C07D 403/04; C07D 403/12; C07D 403/14; C07D 405/14; C07D 413/12; C07D 473/18; C07D 487/04; C07D 491/107; C07D 491/20; C07D 495/04; C07D 513/04; C07D 519/00; C07B 2200/05

(57) ABSTRACT

The present invention provides compounds, compositions thereof, and methods of using the same.

### CROSS-REFERENCE TO RELATED APPLICATIONS

This application claims the benefit of priority to U.S. Provisional Appl. No. 63/373,014, filed Aug. 19, 2022, and U.S. Provisional Appl. No. 63/383,042, filed Nov. 9, 2022, the contents of which is herein incorporated by reference.

### TECHNICAL FIELD OF THE INVENTION

The present invention relates to compounds and methods useful for the modulation of cyclin-dependent kinase 2 (“CDK2”). The invention also provides pharmaceutically acceptable compositions comprising compounds of the present invention and methods of using said compositions in the treatment of various disorders.

### BACKGROUND OF THE INVENTION

An ongoing need exists in the art for effective treatments for disease, especially cancers. Cyclin-dependent kinases (CDKs) are a family of serine/threonine kinases. Heterodimerized with regulatory subunits known as cyclins, such as cyclin E1 (“CCNE1”), CDKs become fully activated and regulate key cellular processes including cell cycle progression and cell division. Uncontrolled proliferation is a hallmark of cancer cells. The deregulation of the CDK activity is associated with abnormal regulation of cell-cycle, and is detected in virtually all forms of human cancers. As such, small molecule therapeutic agents that inhibit target cancer-associated proteins such as cyclin-dependent kinase 2 (“CDK2”) or CDK2 and CCNE1 protein hold promise as therapeutic agents. Accordingly, there remains a need to find compounds that are CDK2 or CDK2 and CCNE1 inhibitors as therapeutic agents.

### SUMMARY OF THE INVENTION

It has now been found that compounds of this invention, and pharmaceutically acceptable compositions thereof, are effective as inhibitors of CDK2 or CDK2 and CCNE1 protein. Such compounds have the general formula I-a or I-b:

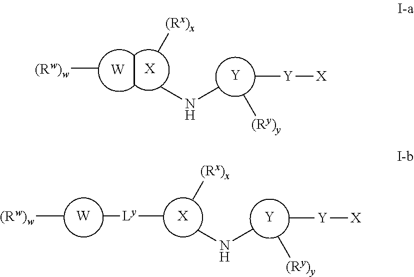

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of CDK2 protein. Such diseases, disorders, or conditions include those described herein.

Compounds provided by this invention are also useful for the study of CDK2 protein in biological and pathological phenomena; and the comparative evaluation of new CDK2 inhibitors, in vitro or in vivo.

### DETAILED DESCRIPTION OF CERTAIN EMBODIMENTS

### 1. General Description of Certain Embodiments of the Invention

Compounds of the present invention, and compositions thereof, are useful as inhibitors of CDK protein. In some embodiments, a provided compound inhibits CDK2 protein. In some embodiments, a provided compound inhibits CDK2 and CCNE1 protein.

In certain embodiments, the present invention provides a compound of formula I-a or I-b:

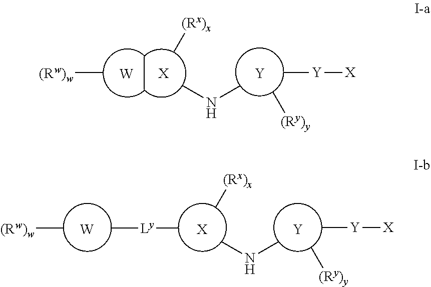

or a pharmaceutically acceptable salt thereof, wherein each variable is as defined and described herein.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic, bicyclic, bridged bicyclic, or spirocyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

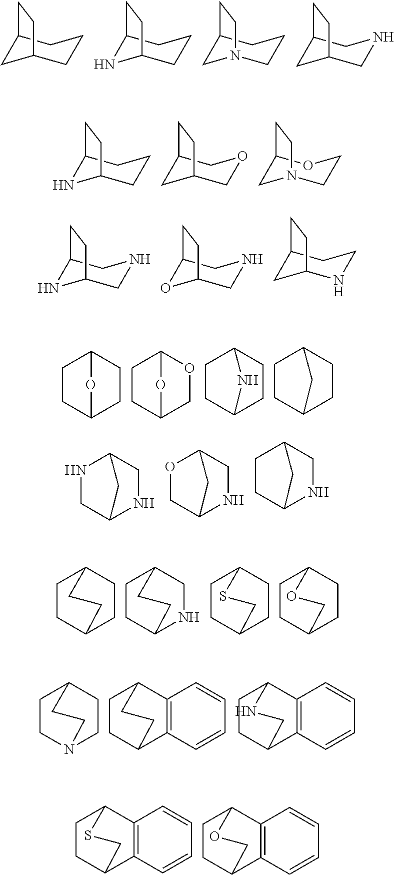

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

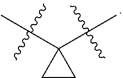 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁— pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —(CH₂)₀₋₄P(O)₂R^{∘}; —(CH₂)₀₋₄P(O)R^{∘}₂; —(CH₂)₀₋₄P(O)(OR^{∘})₂; —(CH₂)₀₋₄OP(O)R^{∘}₂; —(CH₂)₀₋₄OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂)₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromotagraphic purification are contemplated herein and are readily apparent to those having skill in the art.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “provided compound” refers to any genus, subgenus, and/or species set forth herein.

As used herein, the term “inhibitor” is defined as a compound that binds to and/or inhibits CDK2 or CDK2 and CCNE1 with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41:2596-9 and Sun et al., Bioconjugate Chem., 2006, 17:52-7.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in CDK2 or CDK2 and CCNE1 activity between a sample comprising a compound of the present invention, or composition thereof, and CDK2 or CDK2 and CCNE1, and an equivalent sample comprising CDK2 or CDK2 and CCNE1, in the absence of said compound, or composition thereof.

### 3. Description of Exemplary Embodiments

As described above, in certain embodiments, the present invention provides a compound of formula I-a:

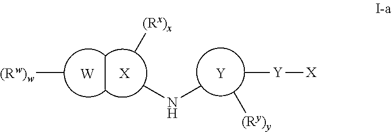

or a pharmaceutically acceptable salt thereof, wherein:
Ring W and Ring X are independently fused rings selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenylenyl, a 4 to 7-membered saturated or partially unsaturated carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
Y is a covalent bond, —S(O)₂—, —S(O)—, —S(O)(NR)—, —P(O)R—, or —P(O)OR—;
X is —NR₂ or an optionally substituted group selected from C₁₋₆ aliphatic, phenyl, a 3 to 12-membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, and —NRS(O)₂R; or
   two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and w, x, and y are independently 0, 1, 2, 3, or 4.

As described above, in certain embodiments, the present invention provides a compound of formula I-b:

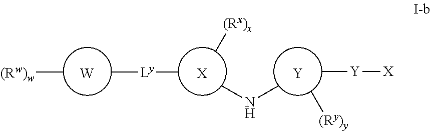

or a pharmaceutically acceptable salt thereof, wherein:
Ring W, Ring X, and Ring Y are independently rings selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Y is a covalent bond, —S(O)₂—, —S(O)—, —S(O)(NR)—, —P(O)R—, or —P(O)OR—;
X is —NR₂ or an optionally substituted group selected from C₁₋₆ aliphatic, phenyl, a 3 to 12-membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, and —NRS(O)₂R; or
   two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
L^{y} is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CF₂—, —CRF—, —NR—, —S—, —S(O)—, —S(O)₂— or —CR═CR—; and w, x, and y are independently 0, 1, 2, 3, or 4.

As described above, in certain embodiments, the present invention provides a compound of formula I-a′:

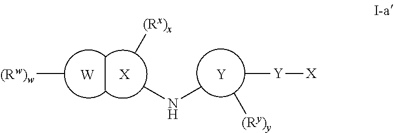

or a pharmaceutically acceptable salt thereof, wherein:
Ring W and Ring X are independently fused rings selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
Y is a covalent bond, —S(O)₂—, —S(O)—, —S(O)(NR)—, —P(O)R—, —P(O)OR— or

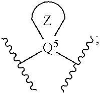

Ring Z is an optionally substituted 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur;
Q⁵ is carbon or sulfur;
X is —NR₂ or an optionally substituted group selected from C₁₋₆ aliphatic, phenyl, a 3 to 12-membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, and —NRS(O)₂R; or
   two R^{w} groups attached to the same or adjacent carbon atom are optionally taken together to form a spiro fused or 1,2-fused ring selected from a 3-12 membered saturated or partially unsaturated carbocyclyl and a 3-12 membered saturated or partially unsaturated heterocyclyl having 1-3 heteroatoms independently selected from nitrogen, oxygen, and sulfur;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form a 4-7 membered saturated, partially unsaturated, or heteroaryl ring having 0-3 heteroatoms, in addition to the atom or atoms to which they are attached, independently selected from nitrogen, oxygen, and sulfur; and
w, x, and y are independently 0, 1, 2, 3, or 4.

As described above, in certain embodiments, the present invention provides a compound of formula I-b′:

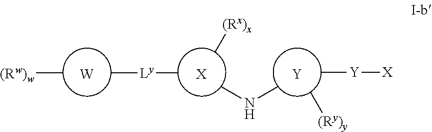

or a pharmaceutically acceptable salt thereof, wherein:
Ring W and Ring X are independently rings selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenyl, a 3 to 12-membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Y is a covalent bond, —S(O)₂—, —S(O)—, —S(O)(NR)—, —P(O)R—, —P(O)OR—, or

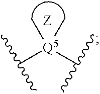

Ring Z is an optionally substituted 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur;
Q⁵ is carbon or sulfur;
X is —NR₂ or an optionally substituted group selected from C₁₋₆ aliphatic, phenyl, a 3 to 12-membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, and —NRS(O)₂R; or
   two R^{w} groups attached to the same or adjacent carbon atom are optionally taken together to form a spiro fused or 1,2-fused ring selected from a 3-12 membered saturated or partially unsaturated carbocyclyl and a 3-12 membered saturated or partially unsaturated heterocyclyl having 1-3 heteroatoms independently selected from nitrogen, oxygen, and sulfur;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form a 4-7 membered saturated, partially unsaturated, or heteroaryl ring having 0-3 heteroatoms, in addition to the atom or atoms to which they are attached, independently selected from nitrogen, oxygen, and sulfur; and
L^{y} is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CF₂—, —CRF—, —NR—, —S—, —S(O)—, —S(O)₂— or —CR═CR—, or:
   L^{y} and one R are optionally taken together with their intervening atoms to form a 5-6 membered saturated, partially unsaturated or heteroaryl ring having 0-3 heteroatoms independently selected from oxygen, nitrogen or sulfur; and
w, x, and y are independently 0, 1, 2, 3, or 4.

As defined generally above, Ring W, Ring X, and Ring Y are independently a ring selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.

In some embodiments, one or more of Ring W, Ring X, and Ring Y is a ring selected from phenyl. In some embodiments, one or more of Ring W, Ring X, and Ring Y is a 4 to 7-membered saturated or partially unsaturated carbocyclyl. In some embodiments, one or more of Ring W, Ring X, and Ring Y is a 4 to 7-membered saturated or partially unsaturated heterocyclyl with 1-3 heteroatoms independently selected from nitrogen, oxygen, and sulfur. In some embodiments, one or more of Ring W, Ring X, and Ring Y is a 5 to 6-membered heteroaryl with 1-4 heteroatoms independently selected from nitrogen, oxygen and sulfur.

As defined generally above, Ring W and Ring X are independently fused rings selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.

In some embodiments, one or more of Ring W and Ring X is benzo. In some embodiments, one or more of Ring W and Ring X is a fused 4 to 7-membered saturated or partially unsaturated carbocyclyl. In some embodiments, one or more of Ring W and Ring X is a fused 4 to 7-membered saturated or partially unsaturated heterocyclyl with 1-3 heteroatoms independently selected from nitrogen, oxygen, and sulfur. In some embodiments, one or more of Ring W and Ring X is a fused 5 to 6-membered heteroaryl with 1-4 heteroatoms independently selected from nitrogen, oxygen and sulfur.

In some embodiments, Ring W is a fused 5 to 6-membered heteroaryl with 1-4 heteroatoms independently selected from nitrogen, oxygen and sulfur. In some embodiments, Ring W is a fused 4 to 7-membered saturated or partially unsaturated heterocyclyl with 1-3 heteroatoms independently selected from nitrogen, oxygen, and sulfur.

In some embodiments, Ring W is a fused 5 to 6-membered heteroaryl with 1-2 nitrogen. In some embodiments, Ring W is a 5 to 6-membered heteroaryl with 1-2 nitrogen. In some embodiments, Ring W is a fused 5 to 6-membered saturated or partially unsaturated heterocyclyl with 1-2 nitrogen. In some embodiments, Ring W is a 5 to 6-membered saturated or partially unsaturated heterocyclyl with 1-2 nitrogen. In some embodiments, Ring W is

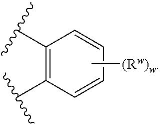

In some embodiments, Ring W is

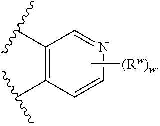

In some embodiments, Ring W is

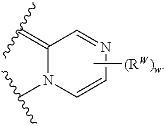

In some embodiments, Ring W is

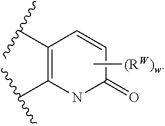

In some embodiments, Ring W is

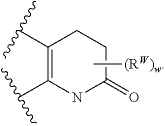

In some embodiments, Ring W is

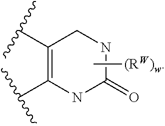

In some embodiments, Ring W is

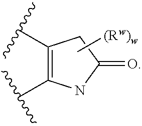

In some embodiments, Ring W is

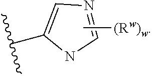

In some embodiments, Ring W is

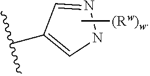

In some embodiments, Ring W is

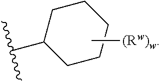

In some embodiments, Ring W is

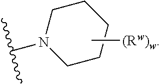

In some embodiments, Ring W is

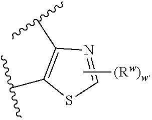

In some embodiments, Ring W is

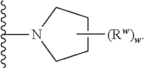

In some embodiments, Ring W is

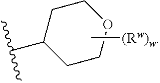

In some embodiments, Ring W is selected from those depicted in Table 1, below.

In some embodiments, Ring X is benzo. In some embodiments, Ring X is a fused 5 to 6-membered heteroaryl with 1-4 heteroatoms independently selected from nitrogen, oxygen and sulfur.

In some embodiments, Ring X is a fused 5 to 6-membered heteroaryl with 1-2 nitrogen. In some embodiments, Ring X is a fused 5 to 6-membered heteroaryl with 1 nitrogen. In some embodiments, Ring X is a fused 5-membered heteroaryl with sulfur or oxygen and optionally 1 nitrogen. In some embodiments, Ring X is

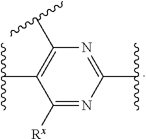

In some embodiments, Ring X is

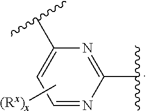

In some embodiments, Ring X is

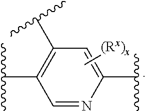

In some embodiments, Ring X is

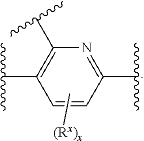

In some embodiments, Ring X is

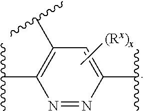

In some embodiments, Ring X is

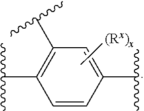

In some embodiments, Ring X is

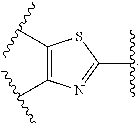 [SMILES: *c1nc(*)c(*)s1]

In some embodiments, Ring X is

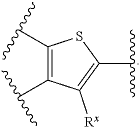

In some embodiments, Ring X is

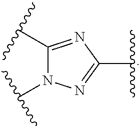 [SMILES: *c1nc(*)n(*)n1]

In some embodiments, Ring X is

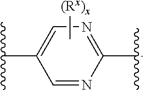

In some embodiments, Ring X is selected from those depicted in Table 1, below.

As defined generally above, Ring Y is a ring selected from phenyl, a 3 to 12-membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur and;

In some embodiments, Ring Y is

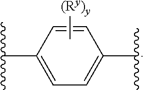

In some embodiments, Ring Y is

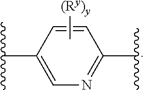

In some embodiments, Ring Y is

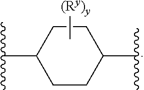

In some embodiments, Ring Y is

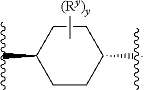

In some embodiments, Ring Y is

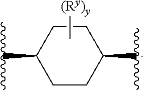

In some embodiments, Ring Y is

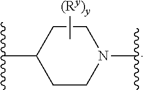

In some embodiments, Ring Y is

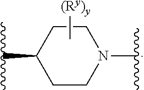

In some embodiments, Ring Y is

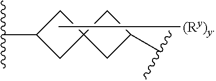

In some embodiments, Ring Y is

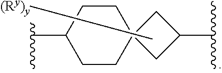

In some embodiments, Ring Y is

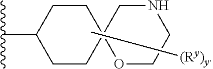

In some embodiments, Ring W, Ring X, and Ring Y are selected from those depicted in Table 1, below.

As defined generally above, Y is a covalent bond, —S(O)₂—, —S(O)—, —S(O)(NR)—, —P(O)R—, —P(O)OR—, or

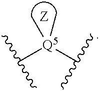

In some embodiments, Y is a covalent bond, —S(O)₂—, —S(O)—, —S(O)(NR)—, —P(O)R—, or —P(O)OR—. In some embodiments, Y is —S(NR)₂—. In some embodiments, Y is —S(O)₂NR—.

In some embodiments, Y is —S(O)₂—, —S(O)—, —S(O)(NR)—, —P(O)R—, or —P(O)OR—.

In some embodiments, Y is a covalent bond. In some embodiments, Y is —S(O)₂—. In some embodiments, Y is —S(O)—. In some embodiments, Y is —S(O)(NR)—

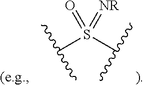

In some embodiments, Y is —P(O)R—. In some embodiments, Y is —P(O)OR—.

In some embodiments, Y is —S(O)₁₋₂—. In some embodiments, Y is —S(O)(NH)—. In some embodiments, Y is —P(O)Me-.

In some embodiments, Y is

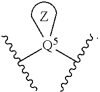

In some embodiments, Y is

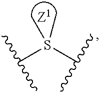 [SMILES: *S(*)(C)C]

wherein Ring Z¹ is an optionally substituted 3-12 membered saturated or partially unsaturated monocyclic, bicyclic, bridged bicyclic or spirocyclic heterocyclyl with an additional 0-2 heteroatoms independently selected from nitrogen, oxygen, and sulfur.

In some embodiments, Y is

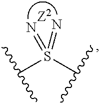 [SMILES: *S(*)(=N)=N]

wherein Ring Z² is an optionally substituted 3-12 membered saturated or partially unsaturated monocyclic, bicyclic, bridged bicyclic or spirocyclic heterocyclyl.

In some embodiments, Y is —S(NR)₂.

In some embodiments, Y is

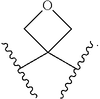 [SMILES: *C1(*)COC1]

In some embodiments, Y is selected from those depicted in Table 1, below.

As defined generally above, Ring Z is an optionally substituted 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur.

In some embodiments, Ring Z is an optionally substituted 3-12 membered saturated or partially unsaturated monocyclic, bicyclic, or spirocyclic carbocyclyl. In some embodiments, Ring Z is an optionally substituted 3-12 membered saturated or partially unsaturated monocyclic, bicyclic, or spirocyclic heterocyclyl with 1-3 heteroatoms independently selected from nitrogen, oxygen, and sulfur. In some embodiments, Ring Z is an optionally substituted 3-12 membered saturated or partially unsaturated monocyclic carbocyclyl. In some embodiments, Ring Z is an optionally substituted 3-12 membered saturated or partially unsaturated bicyclic carbocyclyl. In some embodiments, Ring Z is an optionally substituted 3-12 membered saturated or partially unsaturated bicyclic carbocyclyl. In some embodiments, Ring Z is an optionally substituted 3-12 membered saturated or partially unsaturated spirocyclic carbocyclyl. In some embodiments, Ring Z is an optionally substituted 3-12 membered saturated or partially unsaturated monocyclic heterocyclyl with 1-3 heteroatoms independently selected from nitrogen, oxygen, and sulfur. In some embodiments, Ring Z is an optionally substituted 3-12 membered saturated or partially unsaturated bicyclic heterocyclyl with 1-3 heteroatoms independently selected from nitrogen, oxygen, and sulfur. In some embodiments, Ring Z is an optionally substituted 3-12 membered saturated or partially unsaturated bicyclic heterocyclyl with 1-3 heteroatoms independently selected from nitrogen, oxygen, and sulfur. In some embodiments, Ring Z is an optionally substituted 3-12 membered saturated or partially unsaturated spirocyclic heterocyclyl with 1-3 heteroatoms independently selected from nitrogen, oxygen, and sulfur.

In some embodiments, Ring Z is selected from those depicted in Table 1, below.

As defined generally above, Q⁵ is carbon or sulfur.

In some embodiments, Q⁵ is carbon. In some embodiments, Q⁵ is sulfur.

In some embodiments, Q⁵ is selected from those depicted in Table 1, below.

As defined generally above, X is —NR₂— or an optionally substituted group selected from C₁₋₆ aliphatic, phenyl, a 3 to 12-membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.

In some embodiments, X is —NR₂—. In some embodiments, X is an optionally substituted C₁₋₆ aliphatic (e.g., C₁₋₆ alkyl, C₁₋₆ alkenyl C₁₋₆ alkynyl, etc.). In some embodiments, X is an optionally substituted C₁₋₆ alkyl. In some embodiments, X is methyl. In some embodiments, X is ethyl. In some embodiments, X is benzyl. In some embodiments, X is an optionally substituted phenyl. In some embodiments, X is an optionally substituted 3 to 12-membered saturated or partially unsaturated monocyclic, bicyclic, bridged bicyclic or spirocyclic carbocyclylenyl. In some embodiments, X is an optionally substituted 3 to 12-membered saturated or partially unsaturated monocyclic, bicyclic, bridged bicyclic or spirocyclic heterocyclylenyl with 1-3 heteroatoms independently selected from nitrogen, oxygen, and sulfur. In some embodiments, X is an optionally substituted 5 to 6-membered heteroarylenyl with 1-4 heteroatoms independently selected from nitrogen, oxygen and sulfur.

In some embodiments, Y connects to a carbon atom of X when X is an optionally substituted 3 to 12-membered saturated or partially unsaturated monocyclic, bicyclic, bridged bicyclic or spirocyclic heterocyclyl with 1-3 heteroatoms independently selected from nitrogen, oxygen, and sulfur, or when X is an optionally substituted 5 to 6-membered heteroaryl with 1-4 heteroatoms independently selected from nitrogen, oxygen and sulfur.

In some embodiments, X is an optionally substituted 3 to 12-membered saturated or partially unsaturated bicyclic carbocyclylenyl or heterocyclylenyl with 1-3 heteroatoms independently selected from nitrogen, oxygen. In some embodiments, X is an optionally substituted 3 to 12-membered saturated or partially unsaturated bridged bicyclic carbocyclylenyl or heterocyclylenyl with 1-3 heteroatoms independently selected from nitrogen, oxygen. In some embodiments, X is an optionally substituted 3 to 12-membered saturated or partially unsaturated spirocyclic carbocyclylenyl or heterocyclylenyl with 1-3 heteroatoms independently selected from nitrogen, oxygen. In some embodiments, X is an optionally substituted 3 to 12-membered saturated or partially unsaturated spirocyclic carbocyclylenyl. In some embodiments, X is an optionally substituted 3 to 12-membered saturated or partially unsaturated spirocyclic heterocyclylenyl with 1-3 heteroatoms independently selected from nitrogen, oxygen, and sulfur. In some embodiments, X is an optionally substituted 3 to 12-membered saturated or partially unsaturated spirocyclic carbocyclylenyl. In some embodiments, X is an optionally substituted 3 to 12-membered saturated or partially unsaturated spirocyclic heterocyclylenyl with 1-3 heteroatoms independently selected from nitrogen, oxygen, and sulfur. In some embodiments, X is an ortho-fluoro piperdine. In some embodiments, X is an ortho-methyl piperdine. In some embodiments, X is an meta-fluoro piperdine. In some embodiments, X is an meta-methyl piperdine.

In some embodiments, X is

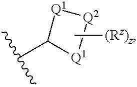

wherein:
each Q¹ is independently —O—, —S—, —C(O)—, —C(S)—, —CR₂—, or —NR—;
Q² is a C₁₋₉ bivalent saturated or unsaturated hydrocarbon chain or spirocyclic fused ring wherein 1-2 methylene units of the chain or ring are independently and optionally replaced with —O—, —S—, —C(O)—, —C(S)—, —CHR—, —CR₂—, —NH—, or —NR—;
each R^{z} is independently hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, or —NRS(O)₂R; and
z is 0, 1, 2, 3, or 4.

In some embodiments, X is

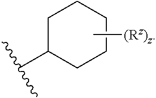

In some embodiments, X is

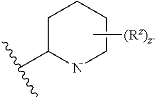

In some embodiments, X is

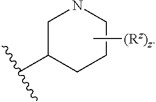

In some embodiments, X is

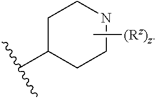

In some embodiments, X is

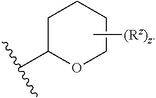

In some embodiments, X is

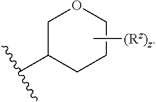

In some embodiments, X is

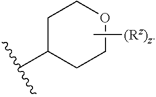

In some embodiments, X is

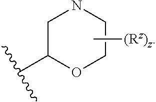

In some embodiments, X is

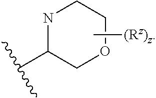

In some embodiments, X is

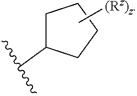

In some embodiments, X is

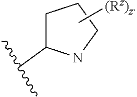

In some embodiments, X is

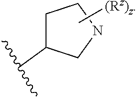

In some embodiments, X is

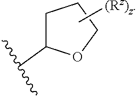

In some embodiments, X is

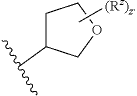

In some embodiments, X is

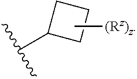

In some embodiments, X is

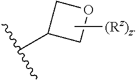

In some embodiments, X is

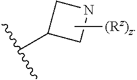

In some embodiments, X is

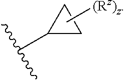

In some embodiments, X is

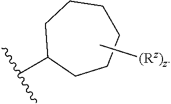

In some embodiments, X is

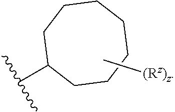

In some embodiments, X is

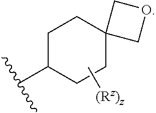

In some embodiments, X is

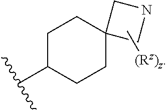

In some embodiments, X is

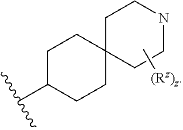

In some embodiments, X is

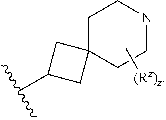

In some embodiments, X is

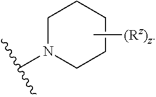

In some embodiments, X is

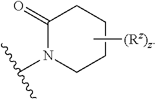

In some embodiments, X is

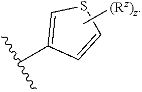

In some embodiments, X is

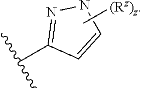

In some embodiments, X is

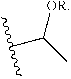

In some embodiments, X is

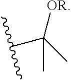

In some embodiments, X is methyl. In some embodiments, X is ethyl. In some embodiments, X is isopropyl. In some embodiments, X is —NHMe. In some embodiments, X is

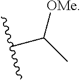 [SMILES: *C(C)OC]

In some embodiments, X is

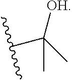 [SMILES: *C(C)(C)O]

In some embodiments, X is

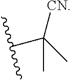 [SMILES: *C(C)(C)C#N]

In some embodiments, X is

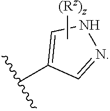

In some embodiments, X is

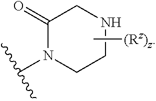

In some embodiments, X is

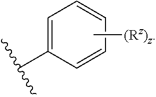

In some embodiments, X is

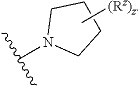

In some embodiments, X is

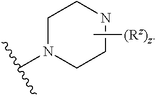

In some embodiments, X is selected from those depicted in Table 1, below.

As defined generally above, R^{w}, R^{x}, and R^{y} are independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, and —NRS(O)₂R, or two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur.

In some embodiments, one or more of R^{w}, R^{x}, and R^{y} is hydrogen. In some embodiments, one or more of R^{w}, R^{x}, and R^{y} is R^{A}. In some embodiments, one or more of R^{w}, R^{x}, and R^{y} is halogen. In some embodiments, one or more of R^{w}, R^{x}, and R^{y} is —CN. In some embodiments, one or more of R^{w}, R^{x}, and R^{y} is —NO₂. In some embodiments, one or more of R^{w}, R^{x}, and R is —OR. In some embodiments, one or more of R^{w}, R^{x}, and R^{y} is —SR. In some embodiments, one or more of R^{w}, R^{x}, and R^{y} is —NR₂. In some embodiments, one or more of R^{w}, R^{x}, and R^{y} is —SiR₃. In some embodiments, one or more of R^{w}, R^{x}, and R^{y} is —S(O)₂R. In some embodiments, one or more of R^{w}, R^{x}, and R^{y} is —S(O)₂NR₂. In some embodiments, one or more of R^{w}, R^{x}, and R^{y} is —S(O)R. In some embodiments, one or more of R^{w}, R^{x}, and R is —C(O)R. In some embodiments, one or more of R^{w}, R^{x}, and R^{y} is —C(O)OR. In some embodiments, one or more of R^{w}, R^{x}, R^{y}, and R^{z} is —C(O)NR₂. In some embodiments, one or more of R^{w}, R^{x}, and R^{y} is —C(O)NROR. In some embodiments, one or more of R^{w}, R^{x}, and R^{y} is —OC(O)R. In some embodiments, one or more of R^{w}, R^{x}, and R^{y} is —OC(O)NR₂. In some embodiments, one or more of R^{w}, R^{x}, R^{y}, and R^{z} is —OP(O)R₂. In some embodiments, one or more of R^{w}, R^{x}, and R^{y} is —OP(O)(OR)₂. In some embodiments, one or more of R^{w}, R^{x}, and R^{y} is —OP(O)(OR)NR₂. In some embodiments, one or more of R^{w}, R^{x}, and R is —OP(O)(NR₂)₂—. In some embodiments, one or more of R^{w}, R^{x}, R^{y}, and R^{z} is —P(O)R₂. In some embodiments, one or more of R^{w}, R^{x}, and R^{y} is —P(O)(OR)₂. In some embodiments, one or more of R^{w}, R^{x}, and R^{y} is —P(O)(OR)NR₂. In some embodiments, one or more of R^{w}, R^{x}, and R^{y} is —P(O)(NR₂)₂—. In some embodiments, one or more of R^{w}, R^{x}, and R^{y} is —NRC(O)OR. In some embodiments, one or more of R^{w}, R^{x}, and R^{y} is —NRC(O)R. In some embodiments, one or more of R^{w}, R^{x}, and R^{y} is —NRC(O)N(R)₂. In some embodiments, one or more of R^{w}, R^{x}, and R^{y} is —NRS(O)₂R. In some embodiments, one or more of R^{w}, R^{x}, and R^{y} is —NP(O)R₂. In some embodiments, one or more of R^{w}, R^{x}, and R^{y} is —NRP(O)(OR)₂. In some embodiments, one or more of R^{w}, R^{x}, and R^{y} is —NRP(O)(OR)NR₂. In some embodiments, one or more of R^{w}, R^{x}, and R^{y} is —NRP(O)(NR₂)₂. In some embodiments, one or more of R^{w}, R^{x}, and R^{y} is —NRS(O)₂R. In some embodiments, two R^{w} groups attached to the same carbon atom are taken together to form a 3-5 membered saturated or partially unsaturated carbocyclic spiro fused ring. In some embodiments, two R^{w} groups attached to the same carbon atom are optionally taken together to form a 3-5 membered saturated or partially unsaturated heterocyclic spiro fused ring having 1-2 heteroatoms independently selected from nitrogen, oxygen, and sulfur.

In some embodiments, one or more R^{w} is selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂, or two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur.

In some embodiments, one or more R^{w} is hydrogen. In some embodiments, one or more R^{w} is R^{A}. In some embodiments, one or more R^{w} is halogen. In some embodiments, one or more R^{w} is —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, or —NRP(O)(NR₂)₂. In some embodiments two R^{w} groups attached to the same carbon atom are taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur.

In some embodiments, two R^{w} groups attached to the same carbon atom are taken together to form a spiro fused 3-5 membered saturated or partially unsaturated carbocyclyl. In some embodiments, two R^{w} groups attached to the same carbon atom are taken together to form a spiro fused 3-5 membered saturated or partially unsaturated heterocyclyl having 1-2 heteroatoms independently selected from nitrogen, oxygen, and sulfur. In some embodiments, two R^{w} groups attached to the same or adjacent carbon atom are optionally taken together to form a spiro fused or 1,2-fused ring selected from a 3-12 membered saturated or partially unsaturated carbocyclyl and a 3-12 membered saturated or partially unsaturated heterocyclyl having 1-3 heteroatoms independently selected from nitrogen, oxygen, and sulfur. In some embodiments, two R^{w} groups attached to the same or adjacent carbon atom are taken together to form a spiro fused or 1,2-fused ring selected from a 3-12 membered saturated or partially unsaturated carbocyclyl and a 3-12 membered saturated or partially unsaturated heterocyclyl having 1-3 heteroatoms independently selected from nitrogen, oxygen, and sulfur. In some embodiments, two R^{w} groups attached to the same carbon atom are taken together to form a spiro fused 3-12 membered saturated or partially unsaturated carbocyclyl.

In some embodiments, two R^{w} groups attached to the same carbon atom are taken together to form a spiro fused 3-12 membered saturated or partially unsaturated heterocyclyl having 1-3 heteroatoms independently selected from nitrogen, oxygen, and sulfur. In some embodiments, two R^{w} groups attached to the adjacent carbon atoms are taken together to form a 1,2-fused 3-12 membered saturated or partially unsaturated carbocyclyl. In some embodiments, two R7 groups attached to the adjacent carbon atom are taken together to form a 1,2-fused 3-12 membered saturated or partially unsaturated heterocyclyl having 1-3 heteroatoms independently selected from nitrogen, oxygen, and sulfur.

In some embodiments, R^{w} is fluoro. In some embodiments, R^{w} is chloro. In some embodiments, R^{w} is bromo. In some embodiments, R^{w} is —CN. In some embodiments, R^{w} is —OH. In some embodiments, R^{w} is —OMe. In some embodiments, R^{w} is -OiPr. In some embodiments, R^{w} is —O— cyclopropyl. In some embodiments, R^{w} is —O-cyclobutyl. In some embodiments, R^{w} is —CONH₂.

In some embodiments, R^{w} is R^{A}. In some embodiments, R^{w} is methyl. In some embodiments, R^{w} is ethyl. In some embodiments, R^{w} is isopropyl. In some embodiments, R^{w} is tert-butyl. In some embodiments, R^{w} is cyclopropyl. In some embodiments, R^{w} is cyclobutyl. In some embodiments, R^{w} is cyclopentyl. In some embodiments, R^{w} is —CHF₂. In some embodiments, R^{w} is —CF₃. In some embodiments, R^{w} is —CH₂CHF₂. In some embodiments, R^{w} is —CH(Me)CF₃. In some embodiments, R^{w} is —CMe₂OH. In some embodiments, R^{w} is

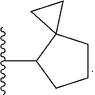 [SMILES: *C1CCCC12CC2]

In some embodiments, R^{w} is

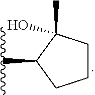 [SMILES: *[C@@H]1CCC[C@@]1(C)O]

In some embodiments, R^{w} is

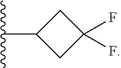 [SMILES: *C1CC(F)(F)C1]

In some embodiments, R^{w} is

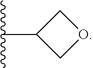 [SMILES: *C1COC1]

In some embodiments, R^{w} is

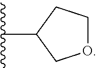 [SMILES: *C1CCOC1]

In some embodiments, R^{w} is

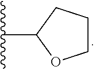 [SMILES: *C1CCCO1]

In some embodiments, R^{w} is

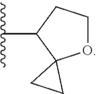 [SMILES: *C1CCOC12CC2]

In some embodiments, R^{w} is

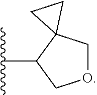 [SMILES: *C1COCC12CC2]

In some embodiments, R^{w} is

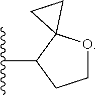 [SMILES: *C1CCOC12CC2]

In some embodiments, R^{w} is

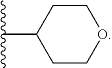 [SMILES: *C1CCOCC1]

In some embodiments, R^{w} is

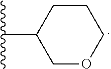 [SMILES: *C1CCCOC1]

In some embodiments, R^{w} is

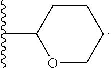 [SMILES: *C1CCCCO1]

In some embodiments, R^{w} is

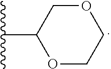 [SMILES: *C1COCCO1]

In some embodiments, R^{w} is

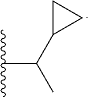 [SMILES: *C(C)C1CC1]

In some embodiments, R^{w} is

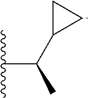 [SMILES: *[C@H](C)C1CC1]

In some embodiments, R^{w} is

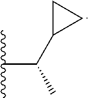 [SMILES: *[C@@H](C)C1CC1]

In some embodiments, R^{w} is

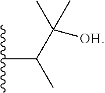 [SMILES: *C(C)C(C)(C)O]

In some embodiments, R^{w} is

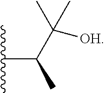 [SMILES: *[C@H](C)C(C)(C)O]

In some embodiments, R^{w} is

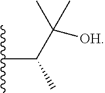 [SMILES: *[C@@H](C)C(C)(C)O]

In some embodiments, R^{w} is

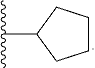 [SMILES: *C1CCCC1]

In some embodiments, R^{w} is

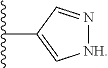 [SMILES: *c1cn[nH]c1]

In some embodiments, R^{w} is

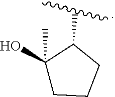 [SMILES: *[C@@H]1CCC[C@@]1(C)O]

In some embodiments, R^{w} is

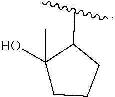 [SMILES: *C1CCCC1(C)O]

In some embodiments, R^{w} is

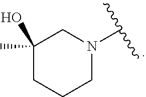 [SMILES: *N1CCC[C@@](C)(O)C1]

In some embodiments, R^{w} is

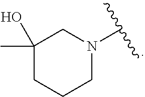 [SMILES: *N1CCCC(C)(O)C1]

In some embodiments, R^{w} is

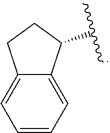 [SMILES: *[C@H]1CCc2ccccc21]

In some embodiments, R^{w} is

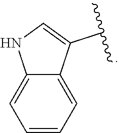 [SMILES: *c1c[nH]c2ccccc12]

In some embodiments, two R^{w} cyclize to form cyclopropylenyl. In some embodiments, two R^{w} cyclize to form an optionally substituted cyclobutylenyl. In some embodiments, two R^{w} cyclize to form cyclobutylenyl. In some embodiments, two R^{w} cyclize to form

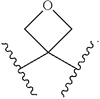 [SMILES: *C1(*)COC1]

In some embodiments, two R^{w} cyclize to form

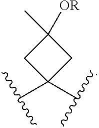

In some embodiments, two R^{w} cyclize to form

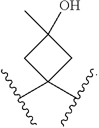 [SMILES: *C1(*)CC(C)(O)C1]

In some embodiments, two R^{w} cyclize to form

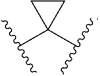 [SMILES: *C1(*)CC1]

In some embodiments, one or more R^{x} is selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂.

In some embodiments, one or more R^{x} is hydrogen. In some embodiments, one or more R^{x} is R^{A}. In some embodiments, one or more R^{x} is halogen. In some embodiments, one or more R^{x} is —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, or —NRP(O)(NR₂)₂.

In some embodiments, R^{x} is fluoro. In some embodiments, R^{x} is bromo. In some embodiments, R^{x} is R^{A}. In some embodiments, R^{x} is —CHF₂. In some embodiments, R^{x} is —CF₃.

In some embodiments, one or more R^{y} is selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂.

In some embodiments, one or more R^{y} is hydrogen. In some embodiments, one or more R^{y} is R^{A}. In some embodiments, one or more R^{y} is halogen. In some embodiments, one or more R^{y} is —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, or —NRP(O)(NR₂)₂.

In some embodiments, R^{y} is R^{A}. In some embodiments, R^{y} is methyl.

In some embodiments, R^{w}, R^{x}, and R^{y} are selected from those depicted in Table 1, below.

As defined generally above, each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is an optionally substituted C₁₋₆ aliphatic. In some embodiments, R^{A} is phenyl. 3-12 membered saturated or partially unsaturated monocyclic, bicyclic, bridged bicyclic or spirocyclic carbocyclyl. In some embodiments, R^{A} is an optionally substituted 3-12 membered saturated or partially unsaturated monocyclic, bicyclic, bridged bicyclic or spirocyclic heterocyclyl with 1-3 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R^{A} is C₁₋₆alkyl (e.g., methyl, ethyl, isopropyl, etc.). In some embodiments, R^{A} is C₁₋₆haloalkyl (e.g., —CF₃, —CHF₂, etc.).

In some embodiments, R^{A} is selected from those depicted in Table 1, below.

As defined generally above, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted a 4-7 membered saturated or partially unsaturated heterocyclic having 1-2 heteroatoms independently selected from nitrogen, oxygen, and sulfur. In some embodiments, R is an optionally substituted a 5-6 membered heteroaryl ring having 1-4 heteroatoms independently selected from nitrogen, oxygen, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur.

In some embodiments, R is C₁₋₆alkyl (e.g., methyl, ethyl, isopropyl, etc.). In some embodiments, R is C₁₋₆haloalkyl (e.g., —CF₃, —CHF₂, etc.).

In some embodiments, R is selected from those depicted in Table 1, below.

As defined generally above, L^{y} is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CF₂—, —CRF—, —NR—, —S—, —S(O)—, —S(O)₂— or —CR═CR—.

In some embodiments, L^{y} is a covalent bond. In some embodiments, L^{y} is a C₁₋₃ bivalent straight or branched saturated or unsaturated hydrocarbon chain wherein 1-2 methylene units of the chain are independently and optionally replaced with —O—, —C(O)—, —C(S)—, —CF₂—, —CRF—, —NR—, —S—, —S(O)—, —S(O)₂— or —CR═CR—.

In some embodiments, L^{y} is selected from those depicted in Table 1, below.

As defined generally above, w, x, and y are independently 0, 1, 2, 3, or 4.

In some embodiments, one or more of w, x, and y is 0. In some embodiments, one or more of w, x, and y is 1. In some embodiments, one or more of w, x, and y is 2. In some embodiments, one or more of w, x, and y is 3. In some embodiments, one or more of w, x, and y is 4.

In some embodiments, w is 0 or 1. In some embodiments, w is 1 or 2. In some embodiments, x is 0 or 1. In some embodiments, x is 1 or 2. In some embodiments, y is 0 or 1. In some embodiments, y is 1 or 2.

In some embodiments, w, x, y, and z are selected from those depicted in Table 1, below.

In some embodiments, the present invention provides the compound of formula I-a or I-a′, wherein Y is —S(O)₁₋₂— and Ring Y is phenylenyl, thereby forming a compound of formula I-a-1:

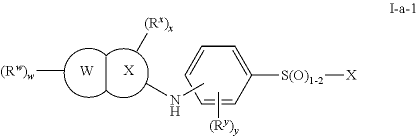

or a pharmaceutically acceptable salt thereof, wherein each of X, R^{w}, R^{x}, R^{y}, Ring W, Ring X, w, x, and y is as defined above and described in embodiments herein, both singly and in combination.

In some embodiments, the present invention provides the compound of formula I-a or I-a′, wherein Y is —S(O)₁₋₂—, Ring Y is phenylenyl and X is C₁₋₆ alkyl, thereby forming a compound of formula I-a-2:

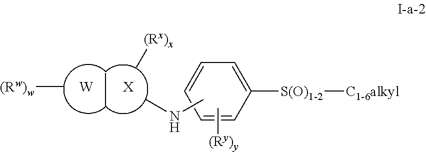

or a pharmaceutically acceptable salt thereof, wherein each of R^{w}, R^{x}, R^{y}, Ring W, Ring X, w, x, and y is as defined above and described in embodiments herein, both singly and in combination.

In some embodiments, the present invention provides the compound of formula I-a or I-a′, wherein Y is —S(O)₁₋₂—, Ring Y is phenylenyl and X is C₃₋₁₂ cycloalkyl, thereby forming a compound of formula I-a-3:

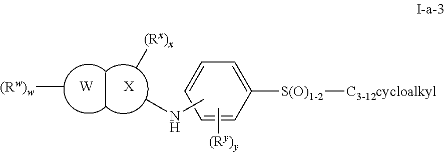

or a pharmaceutically acceptable salt thereof, wherein each of R^{w}, R^{x}, R^{y}, Ring W, Ring X, w, x, and y is as defined above and described in embodiments herein, both singly and in combination.

In some embodiments, the present invention provides the compound of formula I-a or I-a′, wherein Y is —S(O)₁₋₂—, Ring Y is phenylenyl and X is C₃₋₁₂ heterocycloalkyl, thereby forming a compound of formula I-a-4:

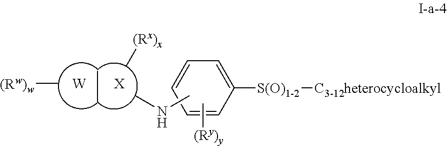

or a pharmaceutically acceptable salt thereof, wherein each of R^{w}, R^{x}, R^{y}, Ring W, Ring X, w, x, and y is as defined above and described in embodiments herein, both singly and in combination.

In some embodiments, the present invention provides the compound of formula I-a or I-a′, wherein Y is —S(O)₁₋₂— and Ring W is

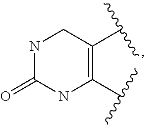 [SMILES: *C1=C(*)NC(=O)NC1]

thereby forming a compound of formula I-a-5:

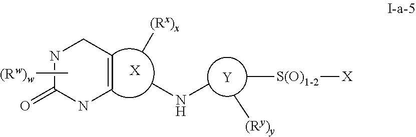

or a pharmaceutically acceptable salt thereof, wherein each of X, R^{w}, R^{x}, R^{y}, Ring X, Ring Y, w, x, and y is as defined above and described in embodiments herein, both singly and in combination.

In some embodiments, the present invention provides the compound of formula I-a or I-a′, wherein Y is —S(O)₁₋₂— and Ring W is

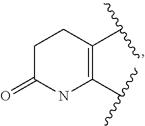 [SMILES: *C1=C(*)NC(=O)CC1]

thereby forming a compound of formula I-a-6:

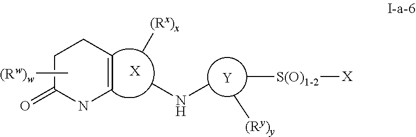

or a pharmaceutically acceptable salt thereof, wherein each of X, R^{w}, R^{x}, R^{y}, Ring X, Ring Y, w, x, and y is as defined above and described in embodiments herein, both singly and in combination.

In some embodiments, the present invention provides the compound of formula I-a or I-a′, wherein Y is —S(O)₁₋₂— and Ring X is pyridylenyl, thereby forming a compound of formula I-a-7:

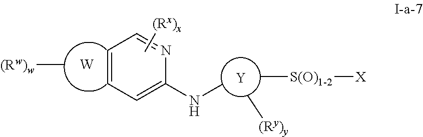

or a pharmaceutically acceptable salt thereof, wherein each of X, R^{w}, R^{x}, R^{y}, Ring X, Ring Y, w, x, and y is as defined above and described in embodiments herein, both singly and in combination.

In some embodiments, the present invention provides the compound of formula I-a or I-a′, wherein Y is —S(O)₁₋₂— and Ring X is pyridylenyl, thereby forming a compound of formula I-a-8:

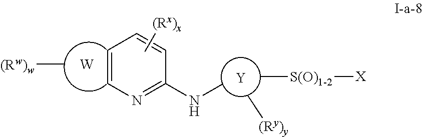

or a pharmaceutically acceptable salt thereof, wherein each of X, R^{w}, R^{x}, R^{y}, Ring X, Ring Y, w, x, and y is as defined above and described in embodiments herein, both singly and in combination.

In some embodiments, the present invention provides the compound of formula I-a or I-a′, wherein Y is —S(O)(NR)— and Ring Y is phenylenyl, thereby forming a compound of formula I-a-9:

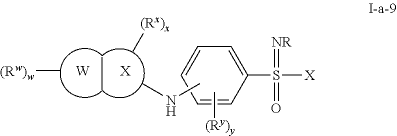

or a pharmaceutically acceptable salt thereof, wherein each of X, R, R^{w}, R^{x}, R^{y}, Ring W, Ring X, w, x, and y is as defined above and described in embodiments herein, both singly and in combination.

In some embodiments, the present invention provides the compound of formula I-a or I-a′, wherein Y is —S(O)₁₋₂— and Ring X is pyridylenyl, thereby forming a compound of formula I-a-10:

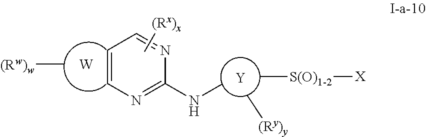

or a pharmaceutically acceptable salt thereof, wherein each of X, R^{w}, R^{x}, R^{y}, Ring X, Ring Y, w, x, and y is as defined above and described in embodiments herein, both singly and in combination.

In some embodiments, the present invention provides the compound of formula I-b or I-b′, wherein Y is —S(O)₁₋₂—, Ring Y is phenylenyl, thereby forming a compound of formula I-b-1:

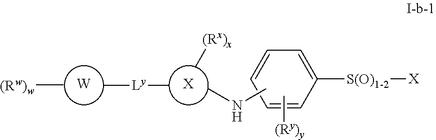

or a pharmaceutically acceptable salt thereof, wherein each of X, R^{w}, R^{x}, R^{y}, Ring W, Ring X, L, w, x, and y is as defined above and described in embodiments herein, both singly and in combination.

In some embodiments, the present invention provides the compound of formula I-b or I-b′, wherein Y is —S(O)₁₋₂—, Ring Y is phenylenyl and X is C₁₋₆ alkyl, thereby forming a compound of formula I-b-2:

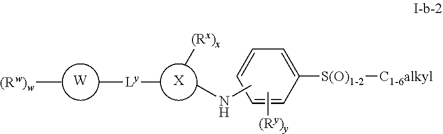

or a pharmaceutically acceptable salt thereof, wherein each of R^{w}, R^{x}, R^{y}, Ring W, Ring X, L, w, x, and y is as defined above and described in embodiments herein, both singly and in combination.

In some embodiments, the present invention provides the compound of formula I-b or I-b′, wherein Y is —S(O)₁₋₂—, Ring Y is phenylenyl and X is C₃₋₁₂ cycloalkyl, thereby forming a compound of formula I-b-3:

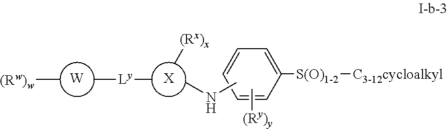

or a pharmaceutically acceptable salt thereof, wherein each of R^{w}, R^{x}, R^{y}, Ring W, Ring X, L, w, x, and y is as defined above and described in embodiments herein, both singly and in combination.

In some embodiments, the present invention provides the compound of formula I-b or I-b′, wherein Y is —S(O)₁₋₂—, Ring Y is phenylenyl and X is C₃₋₁₂ heterocycloalkyl, thereby forming a compound of formula I-b-4:

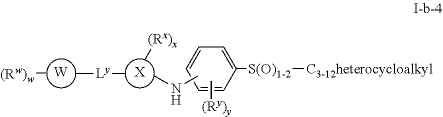

or a pharmaceutically acceptable salt thereof, wherein each of R^{w}, R^{x}, R^{y}, Ring W, Ring X, L, w, x, and y is as defined above and described in embodiments herein, both singly and in combination.

In some embodiments, the present invention provides the compound of formula I-b or I-b′, wherein Y is —S(O)₁₋₂—, L^{y} is a covalent bond, Ring W is cyclohexyl, and Ring Y is phenylenyl, thereby forming a compound of formula I-b-5:

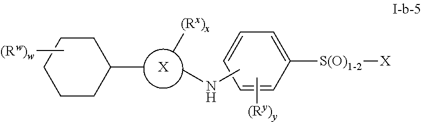

or a pharmaceutically acceptable salt thereof, wherein each of X, R^{w}, R^{x}, R^{y}, Ring X, x, and y is as defined above and described in embodiments herein, both singly and in combination.

In some embodiments, the present invention provides the compound of formula I-b or I-b′, wherein Y is —S(O)(NR)— and Ring Y is phenylenyl, thereby forming a compound of formula I-b-6:

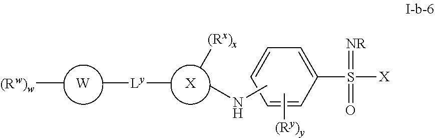

or a pharmaceutically acceptable salt thereof, wherein each of X, R^{w}, R^{x}, R^{y}, Ring W, Ring X, L, w, x, and y is as defined above and described in embodiments herein, both singly and in combination.

Exemplary compounds of the invention are set forth in Table 1, below.

In certain embodiments, the present invention provides a compound of formula I-b or I-b′ forming a compound of formula 1-b-7:

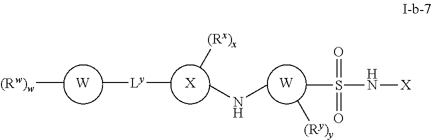

or a pharmaceutically acceptable salt thereof, wherein each of Ring W, Ring X, Ring Y, R^{x}, R^{y}, R^{w}, L^{y}, x, and w is as defined above and described in embodiments herein, both singly and in combination; and wherein X is an optionally substituted ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur.

In some embodiments, X is an optionally substituted 3 to 12-membered saturated or partially unsaturated bicyclic carbocyclylenyl or heterocyclylenyl with 1-3 heteroatoms independently selected from nitrogen, oxygen. In some embodiments, X is an optionally substituted 3 to 12-membered saturated or partially unsaturated bridged bicyclic carbocyclylenyl or heterocyclylenyl with 1-3 heteroatoms independently selected from nitrogen, oxygen. In some embodiments, X is an optionally substituted 3 to 12-membered saturated or partially unsaturated spirocyclic carbocyclylenyl or heterocyclylenyl with 1-3 heteroatoms independently selected from nitrogen, oxygen. In some embodiments, X is an optionally substituted 3 to 12-membered saturated or partially unsaturated spirocyclic carbocyclylenyl. In some embodiments, X is an optionally substituted 3 to 12-membered saturated or partially unsaturated spirocyclic heterocyclylenyl with 1-3 heteroatoms independently selected from nitrogen, oxygen, and sulfur. In some embodiments, X is an optionally substituted 3 to 12-membered saturated or partially unsaturated spirocyclic carbocyclylenyl. In some embodiments, X is an optionally substituted 3 to 12-membered saturated or partially unsaturated spirocyclic heterocyclylenyl with 1-3 heteroatoms independently selected from nitrogen, oxygen, and sulfur.

In certain embodiments, the present invention provides a compound of formula I-b or I-b′ forming a compound of formula 1-bb-1, 1-bb-2, or 1-bb-3:

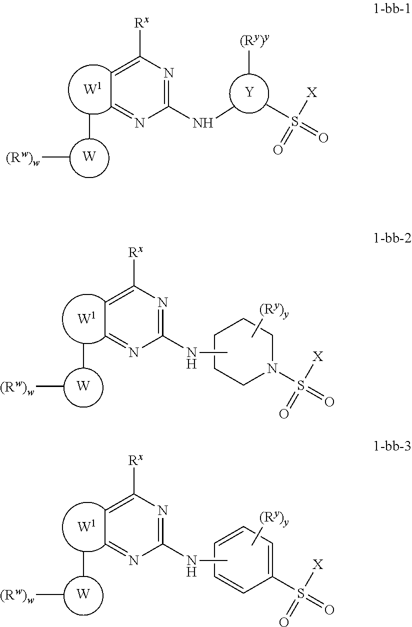

or a pharmaceutically acceptable salt thereof, wherein each of R^{x}, R^{y}, R^{w}, W, X, y and w, is as defined above and described in embodiments herein, both singly and in combination; and wherein L^{y} and one R^{x} are taken together with their intervening atoms to form Ring W¹, wherein Ring W¹ is a 5-6 membered saturated, partially unsaturated or heteroaryl ring having 0-3 heteroatoms independently selected from oxygen, nitrogen or sulfur.

In some embodiments, Ring W is a 4 to 7-membered saturated or partially unsaturated carbocyclyl. In some embodiments, Ring W is a 4 to 7-membered saturated or partially unsaturated heterocyclyl with 1-3 heteroatoms independently selected from nitrogen, oxygen, and sulfur. In some embodiments, Ring W is a 5 to 6-membered saturated or partially unsaturated heterocyclyl with 1-3 heteroatoms independently selected from nitrogen, oxygen, and sulfur. In some embodiments, Ring W is a fused 4 to 7-membered saturated or partially unsaturated heterocyclyl with 1-3 heteroatoms independently selected from nitrogen, oxygen, and sulfur.

TABLE 1
Exemplary Compounds

| I-# | Structure |
|---|---|
| I-1 | 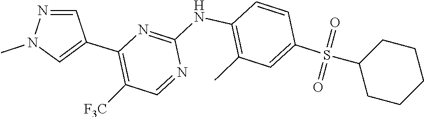 [SMILES: Cc1cc(S(=O)(=O)C2CCCCC2)ccc1Nc1ncc(C(F)(F)F)c(-c2cnn(C)c2)n1] |
| I-2 | 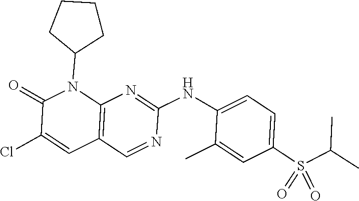 [SMILES: Cc1cc(S(=O)(=O)C(C)C)ccc1Nc1ncc2cc(Cl)c(=O)n(C3CCCC3)c2n1] |
| I-3 | 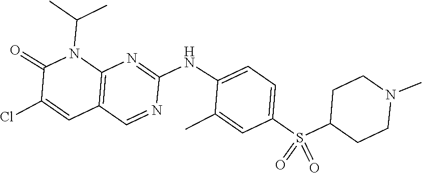 [SMILES: Cc1cc(S(=O)(=O)C2CCN(C)CC2)ccc1Nc1ncc2cc(Cl)c(=O)n(C(C)C)c2n1] |
| I-4 | 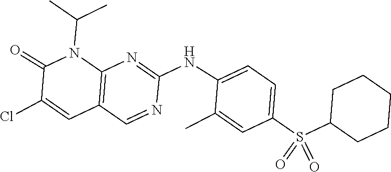 [SMILES: Cc1cc(S(=O)(=O)C2CCCCC2)ccc1Nc1ncc2cc(Cl)c(=O)n(C(C)C)c2n1] |
| I-5 | 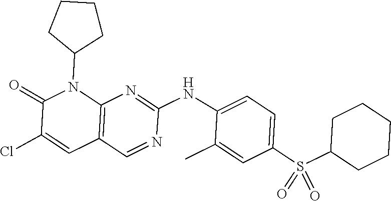 [SMILES: Cc1cc(S(=O)(=O)C2CCCCC2)ccc1Nc1ncc2cc(Cl)c(=O)n(C3CCCC3)c2n1] |
| I-6 | 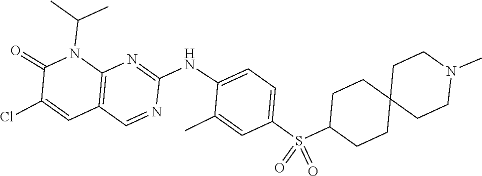 [SMILES: Cc1cc(S(=O)(=O)C2CCC3(CC2)CCN(C)CC3)ccc1Nc1ncc2cc(Cl)c(=O)n(C(C)C)c2n1] |
| I-7 | 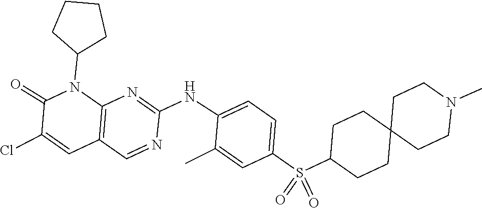 [SMILES: Cc1cc(S(=O)(=O)C2CCC3(CC2)CCN(C)CC3)ccc1Nc1ncc2cc(Cl)c(=O)n(C3CCCC3)c2n1] |
| I-8 | 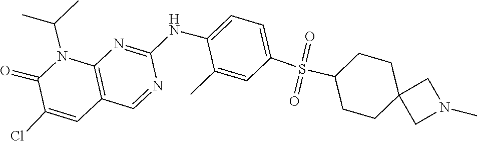 [SMILES: Cc1cc(S(=O)(=O)C2CCC3(CC2)CN(C)C3)ccc1Nc1ncc2cc(Cl)c(=O)n(C(C)C)c2n1] |
| I-9 | 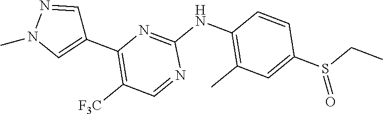 [SMILES: CCS(=O)c1ccc(Nc2ncc(C(F)(F)F)c(-c3cnn(C)c3)n2)c(C)c1] |
| I-10 | 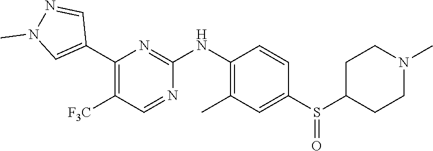 |
| I-11 | 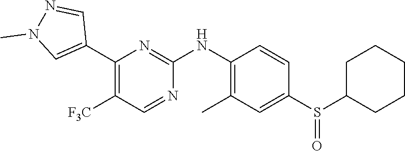 [SMILES: Cc1cc(S(=O)C2CCCCC2)ccc1Nc1ncc(C(F)(F)F)c(-c2cnn(C)c2)n1] |
| I-12 | 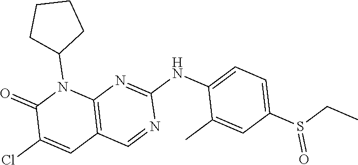 [SMILES: CCS(=O)c1ccc(Nc2ncc3cc(Cl)c(=O)n(C4CCCC4)c3n2)c(C)c1] |
| I-13 | 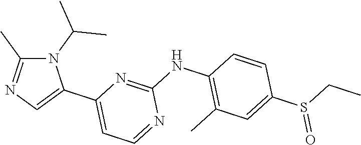 [SMILES: CCS(=O)c1ccc(Nc2nccc(-c3cnc(C)n3C(C)C)n2)c(C)c1] |
| I-14 | 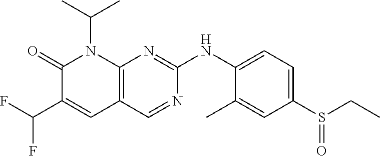 [SMILES: CCS(=O)c1ccc(Nc2ncc3cc(C(F)F)c(=O)n(C(C)C)c3n2)c(C)c1] |
| I-15 | 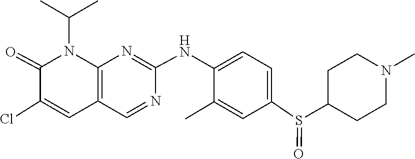 [SMILES: Cc1cc(S(=O)C2CCN(C)CC2)ccc1Nc1ncc2cc(Cl)c(=O)n(C(C)C)c2n1] |
| I-16 | 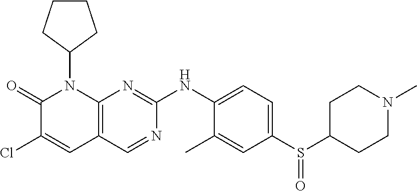 [SMILES: Cc1cc(S(=O)C2CCN(C)CC2)ccc1Nc1ncc2cc(Cl)c(=O)n(C3CCCC3)c2n1] |
| I-17 | 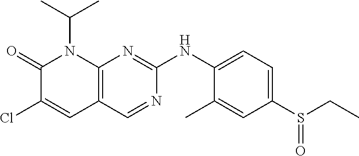 [SMILES: CCS(=O)c1ccc(Nc2ncc3cc(Cl)c(=O)n(C(C)C)c3n2)c(C)c1] |
| I-18 | 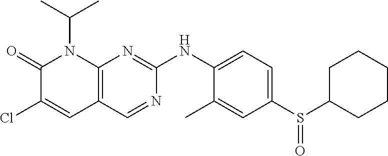 [SMILES: Cc1cc(S(=O)C2CCCCC2)ccc1Nc1ncc2cc(Cl)c(=O)n(C(C)C)c2n1] |
| I-19 | 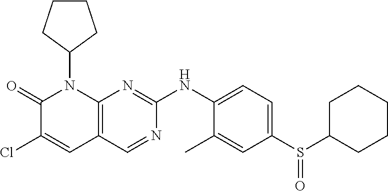 [SMILES: Cc1cc(S(=O)C2CCCCC2)ccc1Nc1ncc2cc(Cl)c(=O)n(C3CCCC3)c2n1] |
| I-20 | 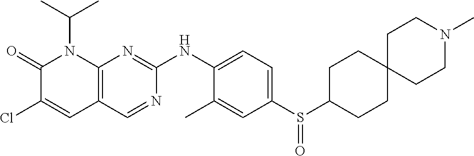 [SMILES: Cc1cc(S(=O)C2CCC3(CC2)CCN(C)CC3)ccc1Nc1ncc2cc(Cl)c(=O)n(C(C)C)c2n1] |
| I-21 | 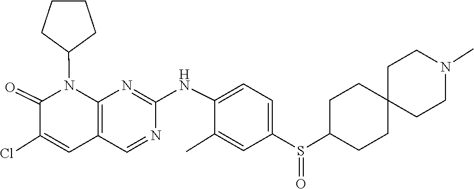 [SMILES: Cc1cc(S(=O)C2CCC3(CC2)CCN(C)CC3)ccc1Nc1ncc2cc(Cl)c(=O)n(C3CCCC3)c2n1] |
| I-22 | 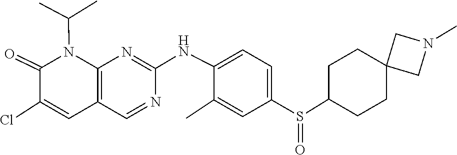 [SMILES: Cc1cc(S(=O)C2CCC3(CC2)CN(C)C3)ccc1Nc1ncc2cc(Cl)c(=O)n(C(C)C)c2n1] |
| I-23 | 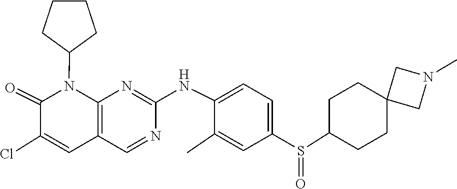 [SMILES: Cc1cc(S(=O)C2CCC3(CC2)CN(C)C3)ccc1Nc1ncc2cc(Cl)c(=O)n(C3CCCC3)c2n1] |
| I-24 | 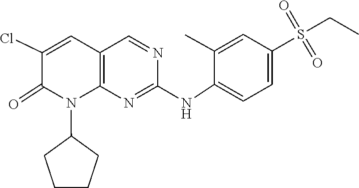 [SMILES: CCS(=O)(=O)c1ccc(Nc2ncc3cc(Cl)c(=O)n(C4CCCC4)c3n2)c(C)c1] |
| I-25 | 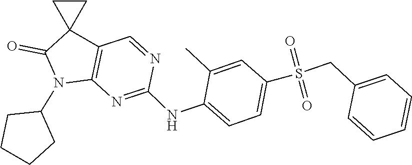 [SMILES: Cc1cc(S(=O)(=O)Cc2ccccc2)ccc1Nc1ncc2c(n1)N(C1CCCC1)C(=O)C21CC1] |
| I-26 | 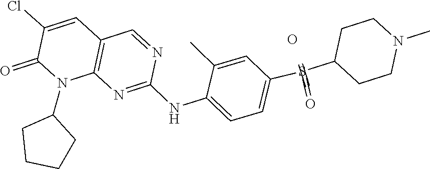 / |
| I-27 | 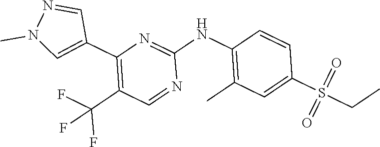 [SMILES: CCS(=O)(=O)c1ccc(Nc2ncc(C(F)(F)F)c(-c3cnn(C)c3)n2)c(C)c1] |
| I-28 | 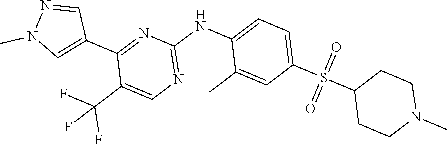 |
| I-29 | 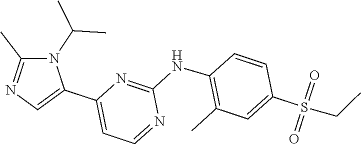 [SMILES: CCS(=O)(=O)c1ccc(Nc2nccc(-c3cnc(C)n3C(C)C)n2)c(C)c1] |
| I-30 | 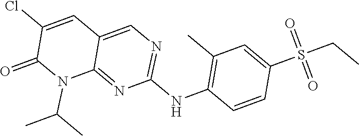 [SMILES: CCS(=O)(=O)c1ccc(Nc2ncc3cc(Cl)c(=O)n(C(C)C)c3n2)c(C)c1] |
| I-31 | 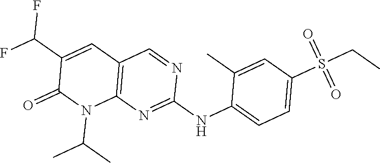 [SMILES: CCS(=O)(=O)c1ccc(Nc2ncc3cc(C(F)F)c(=O)n(C(C)C)c3n2)c(C)c1] |
| I-32 | 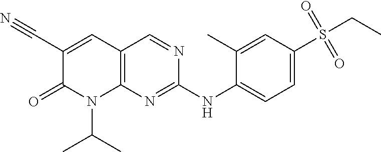 [SMILES: CCS(=O)(=O)c1ccc(Nc2ncc3cc(C#N)c(=O)n(C(C)C)c3n2)c(C)c1] |
| I-33 | 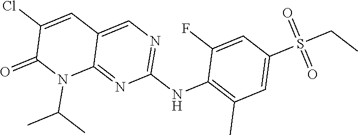 [SMILES: CCS(=O)(=O)c1cc(C)c(Nc2ncc3cc(Cl)c(=O)n(C(C)C)c3n2)c(F)c1] |
| I-34 | 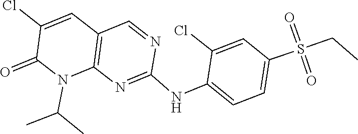 [SMILES: CCS(=O)(=O)c1ccc(Nc2ncc3cc(Cl)c(=O)n(C(C)C)c3n2)c(Cl)c1] |
| I-35 | 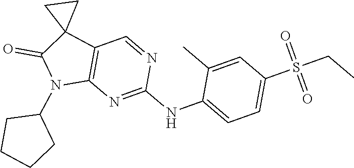 [SMILES: CCS(=O)(=O)c1ccc(Nc2ncc3c(n2)N(C2CCCC2)C(=O)C32CC2)c(C)c1] |
| I-36 | 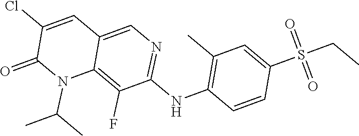 [SMILES: CCS(=O)(=O)c1ccc(Nc2ncc3cc(Cl)c(=O)n(C(C)C)c3c2F)c(C)c1] |
| I-37 | 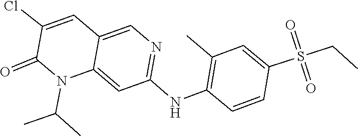 [SMILES: CCS(=O)(=O)c1ccc(Nc2cc3c(cn2)cc(Cl)c(=O)n3C(C)C)c(C)c1] |
| I-38 | 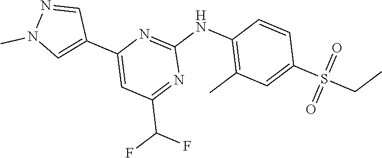 [SMILES: CCS(=O)(=O)c1ccc(Nc2nc(-c3cnn(C)c3)cc(C(F)F)n2)c(C)c1] |
| I-39 | 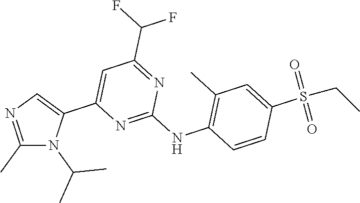 [SMILES: CCS(=O)(=O)c1ccc(Nc2nc(-c3cnc(C)n3C(C)C)cc(C(F)F)n2)c(C)c1] |
| I-40 | 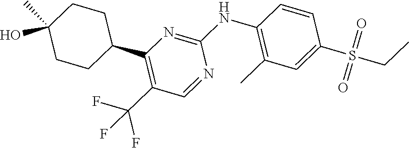 [SMILES: CCS(=O)(=O)c1ccc(Nc2ncc(C(F)(F)F)c([C@H]3CC[C@@](C)(O)CC3)n2)c(C)c1] |
| I-41 | 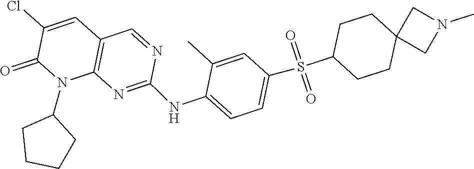 [SMILES: Cc1cc(S(=O)(=O)C2CCC3(CC2)CN(C)C3)ccc1Nc1ncc2cc(Cl)c(=O)n(C3CCCC3)c2n1] |
| I-42 | 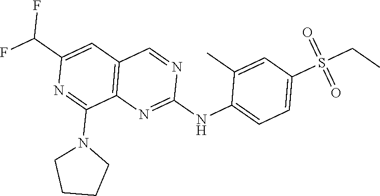 [SMILES: CCS(=O)(=O)c1ccc(Nc2ncc3cc(C(F)F)nc(N4CCCC4)c3n2)c(C)c1] |
| I-43 | 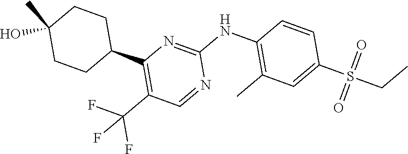 [SMILES: CCS(=O)(=O)c1ccc(Nc2ncc(C(F)(F)F)c([C@H]3CC[C@](C)(O)CC3)n2)c(C)c1] |
| I-44 | 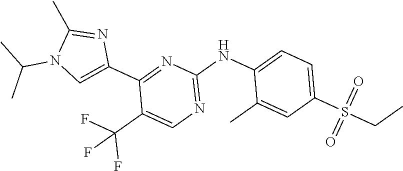 [SMILES: CCS(=O)(=O)c1ccc(Nc2ncc(C(F)(F)F)c(-c3cn(C(C)C)c(C)n3)n2)c(C)c1] |
| I-45 | 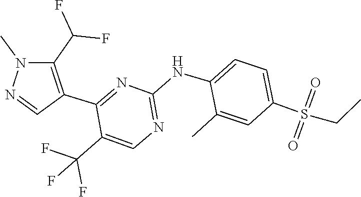 [SMILES: CCS(=O)(=O)c1ccc(Nc2ncc(C(F)(F)F)c(-c3cnn(C)c3C(F)F)n2)c(C)c1] |
| I-46 | 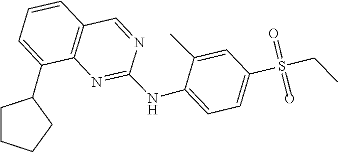 |
| I-47 | 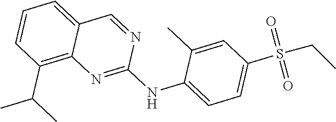 [SMILES: CCS(=O)(=O)c1ccc(Nc2ncc3cccc(C(C)C)c3n2)c(C)c1] |
| I-48 | 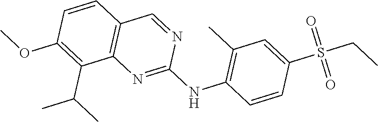 [SMILES: CCS(=O)(=O)c1ccc(Nc2ncc3ccc(OC)c(C(C)C)c3n2)c(C)c1] |
| I-49 | 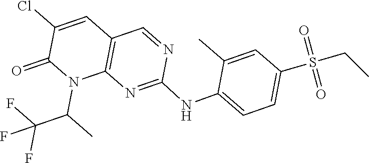 [SMILES: CCS(=O)(=O)c1ccc(Nc2ncc3cc(Cl)c(=O)n(C(C)C(F)(F)F)c3n2)c(C)c1] |
| I-50 | 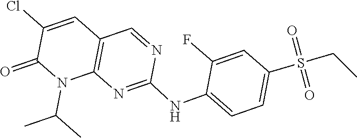 [SMILES: CCS(=O)(=O)c1ccc(Nc2ncc3cc(Cl)c(=O)n(C(C)C)c3n2)c(F)c1] |
| I-51 | 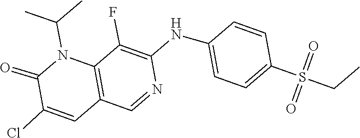 [SMILES: CCS(=O)(=O)c1ccc(Nc2ncc3cc(Cl)c(=O)n(C(C)C)c3c2F)cc1] |
| I-52 | 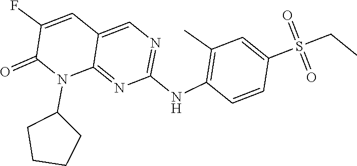 [SMILES: CCS(=O)(=O)c1ccc(Nc2ncc3cc(F)c(=O)n(C4CCCC4)c3n2)c(C)c1] |
| I-53 | 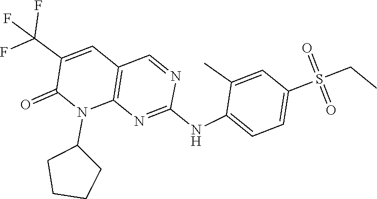 [SMILES: CCS(=O)(=O)c1ccc(Nc2ncc3cc(C(F)(F)F)c(=O)n(C4CCCC4)c3n2)c(C)c1] |
| I-54 | 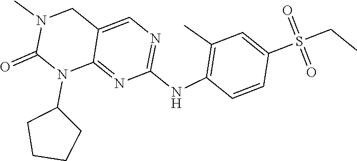 [SMILES: CCS(=O)(=O)c1ccc(Nc2ncc3c(n2)N(C2CCCC2)C(=O)N(C)C3)c(C)c1] |
| I-55 | 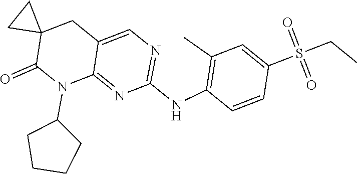 [SMILES: CCS(=O)(=O)c1ccc(Nc2ncc3c(n2)N(C2CCCC2)C(=O)C2(CC2)C3)c(C)c1] |
| I-56 | 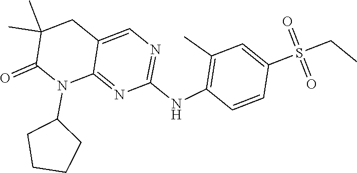 [SMILES: CCS(=O)(=O)c1ccc(Nc2ncc3c(n2)N(C2CCCC2)C(=O)C(C)(C)C3)c(C)c1] |
| I-57 | 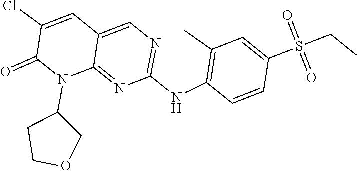 [SMILES: CCS(=O)(=O)c1ccc(Nc2ncc3cc(Cl)c(=O)n(C4CCOC4)c3n2)c(C)c1] |
| I-58 | 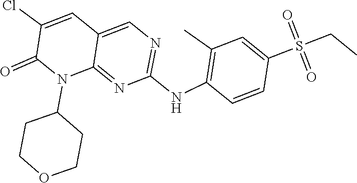 [SMILES: CCS(=O)(=O)c1ccc(Nc2ncc3cc(Cl)c(=O)n(C4CCOCC4)c3n2)c(C)c1] |
| I-59 | 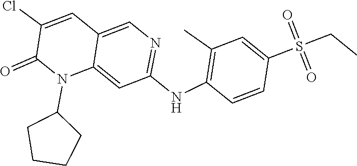 [SMILES: CCS(=O)(=O)c1ccc(Nc2cc3c(cn2)cc(Cl)c(=O)n3C2CCCC2)c(C)c1] |
| I-60 | 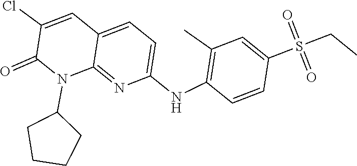 [SMILES: CCS(=O)(=O)c1ccc(Nc2ccc3cc(Cl)c(=O)n(C4CCCC4)c3n2)c(C)c1] |
| I-61 | 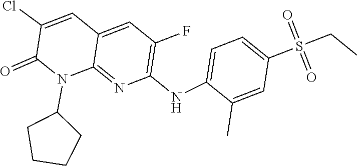 [SMILES: CCS(=O)(=O)c1ccc(Nc2nc3c(cc2F)cc(Cl)c(=O)n3C2CCCC2)c(C)c1] |
| I-62 | 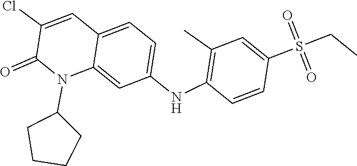 [SMILES: CCS(=O)(=O)c1ccc(Nc2ccc3cc(Cl)c(=O)n(C4CCCC4)c3c2)c(C)c1] |
| I-63 | 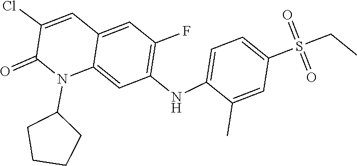 [SMILES: CCS(=O)(=O)c1ccc(Nc2cc3c(cc2F)cc(Cl)c(=O)n3C2CCCC2)c(C)c1] |
| I-64 | 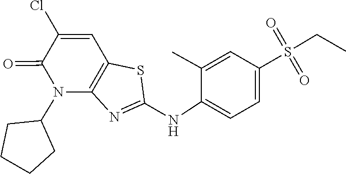 [SMILES: CCS(=O)(=O)c1ccc(Nc2nc3c(cc(Cl)c(=O)n3C3CCCC3)s2)c(C)c1] |
| I-65 | 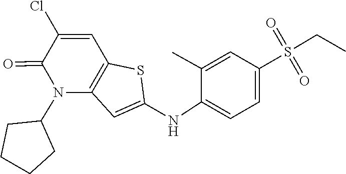 [SMILES: CCS(=O)(=O)c1ccc(Nc2cc3c(cc(Cl)c(=O)n3C3CCCC3)s2)c(C)c1] |
| I-66 | 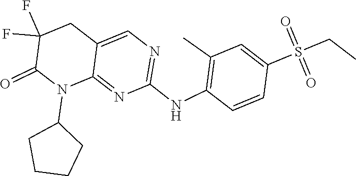 [SMILES: CCS(=O)(=O)c1ccc(Nc2ncc3c(n2)N(C2CCCC2)C(=O)C(F)(F)C3)c(C)c1] |
| I-67 | 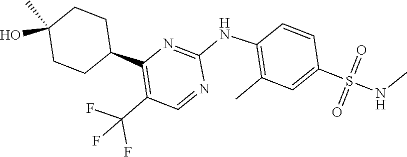 [SMILES: CNS(=O)(=O)c1ccc(Nc2ncc(C(F)(F)F)c([C@H]3CC[C@@](C)(O)CC3)n2)c(C)c1] |
| I-68 | 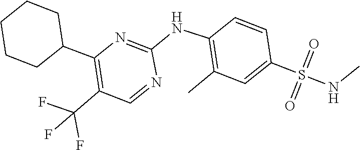 [SMILES: CNS(=O)(=O)c1ccc(Nc2ncc(C(F)(F)F)c(C3CCCCC3)n2)c(C)c1] |
| I-69 | 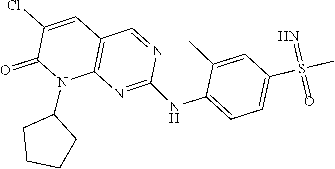 |
| I-70 | 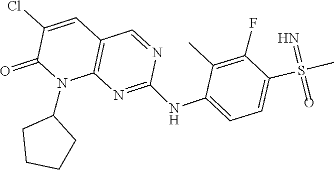 |
| I-71 | 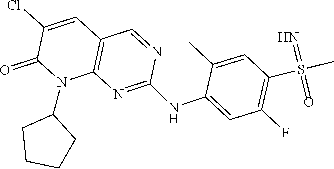 |
| I-72 | 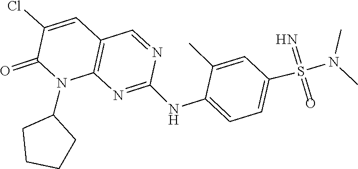 |
| I-73 | 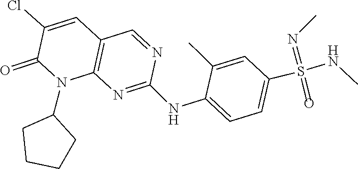 |
| I-74 | 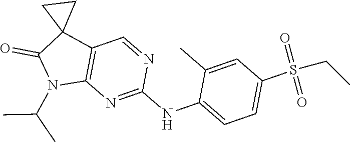 [SMILES: CCS(=O)(=O)c1ccc(Nc2ncc3c(n2)N(C(C)C)C(=O)C32CC2)c(C)c1] |
| I-75 | 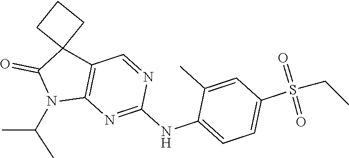 [SMILES: CCS(=O)(=O)c1ccc(Nc2ncc3c(n2)N(C(C)C)C(=O)C32CCC2)c(C)c1] |
| I-76 | 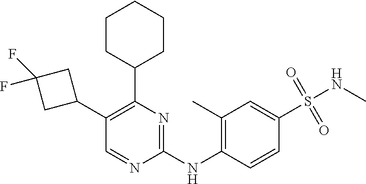 [SMILES: CNS(=O)(=O)c1ccc(Nc2ncc(C3CC(F)(F)C3)c(C3CCCCC3)n2)c(C)c1] |
| I-77 | 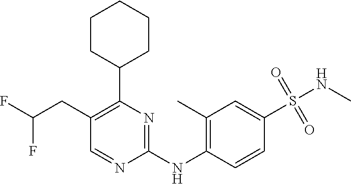 [SMILES: CNS(=O)(=O)c1ccc(Nc2ncc(CC(F)F)c(C3CCCCC3)n2)c(C)c1] |
| I-78 | 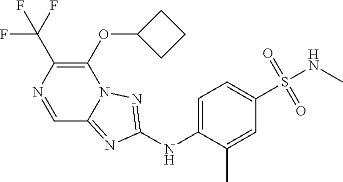 [SMILES: CNS(=O)(=O)c1ccc(Nc2nc3cnc(C(F)(F)F)c(OC4CCC4)n3n2)c(C)c1] |
| I-79 | 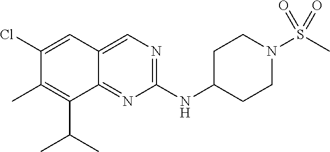 [SMILES: Cc1c(Cl)cc2cnc(NC3CCN(S(C)(=O)=O)CC3)nc2c1C(C)C] |
| I-80 | 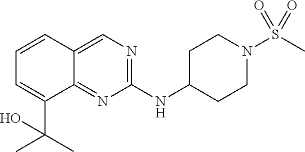 [SMILES: CC(C)(O)c1cccc2cnc(NC3CCN(S(C)(=O)=O)CC3)nc12] |
| I-81 | 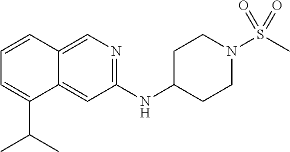 [SMILES: CC(C)c1cccc2cnc(NC3CCN(S(C)(=O)=O)CC3)cc12] |
| I-82 | 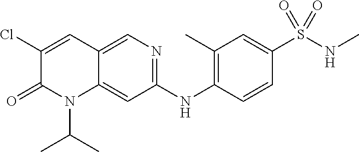 [SMILES: CNS(=O)(=O)c1ccc(Nc2cc3c(cn2)cc(Cl)c(=O)n3C(C)C)c(C)c1] |
| I-83 | 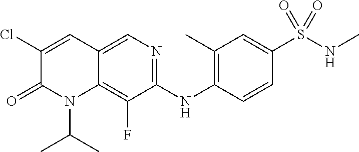 [SMILES: CNS(=O)(=O)c1ccc(Nc2ncc3cc(Cl)c(=O)n(C(C)C)c3c2F)c(C)c1] |
| I-84 | 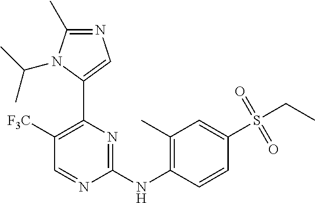 [SMILES: CCS(=O)(=O)c1ccc(Nc2ncc(C(F)(F)F)c(-c3cnc(C)n3C(C)C)n2)c(C)c1] |
| I-85 | 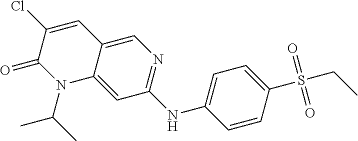 [SMILES: CCS(=O)(=O)c1ccc(Nc2cc3c(cn2)cc(Cl)c(=O)n3C(C)C)cc1] |
| I-86 | 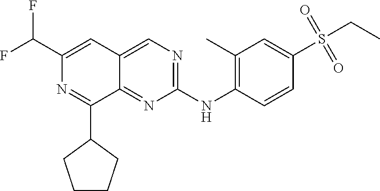 [SMILES: CCS(=O)(=O)c1ccc(Nc2ncc3cc(C(F)F)nc(C4CCCC4)c3n2)c(C)c1] |
| I-87 | 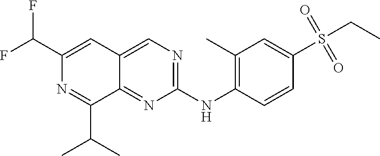 [SMILES: CCS(=O)(=O)c1ccc(Nc2ncc3cc(C(F)F)nc(C(C)C)c3n2)c(C)c1] |
| I-88 | 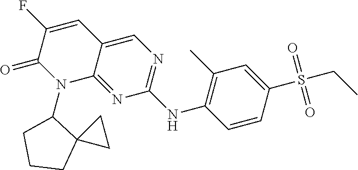 [SMILES: CCS(=O)(=O)c1ccc(Nc2ncc3cc(F)c(=O)n(C4CCCC45CC5)c3n2)c(C)c1] |
| I-89 | 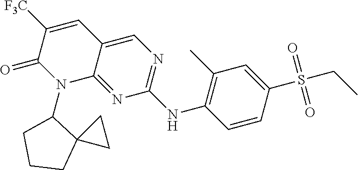 [SMILES: CCS(=O)(=O)c1ccc(Nc2ncc3cc(C(F)(F)F)c(=O)n(C4CCCC45CC5)c3n2)c(C)c1] |
| I-90 | 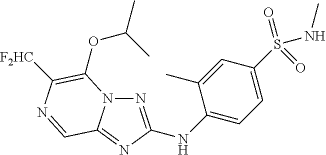 [SMILES: CNS(=O)(=O)c1ccc(Nc2nc3cnc(C(F)F)c(OC(C)C)n3n2)c(C)c1] |
| I-91 | 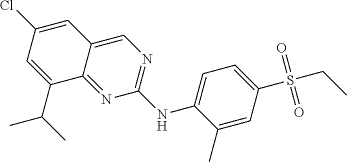 [SMILES: CCS(=O)(=O)c1ccc(Nc2ncc3cc(Cl)cc(C(C)C)c3n2)c(C)c1] |
| I-92 | 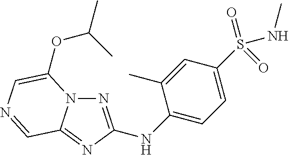 [SMILES: CNS(=O)(=O)c1ccc(Nc2nc3cncc(OC(C)C)n3n2)c(C)c1] |
| I-93 | 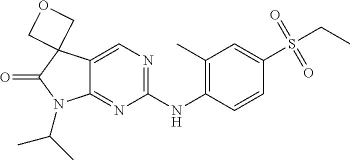 [SMILES: CCS(=O)(=O)c1ccc(Nc2ncc3c(n2)N(C(C)C)C(=O)C32COC2)c(C)c1] |
| I-94 | 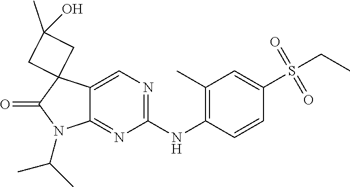 [SMILES: CCS(=O)(=O)c1ccc(Nc2ncc3c(n2)N(C(C)C)C(=O)C32CC(C)(O)C2)c(C)c1] |
| I-95 | 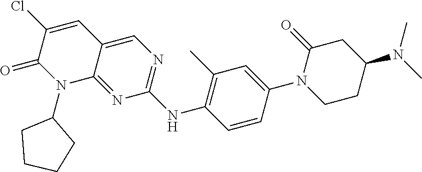 [SMILES: Cc1cc(N2CC[C@H](N(C)C)CC2=O)ccc1Nc1ncc2cc(Cl)c(=O)n(C3CCCC3)c2n1] |
| I-96 | 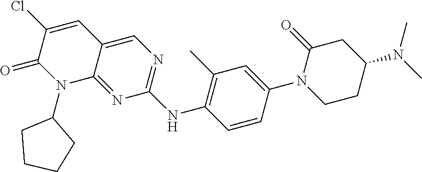 [SMILES: Cc1cc(N2CC[C@@H](N(C)C)CC2=O)ccc1Nc1ncc2cc(Cl)c(=O)n(C3CCCC3)c2n1] |
| I-97 | 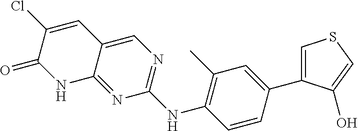 [SMILES: Cc1cc(-c2cscc2O)ccc1Nc1ncc2cc(Cl)c(=O)[nH]c2n1] |
| I-98 | 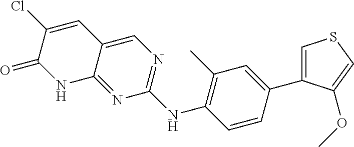 [SMILES: COc1cscc1-c1ccc(Nc2ncc3cc(Cl)c(=O)[nH]c3n2)c(C)c1] |
| I-99 | 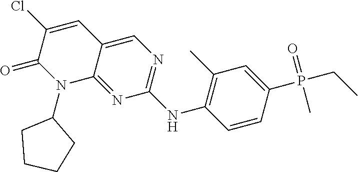 [SMILES: CCP(C)(=O)c1ccc(Nc2ncc3cc(Cl)c(=O)n(C4CCCC4)c3n2)c(C)c1] |
| I- 100 | 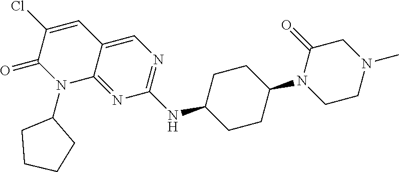 [SMILES: CN1CCN([C@H]2CC[C@@H](Nc3ncc4cc(Cl)c(=O)n(C5CCCC5)c4n3)CC2)C(=O)C1] |
| I- 101 | 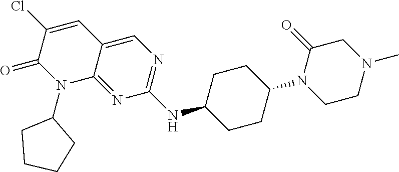 [SMILES: CN1CCN([C@H]2CC[C@H](Nc3ncc4cc(Cl)c(=O)n(C5CCCC5)c4n3)CC2)C(=O)C1] |
| I- 102 | 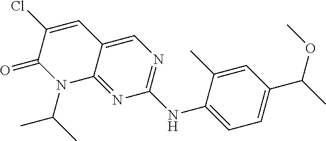 [SMILES: COC(C)c1ccc(Nc2ncc3cc(Cl)c(=O)n(C(C)C)c3n2)c(C)c1] |
| I- 103 | 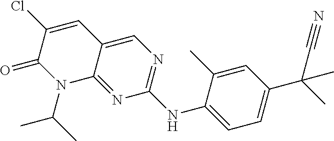 [SMILES: Cc1cc(C(C)(C)C#N)ccc1Nc1ncc2cc(Cl)c(=O)n(C(C)C)c2n1] |
| I- 104 | 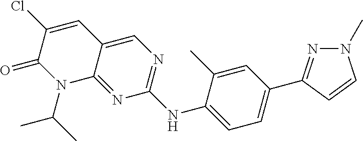 [SMILES: Cc1cc(-c2ccn(C)n2)ccc1Nc1ncc2cc(Cl)c(=O)n(C(C)C)c2n1] |
| I- 105 | 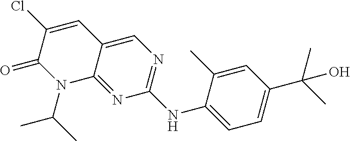 [SMILES: Cc1cc(C(C)(C)O)ccc1Nc1ncc2cc(Cl)c(=O)n(C(C)C)c2n1] |
| I- 106 | 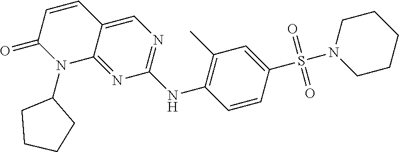 [SMILES: Cc1cc(S(=O)(=O)N2CCCCC2)ccc1Nc1ncc2ccc(=O)n(C3CCCC3)c2n1] |
| I- 107 | 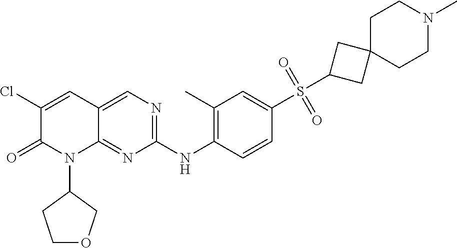 [SMILES: Cc1cc(S(=O)(=O)C2CC3(CCN(C)CC3)C2)ccc1Nc1ncc2cc(Cl)c(=O)n(C3CCOC3)c2n1] |
| I- 108 | 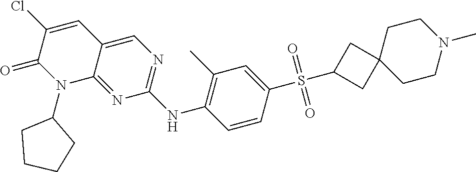 [SMILES: Cc1cc(S(=O)(=O)C2CC3(CCN(C)CC3)C2)ccc1Nc1ncc2cc(Cl)c(=O)n(C3CCCC3)c2n1] |
| I- 109 | 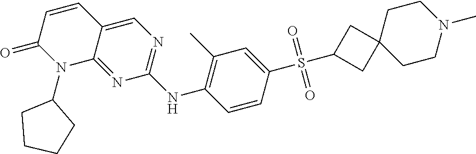 [SMILES: Cc1cc(S(=O)(=O)C2CC3(CCN(C)CC3)C2)ccc1Nc1ncc2ccc(=O)n(C3CCCC3)c2n1] |
| I- 110 | 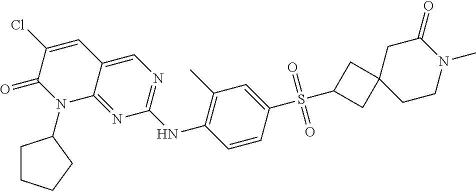 [SMILES: Cc1cc(S(=O)(=O)C2CC3(CCN(C)C(=O)C3)C2)ccc1Nc1ncc2cc(Cl)c(=O)n(C3CCCC3)c2n1] |
| I- 111 | 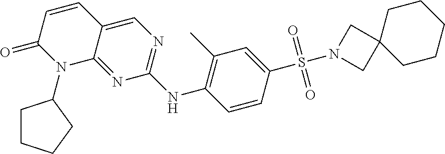 [SMILES: Cc1cc(S(=O)(=O)N2CC3(CCCCC3)C2)ccc1Nc1ncc2ccc(=O)n(C3CCCC3)c2n1] |
| I- 112 | 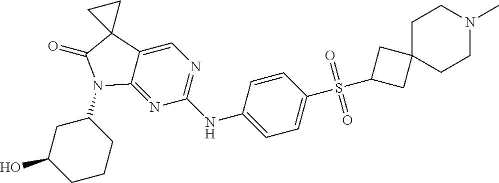 |
| I- 113 | 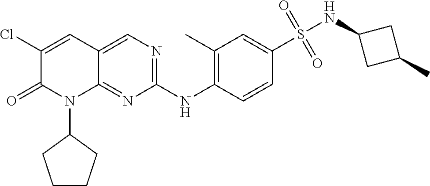 [SMILES: Cc1cc(S(=O)(=O)N[C@H]2C[C@@H](C)C2)ccc1Nc1ncc2cc(Cl)c(=O)n(C3CCCC3)c2n1] |
| I- 114 | 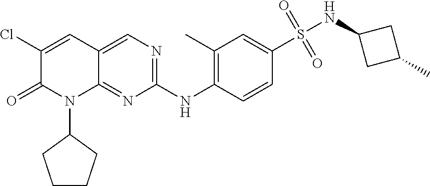 [SMILES: Cc1cc(S(=O)(=O)N[C@H]2C[C@H](C)C2)ccc1Nc1ncc2cc(Cl)c(=O)n(C3CCCC3)c2n1] |
| I- 115 | 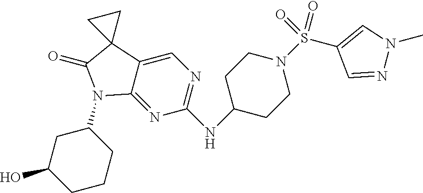 |
| I- 116 | 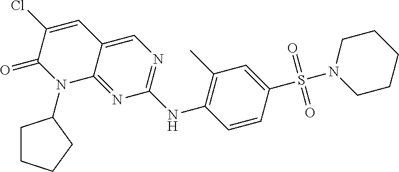 [SMILES: Cc1cc(S(=O)(=O)N2CCCCC2)ccc1Nc1ncc2cc(Cl)c(=O)n(C3CCCC3)c2n1] |
| I- 117 | 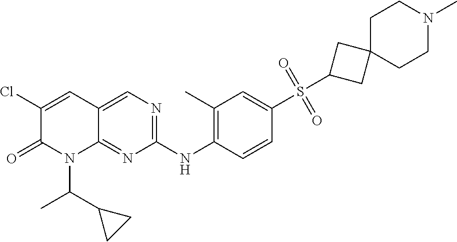 [SMILES: Cc1cc(S(=O)(=O)C2CC3(CCN(C)CC3)C2)ccc1Nc1ncc2cc(Cl)c(=O)n(C(C)C3CC3)c2n1] |
| I- 118 | 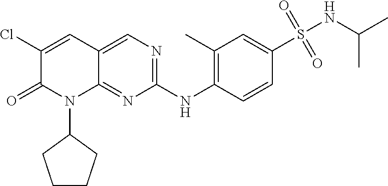 [SMILES: Cc1cc(S(=O)(=O)NC(C)C)ccc1Nc1ncc2cc(Cl)c(=O)n(C3CCCC3)c2n1] |
| I- 119 | 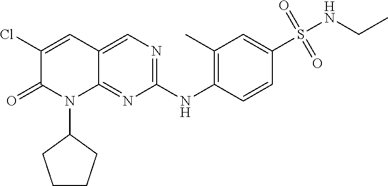 [SMILES: CCNS(=O)(=O)c1ccc(Nc2ncc3cc(Cl)c(=O)n(C4CCCC4)c3n2)c(C)c1] |
| I- 120 | 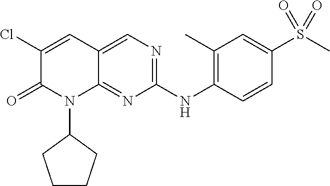 [SMILES: Cc1cc(S(C)(=O)=O)ccc1Nc1ncc2cc(Cl)c(=O)n(C3CCCC3)c2n1] |
| I- 121 | 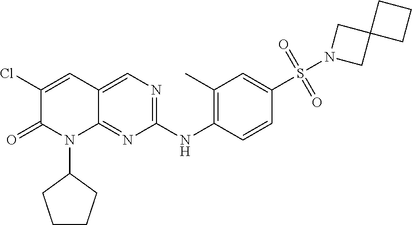 [SMILES: Cc1cc(S(=O)(=O)N2CC3(CCC3)C2)ccc1Nc1ncc2cc(Cl)c(=O)n(C3CCCC3)c2n1] |
| I- 122 | 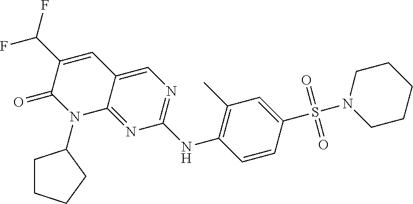 [SMILES: Cc1cc(S(=O)(=O)N2CCCCC2)ccc1Nc1ncc2cc(C(F)F)c(=O)n(C3CCCC3)c2n1] |
| I- 123 | 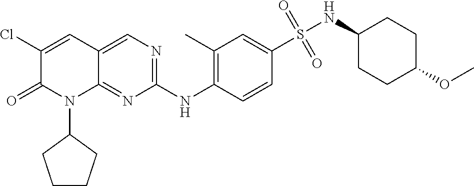 [SMILES: CO[C@H]1CC[C@H](NS(=O)(=O)c2ccc(Nc3ncc4cc(Cl)c(=O)n(C5CCCC5)c4n3)c(C)c2)CC1] |
| I- 124 | 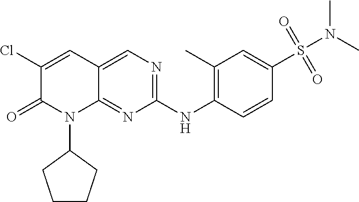 [SMILES: Cc1cc(S(=O)(=O)N(C)C)ccc1Nc1ncc2cc(Cl)c(=O)n(C3CCCC3)c2n1] |
| I- 125 | 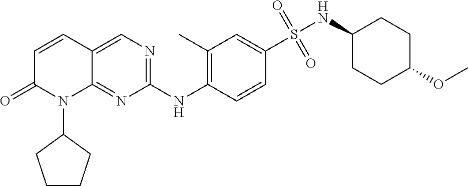 [SMILES: CO[C@H]1CC[C@H](NS(=O)(=O)c2ccc(Nc3ncc4ccc(=O)n(C5CCCC5)c4n3)c(C)c2)CC1] |
| I- 126 | 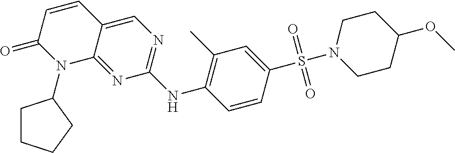 [SMILES: COC1CCN(S(=O)(=O)c2ccc(Nc3ncc4ccc(=O)n(C5CCCC5)c4n3)c(C)c2)CC1] |
| I- 127 | 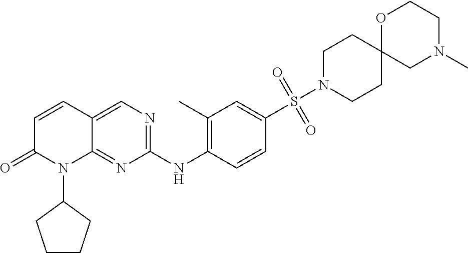 [SMILES: Cc1cc(S(=O)(=O)N2CCC3(CC2)CN(C)CCO3)ccc1Nc1ncc2ccc(=O)n(C3CCCC3)c2n1] |
| I- 128 | 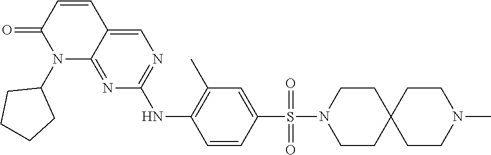 [SMILES: Cc1cc(S(=O)(=O)N2CCC3(CCN(C)CC3)CC2)ccc1Nc1ncc2ccc(=O)n(C3CCCC3)c2n1] |
| I- 129 | 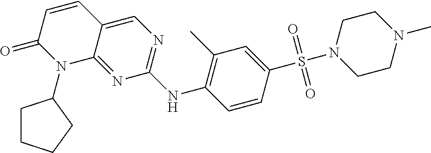 [SMILES: Cc1cc(S(=O)(=O)N2CCN(C)CC2)ccc1Nc1ncc2ccc(=O)n(C3CCCC3)c2n1] |
| I- 130 | 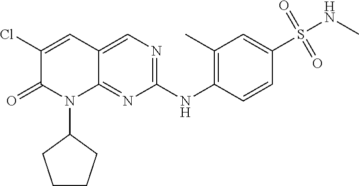 [SMILES: CNS(=O)(=O)c1ccc(Nc2ncc3cc(Cl)c(=O)n(C4CCCC4)c3n2)c(C)c1] |
| I- 131 | 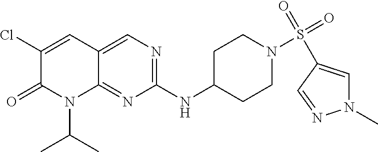 [SMILES: CC(C)n1c(=O)c(Cl)cc2cnc(NC3CCN(S(=O)(=O)c4cnn(C)c4)CC3)nc21] |
| I- 132 | 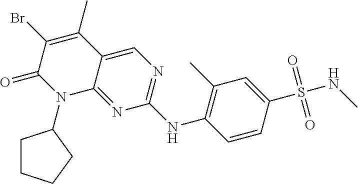 [SMILES: CNS(=O)(=O)c1ccc(Nc2ncc3c(C)c(Br)c(=O)n(C4CCCC4)c3n2)c(C)c1] |
| I- 133 | 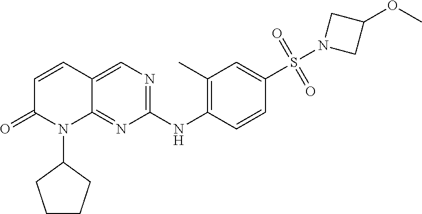 [SMILES: COC1CN(S(=O)(=O)c2ccc(Nc3ncc4ccc(=O)n(C5CCCC5)c4n3)c(C)c2)C1] |
| I- 134 | 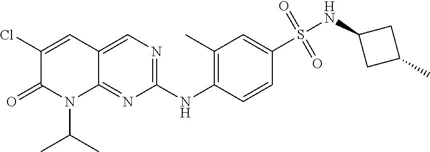 [SMILES: Cc1cc(S(=O)(=O)N[C@H]2C[C@H](C)C2)ccc1Nc1ncc2cc(Cl)c(=O)n(C(C)C)c2n1] |
| I- 135 | 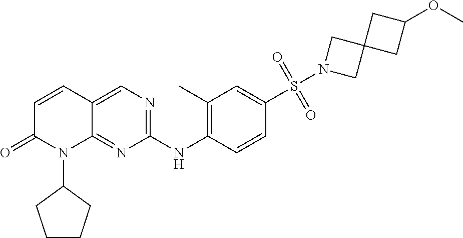 [SMILES: COC1CC2(C1)CN(S(=O)(=O)c1ccc(Nc3ncc4ccc(=O)n(C5CCCC5)c4n3)c(C)c1)C2] |
| I- 136 | 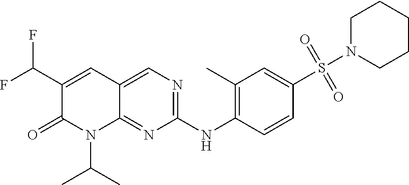 [SMILES: Cc1cc(S(=O)(=O)N2CCCCC2)ccc1Nc1ncc2cc(C(F)F)c(=O)n(C(C)C)c2n1] |
| I- 137 | 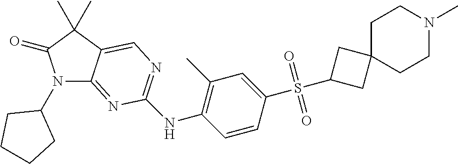 [SMILES: Cc1cc(S(=O)(=O)C2CC3(CCN(C)CC3)C2)ccc1Nc1ncc2c(n1)N(C1CCCC1)C(=O)C2(C)C] |
| I- 138 | 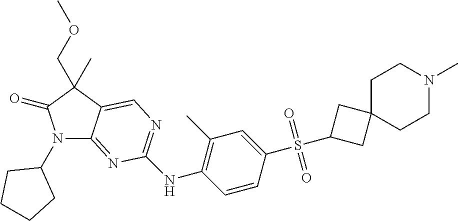 [SMILES: COCC1(C)C(=O)N(C2CCCC2)c2nc(Nc3ccc(S(=O)(=O)C4CC5(CCN(C)CC5)C4)cc3C)ncc21] |
| I- 139 | 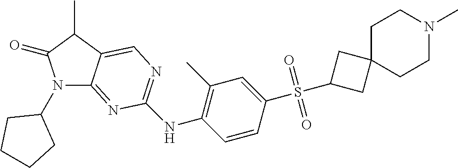 [SMILES: Cc1cc(S(=O)(=O)C2CC3(CCN(C)CC3)C2)ccc1Nc1ncc2c(n1)N(C1CCCC1)C(=O)C2C] |
| I- 140 | 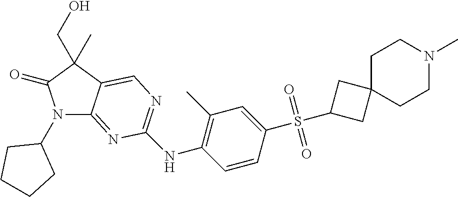 [SMILES: Cc1cc(S(=O)(=O)C2CC3(CCN(C)CC3)C2)ccc1Nc1ncc2c(n1)N(C1CCCC1)C(=O)C2(C)CO] |
| I- 141 | 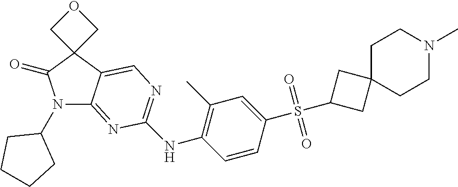 [SMILES: Cc1cc(S(=O)(=O)C2CC3(CCN(C)CC3)C2)ccc1Nc1ncc2c(n1)N(C1CCCC1)C(=O)C21COC1] |
| I- 142 | 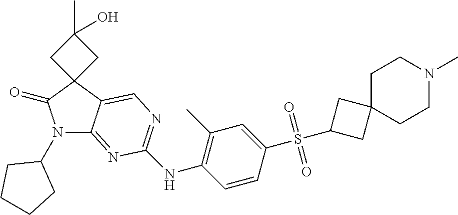 [SMILES: Cc1cc(S(=O)(=O)C2CC3(CCN(C)CC3)C2)ccc1Nc1ncc2c(n1)N(C1CCCC1)C(=O)C21CC(C)(O)C1] |
| I- 143 | 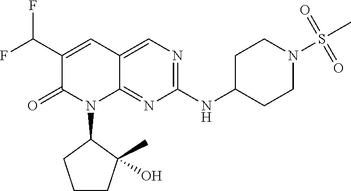 [SMILES: C[C@@]1(O)CCC[C@H]1n1c(=O)c(C(F)F)cc2cnc(NC3CCN(S(C)(=O)=O)CC3)nc21] |
| I- 144 | 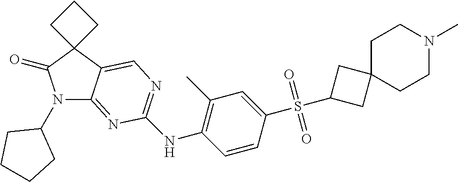 [SMILES: Cc1cc(S(=O)(=O)C2CC3(CCN(C)CC3)C2)ccc1Nc1ncc2c(n1)N(C1CCCC1)C(=O)C21CCC1] |
| I- 145 | 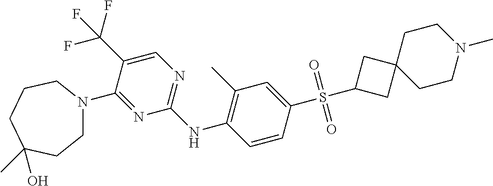 |
| I- 146 | 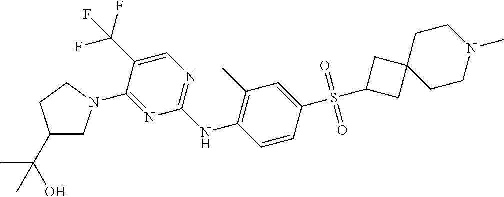 [SMILES: Cc1cc(S(=O)(=O)C2CC3(CCN(C)CC3)C2)ccc1Nc1ncc(C(F)(F)F)c(N2CCC(C(C)(C)O)C2)n1] |
| I- 147 | 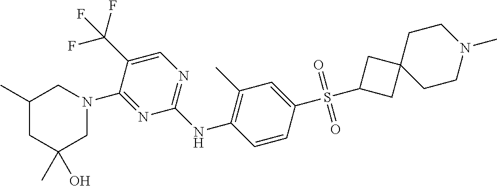 |
| I- 148 | 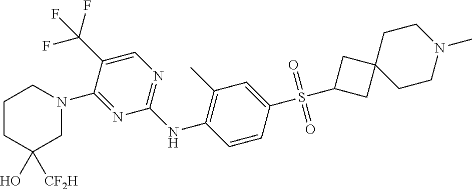 [SMILES: Cc1cc(S(=O)(=O)C2CC3(CCN(C)CC3)C2)ccc1Nc1ncc(C(F)(F)F)c(N2CCCC(O)(C(F)F)C2)n1] |
| I- 149 | 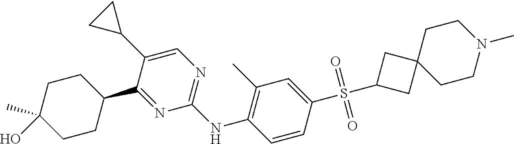 |
| I- 150 | 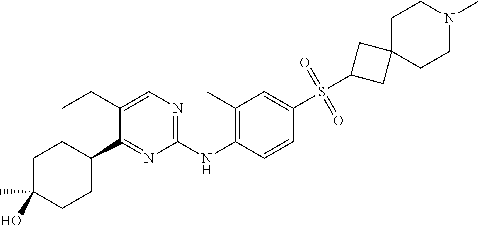 |
| I- 151 | 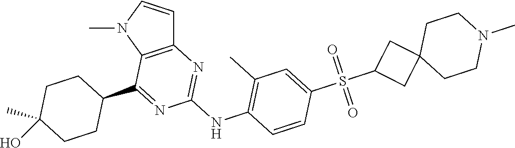 |
| I- 152 | 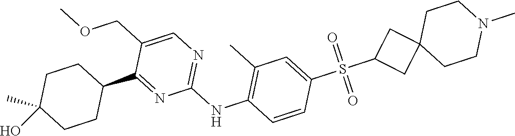 |
| I- 153 | 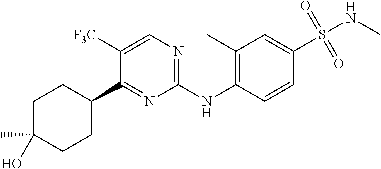 |
| I- 154 | 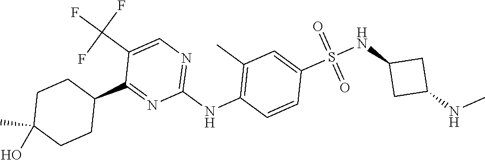 |
| I- 155 | 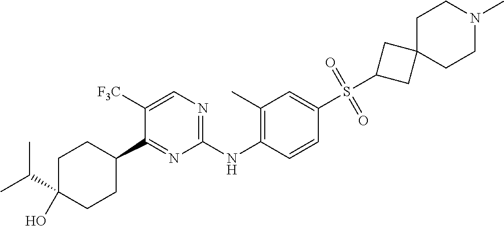 [SMILES: Cc1cc(S(=O)(=O)C2CC3(CCN(C)CC3)C2)ccc1Nc1ncc(C(F)(F)F)c([C@H]2CC[C@](O)(C(C)C)CC2)n1] |
| I- 156 | 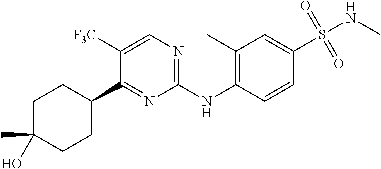 |
| I- 157 | 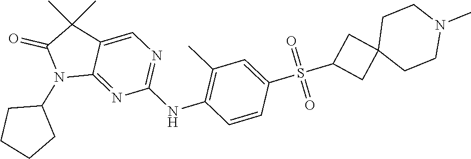 [SMILES: Cc1cc(S(=O)(=O)C2CC3(CCN(C)CC3)C2)ccc1Nc1ncc2c(n1)N(C1CCCC1)C(=O)C2(C)C] |
| I- 158 | 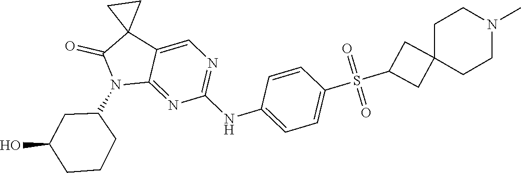 |
| I- 159 | 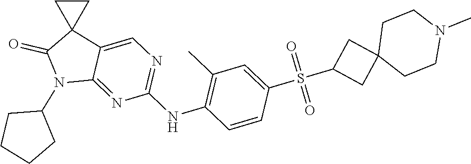 [SMILES: Cc1cc(S(=O)(=O)C2CC3(CCN(C)CC3)C2)ccc1Nc1ncc2c(n1)N(C1CCCC1)C(=O)C21CC1] |
| I- 160 | 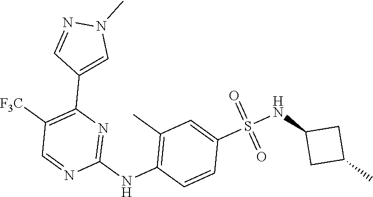 [SMILES: Cc1cc(S(=O)(=O)N[C@H]2C[C@H](C)C2)ccc1Nc1ncc(C(F)(F)F)c(-c2cnn(C)c2)n1] |
| I- 161 | 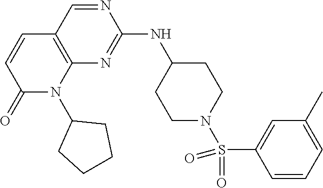 [SMILES: Cc1cccc(S(=O)(=O)N2CCC(Nc3ncc4ccc(=O)n(C5CCCC5)c4n3)CC2)c1] |
| I- 162 | 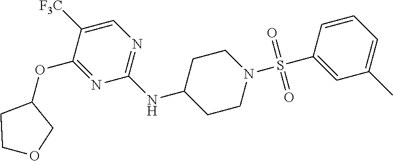 |
| I- 163 | 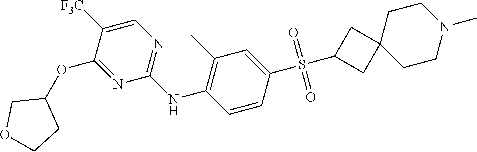 [SMILES: Cc1cc(S(=O)(=O)C2CC3(CCN(C)CC3)C2)ccc1Nc1ncc(C(F)(F)F)c(OC2CCOC2)n1] |
| I- 164 | 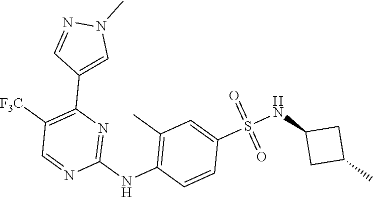 [SMILES: Cc1cc(S(=O)(=O)N[C@H]2C[C@H](C)C2)ccc1Nc1ncc(C(F)(F)F)c(-c2cnn(C)c2)n1] |
| I- 165 | 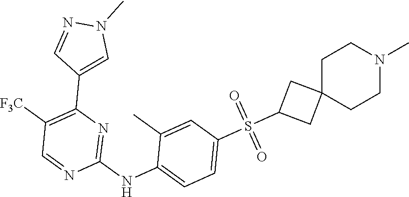 [SMILES: Cc1cc(S(=O)(=O)C2CC3(CCN(C)CC3)C2)ccc1Nc1ncc(C(F)(F)F)c(-c2cnn(C)c2)n1] |
| I- 166 | 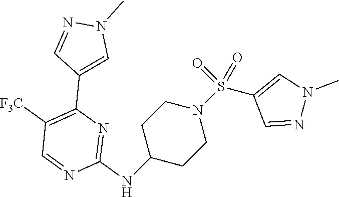 [SMILES: Cn1cc(-c2nc(NC3CCN(S(=O)(=O)c4cnn(C)c4)CC3)ncc2C(F)(F)F)cn1] |
| I- 167 | 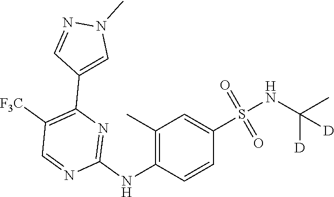 [SMILES: [2H]C([2H])(C)NS(=O)(=O)c1ccc(Nc2ncc(C(F)(F)F)c(-c3cnn(C)c3)n2)c(C)c1] |
| I- 168 | 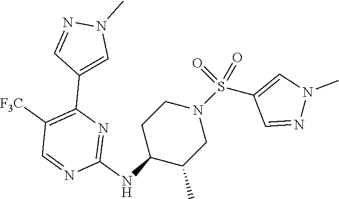 [SMILES: C[C@H]1CN(S(=O)(=O)c2cnn(C)c2)CC[C@@H]1Nc1ncc(C(F)(F)F)c(-c2cnn(C)c2)n1] |
| I- 169 | 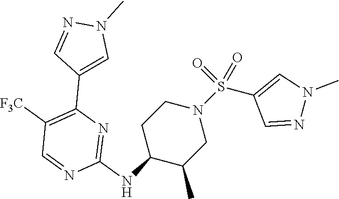 [SMILES: C[C@@H]1CN(S(=O)(=O)c2cnn(C)c2)CC[C@@H]1Nc1ncc(C(F)(F)F)c(-c2cnn(C)c2)n1] |
| I- 170 | 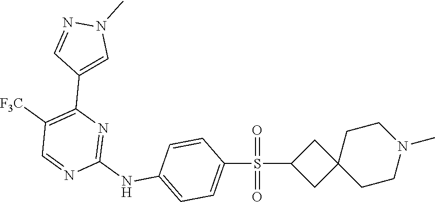 [SMILES: CN1CCC2(CC1)CC(S(=O)(=O)c1ccc(Nc3ncc(C(F)(F)F)c(-c4cnn(C)c4)n3)cc1)C2] |
| I- 171 | 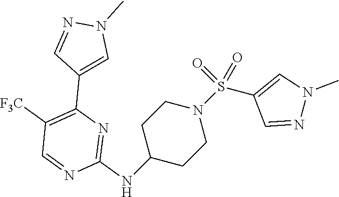 [SMILES: Cn1cc(-c2nc(NC3CCN(S(=O)(=O)c4cnn(C)c4)CC3)ncc2C(F)(F)F)cn1] |
| I- 172 | 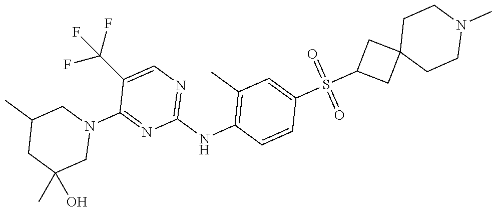 |
| I- 173 | 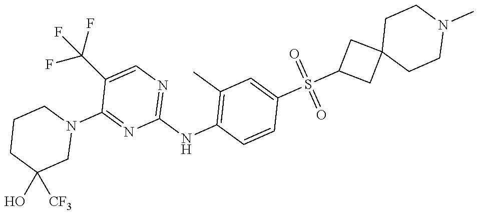 [SMILES: Cc1cc(S(=O)(=O)C2CC3(CCN(C)CC3)C2)ccc1Nc1ncc(C(F)(F)F)c(N2CCCC(O)(C(F)(F)F)C2)n1] |
| I- 174 | 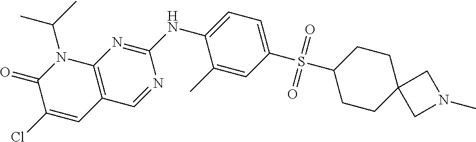 [SMILES: Cc1cc(S(=O)(=O)C2CCC3(CC2)CN(C)C3)ccc1Nc1ncc2cc(Cl)c(=O)n(C(C)C)c2n1] |
| I- 175 | 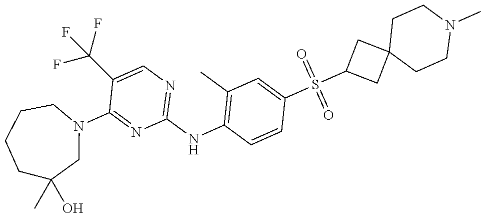 [SMILES: Cc1cc(S(=O)(=O)C2CC3(CCN(C)CC3)C2)ccc1Nc1ncc(C(F)(F)F)c(N2CCCCC(C)(O)C2)n1] |
| I- 176 | 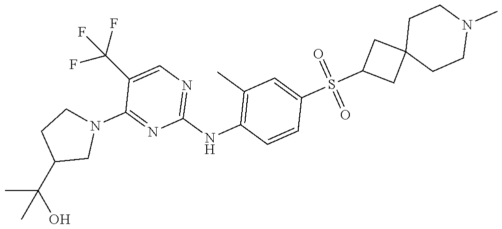 [SMILES: Cc1cc(S(=O)(=O)C2CC3(CCN(C)CC3)C2)ccc1Nc1ncc(C(F)(F)F)c(N2CCC(C(C)(C)O)C2)n1] |
| I- 177 | 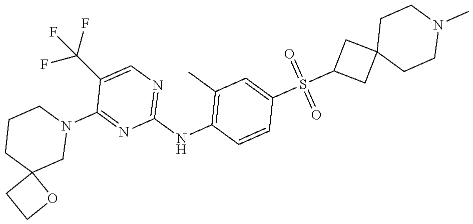 [SMILES: Cc1cc(S(=O)(=O)C2CC3(CCN(C)CC3)C2)ccc1Nc1ncc(C(F)(F)F)c(N2CCCC3(CCO3)C2)n1] |
| I- 178 | 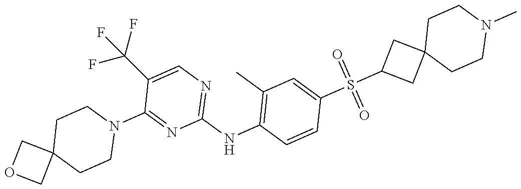 [SMILES: Cc1cc(S(=O)(=O)C2CC3(CCN(C)CC3)C2)ccc1Nc1ncc(C(F)(F)F)c(N2CCC3(CC2)COC3)n1] |
| I- 179 | 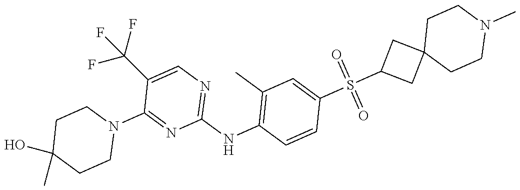 [SMILES: Cc1cc(S(=O)(=O)C2CC3(CCN(C)CC3)C2)ccc1Nc1ncc(C(F)(F)F)c(N2CCC(C)(O)CC2)n1] |
| I- 180 | 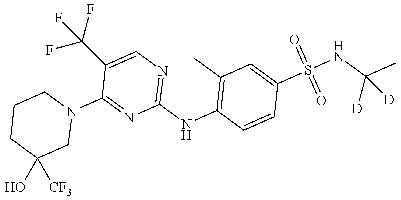 [SMILES: [2H]C([2H])(C)NS(=O)(=O)c1ccc(Nc2ncc(C(F)(F)F)c(N3CCCC(O)(C(F)(F)F)C3)n2)c(C)c1] |
| I- 181 | 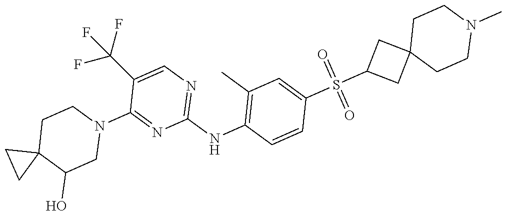 [SMILES: Cc1cc(S(=O)(=O)C2CC3(CCN(C)CC3)C2)ccc1Nc1ncc(C(F)(F)F)c(N2CCC3(CC3)C(O)C2)n1] |
| I- 182 | 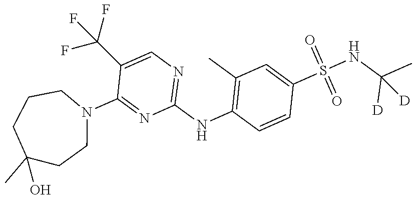 [SMILES: [2H]C([2H])(C)NS(=O)(=O)c1ccc(Nc2ncc(C(F)(F)F)c(N3CCCC(C)(O)CC3)n2)c(C)c1] |
| I- 183 | 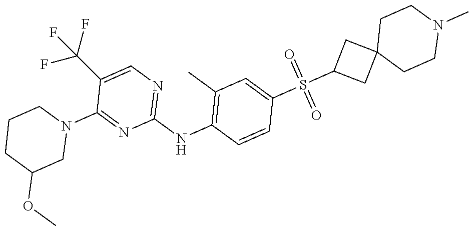 [SMILES: COC1CCCN(c2nc(Nc3ccc(S(=O)(=O)C4CC5(CCN(C)CC5)C4)cc3C)ncc2C(F)(F)F)C1] |
| I- 184 | 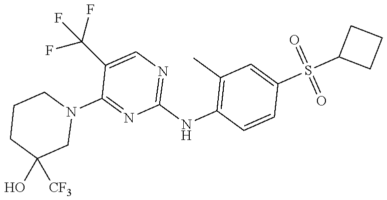 [SMILES: Cc1cc(S(=O)(=O)C2CCC2)ccc1Nc1ncc(C(F)(F)F)c(N2CCCC(O)(C(F)(F)F)C2)n1] |
| I- 185 | 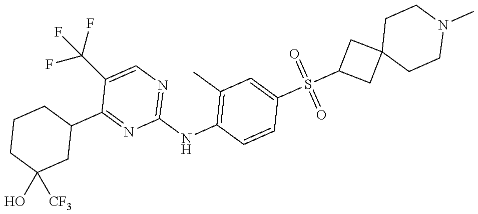 [SMILES: Cc1cc(S(=O)(=O)C2CC3(CCN(C)CC3)C2)ccc1Nc1ncc(C(F)(F)F)c(C2CCCC(O)(C(F)(F)F)C2)n1] |
| I- 186 | 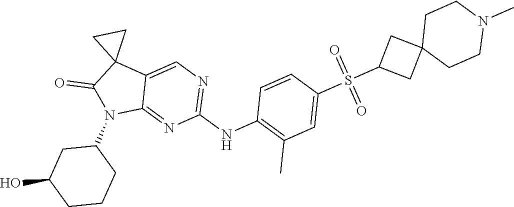 |
| I- 187 | 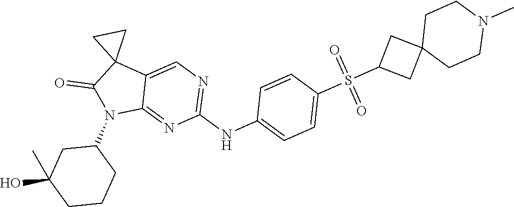 |
| I- 188 | 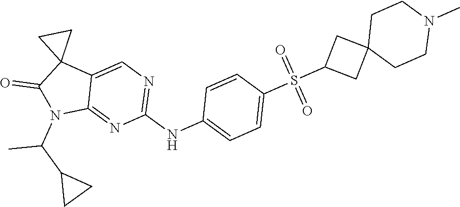 [SMILES: CC(C1CC1)N1C(=O)C2(CC2)c2cnc(Nc3ccc(S(=O)(=O)C4CC5(CCN(C)CC5)C4)cc3)nc21] |
| I- 189 | 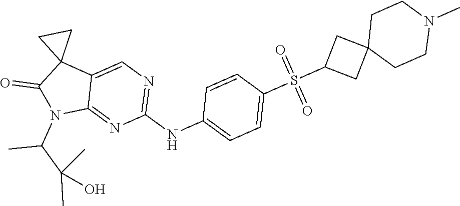 [SMILES: CC(N1C(=O)C2(CC2)c2cnc(Nc3ccc(S(=O)(=O)C4CC5(CCN(C)CC5)C4)cc3)nc21)C(C)(C)O] |
| I- 190 | 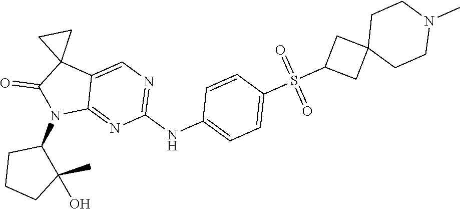 [SMILES: CN1CCC2(CC1)CC(S(=O)(=O)c1ccc(Nc3ncc4c(n3)N([C@@H]3CCC[C@@]3(C)O)C(=O)C43CC3)cc1)C2] |
| I- 191 | 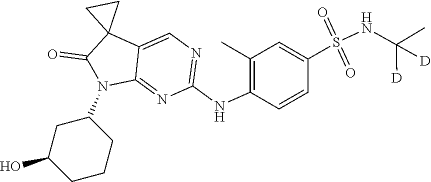 |
| I- 192 | 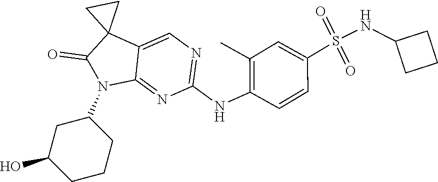 |
| I- 193 | 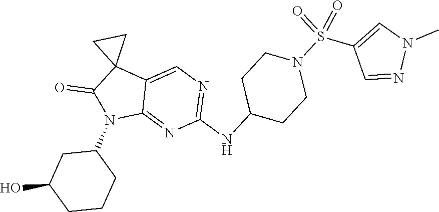 |
| I- 194 | 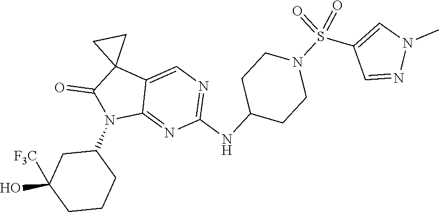 |
| I- 195 | 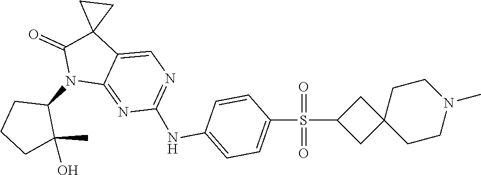 [SMILES: CN1CCC2(CC1)CC(S(=O)(=O)c1ccc(Nc3ncc4c(n3)N([C@@H]3CCC[C@@]3(C)O)C(=O)C43CC3)cc1)C2] |
| I- 196 | 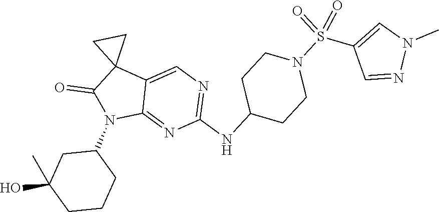 |
| I- 197 | 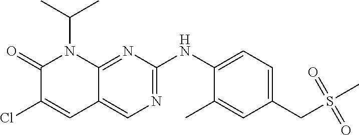 [SMILES: Cc1cc(CS(C)(=O)=O)ccc1Nc1ncc2cc(Cl)c(=O)n(C(C)C)c2n1] |
| I- 198 | 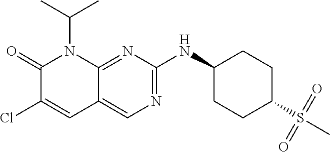 [SMILES: CC(C)n1c(=O)c(Cl)cc2cnc(N[C@H]3CC[C@H](S(C)(=O)=O)CC3)nc21] |
| I- 199 | 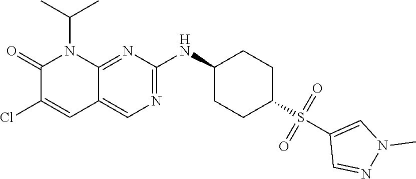 [SMILES: CC(C)n1c(=O)c(Cl)cc2cnc(N[C@H]3CC[C@H](S(=O)(=O)c4cnn(C)c4)CC3)nc21] |
| I- 200 | 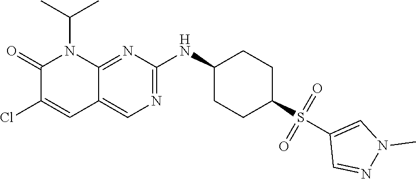 [SMILES: CC(C)n1c(=O)c(Cl)cc2cnc(N[C@H]3CC[C@@H](S(=O)(=O)c4cnn(C)c4)CC3)nc21] |
| I- 204 | 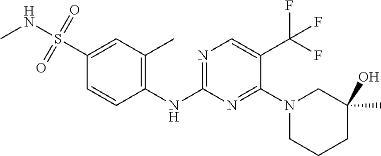 |
| I- 205 | 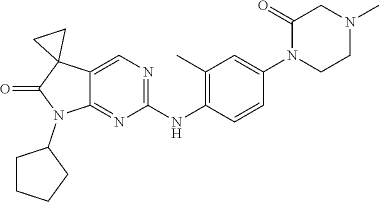 [SMILES: Cc1cc(N2CCN(C)CC2=O)ccc1Nc1ncc2c(n1)N(C1CCCC1)C(=O)C21CC1] |
| I- 206 | 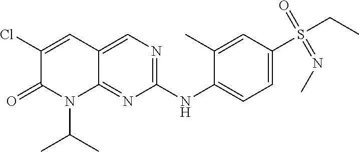 [SMILES: CCS(=O)(=NC)c1ccc(Nc2ncc3cc(Cl)c(=O)n(C(C)C)c3n2)c(C)c1] |
| I- 207 | 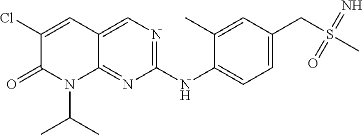 [SMILES: Cc1cc(CS(C)(=N)=O)ccc1Nc1ncc2cc(Cl)c(=O)n(C(C)C)c2n1] |
| I- 208 | 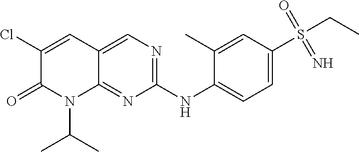 [SMILES: CCS(=N)(=O)c1ccc(Nc2ncc3cc(Cl)c(=O)n(C(C)C)c3n2)c(C)c1] |
| I- 209 | 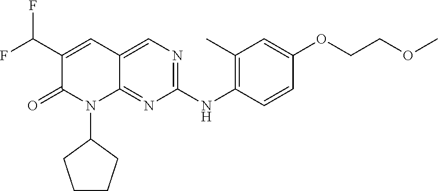 [SMILES: COCCOc1ccc(Nc2ncc3cc(C(F)F)c(=O)n(C4CCCC4)c3n2)c(C)c1] |
| I- 210 | 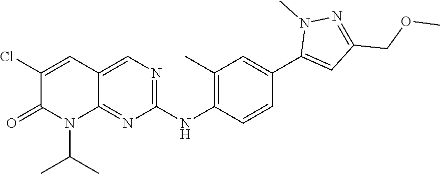 [SMILES: COCc1cc(-c2ccc(Nc3ncc4cc(Cl)c(=O)n(C(C)C)c4n3)c(C)c2)n(C)n1] |
| I- 211 | 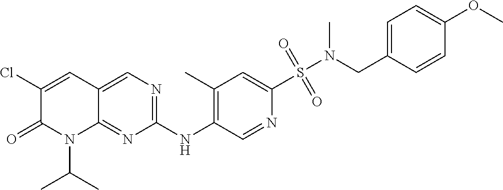 [SMILES: COc1ccc(CN(C)S(=O)(=O)c2cc(C)c(Nc3ncc4cc(Cl)c(=O)n(C(C)C)c4n3)cn2)cc1] |
| I- 212 | 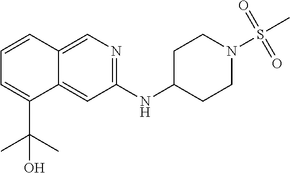 [SMILES: CC(C)(O)c1cccc2cnc(NC3CCN(S(C)(=O)=O)CC3)cc12] |
| I- 213 | 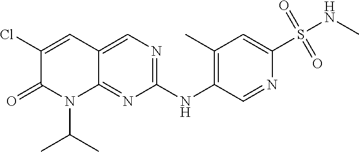 [SMILES: CNS(=O)(=O)c1cc(C)c(Nc2ncc3cc(Cl)c(=O)n(C(C)C)c3n2)cn1] |
| I- 214 | 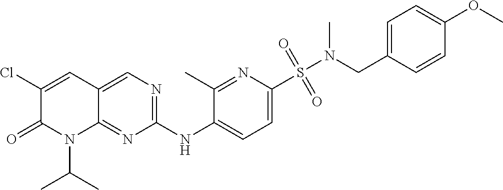 [SMILES: COc1ccc(CN(C)S(=O)(=O)c2ccc(Nc3ncc4cc(Cl)c(=O)n(C(C)C)c4n3)c(C)n2)cc1] |
| I- 215 | 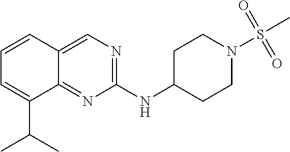 [SMILES: CC(C)c1cccc2cnc(NC3CCN(S(C)(=O)=O)CC3)nc12] |
| I- 216 | 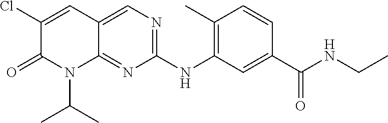 [SMILES: CCNC(=O)c1ccc(C)c(Nc2ncc3cc(Cl)c(=O)n(C(C)C)c3n2)c1] |
| I- 217 | 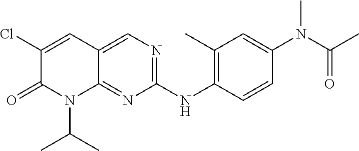 [SMILES: CC(=O)N(C)c1ccc(Nc2ncc3cc(Cl)c(=O)n(C(C)C)c3n2)c(C)c1] |
| I- 218 | 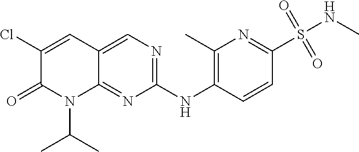 [SMILES: CNS(=O)(=O)c1ccc(Nc2ncc3cc(Cl)c(=O)n(C(C)C)c3n2)c(C)n1] |
| I- 219 | 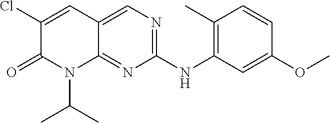 [SMILES: COc1ccc(C)c(Nc2ncc3cc(Cl)c(=O)n(C(C)C)c3n2)c1] |
| I- 220 | 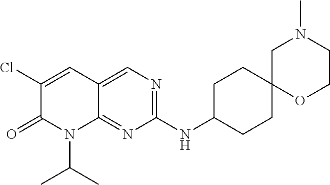 [SMILES: CC(C)n1c(=O)c(Cl)cc2cnc(NC3CCC4(CC3)CN(C)CCO4)nc21] |
| I- 221 | 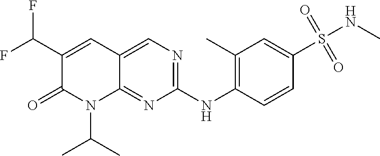 [SMILES: CNS(=O)(=O)c1ccc(Nc2ncc3cc(C(F)F)c(=O)n(C(C)C)c3n2)c(C)c1] |
| I- 222 | 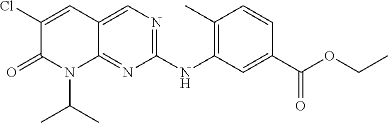 [SMILES: CCOC(=O)c1ccc(C)c(Nc2ncc3cc(Cl)c(=O)n(C(C)C)c3n2)c1] |
| I- 223 | 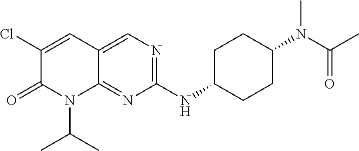 [SMILES: CC(=O)N(C)[C@H]1CC[C@@H](Nc2ncc3cc(Cl)c(=O)n(C(C)C)c3n2)CC1] |
| I- 224 | 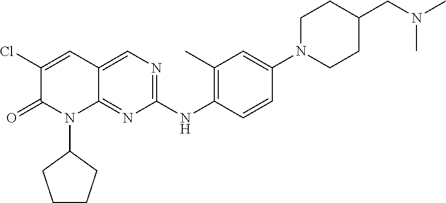 [SMILES: Cc1cc(N2CCC(CN(C)C)CC2)ccc1Nc1ncc2cc(Cl)c(=O)n(C3CCCC3)c2n1] |
| I- 225 | 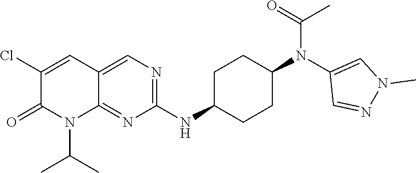 [SMILES: CC(=O)N(c1cnn(C)c1)[C@H]1CC[C@@H](Nc2ncc3cc(Cl)c(=O)n(C(C)C)c3n2)CC1] |
| I- 226 | 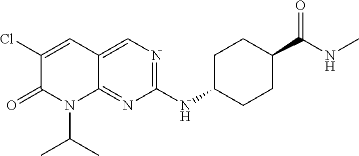 [SMILES: CNC(=O)[C@H]1CC[C@H](Nc2ncc3cc(Cl)c(=O)n(C(C)C)c3n2)CC1] |
| I- 227 | 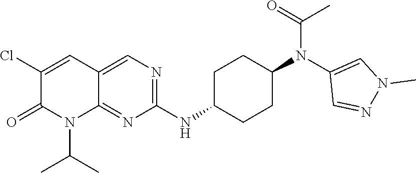 [SMILES: CC(=O)N(c1cnn(C)c1)[C@H]1CC[C@H](Nc2ncc3cc(Cl)c(=O)n(C(C)C)c3n2)CC1] |
| I- 228 | 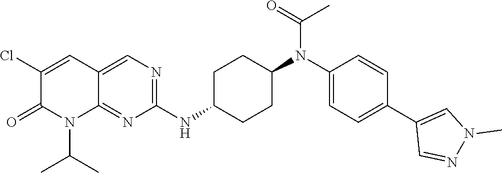 [SMILES: CC(=O)N(c1ccc(-c2cnn(C)c2)cc1)[C@H]1CC[C@H](Nc2ncc3cc(Cl)c(=O)n(C(C)C)c3n2)CC1] |
| I- 229 | 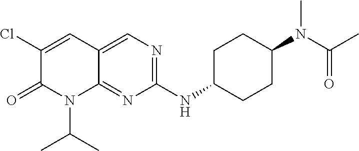 [SMILES: CC(=O)N(C)[C@H]1CC[C@H](Nc2ncc3cc(Cl)c(=O)n(C(C)C)c3n2)CC1] |
| I- 230 | 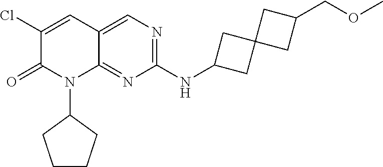 [SMILES: COCC1CC2(C1)CC(Nc1ncc3cc(Cl)c(=O)n(C4CCCC4)c3n1)C2] |
| I- 231 | 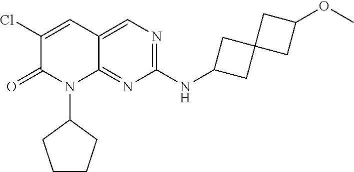 [SMILES: COC1CC2(CC(Nc3ncc4cc(Cl)c(=O)n(C5CCCC5)c4n3)C2)C1] |
| I- 232 | 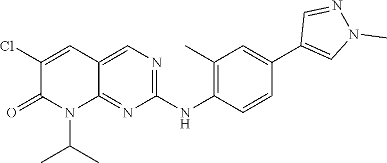 [SMILES: Cc1cc(-c2cnn(C)c2)ccc1Nc1ncc2cc(Cl)c(=O)n(C(C)C)c2n1] |
| I- 233 | 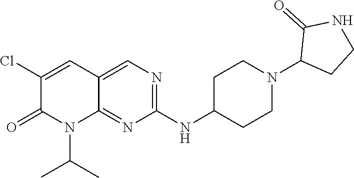 [SMILES: CC(C)n1c(=O)c(Cl)cc2cnc(NC3CCN(C4CCNC4=O)CC3)nc21] |
| I- 234 | 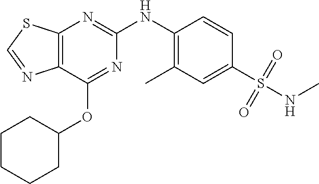 [SMILES: CNS(=O)(=O)c1ccc(Nc2nc(OC3CCCCC3)c3ncsc3n2)c(C)c1] |
| I- 235 | 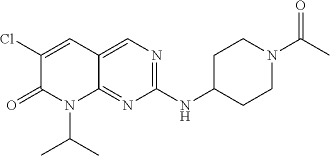 [SMILES: CC(=O)N1CCC(Nc2ncc3cc(Cl)c(=O)n(C(C)C)c3n2)CC1] |
| I- 236 | 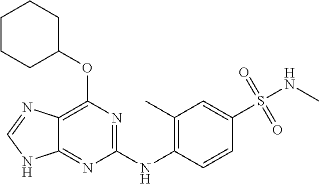 [SMILES: CNS(=O)(=O)c1ccc(Nc2nc(OC3CCCCC3)c3nc[nH]c3n2)c(C)c1] |
| I- 237 | 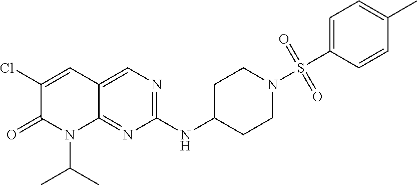 [SMILES: Cc1ccc(S(=O)(=O)N2CCC(Nc3ncc4cc(Cl)c(=O)n(C(C)C)c4n3)CC2)cc1] |
| I- 238 | 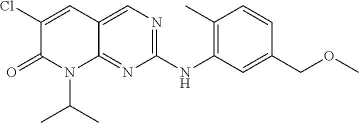 [SMILES: COCc1ccc(C)c(Nc2ncc3cc(Cl)c(=O)n(C(C)C)c3n2)c1] |
| I- 239 | 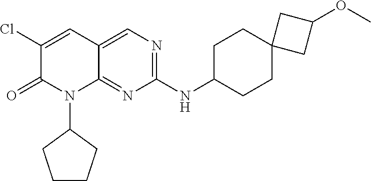 [SMILES: COC1CC2(CCC(Nc3ncc4cc(Cl)c(=O)n(C5CCCC5)c4n3)CC2)C1] |
| I- 240 | 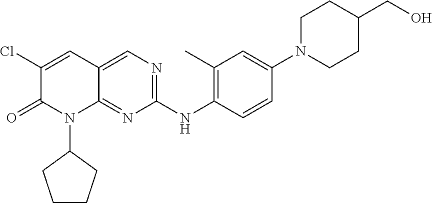 [SMILES: Cc1cc(N2CCC(CO)CC2)ccc1Nc1ncc2cc(Cl)c(=O)n(C3CCCC3)c2n1] |
| I- 241 | 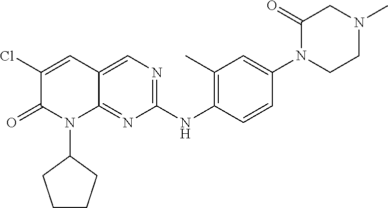 [SMILES: Cc1cc(N2CCN(C)CC2=O)ccc1Nc1ncc2cc(Cl)c(=O)n(C3CCCC3)c2n1] |
| I- 242 | 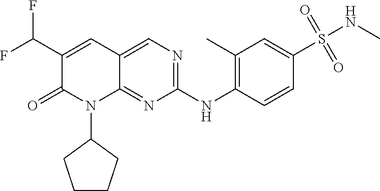 [SMILES: CNS(=O)(=O)c1ccc(Nc2ncc3cc(C(F)F)c(=O)n(C4CCCC4)c3n2)c(C)c1] |
| I- 243 | 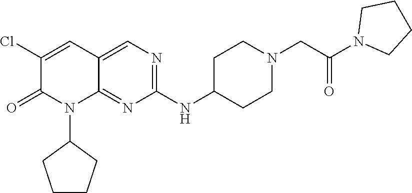 [SMILES: O=C(CN1CCC(Nc2ncc3cc(Cl)c(=O)n(C4CCCC4)c3n2)CC1)N1CCCC1] |
| I- 244 | 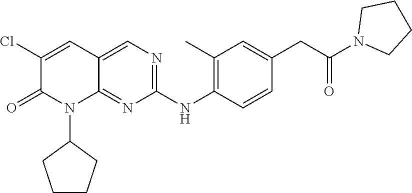 [SMILES: Cc1cc(CC(=O)N2CCCC2)ccc1Nc1ncc2cc(Cl)c(=O)n(C3CCCC3)c2n1] |
| I- 245 | 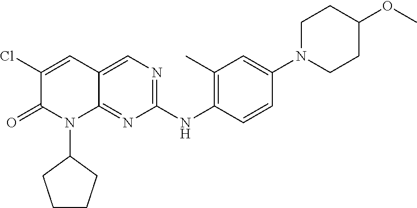 [SMILES: COC1CCN(c2ccc(Nc3ncc4cc(Cl)c(=O)n(C5CCCC5)c4n3)c(C)c2)CC1] |
| I- 246 | 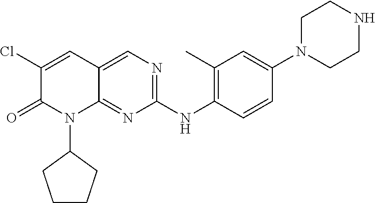 [SMILES: Cc1cc(N2CCNCC2)ccc1Nc1ncc2cc(Cl)c(=O)n(C3CCCC3)c2n1] |
| I- 247 | 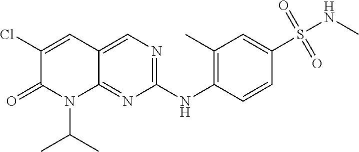 [SMILES: CNS(=O)(=O)c1ccc(Nc2ncc3cc(Cl)c(=O)n(C(C)C)c3n2)c(C)c1] |
| I- 248 | 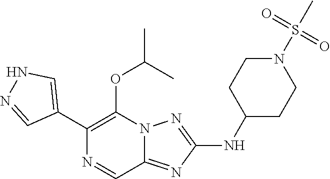 [SMILES: CC(C)Oc1c(-c2cn[nH]c2)ncc2nc(NC3CCN(S(C)(=O)=O)CC3)nn12] |
| I- 249 | 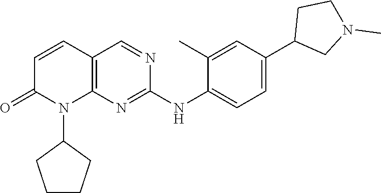 [SMILES: Cc1cc(C2CCN(C)C2)ccc1Nc1ncc2ccc(=O)n(C3CCCC3)c2n1] |
| I- 250 | 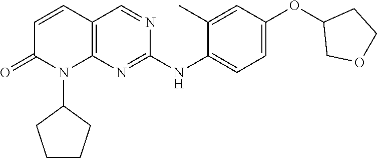 [SMILES: Cc1cc(OC2CCOC2)ccc1Nc1ncc2ccc(=O)n(C3CCCC3)c2n1] |
| I- 251 | 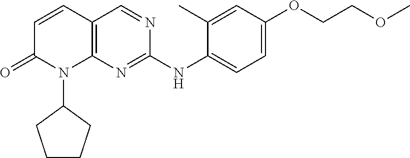 [SMILES: COCCOc1ccc(Nc2ncc3ccc(=O)n(C4CCCC4)c3n2)c(C)c1] |
| I- 252 | 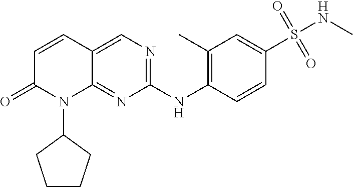 [SMILES: CNS(=O)(=O)c1ccc(Nc2ncc3ccc(=O)n(C4CCCC4)c3n2)c(C)c1] |
| I- 253 | 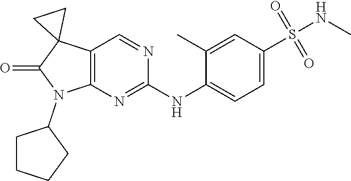 [SMILES: CNS(=O)(=O)c1ccc(Nc2ncc3c(n2)N(C2CCCC2)C(=O)C32CC2)c(C)c1] |
| I- 254 | 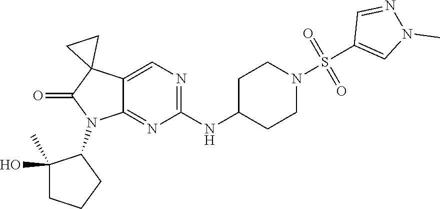 [SMILES: Cn1cc(S(=O)(=O)N2CCC(Nc3ncc4c(n3)N([C@@H]3CCC[C@@]3(C)O)C(=O)C43CC3)CC2)cn1] |
| I- 255 | 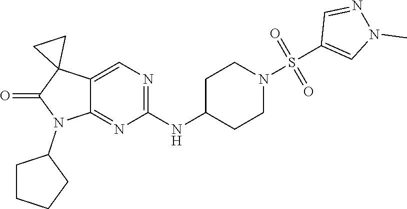 [SMILES: Cn1cc(S(=O)(=O)N2CCC(Nc3ncc4c(n3)N(C3CCCC3)C(=O)C43CC3)CC2)cn1] |
| I- 256 | 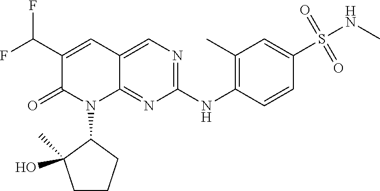 [SMILES: CNS(=O)(=O)c1ccc(Nc2ncc3cc(C(F)F)c(=O)n([C@@H]4CCC[C@@]4(C)O)c3n2)c(C)c1] |
| I- 257 | 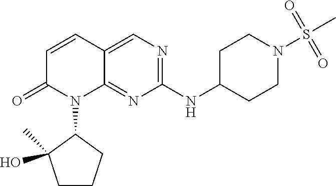 [SMILES: C[C@@]1(O)CCC[C@H]1n1c(=O)ccc2cnc(NC3CCN(S(C)(=O)=O)CC3)nc21] |
| I- 258 | 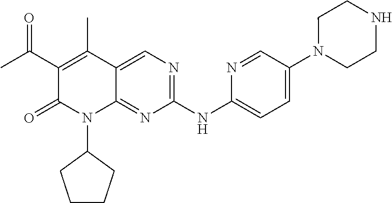 [SMILES: CC(=O)c1c(C)c2cnc(Nc3ccc(N4CCNCC4)cn3)nc2n(C2CCCC2)c1=O] |
| I- 259 | 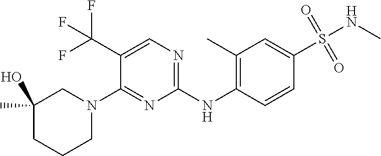 |
| I- 260 | 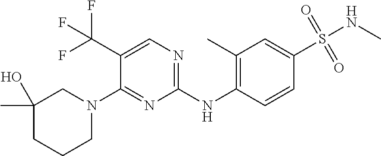 |
| I- 261 | 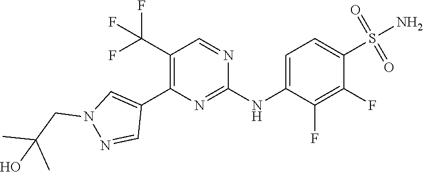 [SMILES: CC(C)(O)Cn1cc(-c2nc(Nc3ccc(S(N)(=O)=O)c(F)c3F)ncc2C(F)(F)F)cn1] |
| I- 262 | 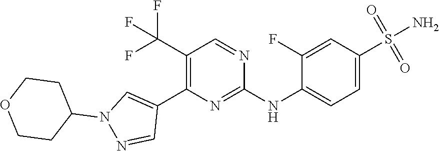 [SMILES: NS(=O)(=O)c1ccc(Nc2ncc(C(F)(F)F)c(-c3cnn(C4CCOCC4)c3)n2)c(F)c1] |
| I- 263 | 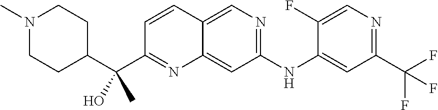 [SMILES: CN1CCC([C@@](C)(O)c2ccc3cnc(Nc4cc(C(F)(F)F)ncc4F)cc3n2)CC1] |
| I- 264 | 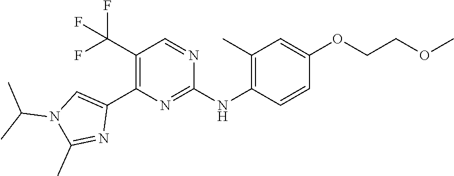 [SMILES: COCCOc1ccc(Nc2ncc(C(F)(F)F)c(-c3cn(C(C)C)c(C)n3)n2)c(C)c1] |
| I- 265 | 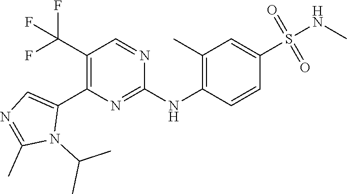 [SMILES: CNS(=O)(=O)c1ccc(Nc2ncc(C(F)(F)F)c(-c3cnc(C)n3C(C)C)n2)c(C)c1] |
| I- 266 | 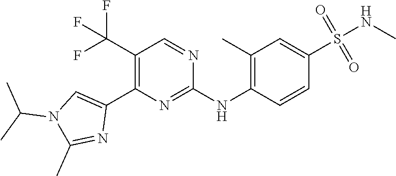 [SMILES: CNS(=O)(=O)c1ccc(Nc2ncc(C(F)(F)F)c(-c3cn(C(C)C)c(C)n3)n2)c(C)c1] |
| I- 267 | 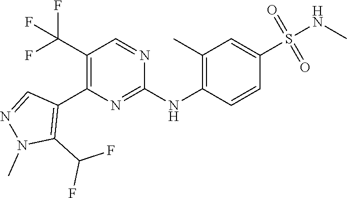 [SMILES: CNS(=O)(=O)c1ccc(Nc2ncc(C(F)(F)F)c(-c3cnn(C)c3C(F)F)n2)c(C)c1] |
| I- 268 | 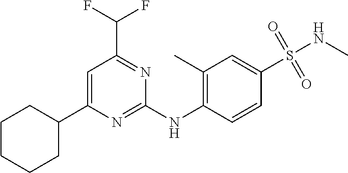 [SMILES: CNS(=O)(=O)c1ccc(Nc2nc(C(F)F)cc(C3CCCCC3)n2)c(C)c1] |
| I- 269 | 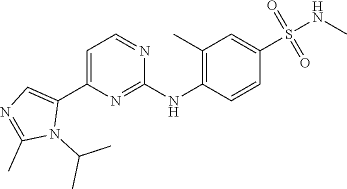 [SMILES: CNS(=O)(=O)c1ccc(Nc2nccc(-c3cnc(C)n3C(C)C)n2)c(C)c1] |
| I- 270 | 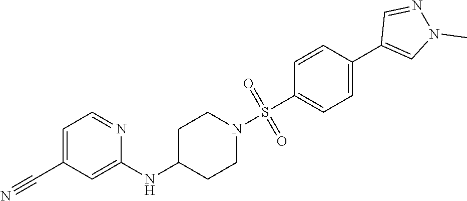 [SMILES: Cn1cc(-c2ccc(S(=O)(=O)N3CCC(Nc4cc(C#N)ccn4)CC3)cc2)cn1] |
| I- 271 | 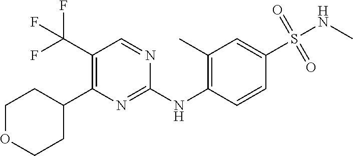 [SMILES: CNS(=O)(=O)c1ccc(Nc2ncc(C(F)(F)F)c(C3CCOCC3)n2)c(C)c1] |
| I- 272 | 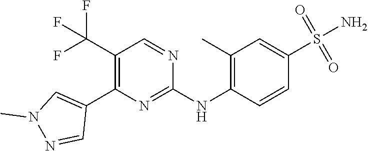 [SMILES: Cc1cc(S(N)(=O)=O)ccc1Nc1ncc(C(F)(F)F)c(-c2cnn(C)c2)n1] |
| I- 273 | 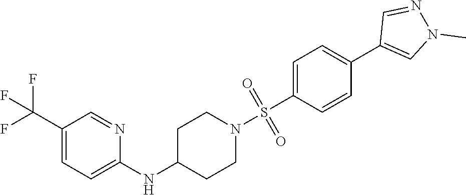 [SMILES: Cn1cc(-c2ccc(S(=O)(=O)N3CCC(Nc4ccc(C(F)(F)F)cn4)CC3)cc2)cn1] |
| I- 274 | 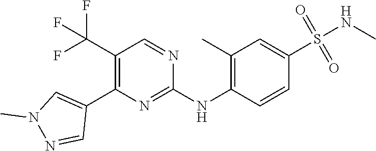 |
| I- 275 | 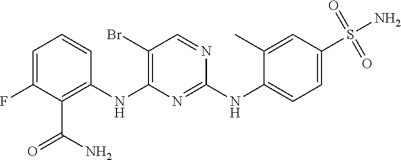 [SMILES: Cc1cc(S(N)(=O)=O)ccc1Nc1ncc(Br)c(Nc2cccc(F)c2C(N)=O)n1] |
| I- 276 | 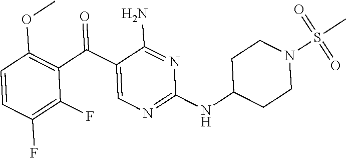 [SMILES: COc1ccc(F)c(F)c1C(=O)c1cnc(NC2CCN(S(C)(=O)=O)CC2)nc1N] |
| I- 277 | 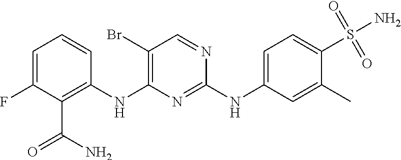 [SMILES: Cc1cc(Nc2ncc(Br)c(Nc3cccc(F)c3C(N)=O)n2)ccc1S(N)(=O)=O] |
| I- 278 | 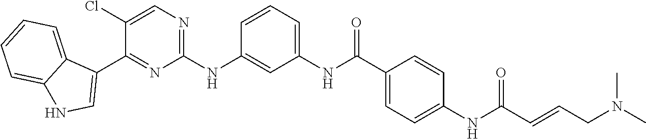 [SMILES: CN(C)C/C=C/C(=O)Nc1ccc(C(=O)Nc2cccc(Nc3ncc(Cl)c(-c4c[nH]c5ccccc45)n3)c2)cc1] |
| I- 279 | 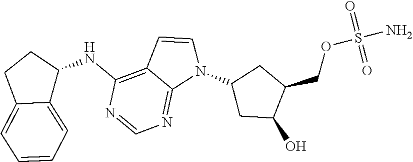 |
| I- 280 | 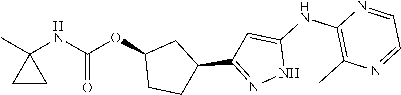 [SMILES: Cc1nccnc1Nc1cc([C@H]2CC[C@@H](OC(=O)NC3(C)CC3)C2)n[nH]1] |
| I- 281 | 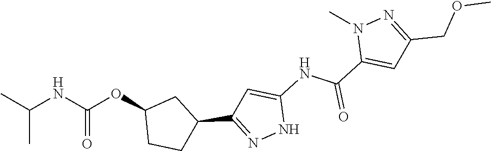 [SMILES: COCc1cc(C(=O)Nc2cc([C@H]3CC[C@@H](OC(=O)NC(C)C)C3)n[nH]2)n(C)n1] |
| I- 282 | 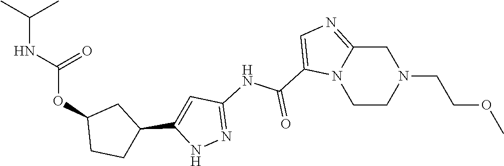 [SMILES: COCCN1CCn2c(C(=O)Nc3cc([C@H]4CC[C@@H](OC(=O)NC(C)C)C4)[nH]n3)cnc2C1] |
| I- 283 | 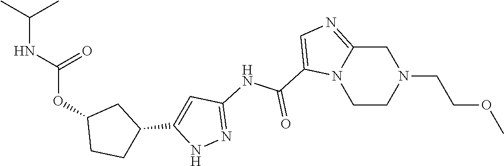 [SMILES: COCCN1CCn2c(C(=O)Nc3cc([C@@H]4CC[C@H](OC(=O)NC(C)C)C4)[nH]n3)cnc2C1] |
| I- 284 | 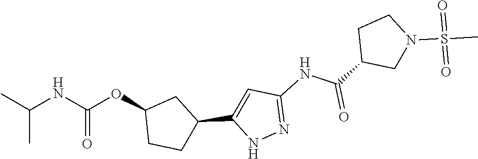 [SMILES: CC(C)NC(=O)O[C@@H]1CC[C@H](c2cc(NC(=O)[C@@H]3CCN(S(C)(=O)=O)C3)n[nH]2)C1] |
| I- 285 | 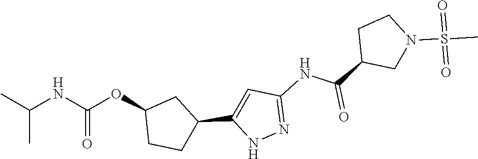 [SMILES: CC(C)NC(=O)O[C@@H]1CC[C@H](c2cc(NC(=O)[C@H]3CCN(S(C)(=O)=O)C3)n[nH]2)C1] |
| I- 286 | 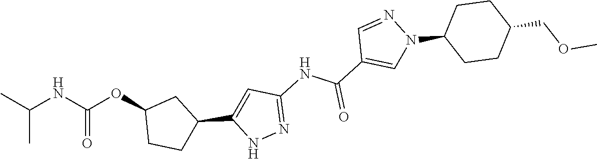 [SMILES: COC[C@H]1CC[C@H](n2cc(C(=O)Nc3cc([C@H]4CC[C@@H](OC(=O)NC(C)C)C4)[nH]n3)cn2)CC1] |
| I- 287 | 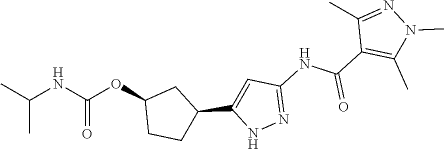 [SMILES: Cc1nn(C)c(C)c1C(=O)Nc1cc([C@H]2CC[C@@H](OC(=O)NC(C)C)C2)[nH]n1] |
| I- 288 | 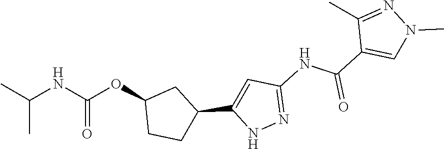 [SMILES: Cc1nn(C)cc1C(=O)Nc1cc([C@H]2CC[C@@H](OC(=O)NC(C)C)C2)[nH]n1] |
| I- 289 | 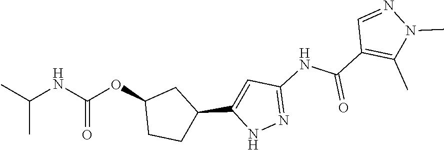 [SMILES: Cc1c(C(=O)Nc2cc([C@H]3CC[C@@H](OC(=O)NC(C)C)C3)[nH]n2)cnn1C] |
| I- 290 | 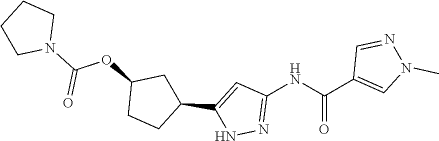 [SMILES: Cn1cc(C(=O)Nc2cc([C@H]3CC[C@@H](OC(=O)N4CCCC4)C3)[nH]n2)cn1] |
| I- 291 | 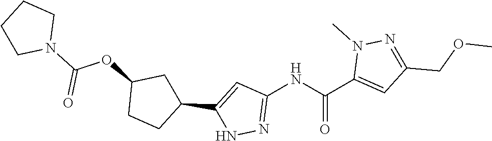 [SMILES: COCc1cc(C(=O)Nc2cc([C@H]3CC[C@@H](OC(=O)N4CCCC4)C3)[nH]n2)n(C)n1] |
| I- 292 | 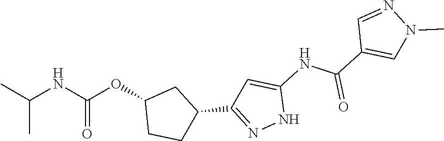 [SMILES: CC(C)NC(=O)O[C@H]1CC[C@@H](c2cc(NC(=O)c3cnn(C)c3)[nH]n2)C1] |
| I- 293 | 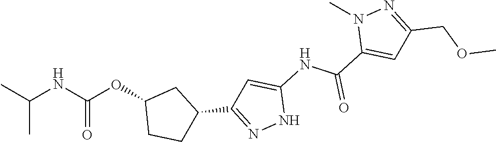 [SMILES: COCc1cc(C(=O)Nc2cc([C@@H]3CC[C@H](OC(=O)NC(C)C)C3)n[nH]2)n(C)n1] |
| I- 294 | 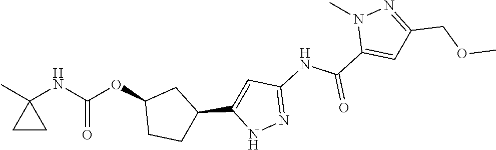 |
| I- 295 | 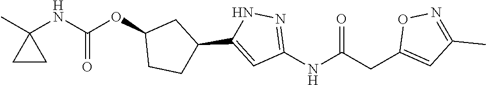 |
| I- 296 | 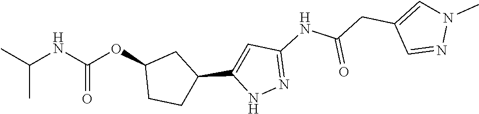 [SMILES: CC(C)NC(=O)O[C@@H]1CC[C@H](c2cc(NC(=O)Cc3cnn(C)c3)n[nH]2)C1] |
| I- 297 | 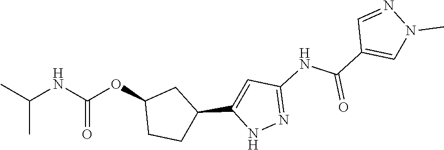 [SMILES: CC(C)NC(=O)O[C@@H]1CC[C@H](c2cc(NC(=O)c3cnn(C)c3)n[nH]2)C1] |
| I- 298 | 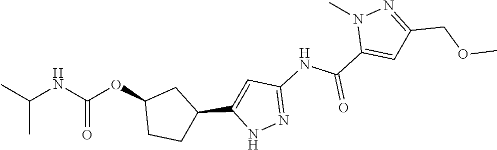 [SMILES: COCc1cc(C(=O)Nc2cc([C@H]3CC[C@@H](OC(=O)NC(C)C)C3)[nH]n2)n(C)n1] |
| I- 299 | 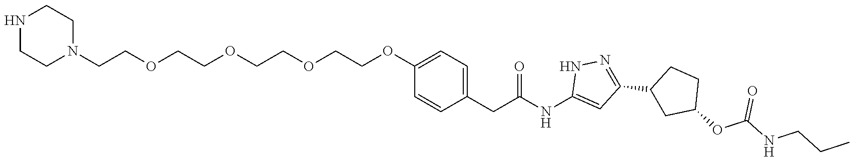 |
| I- 300 | 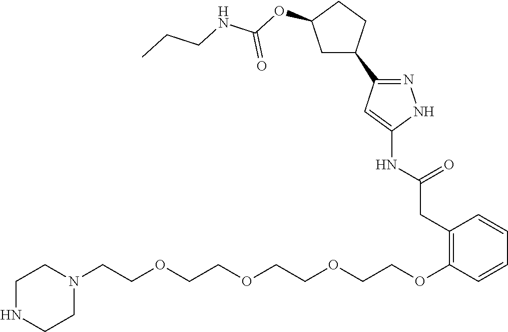 [SMILES: CCCNC(=O)O[C@H]1CC[C@@H](c2cc(NC(=O)Cc3ccccc3OCCOCCOCCOCCN3CCNCC3)[nH]n2)C1] |
| I- 301 | 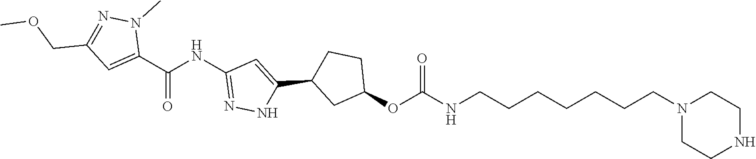 [SMILES: COCc1cc(C(=O)Nc2cc([C@H]3CC[C@@H](OC(=O)NCCCCCCCN4CCNCC4)C3)[nH]n2)n(C)n1] |
| I- 302 | 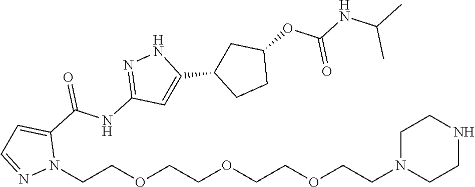 [SMILES: CC(C)NC(=O)O[C@@H]1CC[C@H](c2cc(NC(=O)c3ccnn3CCOCCOCCOCCN3CCNCC3)n[nH]2)C1] |
| I- 303 | 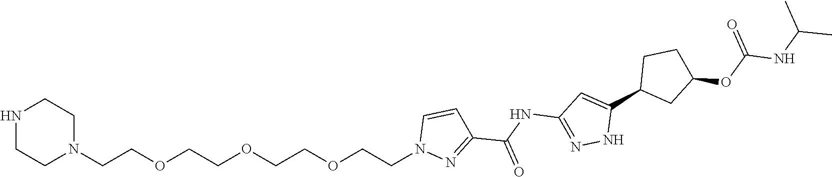 [SMILES: CC(C)NC(=O)O[C@@H]1CC[C@H](c2cc(NC(=O)c3ccn(CCOCCOCCOCCN4CCNCC4)n3)n[nH]2)C1] |

In some embodiments, the present invention provides a compound set forth in Table 1, above, or a pharmaceutically acceptable salt thereof.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that it is effective to measurably inhibit an CDK protein, or a mutant thereof, in a biological sample or in a patient. In certain embodiments, the amount of compound in compositions of this invention is such that it is effective to measurably inhibit a CDK protein, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of an CDK protein, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the inhibition of kinase activity of one or more enzymes.

As used herein, the terms “CDK1-mediated”, “CDK2-mediated”, “CDK4-mediated”, “CDK6-mediated”, “CDK7-mediated”, “CDK8-mediated”, and/or “CDK9-mediated” disorders, diseases, and/or conditions as used herein means any disease or other deleterious condition in which one or more of CDK1, CDK2, CDK4, CDK6, CDK7, CDK8, and/or CDK9 or a mutant thereof, are known to play a role. Accordingly, another embodiment of the present invention relates to treating or lessening the severity of one or more diseases in which one or more of CDK1, CDK2, CDK4, CDK6, CDK7, CDK8, and/or CDK9 or a mutant thereof, are known to play a role.

Compounds of the present disclosure can modulate CDK2 or CDK2 and CCNE1 and therefore are useful for treating diseases wherein the underlying pathology is, wholly or partially, mediated by CDK2. Such diseases include cancer and other diseases with proliferation disorder. In some embodiments, the present disclosure provides treatment of an individual or a patient in vivo using a provided compound or a pharmaceutically acceptable salt thereof such that growth of cancerous tumors is inhibited. A provided compound or a pharmaceutically acceptable salt thereof can be used to inhibit the growth of cancerous tumors with aberrations that activate CDK2 activity. These include, but not limited to, disease (e.g., cancers) that are characterized by amplification or overexpression of CCNE1 such as ovarian cancer, uterine carcinosarcoma and breast cancer and p27 inactivation such as breast cancer and melanomas. Accordingly, in some embodiments of the methods, the patient has been previously determined to have an amplification of the CCNE1 gene and/or an expression level of CCNE1 in a biological sample obtained from the human subject that is higher than a control expression level of CCNE1. Alternatively, a provided compound or a pharmaceutically acceptable salt thereof can be used in conjunction with other agents or standard cancer treatments, as described below. In one embodiment, the present disclosure provides a method for inhibiting growth of tumor cells in vitro. The method includes contacting the tumor cells in vitro with a provided compound or a pharmaceutically acceptable salt thereof. In another embodiment, the present disclosure provides a method for inhibiting growth of tumor cells with CCNE1 amplification and overexpression in an individual or a patient. The method includes administering to the individual or patient in need thereof a therapeutically effective amount of a provided compound or a pharmaceutically acceptable salt thereof.

In some embodiments, provided herein is a method of inhibiting CDK2, comprising contacting the CDK2 with a provided compound or a pharmaceutically acceptable salt thereof. In some embodiments, provided herein is a method of inhibiting CDK2 in a patient, comprising administering to the patient a provided compound or a pharmaceutically acceptable salt thereof.

In some embodiments, provided herein is a method of inhibiting CDK2 and CCNE1, comprising contacting the CDK2 and CCNE1 with a provided compound or a pharmaceutically acceptable salt thereof. In some embodiments, provided herein is a method of inhibiting CDK2 and CCNE1 in a patient, comprising administering to the patient a provided compound or a pharmaceutically acceptable salt thereof.

In some embodiments, provided herein is a method for treating cancer. The method includes administering to a patient (in need thereof), a therapeutically effective amount of a provided compound or a pharmaceutically acceptable salt thereof. In another embodiment, the cancer is characterized by amplification or overexpression of CCNE1. In some embodiments, the cancer is ovarian cancer or breast cancer, characterized by amplification or overexpression of CCNE1.

In some embodiments, provided herein is a method of treating a disease or disorder associated with CDK2 in a patient, comprising administering to the patient a therapeutically effective amount of a provided compound or a pharmaceutically acceptable salt thereof. In some embodiments, the disease or disorder associated with CDK2 is associated with an amplification of the CCNE1 gene and/or overexpression of CCNE1.

In some embodiments, the disease or disorder associated with CDK2 is N-myc amplified neuroblastoma cells (see Molenaar et al., Proc. Natl. Acad. Sci. USA, 2009, 106(31):12968-12973), K-Ras mutant lung cancers (see Hu, S., et al., Mol. Cancer Ther., 2015, 14(11):2576-85), and cancers with FBW7 mutation and CCNE1 overexpression (see Takada et al., Cancer Res., 2017, 77(18):4881-4893).

In some embodiments, the disease or disorder associated with CDK2 is lung squamous cell carcinoma, lung adenocarcinoma, pancreatic adenocarcinoma, breast invasive carcinoma, uterine carcinosarcoma, ovarian serous cystadenocarcinoma, stomach adenocarcinoma, esophageal carcinoma, bladder urothelial carcinoma, mesothelioma, or sarcoma.

In some embodiments, the disease or disorder associated with CDK2 is lung adenocarcinoma, breast invasive carcinoma, uterine carcinosarcoma, ovarian serous cystadenocarcinoma, or stomach adenocarcinoma.

In some embodiments, the disease or disorder associated with CDK2 is an adenocarcinoma, carcinoma, or cystadenocarcinoma.

In some embodiments, the disease or disorder associated with CDK2 is uterine cancer, ovarian cancer, stomach cancer, esophageal cancer, lung cancer, bladder cancer, pancreatic cancer, or breast cancer.

In some embodiments, the disease or disorder associated with CDK2 is a cancer.

In some embodiments, the cancer is characterized by amplification or overexpression of CCNE1. In some embodiments, the cancer is ovarian cancer or breast cancer, characterized by amplification or overexpression of CCNE1.

In some embodiments, the breast cancer is chemotherapy or radiotherapy resistant breast cancer, endocrine resistant breast cancer, trastuzumab resistant breast cancer, or breast cancer demonstrating primary or acquired resistance to CDK4/6 inhibition. In some embodiments, the breast cancer is advanced or metastatic breast cancer.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include melanoma (e.g., metastatic malignant melanoma, BRAF and HSP90 inhibition-resistant melanoma), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer, urothelial cancer (e.g., bladder) and cancers with high microsatellite instability (MSI^{high}). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the compounds of the present disclosure can be used to treat sickle cell disease and sickle cell anemia.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

It is believed that a provided compound or a pharmaceutically acceptable salt thereof may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

Co-Administration with One or More Other Therapeutic Agent(s)

Depending upon the particular condition, or disease, to be treated, additional therapeutic agents that are normally administered to treat that condition, can also be present in the compositions of this invention. As used herein, additional therapeutic agents that are normally administered to treat a particular disease, or condition, are known as “appropriate for the disease, or condition, being treated.”

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

A compound of the current invention can also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides, or in addition, be administered especially for tumor therapy in combination with chemotherapy, radiotherapy, immunotherapy, phototherapy, surgical intervention, or a combination of these. Long-term therapy is equally possible, as is adjuvant therapy in the context of other treatment strategies, as described above. Other possible treatments are therapy to maintain the patient's status after tumor regression, or even chemopreventive therapy, for example in patients at risk.

One or more other therapeutic agent(s) can be administered separately from a compound or composition of the invention, as part of a multiple dosage regimen. Alternatively, one or more other therapeutic agent(s) may be part of a single dosage form, mixed together with a compound of this invention in a single composition. If administered as a multiple dosage regime, one or more other therapeutic agent(s) and a compound or composition of the invention can be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent(s) and a compound or composition of the invention are administered as a multiple dosage regimen within greater than 24 hours apart.

As used herein, the term “combination,” “combined,” and related terms refers to the simultaneous or sequential administration of therapeutic agents in accordance with this invention. For example, a compound of the present invention can be administered with one or more other therapeutic agent(s) simultaneously or sequentially in separate unit dosage forms or together in a single unit dosage form. Accordingly, the present invention provides a single unit dosage form comprising a compound of the current invention, one or more other therapeutic agent(s), and a pharmaceutically acceptable carrier, adjuvant, or vehicle.

The amount of a compound of the invention and one or more other therapeutic agent(s) (in those compositions which comprise an additional therapeutic agent as described above) that can be combined with the carrier materials to produce a single dosage form varies depending upon the host treated and the particular mode of administration. Preferably, a composition of the invention should be formulated so that a dosage of between 0.01-100 mg/kg body weight/day of a compound of the invention can be administered.

In those compositions which comprise one or more other therapeutic agent(s), the one or more other therapeutic agent(s) and a compound of the invention can act synergistically. Therefore, the amount of the one or more other therapeutic agent(s) in such compositions may be less than that required in a monotherapy utilizing only that therapeutic agent. In such compositions a dosage of between 0.01-1,000 g/kg body weight/day of the one or more other therapeutic agent(s) can be administered.

The amount of one or more other therapeutic agent(s) present in the compositions of this invention may be no more than the amount that would normally be administered in a composition comprising that therapeutic agent as the only active agent. Preferably the amount of one or more other therapeutic agent(s) in the presently disclosed compositions ranges from about 50% to 100% of the amount normally present in a composition comprising that agent as the only therapeutically active agent. In some embodiments, one or more other therapeutic agent(s) is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, can also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Other Therapeutic Agents

In some embodiments, one or more other therapeutic agent is a Poly ADP ribose polymerase (PARP) inhibitor. In some embodiments, a PARP inhibitor is selected from olaparib (LYNPARZA®, AstraZeneca); rucaparib (RUBRACA®, Clovis Oncology); niraparib (ZEJULA®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (ZOLINZA®, Merck); romidepsin (ISTODAX®, Celgene); panobinostat (FARYDAK®, Novartis); belinostat (BELEODAQ®, Spectrum Pharmaceuticals); entinostat (SNDX-275, Syndax Pharmaceuticals) (NCT00866333); and chidamide (EPIDAZA®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (IBRANCE®, Pfizer); ribociclib (KISQALI®, Novartis); abemaciclib (Ly2835219, Eli Lilly); and trilaciclib (G1T28, G1 Therapeutics).

In some embodiments, one or more other therapeutic agent is a phosphatidylinositol 3 kinase (PI3K) inhibitor. In some embodiments, a PI3K inhibitor is selected from idelalisib (ZYDELIG®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, one or more other therapeutic agent is a platinum-based therapeutic, also referred to as platins. Platins cause cross-linking of DNA, such that they inhibit DNA repair and/or DNA synthesis, mostly in rapidly reproducing cells, such as cancer cells. In some embodiments, a platinum-based therapeutic is selected from cisplatin (PLATINOL®, Bristol-Myers Squibb); carboplatin (PARAPLATIN®, Bristol-Myers Squibb; also, Teva; Pfizer); oxaliplatin (ELOXITIN® Sanofi-Aventis); nedaplatin (AQUPLA®, Shionogi), picoplatin (Poniard Pharmaceuticals); and satraplatin (JM-216, Agennix).

In some embodiments, one or more other therapeutic agent is a taxane compound, which causes disruption of microtubules, which are essential for cell division. In some embodiments, a taxane compound is selected from paclitaxel (TAXOL®, Bristol-Myers Squibb), docetaxel (TAXOTERE®, Sanofi-Aventis; DOCEFREZ®, Sun Pharmaceutical), albumin-bound paclitaxel (ABRAXANE®; Abraxis/Celgene), cabazitaxel (JEVTANA®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, YONDELIS®, Janssen Oncology), mechlorethamine (alkylating agent, VALCHLOR®, Aktelion Pharmaceuticals); vincristine (ONCOVIN®, Eli Lilly; VINCASAR®, Teva Pharmaceuticals; MARQIBO®, Talon Therapeutics); temozolomide (prodrug to alkylating agent 5-(3-methyltriazen-1-yl)-imidazole-4-carboxamide (MTIC) TEMODAR®, Merck); cytarabine injection (ara-C, antimetabolic cytidine analog, Pfizer); lomustine (alkylating agent, CEENU®, Bristol-Myers Squibb; GLEOSTINE®, NextSource Biotechnology); azacitidine (pyrimidine nucleoside analog of cytidine, VIDAZA®, Celgene); omacetaxine mepesuccinate (cephalotaxine ester) (protein synthesis inhibitor, SYNRIBO®; Teva Pharmaceuticals); asparaginase Erwinia chrysanthemi (enzyme for depletion of asparagine, ELSPAR®, Lundbeck; ERWINAZE®, EUSA Pharma); eribulin mesylate (microtubule inhibitor, tubulin-based antimitotic, HALAVEN®, Eisai); cabazitaxel (microtubule inhibitor, tubulin-based antimitotic, JEVTANA®, Sanofi-Aventis); capacetrine (thymidylate synthase inhibitor, XELODA®, Genentech); bendamustine (bifunctional mechlorethamine derivative, believed to form interstrand DNA cross-links, TREANDA®, Cephalon/Teva); ixabepilone (semi-synthetic analog of epothilone B, microtubule inhibitor, tubulin-based antimitotic, IXEMPRA®, Bristol-Myers Squibb); nelarabine (prodrug of deoxyguanosine analog, nucleoside metabolic inhibitor, ARRANON®, Novartis); clorafabine (prodrug of ribonucleotide reductase inhibitor, competitive inhibitor of deoxycytidine, CLOLAR®, Sanofi-Aventis); and trifluridine and tipiracil (thymidine-based nucleoside analog and thymidine phosphorylase inhibitor, LONSURF®, Taiho Oncology).

In some embodiments, one or more other therapeutic agent is a kinase inhibitor or VEGF-R antagonist. Approved VEGF inhibitors and kinase inhibitors useful in the present invention include: bevacizumab (AVASTIN®, Genentech/Roche) an anti-VEGF monoclonal antibody; ramucirumab (CYRAMZA®, Eli Lilly), an anti-VEGFR-2 antibody and ziv-aflibercept, also known as VEGF Trap (ZALTRAP®; Regeneron/Sanofi). VEGFR inhibitors, such as regorafenib (STIVARGA®, Bayer); vandetanib (CAPRELSA®, AstraZeneca); axitinib (INLYTA®, Pfizer); and lenvatinib (LENVIMA®, Eisai); Raf inhibitors, such as sorafenib (NEXAVAR®, Bayer AG and Onyx); dabrafenib (TAFINLAR®, Novartis); and vemurafenib (ZELBORAF®, Genentech/Roche); MEK inhibitors, such as cobimetanib (COTELLIC®, Exelexis/Genentech/Roche); trametinib (MEKINIST®, Novartis); Bcr-Abl tyrosine kinase inhibitors, such as imatinib (GLEEVEC®, Novartis); nilotinib (TASIGNA®, Novartis); dasatinib (SPRYCEL®, BristolMyersSquibb); bosutinib (BOSULIF®, Pfizer); and ponatinib (INCLUSIG®, Ariad Pharmaceuticals); Her2 and EGFR inhibitors, such as gefitinib (IRESSA®, AstraZeneca); erlotinib (TARCEEVA®, Genentech/Roche/Astellas); lapatinib (TYKERB®, Novartis); afatinib (GILOTRIF®, Boehringer Ingelheim); osimertinib (targeting activated EGFR, TAGRISSO®, AstraZeneca); and brigatinib (ALUNBRIG®, Ariad Pharmaceuticals); c-Met and VEGFR2 inhibitors, such as cabozanitib (COMETRIQ®, Exelexis); and multikinase inhibitors, such as sunitinib (SUTENT®, Pfizer); pazopanib (VOTRIENT®, Novartis); ALK inhibitors, such as crizotinib (XALKORI®, Pfizer); ceritinib (ZYKADIA®, Novartis); and alectinib (ALECENZa®, Genentech/Roche); Bruton's tyrosine kinase inhibitors, such as ibrutinib (IMBRUVICA®, Pharmacyclics/Janssen); and Flt3 receptor inhibitors, such as midostaurin (RYDAPT®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (SUPECT®, IY5511, Il-Yang Pharmaceuticals, S. Korea); ruxolitinib (JAKAFI®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In some embodiments, one or more other therapeutic agent is an mTOR inhibitor, which inhibits cell proliferation, angiogenesis and glucose uptake. In some embodiments, an mTOR inhibitor is everolimus (AFINITOR®, Novartis); temsirolimus (TORISEL®, Pfizer); and sirolimus (RAPAMUNE®, Pfizer).

In some embodiments, one or more other therapeutic agent is a proteasome inhibitor. Approved proteasome inhibitors useful in the present invention include bortezomib (VELCADE®, Takeda); carfilzomib (KYPROLIS®, Amgen); and ixazomib (NINLARO®, Takeda).

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (LARTRUVO®; Eli Lilly). Approved EGFR antagonists which may be used in the present invention include cetuximab (ERBITUX®, Eli Lilly); necitumumab (PORTRAZZA®, Eli Lilly), panitumumab (VECTIBIX®, Amgen); and osimertinib (targeting activated EGFR, TAGRISSO®, AstraZeneca).

In some embodiments, one or more other therapeutic agent is an aromatase inhibitor. In some embodiments, an aromatase inhibitor is selected from exemestane (AROMASIN®, Pfizer); anastazole (ARIMIDEX®, AstraZeneca) and letrozole (FEMARA®, Novartis).

In some embodiments, one or more other therapeutic agent is an antagonist of the hedgehog pathway. Approved hedgehog pathway inhibitors which may be used in the present invention include sonidegib (ODOMZO®, Sun Pharmaceuticals); and vismodegib (ERIVEDGE®, Genentech), both for treatment of basal cell carcinoma.

In some embodiments, one or more other therapeutic agent is a folic acid inhibitor. Approved folic acid inhibitors useful in the present invention include pemetrexed (ALIMTA®, Eli Lilly).

In some embodiments, one or more other therapeutic agent is a CC chemokine receptor 4 (CCR4) inhibitor. CCR4 inhibitors being studied that may be useful in the present invention include mogamulizumab (POTELIGEO®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (RITUXAN®, Genentech/BiogenIdec); ofatumumab (anti-CD20, ARZERRA®, GlaxoSmithKline); obinutuzumab (anti-CD20, GAZYVA®, Genentech), ibritumomab (anti-CD20 and Yttrium-90, ZEVALIN®, Spectrum Pharmaceuticals); daratumumab (anti-CD38, DARZALEX®, Janssen Biotech), dinutuximab (anti-glycolipid GD2, UNITUXIN®, United Therapeutics); trastuzumab (anti-HER2, HERCEPTIN®, Genentech); ado-trastuzumab emtansine (anti-HER2, fused to emtansine, KADCYLA®, Genentech); and pertuzumab (anti-HER2, PERJETA®, Genentech); and brentuximab vedotin (anti-CD30-drug conjugate, ADCETRIS®, Seattle Genetics).

In some embodiments, one or more other therapeutic agent is a topoisomerase inhibitor. Approved topoisomerase inhibitors useful in the present invention include irinotecan (ONIVYDE®, Merrimack Pharmaceuticals); topotecan (HYCAMTIN®, GlaxoSmithKline). Topoisomerase inhibitors being studied which may be used in the present invention include pixantrone (PIXUVRI®, CTI Biopharma).

In some embodiments, one or more other therapeutic agent is an inhibitor of anti-apoptotic proteins, such as BCL-2. Approved anti-apoptotics which may be used in the present invention include venetoclax (VENCLEXTA®, AbbVie/Genentech); and blinatumomab (BLINCYTO®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (XTANDI®, Astellas/Medivation); approved inhibitors of androgen synthesis include abiraterone (ZYTIGA®, Centocor/Ortho); approved antagonist of gonadotropin-releasing hormone (GnRH) receptor (degaralix, FIRMAGON®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (EVISTA®, Eli Lilly).

In some embodiments, one or more other therapeutic agent is an inhibitor of bone resorption. An approved therapeutic which inhibits bone resorption is Denosumab (XGEVA®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (ZOMETA®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFß). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGF-β trap compound (NCT02699515); and (NCT02517398). M7824 is comprised of a fully human IgG1 antibody against PD-L1 fused to the extracellular domain of human TGF-beta receptor II, which functions as a TGF-β “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agents is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, TEMODAL CNF1010, CNF2024, CNF1010 from Conforma Therapeutics; temozolomide (TEMODAL®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name AROMASIN™. Formestane is marketed under the trade name LENTARON™. Fadrozole is marketed under the trade name AFEMA™. Anastrozole is marketed under the trade name ARIMIDEX™. Letrozole is marketed under the trade names FEMARA™ or FEMAr™. Aminoglutethimide is marketed under the trade name ORIMETEN™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name NOLVADEX™ Raloxifene hydrochloride is marketed under the trade name EVISTA™. Fulvestrant can be administered under the trade name FASLODEX™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (CASODEX™). The term “gonadorelin agonist” as used herein includes, but is not limited to abarelix, goserelin, and goserelin acetate. Goserelin can be administered under the trade name ZOLADEX™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g., in the form as it is marketed, e.g., under the trademark CAMPTOSAR™. Topotecan is marketed under the trade name HYCAMPTIN™.

The term “topoisomerase II inhibitor” as used herein includes, but is not limited to the anthracyclines such as doxorubicin (including liposomal formulation, such as CAELYX™), daunorubicin, epirubicin, idarubicin and nemorubicin, the anthraquinones mitoxantrone and losoxantrone, and the podophillotoxines etoposide and teniposide. Etoposide is marketed under the trade name ETOPOPHOS™ Teniposide is marketed under the trade name VM 26-Bristol Doxorubicin is marketed under the trade name ACRIBLASTIN™ or ADRIAMYCIN™. Epirubicin is marketed under the trade name FARMORUBICIN™. Idarubicin is marketed. under the trade name ZAVEDOS™. Mitoxantrone is marketed under the trade name NOVANTRON™.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; colchicine and epothilones and derivatives thereof. Paclitaxel is marketed under the trade name TAXOL™. Docetaxel is marketed under the trade name TAXOTERE™. Vinblastine sulfate is marketed under the trade name VINBLASTIN R.P™. Vincristine sulfate is marketed under the trade name FARMISTIN™.

The term “alkylating agent” as used herein includes, but is not limited to, cyclophosphamide, ifosfamide, melphalan or nitrosourea (BCNU or Gliadel). Cyclophosphamide is marketed under the trade name CYCLOSTIN™. Ifosfamide is marketed under the trade name HOLOXAN™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name XELODA™. Gemcitabine is marketed under the trade name GEMZAR™.

The term “platin compound” as used herein includes, but is not limited to, carboplatin, cis-platin, cisplatinum and oxaliplatin. Carboplatin can be administered, e.g., in the form as it is marketed, e.g., under the trademark CARBOPLAT™. Oxaliplatin can be administered, e.g., in the form as it is marketed, e.g. under the trademark ELOXATIN™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g., BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporne derivatives, such as midostaurin; examples of further compounds include UCN-01, safingol, BAY 43-9006, Bryostatin 1, Perifosine; llmofosine; RO 318220 and RO 320432; GO 6976; 1sis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); l) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (HERCEPTIN™), cetuximab (ERBITUX™), Iressa, Tarceva, OSI-774, Cl-1033, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof, see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g., unrelated to protein or lipid kinase inhibition e.g., thalidomide (THALOMID™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (CELEBREX™), rofecoxib (VIOXX™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name DIDRONEL™. Clodronic acid is marketed under the trade name BONEFOS™ Tiludronic acid is marketed under the trade name Skelid™. Pamidronic acid is marketed under the trade name AREDIA™. Alendronic acid is marketed under the trade name FOSAMAX™. Ibandronic acid is marketed under the trade name BONDRANAT™. Risedronic acid is marketed under the trade name ACTONEL™. Zoledronic acid is marketed under the trade name ZOMETA™. The term “mTOR inhibitors” relates to compounds which inhibit the mammalian target of rapamycin (mTOR) and which possess antiproliferative activity such as sirolimus (RAPAMUNE®), everolimus (CERTICAN™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (ZARNESTRA™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (VELCADE™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g., hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (HERCEPTIN™), Trastuzumab-DM1, erbitux, bevacizumab (AVASTIN™), rituximab (RITUXAN®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; ANGIOSTATIN™; ENDOSTATIN™; anthranilic acid amides; ZD4190; Zd₆474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (AVASTIN™).

Photodynamic therapy as used herein refers to therapy which uses certain chemicals known as photosensitizing compounds to treat or prevent cancers. Examples of photodynamic therapy include treatment with compounds, such as VISUDYNE™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g., Patents International (e.g., IMS World Publications).

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTOR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonist of activating receptors on NK cells. In some embodiments, an immuno-oncology agent is an antagonist of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MED14736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Ikena Oncology, formerly known as Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (OPDIVO®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (POMALYST®, Celgene); lenalidomide (REVLIMID®, Celgene); ingenol mebutate (PICATO®, LEO Pharma).

In some embodiments, an immuno-oncology agent is a cancer vaccine. In some embodiments, the cancer vaccine is selected from sipuleucel-T (PROVENGE®, Dendreon/Valeant Pharmaceuticals), which has been approved for treatment of asymptomatic, or minimally symptomatic metastatic castrate-resistant (hormone-refractory) prostate cancer; and talimogene laherparepvec (IMLYGIC®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (REOLYSIN®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June et al.; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that can be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams et al. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BITE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BITE®) antibody construct. In some embodiments, a bispecific T cell engager (BITE®) antibody construct is a CD19/CD3 bispecific antibody construct. In some embodiments, a bispecific T cell engager (BITE®) antibody construct is an EGFR/CD3 bispecific antibody construct. In some embodiments, a bispecific T cell engager (BITE®) antibody construct activates T cells. In some embodiments, a bispecific T cell engager (BITE®) antibody construct activates T cells, which release cytokines inducing upregulation of intercellular adhesion molecule 1 (ICAM-1) and FAS on bystander cells. In some embodiments, a bispecific T cell engager (BITE®) antibody construct activates T cells which result in induced bystander cell lysis. In some embodiments, the bystander cells are in solid tumors. In some embodiments, the bystander cells being lysed are in proximity to the BITE®-activated T cells. In some embodiments, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In some embodiments, the checkpoint inhibitor is a biologic therapeutic or a small molecule. In some embodiments, the checkpoint inhibitor is a monoclonal antibody, a humanized antibody, a fully human antibody, a fusion protein or a combination thereof. In some embodiments, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor is an immunostimulatory agent, a T cell growth factor, an interleukin, an antibody, a vaccine or a combination thereof. In some embodiments, the interleukin is IL-7 or IL-15. In some embodiments, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors can include small molecule inhibitors or can include antibodies, or antigen binding fragments thereof, that bind to and block or inhibit immune checkpoint receptors or antibodies that bind to and block or inhibit immune checkpoint receptor ligands. Illustrative checkpoint molecules that can be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include, but are not limited to, Tremelimumab (CTLA-4 blocking antibody), anti-OX40, PD-L1 monoclonal Antibody (Anti-B7-H1; MED14736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L 1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (OPDIVO®), ipilimumab (YERVOY®), and pembrolizumab (KEYTRUDA®). In some embodiments, the checkpoint inhibitor is selected from nivolumab (anti-PD-1 antibody, OPDIVO®, Bristol-Myers Squibb); pembrolizumab (anti-PD-1 antibody, KEYTRUDA®, Merck); ipilimumab (anti-CTLA-4 antibody, YERVOY®, Bristol-Myers Squibb); durvalumab (anti-PD-L1 antibody, IMFINZI®, AstraZeneca); and atezolizumab (anti-PD-L1 antibody, TECENTRIQ®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (KEYTRUDA®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (BAVENCIO®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that can be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MED16469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that can be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981); and CTX-471 (Compass Therapeutics), an agonistic anti-CD137 antibody in metastatic or locally advanced malignancies (NCT03881488).

Checkpoint inhibitors that can be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that can be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that can be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that can be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that can be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that can be used in the present invention include CD73 inhibitors. CD73 inhibitors that are being studied in clinical trials include MED19447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that can be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that can be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that can be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### NUMBERED EMBODIMENTS

The following numbered embodiments, while non-limiting, are exemplary of certain aspects of the present disclosure:

Embodiment 1. A compound of formula I-a:

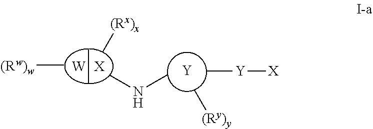

or a pharmaceutically acceptable salt thereof, wherein:
Ring W and Ring X are independently fused rings selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenylenyl, a 4 to 7-membered saturated or partially unsaturated carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
Y is a covalent bond, —S(O)₂—, —S(O)—, —S(O)(NR)—, —P(O)R—, or —P(O)OR—;
X is —NR₂ or an optionally substituted group selected from C₁₋₆ aliphatic, phenyl, a 3 to 12-membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
R^{w}, R^{x}, and R^{y} are independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, and —NRS(O)₂R; or
   two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and w, x, and y are independently 0, 1, 2, 3, or 4.
   Embodiment 2. The compound of embodiment 1, wherein said compound is a compound of any of the following formulae:

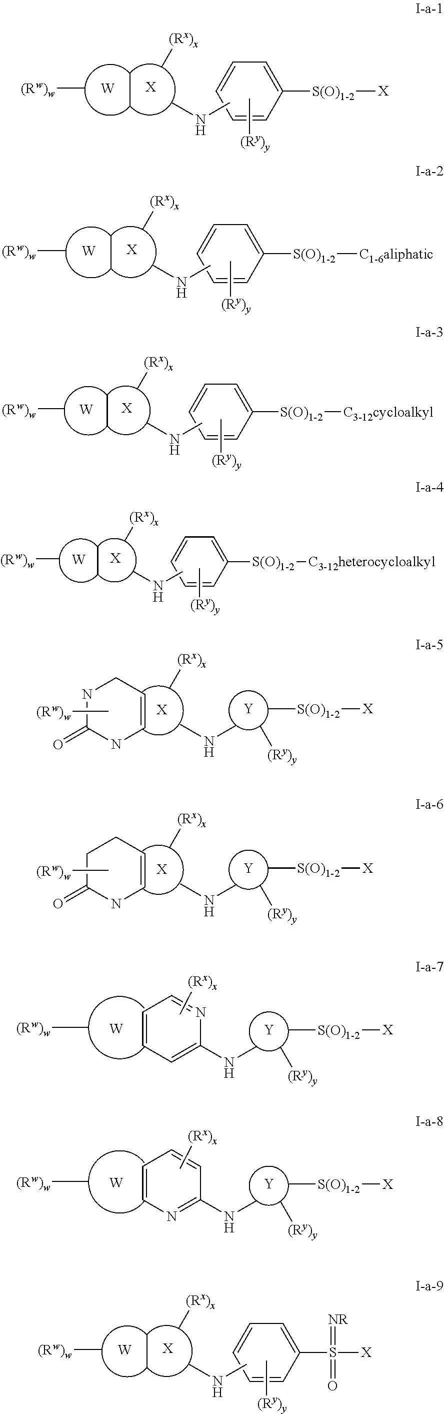

or a pharmaceutically acceptable salt thereof.
Embodiment 3. The compound of embodiment 1 or embodiment 2, wherein Ring W is a fused ring selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.
Embodiment 4. The compound of any one of embodiments 1-3, wherein Ring X is a fused ring selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.
Embodiment 5. The compound of any one of embodiments 1-4, wherein R^{w} is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R.
Embodiment 6. The compound of any one of embodiments 1-5, wherein R^{x} is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R.
Embodiment 7. The compound of any one of embodiments 1-6, wherein R^{y} is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R.
Embodiment 8. A compound of formula I-b:

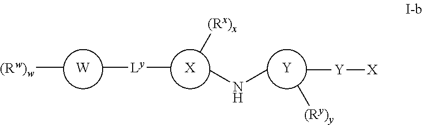

or a pharmaceutically acceptable salt thereof, wherein:
Ring W, Ring X, and Ring Y are independently rings selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Y is a covalent bond, —S(O)₂—, —S(O)—, —S(O)(NR)—, —P(O)R—, or —P(O)OR—;
X is —NR₂ or an optionally substituted group selected from C₁₋₆ aliphatic, phenyl, a 3 to 12-membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
R^{w}, R^{x}, and R^{y} are independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, and —NRS(O)₂R; or
   two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
L^{y} is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CF₂—, —CRF—, —NR—, —S—, —S(O)—, —S(O)₂— or —CR═CR—; and
w, x, and y are independently 0, 1, 2, 3, or 4.
   Embodiment 9. The compound of embodiment 8, wherein said compound is a compound of any of the following formulae:

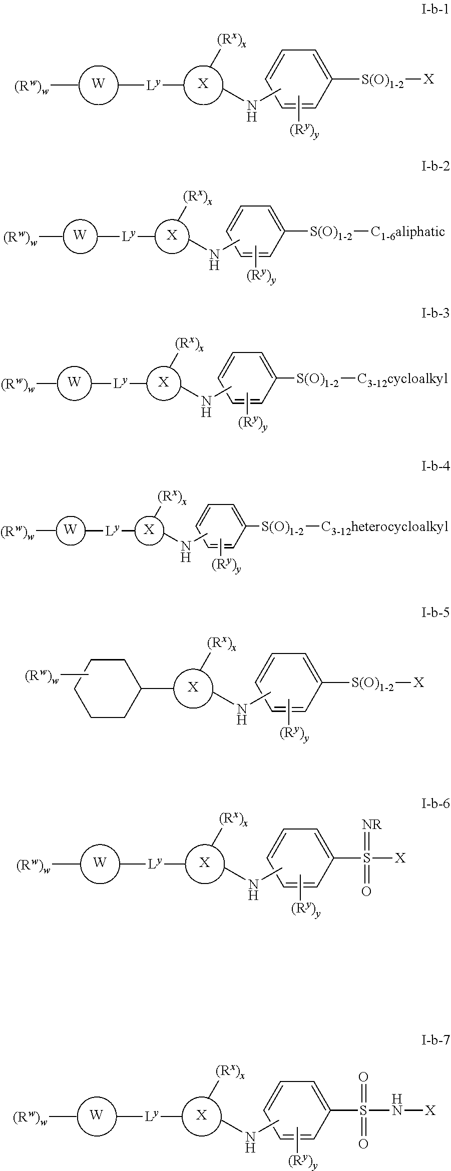

or a pharmaceutically acceptable salt thereof.
Embodiment 10. The compound of embodiment 8 or embodiment 9, wherein Ring W is a ring selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.
Embodiment 11. The compound of any one of embodiments 8-10, wherein Ring X is a ring selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.
Embodiment 12. The compound of any one of embodiments 8-11, wherein R^{w} is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R.
Embodiment 13. The compound of any one of embodiments 8-12, wherein R^{x} is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R.
Embodiment 14. The compound of any one of embodiments 8-13, wherein R^{y} is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R.
Embodiment 15. The compound of any one of embodiments 1-14, wherein said compound is selected from any one of the compounds depicted in Table 1, or a pharmaceutically acceptable salt thereof.
Embodiment 16. A pharmaceutical composition comprising a compound of any one of embodiments 1-15, and a pharmaceutically acceptable carrier, adjuvant, or vehicle.
Embodiment 17. The pharmaceutical composition according to embodiment 16, further comprising an additional therapeutic agent.
Embodiment 18. A method of inhibiting CDK2 or CDK2 and CCNE1 in a patient or biological sample comprising administering to said patient, or contacting said biological sample with a compound according to any one of embodiments 1-15, or a pharmaceutical composition thereof.
Embodiment 19. A method of treating an CDK2-mediated disorder, disease, or condition in a patient comprising administering to said patient a compound according to any of one of embodiments 1-15, or a pharmaceutical composition thereof.
Embodiment 20. The method of embodiment 19, wherein CDK2-mediated disorder, disease, or condition is cancer.
Embodiment 21. The method of embodiment 20, wherein the cancer the cancer is characterized by amplification or overexpression of CCNE1.

### EXEMPLIFICATION

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

Analytical instruments Table:

| | |
|---|---|
| LCMS | Shimadzu UFLC MS: LCMS-2020 |
| | Agilent Technologies 1200 series MS: Agilent Technologies 6110 |
| | Agilent Technologies 1200 series MS: LC/MSD VL |
| NMR | BRUKER AVANCE III/400; Frequency (MHz) 400.13; Nucleus: 1H; |
| | Number of Transients: 8 |
| Prep-HPLC | Gilson GX-281 systems: instruments GX-A, GX-B, GX-C, GX-D, GX-E, |
| | GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH⁺] and equipped with Chromolith Flash RP-18e 25*2.0 mm, eluting with 0.0375 vol % TFA in water (solventA) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH⁺] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH₃·H₂O in water (solvent A) and acetonitrile (solvent B).

HPLC Analytical Method: HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19) mm, 5. Column flow was 16.0 ml/min. Mobile phase were used (A) 0.1% Formic Acid in Water and (B) Acetonitrile Basic method used (A) 5 mM ammonium bicarbonate and 0.1% NH3 in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

### Abbreviations

Ac: acetyl
AcOH: acetic acid
ACN: acetonitrile
Ad: adamantly
AIBN: 2,2′-azo bisisobutyronitrile
Anhyd: anhydrous
Aq: aqueous
B₂Pin₂: bis (pinacolato)diboron-4,4,4′,4′,5,5,5′,5′-octamethyl-2,2′-bi(1,3,2-dioxaborolane)
BINAP: 2,2′-bis(diphenylphosphino)-1,1′-binaphthyl
BH₃: Borane
Bn: benzyl
Boc: tert-butoxycarbonyl
Boc₂O: di-tert-butyl dicarbonate
BPO: benzoyl peroxide
ⁿBuOH: n-butanol
CDI: carbonyldiimidazole
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIBAL-H: diisobutylaluminum hydride
DIPA: diisopropylamine
DIPEA or DIEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO-dimethyl sulfoxide
DPPA: diphenylphosphoryl azide
dppf: 1,1′-bis(diphenylphosphino)ferrocene
EDC or EDCI: 1-(3-dimethylaminopropyl)-3-ethylcarbodiimide hydrochloride
ee: enantiomeric excess
ESI: electrospray ionization
EA: ethyl acetate
EtOAc: ethyl acetate
EtOH: ethanol
FA: formic acid
h or hrs: hours
HATU: N,N,N′,N′-tetramethyl-O-(7-azabenzotriazol-1-yl)uronium hexafluorophosphate
HCl: hydrochloric acid
HPLC: high performance liquid chromatography
HOAc: acetic acid
IBX: 2-iodoxybenzoic acid
IPA: isopropyl alcohol
KHMDS: potassium hexamethyldisilazide
K₂CO₃: potassium carbonate
LAH: lithium aluminum hydride
LDA: lithium diisopropylamide
m-CPBA: meta-chloroperbenzoic acid
M: molar
MeCN: acetonitrile
MeOH: methanol
Me₂S: dimethyl sulfide
MeONa: sodium methylate
MeI: iodomethane
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
nBuLi: n-butyllithium
NaNO₂: sodium nitrite
NaOH: sodium hydroxide
Na₂SO₄: sodium sulfate
NBS: N-bromosuccinimide
NCS: N-chlorosuccinimide
NFSI: N-Fluorobenzenesulfonimide
NMO: N-methylmorpholine N-oxide
NMP: N-methylpyrrolidine
NMR: Nuclear Magnetic Resonance
° C.: degrees Celsius
Pd/C: Palladium on Carbon
Pd(OAc)₂: Palladium Acetate
PBS: phosphate buffered saline
PE: petroleum ether
POCl₃: phosphorus oxychloride
PPh₃: triphenylphosphine
PyBOP: (Benzotriazol-1-yloxy)tripyrrolidinophosphonium hexafluorophosphate
Rel: relative
R.T. or rt: room temperature
sat: saturated
SEMCl: chloromethyl-2-trimethylsilylethyl ether
SFC: supercritical fluid chromatography
SOCl₂: sulfur dichloride
tBuOK: potassium tert-butoxide
TBAB: tetrabutylammonium bromide
TBAI: tetrabutylammonium iodide
TEA: triethylamine
Tf: trifluoromethanesulfonate
TfAA, TFMSA or Tf₂O: trifluoromethanesulfonic anhydride
TFA: trifluoracetic acid
TIPS: triisopropylsilyl
THF: tetrahydrofuran
THP: tetrahydropyran
TLC: thin layer chromatography
TMEDA: tetramethylethylenediamine
pTSA: para-toluenesulfonic acid
wt: weight
Xantphos: 4,5-bis(diphenylphosphino)-9,9-dimethylxanthene

### Example 1: Synthesis of Intermediates

### Synthesis of 4-Benzylsulfanyl-2-methyl-aniline (Intermediate DE)

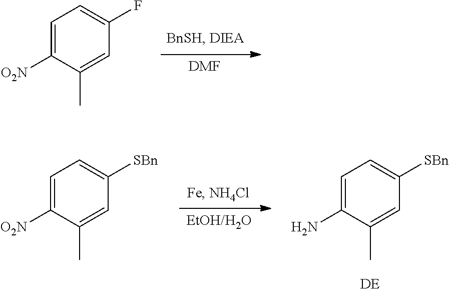

### Step—1—4-Benzylsulfanyl-2-methyl-1-nitro-benzene

A mixture of 4-fluoro-2-methyl-1-nitro-benzene (20.0 g, 128 mmol, CAS #446-33-3), BnSH (18.1 mL, 154 mmol), and DIEA (33.3 g, 257 mmol, 44.9 mL) in DMF (200 mL) was degassed and purged with N₂ for three times. Then the mixture was stirred at 80° C. for 16 hours under N₂ atmosphere. On completion, the reaction mixture was quenched with NaClO (10 mL) at 25° C., and then diluted with H₂O (10 mL) and extracted with EA (10 mL×3). The combined organic layers were washed with brine (10 mL×2), dried over anhydrous Na₂SO₄, filtered and concentrated in vacuo to give a residue. Then the residue was purified by column chromatography (SiO₂, Petroleum ether/Ethyl acetate=100/1 to 10/1) to give the title compound (26.0 g, 76% yield) as a yellow solid. ¹H NMR (400 MHz, CDCl₃) δ 8.05 (d, J=4.0 Hz, 1H) 7.51-7.37 (m, 5H) 7.30-7.25 (m, 2H) 4.34 (s, 2H) 2.69 (s, 3H). LC-MS (ESI⁺) m/z 260.0 (M+H)⁺.

### Step—2—4-Benzylsulfanyl-2-methyl-aniline

A mixture of 4-benzylsulfanyl-2-methyl-1-nitro-benzene (18.0 g, 69.4 mmol), Fe (23.2 g, 416 mmol), NH₄Cl (37.1 g, 694 mmol) in EtOH (180 mL) and H₂O (36 mL) was degassed and purged with N₂ for three times, and then the mixture was stirred at 80° C. for 1.5 hours under N₂ atmosphere. On completion, the reaction mixture was diluted with H₂O 100 mL and extracted with EA (60 mL×3). The combined organic layers were washed with brine (40 mL×2), dried over anhydrous Na₂SO₄, filtered and concentrated in vacuo to give a residue. The residue was purified by column chromatography (SiO₂, Petroleum ether/Ethyl acetate=100/1 to 10/1) to give the title compound (63 g, 98% yield) as a black oil. ¹H NMR (400 MHz, DMSO-d₆) δ 7.29-7.18 (m, 5H) 6.99 (s, 1H) 6.93 (d, J=1.6 Hz, 1H) 6.57 (d, J=8.0 Hz, 1H) 4.99 (s, 2H) 3.96 (s, 2H)

### Synthesis of 7′-Cyclopentyl-2′-methylsulfonyl-spiro[cyclopropane-1,5′-pyrrolo[2,3-d]pyrimidine]-6′-one (Intermediate HB)

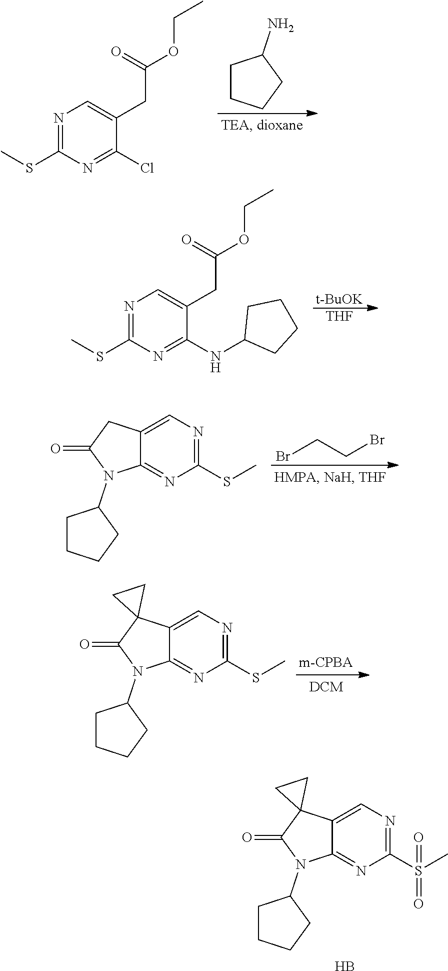

### Step 1—Ethyl 2-[4-(cyclopentylamino)-2-methylsulfanyl-pyrimidin-5-yl]acetate

To a solution of ethyl 2-(4-chloro-2-methylsulfanyl-pyrimidin-5-yl)acetate (1.00 g, 4.05 mmol, CAS #61727-34-2) in dioxane (10 mL) was added cyclopentanamine (690 mg, 8.11 mmol, CAS #1003-03-8) and TEA (820 mg, 8.11 mmol). The mixture was stirred at 60° C. for 1 hr. On completion, the reaction mixture was quenched with H₂O (20 mL) at 25° C., and then extracted with EA (3×10 mL). The combined organic layers were washed with brine (2×20 mL), dried over Na₂SO₄, filtered and concentrated in vacuo to give the title compound (1.10 g, 91% yield) as a white solid. ¹H NMR (400 MHz, CDCl₃) δ 7.81 (s, 1H), 5.73 (d, J=6.0 Hz, 1H), 4.47-4.39 (m, 1H), 4.14 (q, J=7.2 Hz, 2H), 3.31 (s, 2H), 2.51 (s, 3H), 2.12-2.03 (m, 2H), 1.78-1.71 (m, 2H), 1.68-1.61 (m, 2H), 1.53-1.44 (m, 2H), 1.25 (t, J=7.2 Hz, 3H). LC-MS (ESI⁺) m/z 296 (M+H)⁺.

### Step 2—7-Cyclopentyl-2-methylsulfanyl-5H-pyrrolo[2,3-d]pyrimidin-6-one

To a solution of ethyl 2-[4-(cyclopentylamino)-2-methylsulfanyl-pyrimidin-5-yl]acetate (1.00 g, 3.39 mmol) in THF (10 mL) was added t-BuOK (1.14 g, 10.1 mmol). The mixture was stirred at 35° C. for 1 hr. On completion, the reaction mixture was quenched with H₂O (20 mL) at 25° C., and then extracted with EA (3×20 mL). The combined organic layers were washed with brine (2×20 mL), dried over Na₂SO₄, filtered and concentrated in vacuo to give a residue. The residue was purified by column chromatography (SiO₂, Petroleum ether/Ethyl acetate=100/1 to 10/1) to give the title compound (800 mg, 94% yield) as a white solid. LC-MS (ESI⁺) m/z 250.0 (M+H)⁺.

### Step 3—7′-Cyclopentyl-2′-methylsulfanyl-spiro[cyclopropane-1,5′-pyrrolo[2,3-d]pyrimidine]-6′-one

To a suspension of NaH (5.01 g, 125 mmol, 60% dispersion in mineral oil) in THF (5 mL)N-[bis(dimethylamino) phosphoryl]-N-methylmethanamine (11.2 g, 62.5 mmol) was added a solution of 1,2-dibromoethane (11.7 g, 62.5 mmol, CAS #106-93-4) in THF (5 mL) dropwise and the reaction mixture was stirred at 0° C. for 30 mins. Next, 7-cyclopentyl-2-methylsulfanyl-5-Hpyrrolo[2,3-d]pyrimidin-6-one (7.80 g, 31.2 mmol) was added and the reaction mixture was heated to 50° C. for 1 hr. On completion, the reaction mixture was quenched with 1M HCl aq. (10 mL) at 25° C., and then extracted with EA (3×10 mL). The combined organic layers were washed with brine (2×10 mL), dried over Na₂SO₄, filtered and concentrated in vacuo to give a residue. Then the residue was purified by column chromatography (SiO₂, Petroleum ether/Ethyl acetate=100/1 to 10/1) to give the title compound (7.5 g, 87% yield) as pink oil. LC-MS (ESI⁺) m/z 276.0 (M+1)⁺. ¹H NMR (400 MHz, CDCl₃) δ 7.77 (s, 1H), 4.89-4.80 (m, 1H), 2.57 (s, 3H), 2.29-2.20 (m, 2H), 2.02-1.89 (m, 4H), 1.79 (q, J=4.4 Hz, 2H), 1.71-1.63 (m, 2H), 1.58 (q, J=4.0 Hz, 2H).

### Step 4—7′-Cyclopentyl-2′-methylsulfonyl-spiro[cyclopropane-1,5′-pyrrolo[2,3-d]pyrimidine]-6′-one

To a solution of 7′-cyclopentyl-2′-methylsulfanyl-spiro[cyclopropane-1,5′-pyrrolo[2,3-d]pyrimidine]-6′-one (5.15 g, 18.7 mmol) in DCM (50 mL) was added m-CPBA (11.3 g, 56.1 mmol, 85%) The mixture was stirred at 40° C. for 16 hrs. On completion, the reaction mixture was quenched with H₂O (30 mL) at 25° C., and then extracted with EA (3×30 mL). The combined organic layers were washed with brine (2×20 mL), dried over Na₂SO₄, filtered and concentrated under reduced pressure to give a residue. Then the residue was purified by column chromatography (SiO₂, Petroleum ether/Ethyl acetate=100/1 to 0/1) to give the title compound (3.00 g, 52% yield) as a white solid. ¹H NMR (400 MHz, CDCl₃) δ 8.05 (s, 1H), 4.96-488 (m, 1H), 3.34 (s, 3H), 2.23-2.16 (m, 2H), 2.05-2.00 (m, 2H), 2.00-1.95 (m, 4H), 1.82-1.78 (m, 2H), 1.72-1.67 (m, 2H). LC-MS (ESI⁺) m/z 307.7 (M+1)⁺.

### Synthesis of 4-[(7′-Cyclopentyl-6′-oxo-spiro[cyclopropane-1,5′-pyrrolo[2,3-d]pyrimidine]-2′-yl)amino]-3-methyl-benzenesulfonylchloride (Intermediate HC)

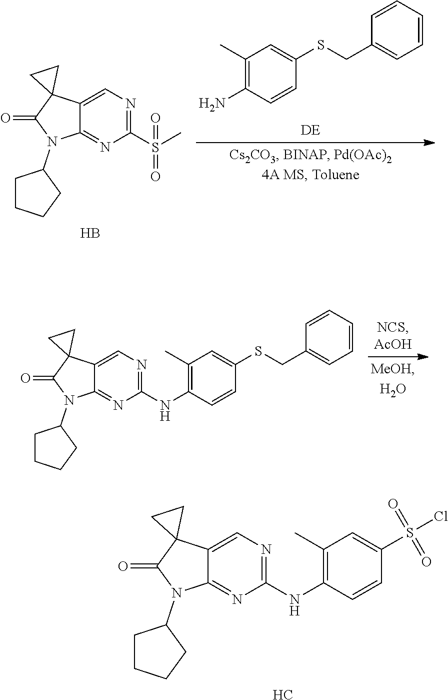

### Step 1—2′-(4-Benzylsulfanyl-2-methyl-anilino)-7′-cyclopentyl-spiro[cyclopropane-1,5′-pyrrolo[2,3-d]pyrimidine]-6′-one

A mixture of 7′-cyclopentyl-2′-methylsulfonyl-spiro[cyclopropane-1,5′-pyrrolo[2,3-d]pyrimidine]-6′-one (1.00 g, 3.25 mmol, Intermediate HB), 4-benzylsulfanyl-2-methyl-aniline (895 mg, 3.90 mmol, Intermediate DE), 4 Å molecular sieves (3.25 mmol), Cs₂CO₃ (3.18 g, 9.76 mmol), Pd(OAc)₂ (73.0 mg, 325 μmol) and BINAP (405 mg, 650 μmol) in toluene (10 mL) was degassed and purged with N₂ three times. Then the mixture was stirred at 100° C. for 12 hours under N₂ atmosphere. On completion, the reaction mixture was filtered and concentrated in vacuo to give a residue. Then the residue was purified by prep-HPLC (column: Phenomenex luna C18 150*25 mm*10 um; mobile phase: [water(FA)-ACN]; B %: 55%-85%, 17 min) to give the title compound (300 mg, 20% yield) as a white solid. LC-MS (ESI⁺) m/z 457.2 (M+1)⁺.

### Step 2—4-[(7′-Cyclopentyl-6′-oxo-spiro[cyclopropane-1,5′-pyrrolo[2,3-d]pyrimidine]-2′-yl)amino]-3-methyl-benzenesulfonylchloride

To a solution of 2′-(4-benzylsulfanyl-2-methyl-anilino)-7′-cyclopentyl-spiro[cyclopropane-1,5′-pyrrolo [2,3-d]pyrimidine]-6′-one (85.0 mg, 186 μmol) in H₂O (0.12 mL), ACN (2 mL) and AcOH (0.2 mL) was added NCS (74.5 mg, 558 μmol). The mixture was stirred at 25° C. for 1 hr. On completion, the reaction mixture was quenched with H₂O (10 mL) at 25° C., and then extracted with EA (3×10 mL). The combined organic layers were washed with brine (2×10 mL), dried over Na₂SO₄, filtered and concentrated in vacuo to give a residue. Then the residue was purified by column chromatography (SiO₂, Petroleum ether/Ethyl acetate=100/1 to 1/1) to give the title compound (80 mg, 99% yield) as a white solid. LC-MS (ESI⁺) m/z 433.0 (M+1)⁺.

### Synthesis of Ethylsulfanyl-2-methyl-aniline (Intermediate LK)

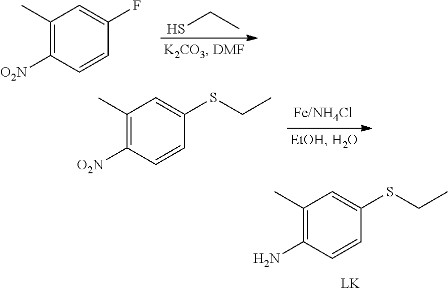

### Step 1—4-Ethylsulfanyl-2-methyl-1-nitro-benzene

To a solution of 4-fluoro-2-methyl-1-nitro-benzene (1.00 g, 6.45 mmol, CAS #446-33-3) in DMF (10 mL) was added EtSH (1.05 g, 16.9 mmol, CAS #75-08-1) and K₂CO₃ (2.67 g, 19.3 mmol). The mixture was stirred at 25° C. for 16 hrs. On completion, the mixture was diluted with H₂O (50 mL), extracted with EA (3×50 mL), washed with brine (3×50 mL), dried with anhydrous Na₂SO₄, filtered and concentrated in vacuo to give the title compound (1.00 g, 78% yield) as a colorless oil. ¹H NMR (400 MHz, DMSO-d₆) δ 7.96 (d, J=8.4 Hz, 1H), 7.36 (d, J=1.6 Hz, 1H), 7.31 (dd, J=2.0, 8.4 Hz, 1H), 3.11 (q, J=7.2 Hz, 2H), 2.53 (s, 3H), 1.29 (t, J=7.2 Hz, 3H). LC-MS (ESI⁺) m/z 198.0 (M+H)⁺.

### Step 2—4-Ethylsulfanyl-2-methyl-aniline

To a solution of 4-ethylsulfanyl-2-methyl-1-nitro-benzene (1.00 g, 5.07 mmol) in mixture solution of H₂O (10 mL) and EtOH (10 mL) was added Fe (1.70 g, 30.4 mmol) and NH₄Cl (2.71 g, 50.7 mmol). The mixture was stirred at 80° C. for 4 hrs. On completion, the mixture was filtered and concentrated in vacuo, then dissolved with EA (20 mL), diluted with H₂O (20 mL), and extracted with EA (3×20 mL). The combined organic layer was washed with brine (3×20 mL), dried with anhydrous Na₂SO₄, filtered and concentrated in vacuo to give the title compound (840 mg, 99% yield) as a black oil. ¹H NMR (400 MHz, DMSO-d₆) δ 7.00 (s, 1H), 6.96 (dd, J=2.0, 8.4 Hz, 1H), 6.55 (d, J=8.0 Hz, 1H), 4.96 (s, 2H), 2.69 (q, J=7.2 Hz, 2H), 2.02 (s, 3H), 1.11 (t, J=7.2 Hz, 3H). LC-MS (ESI⁺) m/z 168.1 (M+H)⁺.

### Synthesis of 8-Cyclopentyl-2-methylsulfanyl-pyrido[2,3-d]pyrimidin-7-one (Intermediate HN)

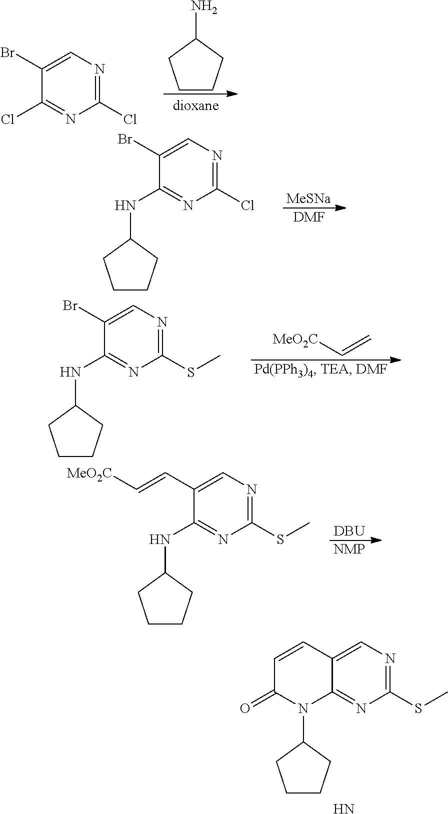

### Step 1—5-Bromo-2-chloro-N-cyclopentyl-pyrimidin-4-amine

To a solution of 5-bromo-2,4-dichloro-pyrimidine (5.00 g, 21.9 mmol, CAS #36082-50-5) in dioxane (100 mL) was added cyclopentanamine (2.24 g, 26.3 mmol, CAS #1003-03-8). The mixture was stirred at 25° C. for 6 hrs. On completion, the reaction mixture was with H₂O (100 mL) at 25° C., and then extracted with ethyl acetate (3×100 mL). The combined organic layers were washed with brine (2×100 mL), dried over Na₂SO₄, filtered and concentrated in vacuo to give a residue. Then the residue was purified by column chromatography (SiO₂, Petroleum ether/Ethyl acetate=100/1 to 1/1) to give the title compound (7.50 g, 61% yield) as a white solid. ¹H NMR (400 MHz, DMSO-d₆) δ 8.22 (s, 1H), 7.36 (d, J=7.2 Hz, 1H), 4.35-4.26 (m, 1H), 1.92-1.88 (m, 2H), 1.72-1.65 (m, 2H), 1.63-1.51 (m, 4H).

### Step 2—5-Bromo-N-cyclopentyl-2-methylsulfanyl-pyrimidin-4-amine

To a solution of 5-bromo-2-chloro-N-cyclopentyl-pyrimidin-4-amine (2.00 g, 7.23 mmol) in DMF (20 mL) was degassed and purged with N₂ three times, then NaSMe (1.29 g, 18.4 mmol) was added to the mixture. The mixture was stirred at 25° C. for 16 hrs under N₂ atmosphere. On completion, the reaction mixture was quenched with H₂O (20 mL) at 25° C., and then extracted with EA (3×20 mL). The combined organic layers were washed with brine (2×10 mL), dried over Na₂SO₄, filtered and concentrated in vacuo to give a residue. Then the crude product was purified by reversed-phase HPLC (0.1% FA condition) to give the title compound (2.00 g, 95% yield) as off-white oil. ¹H NMR (400 MHz, CDCl₃) δ 8.05 (s, 1H), 5.27 (d, J=4.0 Hz, 1H), 4.44-4.36 (m, 1H), 2.50 (s, 3H), 2.15-2.07 (m, 2H), 1.78-1.63 (m, 4H), 1.53-1.45 (m, 2H).

### Step 3—Methyl (E)-3-[4-(cyclopentylamino)-2-methylsulfanyl-pyrimidin-5-yl]prop-2-enoate

A mixture of 5-bromo-N-cyclopentyl-2-methylsulfanyl-pyrimidin-4-amine (2.00 g, 6.94 mmol), TEA (2.11 g, 20.8 mmol), Pd(PPh₃)₄ (801 mg, 693 μmol) in DMF (20 mL) was degassed and purged with N₂ three times. Then methyl prop-2-enoate (3.11 g, 36.1 mmol, CAS #96-33-3) was added to the mixture, and then the mixture was stirred at 90° C. for 16 hours under N₂ atmosphere. On completion, the reaction mixture was quenched with H₂O (20 mL) at 25° C., and then extracted with EA (3×10 mL). The combined organic layers were washed with brine (2×10 mL), dried over Na₂SO₄ filtered and concentrated under reduced pressure to give a residue. Then the crude product was purified by reversed-phase HPLC (0.1% FA condition) to give the title compound (1.37 g, 67% yield) as a yellow solid. ¹H NMR (400 MHz, CDCl₃) δ 8.14 (s, 1H), 7.49 (d, J=16.0 Hz, 1H), 6.27 (dd, J=1.2, 15.6 Hz, 1H), 5.08 (d, J=6.0 Hz, 1H), 4.52-4.44 (m, 1H), 3.83-3.79 (m, 3H), 2.58-2.51 (m, 3H), 2.19-2.08 (m, 2H), 1.81-1.62 (m, 4H), 1.53-1.45 (m, 2H).

### Step 4—8-Cyclopentyl-2-methylsulfanyl-pyrido[2,3-d]pyrimidin-7-one

To a solution of methyl (E)-3-[4-(cyclopentylamino)-2-methylsulfanyl-pyrimidin-5-yl]prop-2-enoate (1.00 g, 3.41 mmol) in NMP (10 mL) was added DBU (2.59 g, 17.0 mmol). The mixture was stirred at 120° C. for 1 hr. On completion, the reaction mixture was quenched with H₂O (20 mL) at 25° C., and then extracted with EA (3×20 mL). The combined organic layers were washed with brine (2×20 mL), dried over Na₂SO₄, filtered and concentrated in vacuo to give a residue. Then the residue was purified by column chromatography (SiO₂, Petroleum ether/Ethyl acetate=100/1 to 10/1) to give the title compound (484 mg, 54% yield) as a yellow solid. LC-MS (ESI⁺) m/z 262.0 (M+1)⁺.

### Synthesis of 6-Chloro-8-cyclopentyl-2-methylsulfonyl-pyrido[2,3-d]pyrimidin-7-one (Intermediate KM)

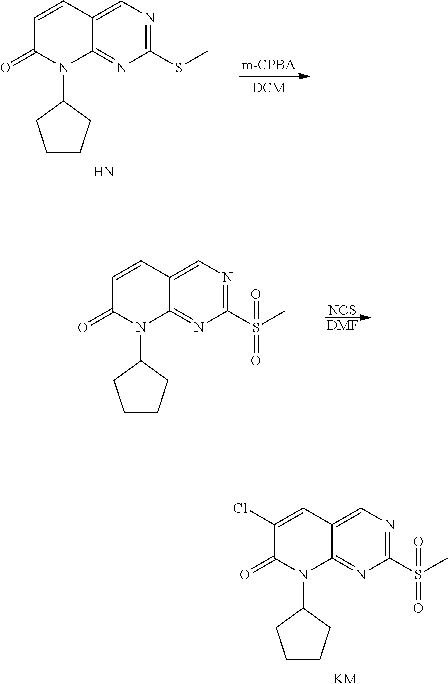

### Step 1—8-Cyclopentyl-2-methylsulfonyl-pyrido[2,3-d]pyrimidin-7-one

To a solution of 8-cyclopentyl-2-methylsulfanyl-pyrido[2,3-d]pyrimidin-7-one (2.00 g, 7.65 mmol, Intermediate HN) in DCM (20 mL) was added m-CPBA (6.21 g, 30.6 mmol, 85% solution). The mixture was stirred at 40° C. for 3 hrs. On completion, the reaction mixture was quenched with Na₂CO₃ aq. (10 mL) at 25° C., and then extracted with EA (3×30 mL). The combined organic layers were washed with brine (2×20 mL), dried over Na₂SO₄, filtered and concentrated in vacuo to give the title compound (2.00 g, 89% yield) as a yellow oil. LC-MS (ESI⁺) m/z 293.9 (M+1)⁺.

### Step 2—6-Chloro-8-cyclopentyl-2-methylsulfonyl-pyrido[2,3-d]pyrimidin-7-one

To a solution of 8-cyclopentyl-2-methylsulfonyl-pyrido[2,3-d]pyrimidin-7-one (100 mg, 340.9 μmol) in DMF (1 mL) was added NCS (500 mg, 3.75 mmol). The mixture was stirred at 70° C. for 16 hrs. On completion, the reaction mixture was filtered and concentrated in vacuo to give a residue. Then the crude product was purified by reversed-phase HPLC (0.10% FA condition) to give the title compound (280 mg, 24% yield) as a brown solid. LC-MS (ESI⁺) m/z 327.9 (M+1)⁺. ¹H NMR (400 MHz, CDCl₃) δ 8.94 (s, 1H), 7.96 (s, 1H), 6.08-5.92 (m, 1H), 3.40 (s, 3H), 2.32-2.23 (m, 2H), 2.22-2.13 (m, 2H), 2.04-1.96 (m, 2H), 1.77-1.70 (m, 2H).

### Synthesis of 6-Chloro-8-cyclopentyl-2-(4-ethylsulfanyl-2-methyl-anilino)pyrido[2,3-d]pyrimidin-7-one (Intermediate LL)

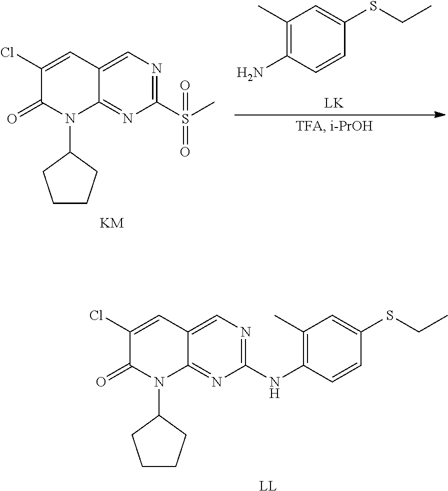

4-Ethylsulfanyl-2-methyl-aniline (53.5 mg, 320 μmol, Intermediate LK), 6-chloro-8-cyclopentyl-2-methylsulfonyl-pyrido[2,3-d]pyrimidin-7-one (70.0 mg, 213 μmol, Intermediate KM) and TFA (243 mg, 2.14 mmol) were placed in a microwave tube in i-PrOH (1 mL). The sealed tube was heated at 120° C. for 2 hrs under microwave. On completion, the mixture was cooled, diluted with H₂O (10 mL), and extracted with EA (3×10 mL). The combined organic phases were washed with brine (2×10 mL), dried over Na₂SO₄, filtered and concentrated in vacuo. The residue was purified by prep-TLC (SiO₂, PE/EA=2/1) to give the title compound (45.0 mg, 50% yield) as a yellow oil. ¹H NMR (400 MHz, DMSO-d₆) δ 9.46 (s, 1H), 8.68 (s, 1H), 8.13 (s, 1H), 7.31 (d, J=8.4 Hz, 1H), 7.25 (d, J=2.0 Hz, 1H), 7.18 (dd, J=2.0, 8.4 Hz, 1H), 5.74-5.60 (m, 1H), 2.96 (q, J=7.2 Hz, 2H), 2.19 (s, 3H), 2.14-2.05 (m, 2H), 1.67-1.37 (m, 6H), 1.23 (t, J=7.2 Hz, 3H). LC-MS (ESI⁺) m/z 415.2 (M+H)⁺.

### Synthesis of 4-(3-Methyl-4-nitro-phenyl)sulfonylpiperidine (Intermediate LM)

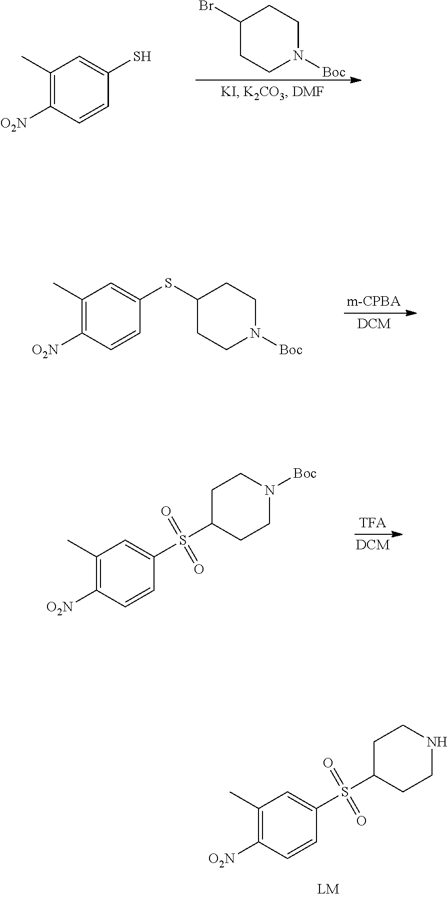 [SMILES: Cc1cc(S)ccc1[N+](=O)[O-] ; CC(C)(C)OC(=O)N1CCC(Br)CC1 ; Cc1cc(SC2CCN(C(=O)OC(C)(C)C)CC2)ccc1[N+](=O)[O-] ; Cc1cc(S(=O)(=O)C2CCN(C(=O)OC(C)(C)C)CC2)ccc1[N+](=O)[O-] ; Cc1cc(S(=O)(=O)C2CCNCC2)ccc1[N+](=O)[O-]]

### Step 1—Tert-butyl 4-(3-methyl-4-nitro-phenyl)sulfanylpiperidine-1-carboxylate

A mixture of 3-methyl-4-nitro-benzenethiol (900 mg, 5.32 mmol, CAS #53827-87-5), tert-butyl 4-bromopiperidine-1-carboxylate (2.11 g, 7.98 mmol, CAS #180695-79-8), K₂CO₃ (808 mg, 5.85 mmol), and KI (88.3 mg, 531 μmol) in DMF (2 mL) was degassed and purged with N₂ three times, and then the mixture was stirred at 60° C. for 40 hrs under N₂ atmosphere. On completion, the residue was diluted with H₂O (50 mL) and extracted with EA (3×100 mL). The combined organic layers were washed with brine (2×100 mL), dried over with anhydrous Na₂SO₄, filtered and concentrated under reduced pressure and purified by column chromatography (SiO₂, Petroleum ether/Ethyl acetate=100/1 to 15/1, P1 Rf=0.60) to afford the title compound (140 mg, 7% yield) as white oil. ¹H NMR (400 MHz, CDCl₃) δ 5.81 (dt, J=2.0, 4.0 Hz, 1H), 5.64 (d, J=9.2 Hz, 1H), 3.48 (t, J=5.6 Hz, 2H), 3.34-3.25 (m, 1H), 2.16-2.05 (m, 3H), 1.46 (s, 9H). LC-MS (ESI⁺) m/z 337.9 (M+H)⁺.

### Step 2—6-(Methoxymethyl)-N,N-bis[(4-methoxyphenyl)methyl]spiro[3.3]heptan-2-amine

To a solution of tert-butyl 4-(3-methyl-4-nitro-phenyl) sulfanylpiperidine-1-carboxylate (140 mg, 397 μmol) in DCM (2 mL) was added m-CPBA (342 mg, 1.19 mmol, 60% dispersion in mineral oil) at 0° C. The mixture was stirred at 25° C. for 2 hrs. On completion, the reaction mixture was quenched with Na₂S₂O₃ aq (10 mL) at 25° C., and then extracted with DCM (3×50 mL). The combined organic layers were washed with NaHCO₃ (2×50 mL), dried over with anhydrous Na₂SO₄, filtered and concentrated in vacuo to give a residue. Then the crude was purified by prep-TLC (SiO₂, PE:EA=5:1, Rf=0.50) to afford the title compound (60 mg, 50% yield) as a yellow solid. ¹HNMR (400 MHz, DMSO-d₆) δ 7.38-7.07 (m, 4H), 6.88 (s, 4H), 3.73 (s, 6H), 3.29-3.26 (m, 2H), 3.19 (d, J=6.4 Hz, 3H), 3.17 (s, 3H), 3.01-2.80 (m, 1H), 2.39-2.25 (m, 2H), 2.02-1.83 (m, 4H), 1.79-1.54 (m, 4H). LC-MS (ESI⁺) m/z 328.9 (M+H)⁺.

### Step 3—4-(3-Methyl-4-nitro-phenyl)sulfonylpiperidine

To a solution of tert-butyl 4-(3-methyl-4-nitro-phenyl) sulfonylpiperidine-1-carboxylate (60 mg, 156 μmol) in DCM (1 mL) was added TFA (1.54 g, 13.5 mmol). The mixture was stirred at 25° C. for 0.5 hr. On completion, the reaction mixture was concentrated in vacuo to afford the title compound (60 mg, 96% yield, TFA) as yellow oil. LC-MS (ESI⁺) m/z 285.0 (M+H)⁺.

### Synthesis of 2-Methyl-4-[(1-methyl-4-piperidyl)sulfonyl]aniline (Intermediate LN)

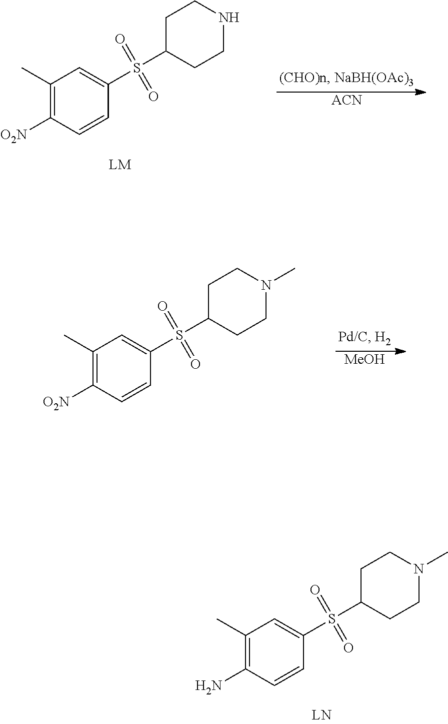

### Step 1—1-Methyl-4-(3-methyl-4-nitro-phenyl) sulfonyl-piperidine

To a solution of 4-(3-methyl-4-nitro-phenyl)sulfonylpiperidine (190 mg, 476 μmol TFA, Intermediate LM) and formaldehyde (143 mg, 4.77 mmol) in ACN (2 mL) was added NaBH(OAc)₃ (323 mg, 1.53 mmol). The mixture was stirred at 25° C. for 12 hrs. On completion, the reaction mixture was concentrated in vacuo to give a residue and the crude product was purified by prep-HPLC (column: Phenomenex Luna C8 250*50 mm*10 um; mobile phase: [water(FA)-ACN]; B %: 0%-30%, 9 min) to give the title compound (130 mg, 91% yield) as colorless oil. LC-MS (ESI⁺) m/z 299.1 (M+H)⁺.

### Step 2—2-Methyl-4-[(1-methyl-4-piperidyl)sulfonyl]aniline

To a solution of 1-methyl-4-(3-methyl-4-nitro-phenyl)sulfonyl-piperidine (120 mg, 402 μmol) in MeOH (1 mL) was added Pd/C (4.02 μmol, 10 wt %) and purged with H₂ three times, and then the mixture was stirred at 25° C. for 2 hours under H₂ (15 PSI). On completion, the reaction mixture was filtered and concentrated in vacuo to give the title compound (100 mg, 92% yield) as colorless oil. ¹H NMR (400 MHz, DMSO-d₆) δ 7.30-7.26 (m, 2H), 6.67 (d, J=8.4 Hz, 1H), 5.91 (s, 2H), 3.05-2.97 (m, 3H), 2.34-2.29 (m, 3H), 2.26-2.19 (m, 2H), 2.06 (s, 3H), 1.84 (d, J=12.4 Hz, 2H), 1.57-1.46 (m, 2H).

### Synthesis of 5-Bromo-2-chloro-N-isopropyl-pyrimidin-4-amine (Intermediate DF)

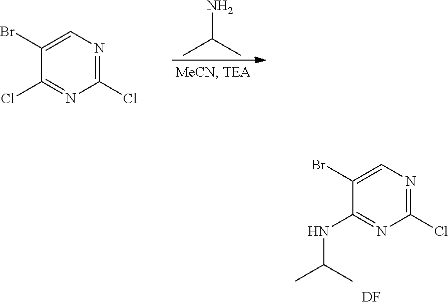

To a solution of 5-bromo-2,4-dichloro-pyrimidine (10.0 g, 43.8 mmol, 5.62 mL, CAS #36082-50-5) in ACN (250 mL) was added TEA (5.77 g, 57.0 mmol, 7.94 mL) and propan-2-amine (3.37 g, 57.0 mmol, 4.90 mL) at 0° C. for 30 min. Then the mixture was stirred for 15.5 hours at 25° C. On completion, the reaction mixture was diluted with H₂O (200 mL) and extracted with EA (3×100 mL). The combined organic layers were washed with brine (2×100 mL), dried over Na₂SO₄, filtered and concentrated under reduced pressure to give the title compound (10 g, 90% yield) as a white solid. LC-MS (ESI⁺) m/z 251.8 (M+H)⁺.

### Synthesis of 8-isopropyl-2-methylsulfanyl-pyrido[2,3-d]pyrimidin-7-one (Intermediate DN)

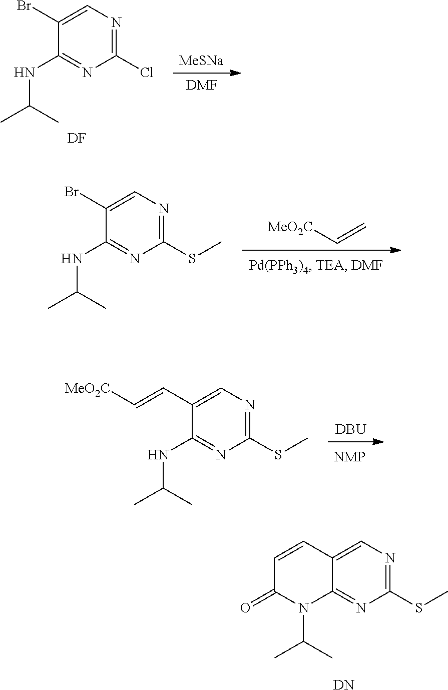

### Step 1—5-Bromo-N-isopropyl-2-methylsulfanyl-pyrimidin-4-amine

To a solution of 5-bromo-2-chloro-N-isopropyl-pyrimidin-4-amine (10.0 g, 39.9 mmol, Intermediate DF) in DMF (110 mL) was added NaSMe (7.12 g, 101 mmol, 6.47 mL). The mixture was stirred at 25° C. for 16 hrs under N₂. On completion, the reaction mixture was quenched with H₂O (100 mL) at 25° C., and then extracted with EA (100 mL×3). The combined organic layers were washed with brine (100 mL×2), dried over anhydrous Na₂SO₄, filtered and concentrated in vacuo to give the title compound (9.50 g, 90% yield) as a yellow solid. ¹H NMR (400 MHz, DMSO-d₆) δ 8.12 (s, 1H), 7.95 (s, 1H), 4.32-4.25 (m, 1H), 2.89 (s, 3H), 2.73 (s, 3H), 2.41 (s, 3H). LC-MS (ESI⁺) m/z 263.8 (M+H)⁺.

### Step 2—Methyl (E)-3-[4-(isopropylamino)-2-methylsulfanyl-pyrimidin-5-yl]prop-2-enoate

A mixture of 5-bromo-N-isopropyl-2-methylsulfanyl-pyrimidin-4-amine (9.50 g, 36.2 mmol), methyl prop-2-enoate (22.3 g, 259 mmol, 23.3 mL, CAS #96-33-3), Pd(PPh₃)₄ (4.19 g, 3.62 mmol), and TEA (11.0 g, 108 mmol, 15.0 mL) in DMF (100 mL) was degassed and purged with N₂ three times. Then the mixture was stirred at 90° C. for 32 hrs under N₂ atmosphere. On completion, the reaction mixture was quenched with H₂O (100 mL) at 25° C., and then extracted with EA (100 mL×3). The combined organic layers were washed with brine (100 mL×2), dried over anhydrous Na₂SO₄, filtered and concentrated in vacuo to give a residue. The residue was purified by column chromatography (SiO₂, Petroleum ether/Ethyl acetate=10/1 to 3/1) (Rf-0.40, PE:EA=1:1) to give the title compound (5.80 g, 59% yield) as a yellow solid. ¹H NMR (400 MHz, DMSO-d₆) δ 8.43-8.30 (m, 1H), 7.79 (d, J=15.6 Hz, 1H), 7.49 (d, J=7.2 Hz, 1H), 6.55-6.43 (m, 1H), 4.35 (d, J=6.8, 13.4 Hz, 1H), 3.71 (s, 3H), 2.44 (s, 3H), 1.19 (d, J=6.4 Hz, 6H). LC-MS (ESI⁺) m/z 268.1 (M+H)⁺.

### Step 3—8-Isopropyl-2-methylsulfanyl-pyrido[2,3-d]pyrimidin-7-one

A mixture of methyl (E)-3-[4-(isopropylamino)-2-methylsulfanyl-pyrimidin-5-yl]prop-2-enoate (5.73 g, 21.4 mmol), DBU (16.3 g, 107 mmol, 16.1 mL) in NMP (50.0 mL) was degassed and purged with N₂ three times. Then the mixture was stirred at 120° C. for 1 hr under N₂ atmosphere. On completion, the mixture was diluted with H₂O (300 mL), and extracted with DCM (3×100 mL). The combined organic layer was washed with brine (3×100 mL), then dried with anhydrous Na₂SO₄, filtered and the filtrate was concentrated in vacuo. The mixture was purified by reversed phase (0.1% FA) to give the title compound (4.20 g, 83% yield) as a white solid. ¹H NMR (400 MHz, DMSO-d₆) δ 8.84 (s, 1H), 7.86 (d, J=9.6 Hz, 1H), 6.56 (d, J=9.6 Hz, 1H), 5.75-5.56 (m, 1H), 2.59 (s, 3H), 1.53 (d, J=6.8 Hz, 6H). LC-MS (ESI⁺) m/z 236.1 (M+H)⁺.

### Synthesis of 6-Chloro-8-isopropyl-2-methylsulfonyl-pyrido[2,3-d]pyrimidin-7-one (Intermediate KP)

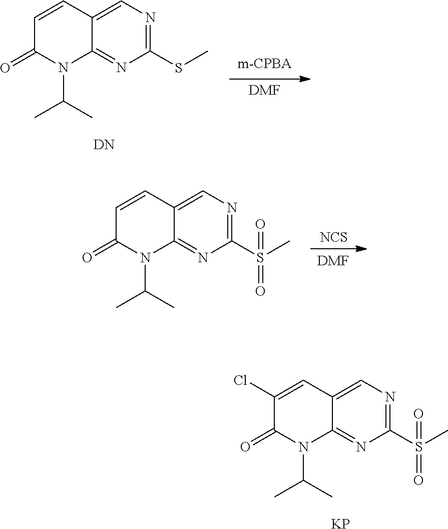

### Step 1—8-Isopropyl-2-methylsulfonyl-pyrido[2,3-d]pyrimidin-7-one

To a solution of 8-isopropyl-2-methylsulfanyl-pyrido[2,3-d]pyrimidin-7-one (2.20 g, 9.35 mmol, Intermediate DN) in DCM (20.0 mL) was added m-CPBA (7.59 g, 37.0 mmol, 85% solution). The mixture was stirred at 40° C. for 3 hrs. On completion, the reaction mixture was quenched with Na₂CO₃ aq. (100 mL) at 25° C., and then extracted with EA (3×100 mL). The combined organic layers were washed with brine (2×100 mL), dried over anhydrous Na₂SO₄, filtered and concentrated in vacuo to give the title compound (2.10 g, 84% yield) as a yellow solid. ¹H NMR (400 MHz, DMSO-d₆) δ 9.27 (s, 1H), 8.07 (d, J=9.6 Hz, 1H), 6.87 (d, J=9.6 Hz, 1H), 5.65 (td, J=6.8, 13.6 Hz, 1H), 3.46 (s, 3H), 1.56 (d, J=7.2 Hz, 6H). LC-MS (ESI⁺) m/z 267.9 (M+H)⁺.

### Step 2—6-Chloro-8-isopropyl-2-methylsulfonyl-pyrido[2,3-d]pyrimidin-7-one

To a solution of 8-isopropyl-2-methylsulfonyl-pyrido[2,3-d]pyrimidin-7-one (100 mg, 374 μmol) in DMF (1.50 mL) was added NCS (149 mg, 1.12 mmol). The mixture was stirred at 70° C. for 16 hrs. On completion, the mixture was concentrated in vacuo. The mixture was purified by reversed phase (0.1% FA) to give the title compound (74.0 mg, 65% yield) as a yellow solid. ¹H NMR (400 MHz, DMSO-d₆) δ 9.27-9.25 (m, 1H), 8.68-8.37 (m, 1H), 5.90-5.58 (m, 1H), 3.48 (d, J=2.4 Hz, 3H), 1.58 (s, 6H). LC-MS (ESI⁺) m/z 301.8 (M+H)⁺.

### Synthesis of 4-Methylsulfonylcyclohexanamine (Intermediate LO)

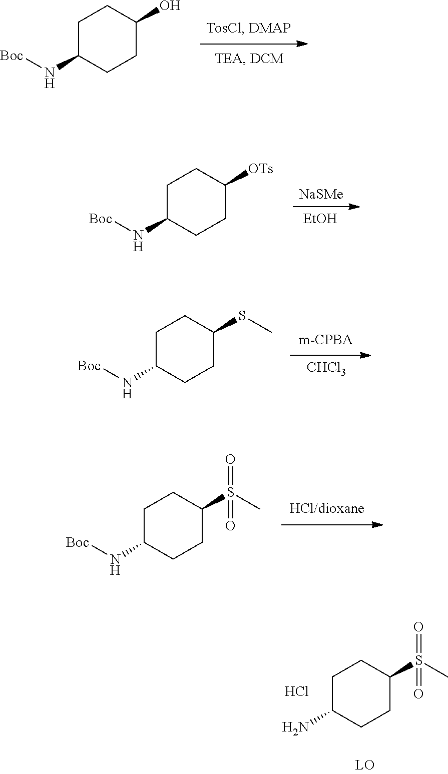

### Step 1—[4-(Tert-butoxycarbonylamino)cyclohexyl] 4-methylbenzenesulfonate

To a solution of tert-butyl N-(4-hydroxycyclohexyl)carbamate (2.00 g, 9.29 mmol, CAS #167081-25-6) in DCM (20 mL) was added TEA (4.70 g, 46.4 mmol) and TosCl (2.30 g, 12.0 mmol) at 0° C., then the mixture was stirred at 25° C. for 2 hrs. Next, DMAP (113 mg, 928 μmol) was added dropwise at 25° C., then the mixture was stirred at 25° C. for 14 hrs. On completion, the reaction mixture was concentrated in vacuo. The residue was diluted with H₂O (60 mL) and extracted with EA (30 mL×2). The combined organic layers were washed with brine (30 mL×2), dried over Na₂SO₄, filtered and concentrated under reduced pressure to give the title compound (3.4 g, 99% yield) as a white solid. ¹H NMR (400 MHz, DMSO-d₆) δ 7.78 (d, J=8.4 Hz, 2H), 7.47 (d, J=8.4 Hz, 2H), 4.58 (s, 1H), 3.66 (s, 1H), 2.42 (s, 3H), 1.72-1.64 (m, 2H), 1.36 (d, J=4.0 Hz, 15H).

### Step 2—Tert-butyl N-(4-methylsulfanylcyclohexyl)carbamate

To a solution of [4-(tert-butoxycarbonylamino)cyclohexyl] 4-methylbenzenesulfonate (500 mg, 1.35 mmol) in EtOH (5 mL) was added NaSMe (140 mg, 2.00 mmol) at 25° C. The mixture was then warmed to 70° C. and stirred for 20 hrs. On completion, the reaction mixture was concentrated in vacuo. The residue was diluted with water (60 mL) and extracted with EA (40 mL×2). The combined organic layer was dried over Na₂SO₄, filtered and concentrated in vacuo. The residue was purified by column chromatography (SiO₂, PE:EA=50:1 to 40:1, PE:EA=5:1, P1:Rf=0.5) to give the title compound (85.0 mg, 26% yield) as a white solid. ¹H NMR (400 MHz, CDCl₃) δ 4.38 (s, 1H), 3.43 (d, J=3.6 Hz, 1H), 2.50-2.40 (m, 1H), 2.09 (s, 3H), 2.09-2.04 (m, 3H), 1.44 (s, 9H), 1.42-1.33 (m, 2H), 1.21-1.09 (m, 2H).

### Step 3—Tert-butyl N-(4-methylsulfonylcyclohexyl)carbamate

To a solution of tert-butyl N-(4-methylsulfanylcyclohexyl)carbamate (80.0 mg, 326 μmol) in CHCl₃ (2 mL) was added m-CPBA (264 mg, 1.30 mmol, 85% solution). The mixture was stirred at 40° C. for 2 hrs. On completion, the reaction was diluted with water (30 mL) and extracted with DCM (20 mL×2). The combined organic layers were washed with K₂CO₃ aqueous solution (20 mL×3), dried over Na₂SO₄, filtered and concentrated in vacuo to give the title compound (65.0 mg, 72% yield) as a white solid. ¹H NMR (400 MHz, CDCl₃) δ 4.57-4.33 (m, 1H), 3.57-3.37 (m, 1H), 2.85 (s, 3H), 2.34-2.19 (m, 4H), 1.74-1.62 (m, 2H), 1.46 (s, 9H), 1.34-1.14 (m, 2H).

### Step 4—4-Methylsulfonylcyclohexanamine

A solution of tert-butyl N-(4-methylsulfonylcyclohexyl)carbamate (65.0 mg, 234 μmol) in HCl/dioxane (2 mL) was stirred at 25° C. for 1 hr. On completion, the reaction mixture was concentrated in vacuo to give the title compound (45.0 mg, 89% yield) as white solid. LCMS (ESI⁺) m/z 178.1 (M+H)⁺.

### Synthesis of 4-Ethylsulfonyl-2-methyl-aniline (Intermediate LP)

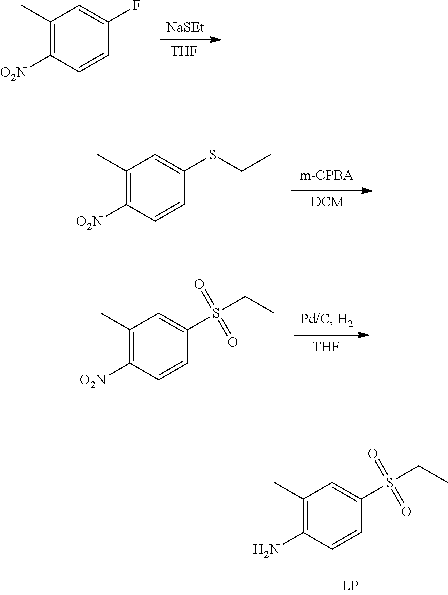

### Step 1—4-Ethylsulfanyl-2-methyl-1-nitro-benzene

To a solution of 4-fluoro-2-methyl-1-nitro-benzene (500 mg, 3.22 mmol, CAS #446-33-3) in THF (5 mL) was added NaSEt (813 mg, 9.67 mmol, CAS #811-51-8). The mixture was stirred at 25° C. for 3 hrs. The reaction mixture was partitioned between H₂O (20 mL) and DCM (20 mL). The organic phase was separated, washed with brine (20 mL), dried over anhydrous Na₂SO₄, filtered and concentrated in vacuo to give the title compound (630 mg, 99% yield) as a yellow oil. LCMS (ESI⁺) m/z 197.9 (M+H)⁺.

### Step 2—4-Ethylsulfonyl-2-methyl-1-nitro-benzene

To a solution of 4-ethylsulfanyl-2-methyl-1-nitro-benzene (200 mg, 1.01 mmol) in DCM (2 mL) was added m-CPBA (714 mg, 4.06 mmol). The mixture was stirred at 40° C. for 2 hrs. The reaction mixture was partitioned between Na₂SO₃ (20 mL) and DCM (20 mL). The organic phase was separated, washed with brine (20 mL), dried over anhydrous Na₂SO₄, filtered and concentrated in vacuo to give the title compound (230 mg, 98% yield) as a yellow oil. LCMS (ESI⁺) m/z 230.1 (M+H)⁺.

### Step 3—4-Ethylsulfonyl-2-methyl-aniline

To a solution of 4-ethylsulfonyl-2-methyl-1-nitro-benzene (230 mg, 1.00 mmol) in THF (2 mL) was added Pd/C (118 mg, 100 μmol, 10 wt %) under N₂. The suspension was degassed in vacuo and purged with H₂ three times. The mixture was stirred at 25° C. for 16 hours under H₂ (15 psi). The reaction mixture was filtered and concentrated in vacuo to give the title compound (190 mg, 95% yield) as a yellow oil. LCMS (ESI⁺) m/z 199.9 (M+H)⁺.

### Synthesis of 2-Chloro-4-(1-methylpyrazol-4-yl)-5-(trifluoromethyl)pyrimidine (Intermediate LQ)

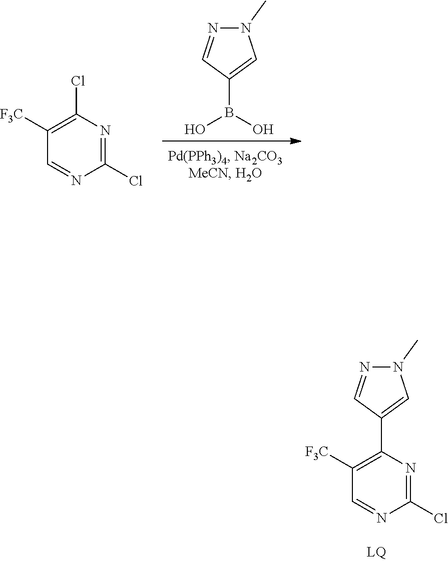

To a solution of 2,4-dichloro-5-(trifluoromethyl)pyrimidine (3.50 g, 16.1 mmol, CAS #3932-97-6) in ACN (35 mL) and H₂O (15 mL) was added (1-methylpyrazol-4-yl)boronic acid (2.03 g, 16.1 mmol, CAS #847818-55-7) and Na₂CO₃ (5.13 g, 48.3 mmol). Then Pd(PPh₃)₄ (1.86 g, 1.61 mmol) was added, and the mixture was stirred at 80° C. for 2 hrs under N₂ atmosphere. On completion, the mixture was filtered and the filtrate was diluted with water (50 mL) and extracted with EA (50 mL×3). The combined organic layers were dried over anhydrous Na₂SO₄, then concentrated in vacuo to give a residue. The residue was purified by column chromatography (SiO₂, PE:EA=20:1 to 8:1) to give the title compound (1.60 g, 37% yield) as a white solid. ¹H NMR (400 MHz, DMSO-d₆) δ 9.04 (s, 1H), 8.49 (s, 1H), 8.02 (s, 1H), 3.95 (s, 3H). LC-MS (ESI⁺) m/z 262.7 (M+H)⁺.

### Synthesis of N-isopropylacetamidine (Intermediate DW)

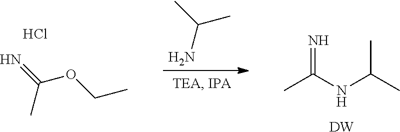

To a solution of ethyl ethanimidate hydrochloride (10.0 g, 80.9 mmol, CAS #2208-07-3) in IPA (60 mL) was added TEA (8.19 g, 80.9 mmol) and propan-2-amine (4.78 g, 80.9 mmol, CAS #4432-77-3). The mixture was stirred at 25° C. for 1 hour. On completion, the reaction mixture was concentrated in vacuo to give the title compound (5.5 g, 67% yield) as a colorless oil.

### Synthesis of 3-Chloro-4,4-diethoxy-butan-2-one (Intermediate DX)

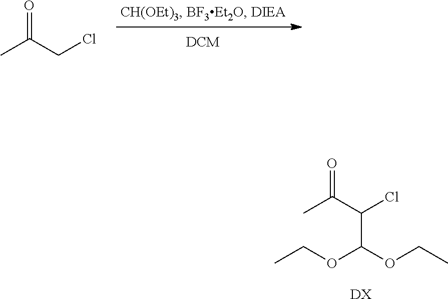

To a stirred solution of diethoxymethoxyethane (16.0 g, 108 mmol, CAS #122-51-0) in DCM (150 mL) was added diethyloxonio(trifluoro)boranuide (32.6 g, 108 mmol, 47% solution) at −30° C. under N₂ atmosphere. The reaction mixture was allowed to stir at 25° C. for 1 hr. Then 1-chloropropan-2-one (5.00 g, 54.0 mmol, CAS #78-95-5) was added rapidly at −78° C. followed by DIPEA (20.9 g, 162 mmol). Then the reaction mixture was allowed to stir at −78° C. for 1 hr. The reaction mass was added saturated NaHCO₃ (100 mL) and stirred for 15 mins and the layer was separated. The aqueous phase was extracted with DCM (2×100 mL). The combined organic layer was washed with H₂SO₄:H₂O (1:10) ratio followed by water (2×100 mL). The organic layer was dried over anhydrous Na₂SO₄ and concentrated under reduced pressure to give the title compound (10.0 g, 47% yield) as a red oil.

### Synthesis of [4-(3-Isopropyl-2-methyl-imidazol-4-yl)pyrimidin-2-yl] trifluoromethanesulfonate (Intermediate DY)

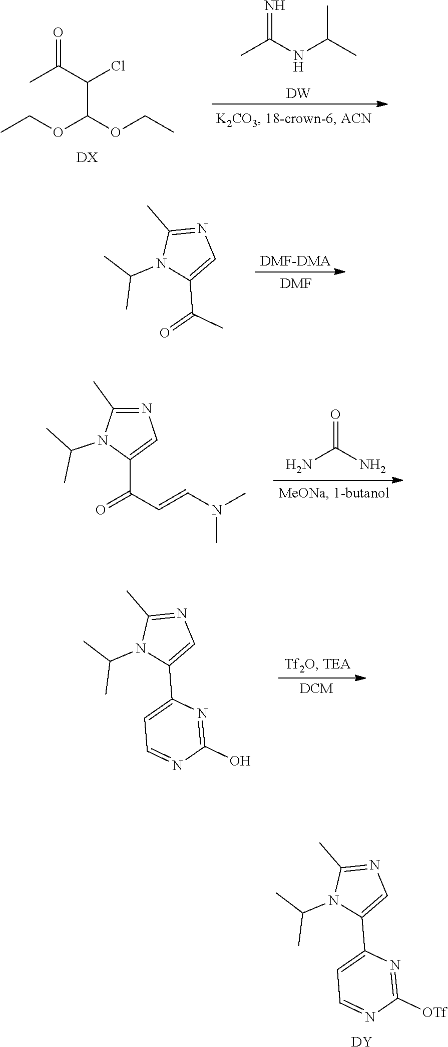

### Step 1—1-(3-Isopropyl-2-methyl-imidazol-4-yl)ethanone

A mixture of 3-chloro-4,4-diethoxy-butan-2-one (10.0 g, 51.3 mmol, Intermediate DX), N-isopropylacetamidine (5.15 g, 51.3 mmol, Intermediate DW), K₂CO₃ (21.3 g, 154 mmol) and 18-CROWN-6 (678 mg, 2.57 mmol) in ACN (100 mL) was degassed and purged with N₂ three times. Then the mixture was stirred at 80° C. for 16 hrs under N₂ atmosphere. The reaction mixture was partitioned between H₂O (100 mL) and EA (2×100 mL). The organic phase was separated, washed with brine (100 mL), dried over anhydrous Na₂SO₄, filtered and concentrated in vacuo to give a residue. The residue was purified by column chromatography (SiO₂, DCM/IPA=100/1 to 10/1) to give the title compound (5.00 g, 58% yield) as a red oil. ¹H NMR (400 MHz, CDCl₃) δ 7.72 (s, 1H), 5.43-5.22 (m, 1H), 2.54 (s, 3H), 2.46 (s, 3H), 1.52 (s, 3H), 1.50 (s, 3H). LCMS (ESI⁺) m/z 167.1 (M+H)⁺.

### Step 2—(E)-3-(Dimethylamino)-1-(3-isopropyl-2-methyl-imidazol-4-yl)prop-2-en-1-one

To a solution of 1-(3-isopropyl-2-methyl-imidazol-4-yl)ethanone (5.00 g, 30.0 mmol) in DMF (30 mL) was added DMF-DMA (3.94 g, 33.0 mmol, CAS #4637-24-5). The mixture was stirred at 130° C. for 16 hrs. The reaction mixture was concentrated in vacuo to remove solvent. The residue was purified by column chromatography (SiO₂, DCM/IPA=100/1 to 10/1) to give the title compound (3.00 g, 45% yield) as a red solid. ¹H NMR (400 MHz, CDCl₃) δ 7.66 (d, J=12.4 Hz, 1H), 7.48 (s, 1H), 5.49 (d, J=12.4 Hz, 1H), 5.47-5.40 (m, 1H), 3.14-2.87 (m, 6H), 2.60 (s, 3H), 1.56 (s, 3H), 1.54 (s, 3H). LCMS (ESI⁺) m/z 222.2 (M+H)⁺.

### Step 3—4-(3-Isopropyl-2-methyl-imidazol-4-yl)pyrimidin-2-ol

To a solution of (E)-3-(dimethylamino)-1-(3-isopropyl-2-methyl-imidazol-4-yl)prop-2-en-1-one (2.00 g, 9.04 mmol), CH₃ONa (1.95 g, 36.1 mmol) and urea (1.36 g, 22.5 mmol, CAS #506-89-8) in 1-butanol (20 mL) was stirred at 140° C. for 16 hrs. On completion, the reaction mixture was concentrated in vacuo to remove the solvent. The residue was purified by prep-HPLC (column: Phenomenex C18 250*50 mm*10 um; mobile phase: [water (ammonia hydroxide v/v)-ACN]; B %: 0%-20%, 8 min) to give the title compound (1.10 g, 55% yield) as a white solid. LCMS (ESI⁺) m/z 219.0 (M+H)⁺.

### Step 4—[4-(3-Isopropyl-2-methyl-imidazol-4-yl)pyrimidin-2-yl] trifluoromethanesulfonate

To a solution of 4-(3-isopropyl-2-methyl-imidazol-4-yl)pyrimidin-2-ol (550 mg, 2.52 mmol) in DCM (5 mL) was added TEA (509 mg, 5.04 mmol) and Tf₂O (746 mg, 2.65 mmol). The mixture was stirred at 0° C. for 1 hr. On completion, the reaction mixture was partitioned between H₂O (50 mL) and DCM (50 mL). The organic phase was separated, washed with brine (50 mL), dried over anhydrous Na₂SO₄, filtered and concentrated in vacuo to give a residue to give the title compound (780 mg, 88% yield) as a red solid. LCMS (ESI⁺) m/z 350.9 (M+H)⁺.

### Synthesis of 6-Chloro-2-(4-ethylsulfanyl-2-methyl-anilino)-8-isopropyl-pyrido[2,3-d]pyrimidin-7-one (Intermediate LR)

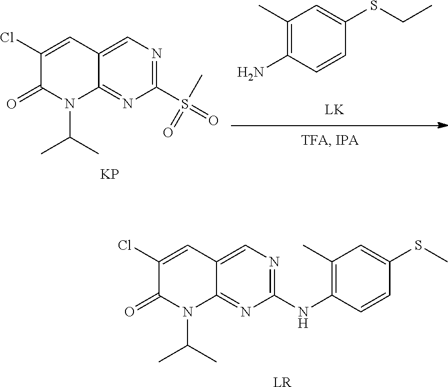

4-Ethylsulfanyl-2-methyl-aniline (83.1 mg, 497 μmol, Intermediate LK), 6-chloro-8-isopropyl-2-methylsulfonyl-pyrido[2,3-d]pyrimidin-7-one (100 mg, 331 μmol, Intermediate KP) and TFA (377 mg, 3.31 mmol) were placed in a microwave tube in IPA (1.5 mL). The sealed tube was heated at 120° C. for 2 hrs under microwave. On completion, the mixture was cooled, diluted with H₂O (10 mL), and extracted with EA (3×10 mL). The combined organic phases were washed with brine (10 mL), dried over anhydrous Na₂SO₄, filtered and concentrated in vacuo. The residue was purified by prep-TLC (SiO₂, PE:EA=2:1) to give the title compound (65 mg, 50% yield) as a yellow solid. LCMS (ESI⁺) m/z 375.2 (M+H)⁺.

### Synthesis of 6-(Difluoromethyl)-8-isopropyl-2-methylsulfonyl-pyrido[2,3-d]pyrimidin-7-one (Intermediate LS)

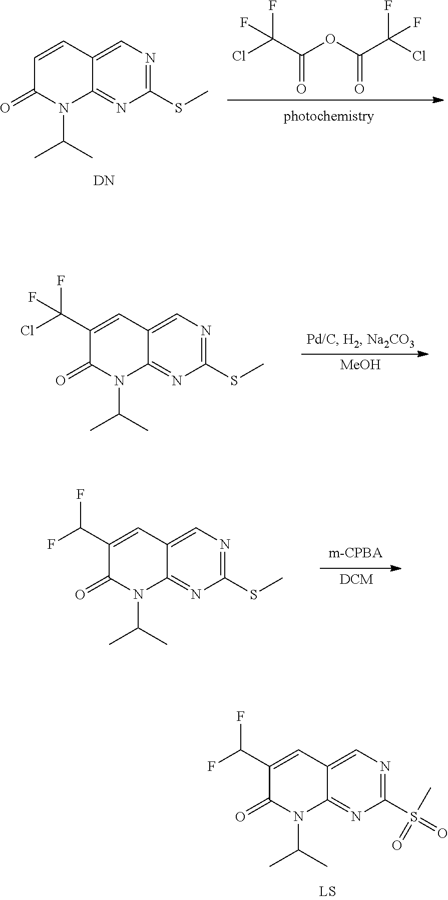

### Step 1—6-[Chloro(difluoro)methyl]-8-isopropyl-2-methylsulfanyl-pyrido[2,3-d]pyrimidin-7-one

To an 15 mL vial equipped with a stir bar was added 4-phenylpyridine N-Oxide (3.64 g, 21.0 mmol), 8-isopropyl-2-methylsulfanyl-pyrido[2,3-d]pyrimidin-7-one (2.00 g, 8.50 mmol, Intermediate DN), and Ru(bpy)₃Cl₂·6H₂O (63.6 mg, 85.0 μmol) in dry ACN (20 mL), then (2-chloro-2,2-difluoro-acetyl) 2-chloro-2,2-difluoro-acetate (5.16 g, 21.0 mmol, CAS #2834-28-3) was added. The vial was sealed and placed under nitrogen was added. The reaction was stirred and irradiated with a 34 W blue LED lamp (2 cm away), with cooling water to keep the reaction temperature at 25° C. for 16 hrs. On completion, the mixture was concentrated in vacuo. The residue was purified by column chromatography (SiO₂, Petroleum ether/Ethyl acetate=20/1 to 10/1) (Rf=0.55, PE:EA=1:1) to give the title compound (1.37 g, 50% yield) as a yellow solid. ¹H NMR (400 MHz, DMSO-d₆) δ 9.02 (s, 1H), 8.50 (s, 1H), 5.82-5.64 (m, 1H), 2.63 (s, 3H), 1.57 (d, J=6.8 Hz, 6H). LC-MS (ESI⁺) m/z 319.6 (M+H)⁺.

### Step 2—6-(Difluoromethyl)-8-isopropyl-2-methylsulfanyl-pyrido[2,3-d]pyrimidin-7-one

A mixture of 6-[chloro(difluoro)methyl]-8-isopropyl-2-methylsulfanyl-pyrido[2,3-d]pyrimidin-7-one (200 mg, 625 μmol), Pd/C (10.0 mg, 6.25 μmol, 10 wt %), Na₂CO₃ (99.0 mg, 938 μmol) in THF (2 mL) was degassed and purged with H₂ three times. Then the mixture was stirred at 25° C. for 2 hours under H₂ atmosphere. On completion, the mixture was filtered, and the filtrate was concentrated in vacuo. The residue was purified by column chromatography (SiO₂, Petroleum ether/Ethyl acetate=30/1 to 10/1) (Rf=0.70, PE:EA=3:1) to give the title compound (70.0 mg, 39% yield) as a white solid. ¹H NMR (400 MHz, DMSO-d₆) δ 9.00 (s, 1H), 8.28 (s, 1H), 5.87-5.59 (m, 1H), 3.36-3.26 (m, 1H), 2.62 (s, 3H), 1.56 (d, J=6.8 Hz, 6H). LC-MS (ESI⁺) m/z 286.0 (M+H)⁺.

### Step 3—6-(Difluoromethyl)-8-isopropyl-2-methylsulfonyl-pyrido[2,3-d]pyrimidin-7-one

To a solution of 6-(difluoromethyl)-8-isopropyl-2-methylsulfanyl-pyrido[2,3-d]pyrimidin-7-one (260 mg, 911 μmol) in DCM (2 mL) was added m-CPBA (740 mg, 3.65 mmol, 85% solution). The mixture was stirred at 40° C. for 3 hrs. On completion, the mixture was quenched with NaHCO₃ (10 mL), then extracted with EA (3×10 mL). The combined organic layers were washed with brine (3×10 mL), dried with anhydrous Na₂SO₄, filtered and the filtrate was concentrated in vacuo. The residue was purified by column chromatography (SiO₂, Petroleum ether/Ethyl acetate=30/1 to 10/1) to give the title compound (100 mg, 34% yield) as a yellow solid. LC-MS (ESI⁺) m/z 317.9 (M+H)⁺.

### Synthesis of 6-(Difluoromethyl)-2-(4-ethylsulfanyl-2-methyl-anilino)-8-isopropyl-pyrido[2,3-d]pyrimidin-7-one (Intermediate LT)

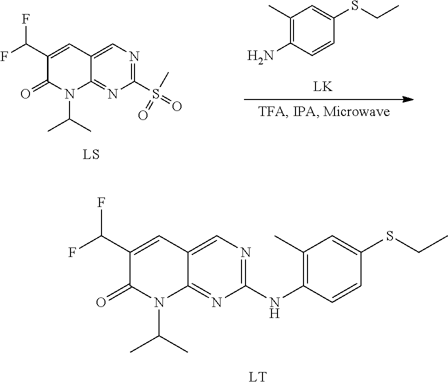 [SMILES: CC(C)n1c(=O)c(C(F)F)cc2cnc(S(C)(=O)=O)nc21 ; CCSc1ccc(N)c(C)c1 ; CCSc1ccc(Nc2ncc3cc(C(F)F)c(=O)n(C(C)C)c3n2)c(C)c1]

A mixture of 6-(difluoromethyl)-8-isopropyl-2-methylsulfonyl-pyrido[2,3-d]pyrimidin-7-one (200 mg, 630 μmol, Intermediate LS), 4-ethylsulfanyl-2-methyl-aniline (210 mg, 1.26 mmol, Intermediate LK), and TFA (718 mg, 6.30 mmol, 466 uL) in IPA (2 mL) stirred at 120° C. for 2 hrs under microwave. On completion, the mixture was concentrated in vacuo. The residue was purified by prep-TLC (SiO₂, PE:EA=3:1) (Rf=0.35, PE:EA=3:1) to give the title compound (60.0 mg, 23% yield) as a yellow solid. LC-MS (ESI⁺) m/z 405.2 (M+H)⁺.

### Synthesis of 2-Chloro-4-ethylsulfanyl-aniline (Intermediate LU)

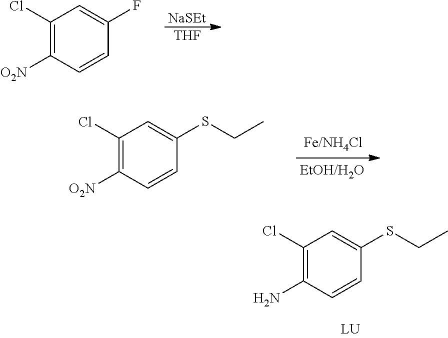

### Step 1—2-Chloro-4-ethylsulfanyl-1-nitro-benzene

To a solution of 2-chloro-4-fluoro-1-nitro-benzene (1.00 g, 5.70 mmol, CAS #2106-50-5) in THF (10 mL) was added NaSEt (718 mg, 8.54 mmol). The mixture was stirred at 25° C. for 3 hrs. On completion, the reaction mixture was partitioned between H₂O (30 mL) and EA (3×20 mL). The organic phase was separated, washed with brine (60 mL), dried over anhydrous Na₂SO₄, filtered and concentrated in vacuo to give the title compound (0.99 g, 80% yield) as yellow oil. LCMS (ESI⁺) m/z 218.0 (M+H)⁺.

### Step 2—2-Chloro-4-ethylsulfanyl-aniline

To a solution of 2-chloro-4-ethylsulfanyl-1-nitro-benzene (0.99 g, 4.57 mmol) in a mixture solution of EtOH (10 mL) and H₂O (10 mL) was added Fe (1.28 g, 22.8 mmol) and NH₄Cl (2.44 g, 45.6 mmol). The mixture was stirred at 80° C. for 2 hrs. The reaction mixture was partitioned between H₂O (30 mL) and EA (3×20 mL). The organic phase was separated, washed with brine (60 mL), dried over anhydrous Na₂SO₄, filtered and concentrated in vacuo to give a residue. The residue was purified by prep-HPLC (column: Phenomenex luna C18 250*50 mm*15 um; mobile phase: [water(FA)-ACN]; B %: 35%-65%, 26 min) to give the title compound (220 mg, 25% yield) as black oil. ¹H NMR (400 MHz, CDCl₃) δ 7.35 (d, J=2.0 Hz, 1H), 7.15 (dd, J=2.0, 8.4 Hz, 1H), 6.71 (d, J=8.4 Hz, 1H), 4.39-3.62 (m, 2H), 2.80 (q, J=7.2 Hz, 2H), 1.24 (t, J=7.2 Hz, 3H). LCMS (ESI⁺) m/z 187.8 (M+H)⁺.

### Synthesis of 6-Chloro-2-(2-chloro-4-ethylsulfanyl-anilino)-8-isopropyl-pyrido[2,3-d]pyrimidin-7-one (Intermediate LV)

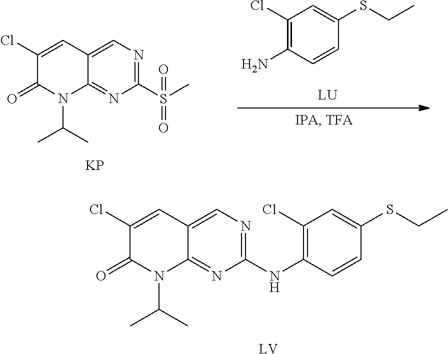

2-Chloro-4-ethylsulfanyl-aniline (93.3 mg, 497 μmol, Intermediate LU), 6-chloro-8-isopropyl-2-methylsulfonyl-pyrido[2,3-d]pyrimidin-7-one (100 mg, 331 μmol, Intermediate KP) and TFA (377 mg, 3.31 mmol) were placed in a microwave tube in IPA (1.5 mL). The sealed tube was heated at 120° C. for 2 hrs under microwave. The reaction mixture was partitioned between H₂O (10 mL) and EA (3×10 mL). The organic phase was separated, washed with brine (30 mL), dried over anhydrous Na₂SO₄, filtered and concentrated in vacuo to give a residue. The residue was purified by prep-TLC (SiO₂, PE:EA=3:1) to give the title compound (20 mg, 14% yield) as yellow oil. LCMS (ESI⁺) m/z 408.8 (M+H)⁺.

### Synthesis of 4-(1-Methylpyrazol-4-yl)sulfonylcyclohexanamine (Intermediate LW)

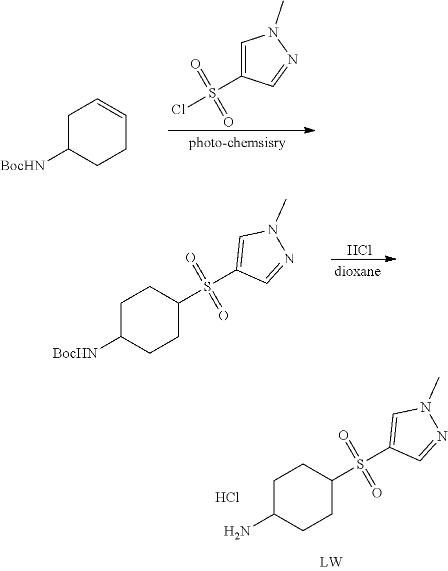

### Step 1—Tert-butyl N-[4-(1-methylpyrazol-4-yl)sulfonylcyclohexyl]carbamate

To an oven-dried 40 mL vial equipped with magnetic stir bar was charged with tert-butyl N-cyclohex-3-en-1-ylcarbamate (1.50 g, 7.60 mmol, CAS #135262-85-0), 1-methylpyrazole-4-sulfonyl chloride (3.43 g, 19.0 mmol, CAS #288148-34-5), IR(PPY)₃ (24.9 mg, 38.0 μmol), bis(trimethylsilyl)silyl-trimethyl-silane (3.78 g, 15.2 mmol, 4.69 mL) and 4-sulfanylphenol (192 mg, 1.52 mmol) in ACN (20 mL). The vial was sealed and placed under nitrogen. The reaction was stirred and irradiated with a 10 W blue LED lamp (3 cm away), with cooling water to keep the reaction temperature at 25° C. for 14 hrs. On completion, the mixture was concentrated in vacuo to give a residue. The residue was purified by prep-HPLC (column: Waters xbridge 150*25 mm 10 um; mobile phase: [water(NH₄HCO₃)-ACN]; B %: 18%-48%, 8 min) to give the title compound (470 mg, 16% yield) as a white solid.

¹H NMR (400 MHz, DMSO-d₆) δ 8.35-8.33 (m, 1H), 7.79-7.77 (m, 1H), 3.91-3.90 (m, 3H), 3.86 (d, J=2.4 Hz, 1H), 3.30 (s, 1H), 1.94-1.83 (m, 2H), 1.81-1.63 (m, 1H), 1.60-1.44 (m, 3H), 1.37 (s, 9H), 1.28-1.04 (m, 2H). LC-MS (ESI⁺) m/z 244.1 (M+H)⁺.

### Step 2—4-(1-Methylpyrazol-4-yl)sulfonylcyclohexanamine

A mixture of tert-butyl N-[4-(1-methylpyrazol-4-yl)sulfonylcyclohexyl]carbamate (200 mg, 582 μmol) in HCl/dioxane (1 mL) and DCM (1 mL) was stirred at 20° C. for 0.5 hour. On completion, the mixture was concentrated to give the title compound (130 mg, HCl salt) as a white solid. LC-MS (ESI⁺) m/z 285.0 (M+H)⁺.

### Synthesis of 6-Bromo-8-isopropyl-2-methylsulfanyl-pyrido[2,3-d]pyrimidin-7-one (Intermediate LX)

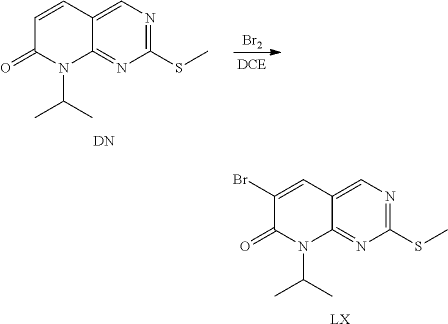

To a solution of 8-isopropyl-2-methylsulfanyl-pyrido[2,3-d]pyrimidin-7-one (1.00 g, 4.25 mmol, Intermediate DN) in DCE (10 mL) was added Br₂ (2.72 g, 17.0 mmol) at 0° C. The mixture was then stirred at 25° C. for 16 hrs. On completion, the reaction mixture was quenched with Na₂SO₃ (30 mL), then the solution was adjusted pH to 8 by NaHCO₃, and extracted with EtOAc (3×10 mL). The combined organic layers were dried over anhydrous Na₂SO₄, filtered and concentrated in vacuo to give a residue. The residue was purified by column chromatography (SiO₂, Petroleum ether/Ethyl acetate=50/1 to 10/1) to give the title compound (1.00 g, 74% yield) as a white solid. ¹H NMR (400 MHz, CDCl₃) δ 8.58 (s, 1H), 8.00 (s, 1H), 6.00-5.82 (m, 1H), 2.65 (s, 3H), 1.65 (d, J=7.2 Hz, 6H). LCMS (ESI⁺) m/z 315.6 (M+H)⁺.

### Synthesis of 8-Isopropyl-2-methylsulfonyl-7-oxo-pyrido[2,3-d]pyrimidine-6-carbonitrile (Intermediate LY)

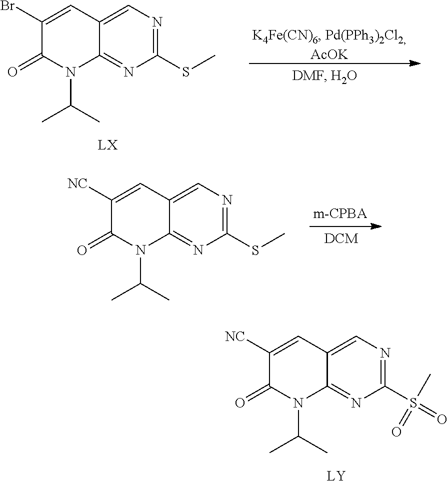

### Step 1—8-Isopropyl-2-methylsulfanyl-7-oxo-pyrido[2,3-d]pyrimidine-6-carbonitrile

To a mixture of 6-bromo-8-isopropyl-2-methylsulfanyl-pyrido[2,3-d]pyrimidin-7-one (1.00 g, 3.18 mmol, Intermediate LX), Pd(PPh₃)₂Cl₂ (670 mg, 954 μmol), AcOK (156 mg, 1.59 mmol) and tetrapotassium; hexacyanoiron (586 mg, 1.59 mmol) in DMF (10 mL) and H₂O (1 mL), then the mixture was stirred at 100° C. for 16 hrs at N₂. On completion, the mixture was added to water (20 mL) and extracted with ethyl acetate (3×20 mL). The separated organic layer was washed with brine (20 mL), dried over anhydrous sodium sulfate, filtered and concentrated in vacuo to give a residue. The residue was purified by column chromatography (SiO₂, Petroleum ether/Ethyl acetate=20/1 to 3/1) to give the title compound (700 mg, 84% yield) as a white solid. LCMS (ESI⁺) m/z 260.8 (M+H)⁺.

### Step 2—8-Isopropyl-2-methylsulfonyl-7-oxo-pyrido[2,3-d]pyrimidine-6-carbonitrile

To a solution of 8-isopropyl-2-methylsulfanyl-7-oxo-pyrido[2,3-d]pyrimidine-6-carbonitrile (150 mg, 576 μmol) in DCM (5 mL) was added m-CPBA (467 mg, 2.30 mmol, 85% solution). The mixture was stirred at 25° C. for 1 hr. On completion, the reaction mixture was partitioned between aq. NaHCO₃ (20 mL) and DCM (20 mL). The organic phase was separated, washed with brine (20 mL), dried over anhydrous Na₂SO₄, filtered and concentrated in vacuo to give the title compound (150 mg, 89% yield) as a white solid. LCMS (ESI⁺) m/z 292.6 (M+H)⁺.

### Synthesis of 2—(4-Ethylsulfanyl-2-methyl-anilino)-8-isopropyl-7-oxo-pyrido[2,3-d]pyrimidine-6-carbonitrile (Intermediate LZ)

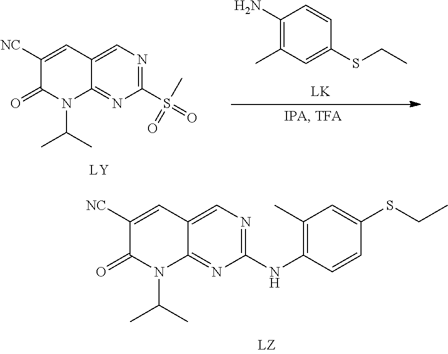

8-Isopropyl-2-methylsulfonyl-7-oxo-pyrido[2,3-d]pyrimidine-6-carbonitrile (150 mg, 513 μmol, Intermediate LY), 4-ethylsulfanyl-2-methyl-aniline (85.8 mg, 513 μmol, Intermediate LK) and TFA (585 mg, 5.13 mmol) were placed in a microwave tube in IPA (1.5 mL). The sealed tube was heated at 120° C. for 2 hrs under microwave. On completion, the mixture was cooled, diluted with H₂O (10 mL), and extracted with EA (3×10 mL). The combined organic phases were washed with brine (10 mL), dried over Na₂SO₄, filtered and concentrated in vacuo. The residue was purified by prep-TLC (SiO₂, PE:EA=1:1) to give the title compound (100 mg, 51% yield) as a white solid. LCMS (ESI⁺) m/z 380.2 (M+H)⁺.

### Synthesis of 2-Chloro-4-(difluoromethyl)-6-(1-methylpyrazol-4-yl)pyrimidine (Intermediate MA)

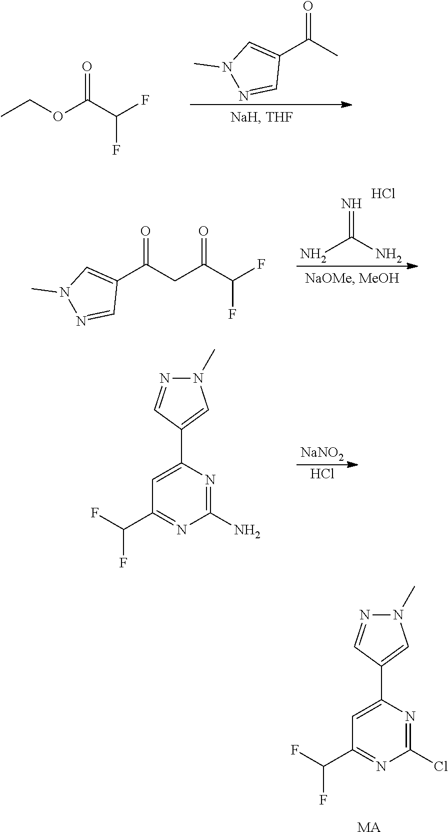

### Step 1—4,4-Difluoro-1-(1-methylpyrazol-4-yl)butane-1,3-dione

To a solution of 1-(1-methylpyrazol-4-yl)ethanone (1.00 g, 8.06 mmol, CAS #37687-18-6) in THF (10 mL) was added NaH (483 mg, 12.0 mmol, 60% dispersion in mineral oil) at 0° C. for 0.5 hrs, and then ethyl 2,2-difluoroacetate (2.00 g, 16.1 mmol, CAS #454-31-9) was added. The mixture was stirred at 25° C. for 1.5 hrs. On completion, the reaction mixture was quenched with H₂O (10 mL) at 25° C., and then extracted with EA (3×50 mL). The combined organic layers were washed with brine (2×50 mL), dried over anhydrous Na₂SO₄ filtered and concentrated in vacuo to give the title compound (1.60 g, 98% yield) as a yellow solid. LCMS (ESI⁺) m/z 202.8 (M+H)⁺.

### Step 2—4-(Difluoromethyl)-6-(1-methylpyrazol-4-yl)pyrimidin-2-amine

To a solution of 4,4-difluoro-1-(1-methylpyrazol-4-yl)butane-1,3-dione (1.10 g, 5.44 mmol) in MeOH (15 mL) was added guanidine-hydrochloride (1.30 g, 13.6 mmol) and NaOMe (587 mg, 10.8 mmol). The mixture was then stirred at 40° C. for 16 hrs. On completion, the reaction mixture was concentrated in vacuo to give a residue. The residue was purified by column chromatography (SiO₂, DCM/MeOH=150/1 to 80/1) to give the title compound (260 mg, 21% yield) as a white solid. ¹H NMR (400 MHz, DMSO-d₆) δ 8.38 (s, 1H), 8.05 (s, 1H), 7.04 (s, 1H), 6.88 (s, 2H), 6.80-6.49 (m, 1H), 3.92-3.86 (m, 3H). LCMS (ESI⁺) m/z 225.9 (M+H)⁺.

### Step 3—2-Chloro-4-(difluoromethyl)-6-(1-methylpyrazol-4-yl)pyrimidine

To a solution of 4-(difluoromethyl)-6-(1-methylpyrazol-4-yl)pyrimidin-2-amine (240 mg, 1.07 mmol) in HCl (10 mL) was added NaNO₂ (735 mg, 10.6 mmol) in water slowly drop by drop at 0° C. The mixture was stirred at 25° C. for 3 hrs. On completion, the reaction mixture was quenched with 10 N sodium hydroxide aqueous solution to adjust the pH=7. Then the resulting solution was filtered and the filter cake was dried in vacuo to give the title compound (80.0 mg, 30% yield) as a white solid. ¹H NMR (400 MHz, DMSO-d₆) δ 8.68 (s, 1H), 8.30 (s, 1H), 8.04 (s, 1H), 7.12-6.80 (m, 1H), 3.92 (s, 3H). LCMS (ESI⁺) m/z 244.7 (M+H)⁺.

### Synthesis of 2-Chloro-8-cyclopentyl-quinazoline (Intermediate MB)

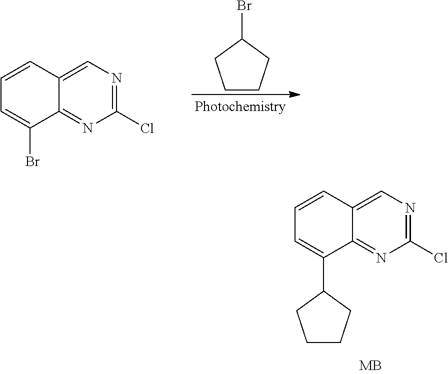 [SMILES: Clc1ncc2cccc(Br)c2n1 ; BrC1CCCC1 ; Clc1ncc2cccc(C3CCCC3)c2n1]

To a 15 mL vial equipped with a stir bar was added 8-bromo-2-chloro-quinazoline (100 mg, 410 μmol, CAS #956100-63-3), bromocyclopentane (79.5 mg, 533 μmol, CAS #137-43-9), Ir[dF(CF₃)ppy]₂(dtbpy)(PF₆) (4.61 mg, 4.11 μmol), NiCl₂·dtbbpy (2.45 mg, 6.16 μmol), TTMSS (102 mg, 410 μmol, 126 uL), and Na₂CO₃ (87.0 mg, 821 μmol) in DME (2 mL). The vial was sealed and placed under nitrogen was added. The reaction was stirred and irradiated with a 10 W blue LED lamp (3 cm away), with cooling water to keep the reaction temperature at 25° C. for 14 hrs. On completion, the reaction mixture was diluted with H₂O (10 mL) and extracted with EA (3×10 mL). The combined organic layers were washed with brine (2×10 mL), dried over Na₂SO₄, filtered and concentrated in vacuo to give a residue. Then the residue was purified by column chromatography (SiO₂, Petroleum ether/Ethyl acetate=100/1 to 5/1) and prep-TLC (SiO₂, PE:EA=5:1) to give the title compound (45 mg, 11% yield) as a white solid. ¹H NMR (400 MHz, CDCl₃) δ 9.26 (s, 1H), 7.88-7.86 (m, 1H), 7.79-7.76 (m, 1H), 7.66-7.59 (m, 1H), 4.22-4.10 (m, 1H), 2.29-2.18 (m, 2H), 1.92-1.79 (m, 4H), 1.73-1.62 (m, 2H), LC-MS (ESI⁺) m/z 233.0 (M+H)⁺.

### Synthesis of 2-Chloro-8-isopropyl-quinazoline (Intermediate MC)

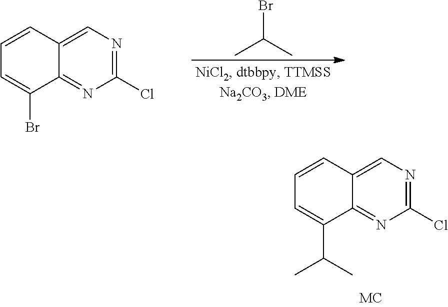

To a mixture of 8-bromo-2-chloro-quinazoline (1.00 g, 4.11 mmol, CAS #956100-63-3) and 2-bromopropane (757 mg, 6.16 mmol) in DME (10 mL) was added NiCl₂·dtbbpy (24.5 mg, 61.6 μmol), TTMSS (1.02 g, 4.11 mmol, 1.27 mL), and Na₂CO₃ (870 mg, 8.21 mmol). The reaction mixture was stirred at 25° C. for 14 hrs. On completion, the reaction mixture was filtered and filtrate was concentrated in vacuo to give the residue. The residue was purified by column chromatography to give the title compound (0.700 g, 82% yield) as yellow oil. LC-MS (ESI⁺) m/z 206.8 (M+H)⁺.

### Synthesis of Tert-butyl 4-(4-amino-3-methyl-phenyl) sulfonylpiperidine-1-carboxylate (Intermediate KZ)

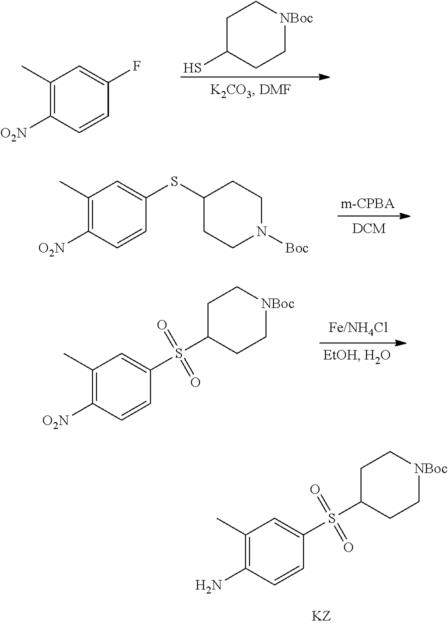

### Step 1—Tert-butyl 4-(3-methyl-4-nitro-phenyl)sulfanylpiperidine-1-carboxylate

To a solution of 4-fluoro-2-methyl-1-nitro-benzene (2.14 g, 13.8 mmol, CAS #446-33-3) and tert-butyl 4-sulfanylpiperidine-1-carboxylate (2.50 g, 11.5 mmol, CAS #134464-79-2) in DMF (30 mL) was added K₂CO₃ (3.18 g, 23.0 mmol), then the mixture was stirred at 25° C. for 8 hrs. On completion, the mixture was diluted with water (30 mL) and extracted with EA (20 mL×3). The combined organic layer was dried over anhydrous Na₂SO₄, filtered and concentrated in vacuo to give the residue. The residue was purified by column chromatography (SiO₂, PE:EA=15:1 to 7:1) to give the title compound (3.60 g, 88% yield) as yellow solid. ¹H NMR (400 MHz, DMSO-d₆) δ 7.96 (d, J=8.8 Hz, 1H), 7.44 (d, J=1.6 Hz, 1H), 7.39-7.38 (m, 1H), 3.83 (d, J=13.6 Hz, 2H), 3.78-3.70 (m, 1H), 3.09-2.92 (m, 2H), 2.52 (s, 3H), 2.02-1.90 (m, 2H), 1.46-1.40 (m, 2H), 1.39 (s, 9H).

### Step 2—Tert-butyl 4-(3-methyl-4-nitro-phenyl)sulfonylpiperidine-1-carboxylate

To a solution of tert-butyl 4-(3-methyl-4-nitro-phenyl)sulfanylpiperidine-1-carboxylate (1.00 g, 2.84 mmol) in DCM (10 mL) was added MCPBA (2.45 g, 14.1 mmol) at 0° C., then the mixture was stirred at 25° C. for 1 hr. On completion, the mixture was quenched with Na₂SO₃ (10 mL) and Na₂CO₃ (8 mL) at 0° C., diluted with water (8 mL) and extracted with DCM (8 mL×3). The combined organic layer was dried over anhydrous Na₂SO₄, filtered and concentrated in vacuo to give the residue. The residue was purified by column chromatography (SiO₂, PE:EA=4:1 to 1:1) to give the title compound (900 mg, 82% yield) as yellow solid. ¹H NMR (400 MHz, DMSO-d₆) δ 8.20 (d, J=8.4 Hz, 1H), 8.01 (d, J=1.2 Hz, 1H), 7.90-7.89 (m, 1H), 4.01 (d, J=11.6 Hz, 2H), 3.73-3.54 (m, 1H), 2.75-2.64 (m, 2H), 2.58 (s, 3H), 1.84 (d, J=11.6 Hz, 2H), 1.45-1.38 (m, 2H), 1.37 (s, 9H).

### Step 3—Tert-butyl 4-(4-amino-3-methyl-phenyl) sulfonylpiperidine-1-carboxylate

To a solution of tert-butyl 4-(3-methyl-4-nitro-phenyl)sulfonylpiperidine-1-carboxylate (0.400 g, 1.04 mmol) in EtOH (10 mL) and H₂O (2 mL) was added Fe (348 mg, 6.24 mmol) and NH₄Cl (556 mg, 10.4 mmol). The reaction mixture was stirred at 80° C. for 2 hrs. On completion, the reaction mixture was filtered and filtrate was concentrated in vacuo. The residue was diluted with water (10 mL), then extracted with EA (3×20 mL). The combined organic layer was dried over Na₂SO₄, filtered and filtrate was concentrated in vacuo. The residue was purified by column chromatography to give the title compound (300 mg, 81% yield) as yellow solid. LC-MS (ESI⁺) m/z 298.9 (M-56)⁺.

### Synthesis of 2-Methyl-4-[(1-methyl-4-piperidyl)sulfonyl]aniline (Intermediate MD)

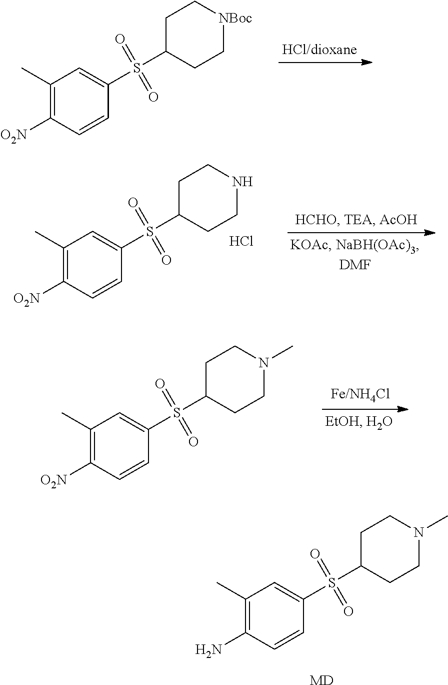

### Step 1—4-(3-Methyl-4-nitro-phenyl)sulfonylpiperidine

A solution of tert-butyl 4-(3-methyl-4-nitro-phenyl)sulfonylpiperidine-1-carboxylate (800 mg, 2.08 mmol, synthesized via Steps 1-2 of Intermediate KZ) in HCl/dioxane (8 mL) was stirred at 25° C. for 10 mins. On completion, the mixture was concentrated in vacuo to give the title compound (590 mg, 88% yield, HCl) as white solid. LC-MS (ESI⁺) m/z 284.9 (M+H)⁺.

### Step 2—1-Methyl-4-(3-methyl-4-nitro-phenyl)sulfonyl-piperidine

To a solution of 4-(3-methyl-4-nitro-phenyl)sulfonylpiperidine (590 mg, 1.84 mmol, HCl) in DMF (8 mL) was added TEA (186 mg, 1.84 mmol) and formaldehyde (746 mg, 9.20 mmol, 37% solution), then AcOH (110 mg, 1.84 mmol) was added and the mixture was stirred at −10° C. for 0.5 hr. Next, NaBH(OAc)₃ (584 mg, 2.76 mmol) was added and the mixture was stirred at −10° C. for 0.5 hr. On completion, the mixture was quenched with water (0.05 mL) at −10° C., filtered and concentrated in vacuo to give the residue. The residue was purified by prep-HPLC (column: Phenomenex luna C18 150*25 mm*10 um; mobile phase: [water (FA)-ACN]; B %: 0%-28%, 10 min) to give the title compound (380 mg, 69% yield) as a white solid. ¹H NMR (400 MHz, DMSO-d₆) δ 8.20 (d, J=8.4 Hz, 1H), 8.14 (s, 1H), 8.02 (d, J=1.6 Hz, 1H), 7.91 (dd, J=2.0, 8.4 Hz, 1H), 3.42-3.34 (m, 1H), 2.91 (d, J=11.6 Hz, 2H), 2.58 (s, 3H), 2.20 (s, 3H), 2.04-1.92 (m, 2H), 1.83 (d, J=12.0 Hz, 2H), 1.66-1.52 (m, 2H). LC-MS (ESI⁺) m/z 299.0 (M+H)⁺.

### Step 3—2-Methyl-4-[(1-methyl-4-piperidyl)sulfonyl]aniline

To a solution of 1-methyl-4-(3-methyl-4-nitro-phenyl)sulfonyl-piperidine (330 mg, 1.11 mmol) in EtOH (4 mL) and H₂O (0.5 mL) was added Fe (370 mg, 6.64 mmol) and NH₄Cl (591 mg, 11.0 mmol), then the mixture was stirred at 80° C. for 2 hrs. On completion, the mixture was filtered and concentrated in vacuo to give the residue. The residue was purified by prep-HPLC (column: Phenomenex C18 250*50 mm*10 um; mobile phase: [water (NH₄HCO₃)-ACN]; B %: 4%-34%, 8 min) to give the title compound (290 mg, 97% yield) as white solid. ¹H NMR (400 MHz, DMSO-d₆) δ 7.33-7.27 (m, 2H), 6.69 (d, J=8.0 Hz, 1H), 5.90 (s, 2H), 2.91-2.83 (m, 1H), 2.77 (d, J=11.6 Hz, 2H), 2.09 (s, 6H), 1.83-1.70 (m, 4H), 1.48-1.38 (m, 2H). LC-MS (ESI⁺) m/z 269.0 (M+H)⁺.

### Synthesis of Tributyl-(3-isopropyl-2-methyl-imidazol-4-yl)stannane (Intermediate KI)

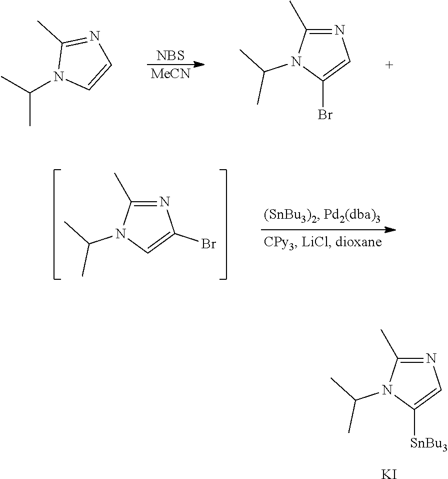

### Step 1—5-Bromo-1-isopropyl-2-methyl-imidazole and 4-bromo-1-isopropyl-2-methyl-imidazole

To a mixture of 1-isopropyl-2-methyl-imidazole (10.0 g, 80.5 mmol, CAS #87606-45-1) in ACN (100 mL) was added NBS (14.3 g, 80.5 mmol) at 0° C., then the reaction mixture was stirred at 25° C. for 16 hours. On completion, the residue was diluted with water (80 mL), and extracted with EA (3×100 mL). The combined organic layer was dried over Na₂SO₄, filtered and concentrated in vacuo to give 5-bromo-1-isopropyl-2-methyl-imidazole (8.00 g, 49% yield, ¹H NMR (400 MHz, DMSO-d₆) δ 6.79 (s, 1H), 4.57 (d, J=7.0, 14.0 Hz, 1H), 2.37 (s, 3H), 1.46 (d, J=7.2 Hz, 6H) as white oil and 4-bromo-1-isopropyl-2-methyl-imidazole (2.00 g, 12% yield, ¹H NMR (400 MHz, DMSO-d₆) δ=7.29 (s, 1H), 4.34 (td, J=6.4, 13.2 Hz, 1H), 2.27 (s, 3H), 1.32 (d, J=6.4 Hz, 6H) as white oil.

### Step 2—Tributyl-(3-isopropyl-2-methyl-imidazol-4-yl)stannane

To a mixture of 5-bromo-1-isopropyl-2-methyl-imidazole (1.50 g, 7.39 mmol) in dioxane (30 mL) was added Pd₂(dba)₃ (676 mg, 738 μmol) and LiCl (939 mg, 22.1 mmol) and tricyclohexylphosphane (2.07 g, 7.39 mmol) and tributyl(tributylstannyl)stannane (42.8 g, 73.8 mmol), then the reaction mixture was stirred at 100° C. for 12 hrs. On completion, the reaction mixture was quenched with CsF(aq) (50 mL) and concentrated in vacuo to give the title compound (3 g, 98% yield) as brown oil. LC-MS (ESI⁺) m/z 412.6 (M+H)⁺.

### Synthesis of 1-Isopropyl-2-methyl-4-(4,4,5,5-tetramethyl-1,3,2-dioxaborolan-2-yl)imidazole (Intermediate ME)

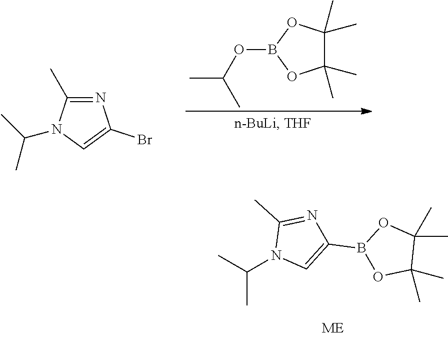

To a mixture of 4-bromo-1-isopropyl-2-methyl-imidazole (650 mg, 3.20 mmol, synthesized via Step 1 of Intermediate KI) and 2-isopropoxy-4,4,5,5-tetramethyl-1,3,2-dioxaborolane (1.79 g, 9.60 mmol, 1.96 ml, CAS #61676-62-8) in THF (10 mL) was added n-BuLi (2.5 M, 3.84 mL) slowly at −78° C. under N₂. The reaction was stirred at −78° C. for 1 hr and then warmed up to 25° C. for 2 hr under N₂. On completion, the reaction mixture was quenched with FA (0.05 mL) filtered and filtrate was concentrated in vacuo to give the title compound (800 mg, 3.20 mmol, 99% yield) as yellow oil. LC-MS (ESI⁺) m/z 251.2 (M+H)⁺.

### Synthesis of 4-chloro-N-(4-ethylsulfanyl-2-methyl-phenyl)-5-(trifluoromethyl)pyrimidin-2-amine (Intermediate MF)

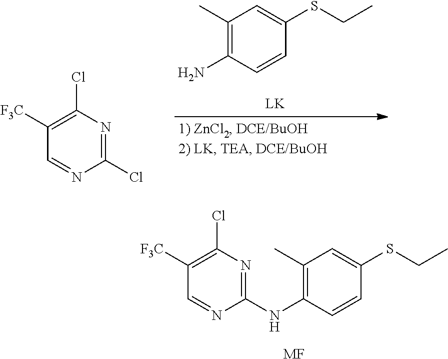

To a mixture 2,4-dichloro-5-(trifluoromethyl)pyrimidine (4.63 g, 21.3 mmol, CAS #3932-97-6) in t-BuOH (50 mL) and DCE (50 mL) was added ZnCl₂ (3.32 g, 24.3 mmol, 1.43 mL) at 0° C. and the mixture was stirred for 1 hour. Next, 4-ethylsulfanyl-2-methyl-aniline (3.40 g, 20.3 mmol, Intermediate LK) and TEA (2.26 g, 22.3 mmol) was added, then the reaction mixture was stirred at 25° C. for 16 hrs. On completion, the residue was diluted with water (50 mL), then the residue was extracted with EA (3×100 mL). The combined organic layer was dried over Na₂SO₄, filtered and concentrated in vacuo to give the residue. The residue was purified by column chromatography (SiO₂, PE:EA=1:0 to PE:EA=50:1, PE:EA=10:1, P1:Rf=0.3) to give the title compound (2.2 g, 31% yield) as white solid. ¹H NMR (400 MHz, DMSO-d₆) δ 10.07 (s, 1H), 7.28 (d, J=8.4 Hz, 1H), 7.23 (d, J=1.6 Hz, 1H), 7.18-7.13 (m, 1H), 2.97 (q, J=7.2 Hz, 2H), 2.18 (s, 3H), 1.23 (t, J=7.2 Hz, 3H); LC-MS (ESI⁺) m/z 347.7 (M+H)⁺.

### Synthesis of N-(4-ethylsulfanyl-2-methyl-phenyl)-4-(1-isopropyl-2-methyl-imidazol-4-yl)-5-(trifluoromethyl)pyrimidin-2-amine (Intermediate MG)

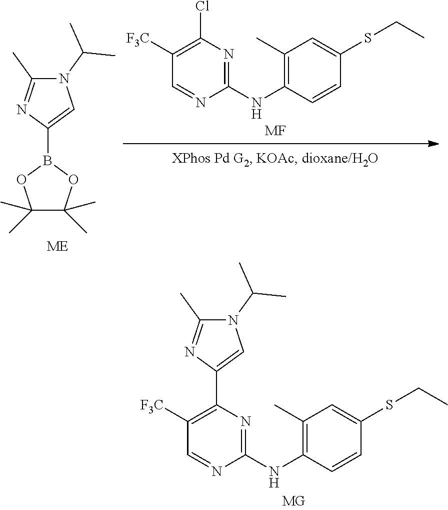

To a mixture of 1-isopropyl-2-methyl-4-(4,4,5,5-tetramethyl-1,3,2-dioxaborolan-2-yl)imidazole (1.00 g, 4.00 mmol, Intermediate ME), 4-chloro-N-(4-ethylsulfanyl-2-methyl-phenyl)-5-(trifluoromethyl)pyrimidin-2-amine (834 mg, 2.40 mmol, Intermediate MF) in dioxane (25 mL) was added K₂CO₃ (1.66 g, 12.0 mmol) and [2-(2-aminophenyl)phenyl]-chloro-palladium; dicyclohexyl-[3-(2,4,6-triisopropylphenyl)phenyl]phosphane (315 mg, 400 μmol), then the mixture was stirred at 90° C. for 12 hour under N₂. On completion, the reaction mixture was filtered and filtrate was concentrated in vacuo. The residue was purified by prep-HPLC (column: Phenomenex luna C18 250*50 mm*10 um; mobile phase: [water(FA)-ACN]; B %: 30%-50%, 12 min) to give the title compound (200 mg, 11% yield) as a yellow oil. LCMS (ESI⁺) m/z 436.1 (M+H)⁺.

### Synthesis of 4-[2-(4-ethylsulfanyl-2-methyl-anilino)-5-(trifluoromethyl)pyrimidin-4-yl]cyclohexanone (Intermediate MH)

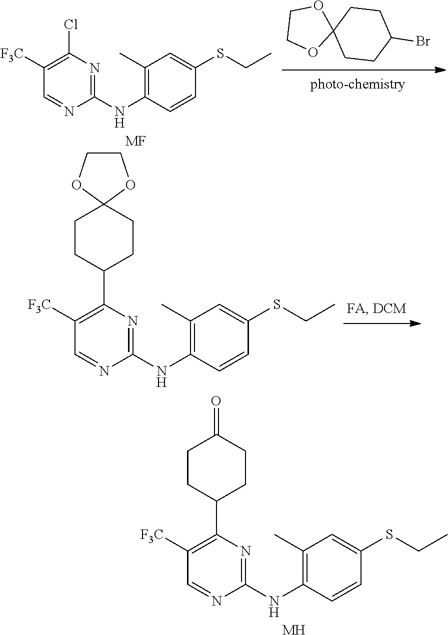

### Step 1—4-(1,4-Dioxaspiro[4.5]decan-8-yl)-N-(4-ethylsulfanyl-2-methyl-phenyl)-5-(trifluoromethyl) pyrimidin-2-amine

To a 15 mL vial equipped with a stir bar was added 4-chloro-N-(4-ethylsulfanyl-2-methyl-phenyl)-5-(trifluoromethyl)pyrimidin-2-amine (1 g, 3 mmol, Intermediate MF), 8-bromo-1,4-dioxaspiro[4.5]decane (826 mg, 3.74 mmol, CAS #68278-51-3), NiCl₂·dtbbpy (17.1 mg, 43.1 μmol), Na₂CO₃ (609 mg, 5.75 mmol), TTMSS (714 mg, 2.88 mmol), and Ir[dF(CF₃)ppy]₂[d(m-CF₃)bpy](PF₆) (32.9 mg, 28.7 μmol) in DME (2 mL). The vial was sealed and placed under nitrogen. The reaction was stirred and irradiated with a 10 W blue LED lamp (3 cm away), with cooling water to keep the reaction temperature at 25° C. for 14 hrs. On completion, the residue was diluted with water (20 mL), then the residue was extracted with EA (3×50 mL). The combined organic layer was dried over Na₂SO₄, filtered and concentrated in vacuo to give a residue. The residue was purified by column chromatography (SiO₂, PE:EA=50:1 to PE:EA=10:1, PE:EA=3:1, P1:Rf=0.3) to give the title compound (1 g, 77% yield) as yellow solid. ¹H NMR (400 MHz, DMSO-d₆) δ 9.46 (s, 1H), 8.52 (s, 1H), 7.32 (d, J=8.4 Hz, 1H), 7.21 (d, J=2.0 Hz, 1H), 7.13 (d, J=2.1, 8.4 Hz, 1H), 3.86 (s, 4H), 2.95 (q, J=7.2 Hz, 2H), 2.79 (t, J=11.2 Hz, 1H), 2.18 (s, 3H), 1.98-1.85 (m, 2H), 1.81-1.66 (m, 4H), 1.61-1.48 (m, 2H), 1.23 (t, J=7.2 Hz, 3H); LC-MS (ESI⁺) m/z 454.4 (M+H)⁺.

### Step 2—4-[2-(4-Ethylsulfanyl-2-methyl-anilino)-5-(trifluoromethyl)pyrimidin-4-yl]cyclohexanone

A solution of 4-(1,4-dioxaspiro[4.5]decan-8-yl)-N-(4-ethylsulfanyl-2-methyl-phenyl)-5-(trifluoromethyl)pyrimidin-2-amine (200 mg, 440 μmol) in HCOOH (2 mL) was stirred at 25° C. for 16 hrs. On completion, the reaction mixture was concentrated in vacuo to give the title compound (120 mg, 66% yield) as a yellow solid. LC-MS (ESI⁺) m/z 410.2 (M+H)⁺.

### Synthesis of 4-(2-((4-(ethylthio)-2-methylphenyl)amino)-5-(trifluoromethyl)pyrimidin-4-yl)-1-methylcyclohexan-1-ol (Intermediate MI) and 4-(2-((4-(ethylthio)-2-methylphenyl)amino)-5-(trifluoromethyl)pyrimidin-4-yl)-1-methylcyclohexan-1-ol (Intermediate MJ)

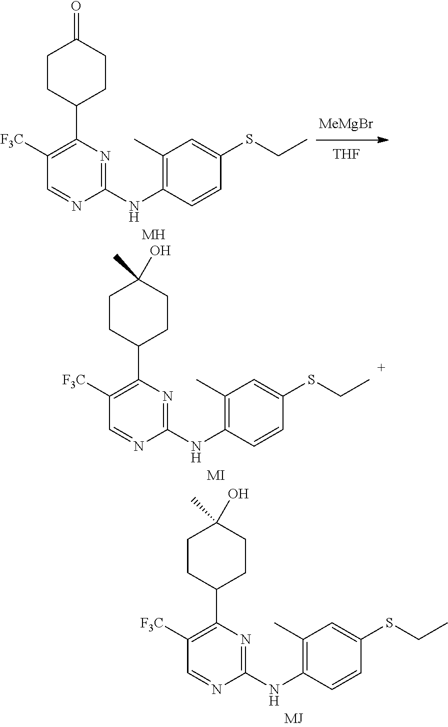

To a solution of 4-[2-(4-ethylsulfanyl-2-methyl-anilino)-5-(trifluoromethyl)pyrimidin-4-yl]cyclohexanone (100 mg, 244 μmol, Intermediate MH) in THF (2 mL) was added MeMgBr (3 M, 407 uL) at 0° C. The mixture was then stirred at 25° C. for 2 hrs. On completion, the reaction mixture was quenched with water (0.5 mL) and concentrated in vacuo. The residue was purified by prep-HPLC (column: Phenomenex luna C18 150*25 mm*10 um; mobile phase: [water(FA)-ACN]; B %: 62%-92%, 10 min) to give 4-(2-((4-(ethylthio)-2-methylphenyl)amino)-5-(trifluoromethyl)pyrimidin-4-yl)-1-methylcyclohexan-1-ol (36 mg, 35% yield) as a white solid and 4-(2-((4-(ethylthio)-2-methylphenyl)amino)-5-(trifluoromethyl)pyrimidin-4-yl)-1-methylcyclohexan-1-ol (30 mg, 29% yield) as a white solid. LC-MS (ESI⁺) m/z for both enantiomers: 426.0 (M+H)⁺. Absolute stereochemistry of the enantiomers was assigned arbitrarily.

### Synthesis of 4-Bromo-5-(difluoromethyl)-1-methyl-pyrazole (Intermediate MK)

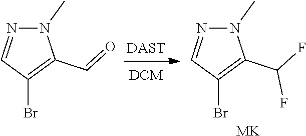

To a solution of 4-bromo-2-methyl-pyrazole-3-carbaldehyde (8.80 g, 46.5 mmol, CAS #473528-88-0) in DCM (240 mL) was added DAST (22.5 g, 139 mmol) at 0° C., then the mixture was stirred at 25° C. for 12 hrs. On completion, the mixture was quenched with saturated NaHCO₃ (100 mL), then extracted with DCM (200 mL×3). The combined organic layer was dried over anhydrous Na₂SO₄, filtered and concentrated in vacuo to give the title compound (9.50 g, 96% yield) as brown solid. ¹H NMR (400 MHz, DMSO-d₆) δ 7.69 (s, 1H), 7.40-7.13 (m, 1H), 3.95 (s, 3H). LC-MS (ESI⁺) m/z 212.8 (M+H)⁺.

### Synthesis of 5-(Difluoromethyl)-1-methyl-4-(4,4,5,5-tetramethyl-1,3,2-dioxaborolan-2-yl)pyrazole (Intermediate ML)

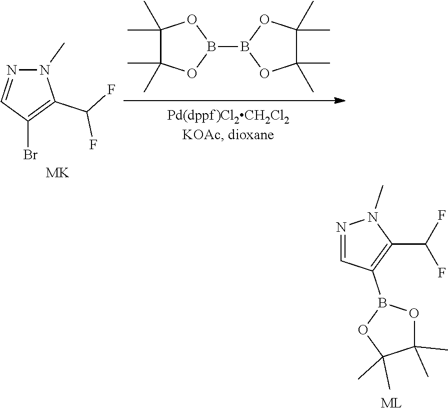

To a solution of 4-bromo-5-(difluoromethyl)-1-methyl-pyrazole (1.00 g, 4.74 mmol, Intermediate MK) and 4,4,5,5-tetramethyl-2-(4,4,5,5-tetramethyl-1,3,2-dioxaborolan-2-yl)-1,3,2-dioxaborolane (2.41 g, 9.48 mmol, CAS #73183-34-3) in dioxane (10 mL) was added cyclopentyl(diphenyl)phosphane; dichloromethane; dichloropalladium; iron (387 mg, 473 μmol) and KOAc (1.40 g, 14.2 mmol). Then the mixture was stirred at 80° C. for 2 hrs under N₂ atmosphere. On completion, the reaction mixture was concentrated and the residue was washed with water (30 mL) and extracted with EtOAc (3×20 mL). The organic phase was dried over Na₂SO₄, filtered, and the filtrate was concentrated in vacuo to give the title compound (400 mg, 32% yield) as black brown liquid. LC-MS (ESI⁺) m/z 258.6 (M+H)⁺.

### Synthesis of 4-[5-(Difluoromethyl)-1-methyl-pyrazol-4-yl]-N-(4-ethylsulfanyl-2-methyl-phenyl)-5-(trifluoromethyl)pyrimidin-2-amine (Intermediate MM)

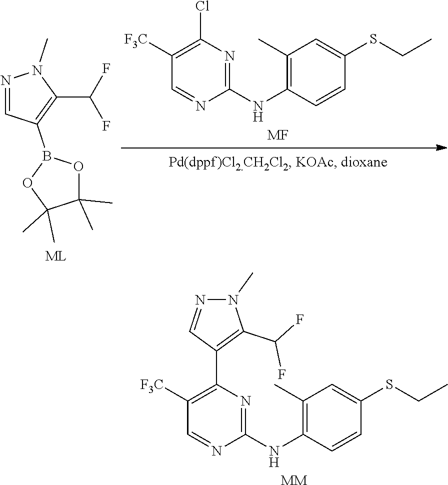

To a solution of 5-(difluoromethyl)-1-methyl-4-(4,4,5,5-tetramethyl-1,3,2-dioxaborolan-2-yl)pyrazole (400 mg, 1.55 mmol, Intermediate ML) and 4-chloro-N-(4-ethylsulfanyl-2-methyl-phenyl)-5-(trifluoromethyl)pyrimidin-2-amine (539 mg, 1.55 mmol, Intermediate MF) in dioxane (5 mL) and H₂O (1 mL) was added Pd(dppf)Cl₂·CH₂Cl₂ (126 mg, 155 μmol) and KOAc (456 mg, 4.65 mmol). Then the mixture was stirred at 80° C. for 2 hrs under N₂ atmosphere. On completion, the mixture was filtered, diluted with water (20 mL) and extracted with EA (15 mL×3). The combined organic layer was dried over anhydrous Na₂SO₄, filtered and concentrated in vacuo to give the residue. The residue was purified by prep-HPLC (column: Phenomenex luna C18 150*25 mm*10 um; mobile phase: [water (FA)-ACN]; B %: 60%-90%, 58 min) to give the title compound (35.0 mg, 5% yield) as yellow solid. LC-MS (ESI⁺) m/z 444.2 (M+H)⁺.

### Synthesis of 3,7-dichloro-8-fluoro-1-isopropyl-1,6-naphthyridin-2-one (Intermediate MN)

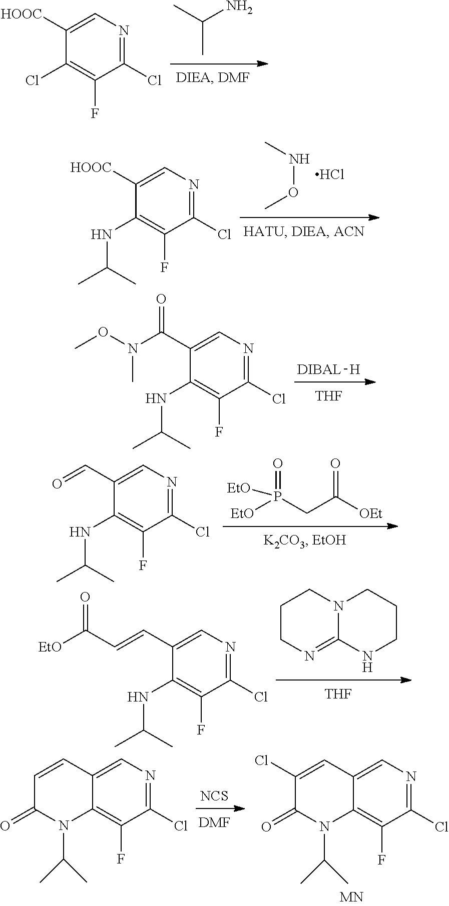

### Step 1—6-Chloro-5-fluoro-4-(isopropylamino)pyridine-3-carboxylic acid

To a solution of 4,6-dichloro-5-fluoro-pyridine-3-carboxylic acid (10 g, 47.6 mmol, CAS #154012-18-7) in DMF (100 mL) was added DIEA (6.15 g, 47.6 mmol) and propan-2-amine (3.38 g, 57.1 mmol, CAS #4432-77-3) at 0° C. The reaction mixture was stirred at 100° C. for 16 hrs. On completion, the reaction mixture concentrated in vacuo to give a residue, which was purified by column chromatography (SiO₂, petroleum ether/ethyl acetate=20/1 to 1/1) to give the title compound (4 g, 36% yield) as a brown solid. ¹H NMR (400 MHz, DMSO-d₆) δ 13.7 (s, 1H), 8.40 (s, 1H), 4.11-4.06 (m, 1H), 3.34-3.32 (m, 3H), 1.23 (s, 3H), 1.21 (s, 3H).

### Step 2—6-Chloro-5-fluoro-4-(isopropylamino)-N-methoxy-N-methyl-pyridine-3-carboxamide

To a solution of 6-chloro-5-fluoro-4-(isopropylamino)pyridine-3-carboxylic acid (3.2 g, 13.7 mmol) and HATU (6.28 g, 16.5 mmol) in ACN (200 mL) was added N-methoxymethanamine (1.41 g, 14.4 mmol, HCl, CAS #1117-97-1) and DIEA (5.33 g, 41.2 mmol). The mixture was stirred at 25° C. for 2 hrs. On completion, the reaction mixture was concentrated in vacuo to give a residue, which was purified by reversed-phase HPLC (0.1% FA condition) to give the title compound (2.4 g, 62% yield) as a yellow oil. ¹H NMR (400 MHz, DMSO-d₆) δ 7.83 (s, 1H), 6.38 (dd, J=2.4, 9.2 Hz, 1H), 3.81-3.69 (m, 1H), 3.55 (s, 3H), 3.26 (s, 3H), 1.15 (d, J=6.4 Hz, 6H). LC-MS (ESI⁺) m/z 277.4 (M+H)⁺.

### Step 3—6-Chloro-5-fluoro-4-(isopropylamino)pyridine-3-carbaldehyde

To a solution of 6-chloro-5-fluoro-4-(isopropylamino)-N-methoxy-N-methyl-pyridine-3-carboxamide (2.4 g, 8.7 mmol) in THF (30 mL) was added diisobutylalumane (1 M, 20.9 mL, CAS #1191-15-7) at 0° C. The mixture was stirred at 25° C. for 2 hrs. The reaction mixture was quenched with Na₂SO₄·10H₂O (5 g) before being filtered. The filtrate was diluted with water (30 mL) and extracted with EtOAc (3×30 mL). The combined organic phase was washed with brine (30 mL), dried over anhydrous Na₂SO₄, and concentrated in vacuo to give the title compound (1.72 g) as a yellow solid. LC-MS (ESI⁺) m/z 216.7 (M+H)⁺.

### Step 4—Ethyl (E)-3-[6-chloro-5-fluoro-4-(isopropylamino)-3-pyridyl]prop-2-enoate

A mixture of 6-chloro-5-fluoro-4-(isopropylamino)pyridine-3-carbaldehyde (1.45 g, 6.69 mmol), ethyl 2-diethoxyphosphorylacetate (4.50 g, 20.1 mmol) and K₂CO₃ (1.85 g, 13.4 mmol) in EtOH (120 mL) was degassed and purged with N₂ three times. Then the mixture was stirred at 25° C. for 16 hrs under N₂ atmosphere. On completion, the reaction mixture was diluted with water (100 mL) and extracted with EtOAc (3×100 mL). The combined organic phase was washed with brine (100 mL), dried over anhydrous Na₂SO₄, and concentrated in vacuo to give a residue. The residue was purified by column chromatography (SiO₂, petroleum ether/ethyl acetate=30/1 to 20/1) to give the title compound (0.98 g, 50% yield) as a yellow oil. LC-MS (ESI⁺) m/z 287.7 (M+H)⁺.

### Step 5—7-Chloro-8-fluoro-1-isopropyl-1,6-naphthyridin-2-one

To a solution of ethyl (E)-3-[6-chloro-5-fluoro-4-(isopropylamino)-3-pyridyl]prop-2-enoate (1.09 g, 3.79 mmol) in THF (80 mL) was added 3,4,6,7,8,9-hexahydro-2H-pyrimido[1,2-a]pyrimidine (2.64 g, 18.97 mmol, CAS #5807-14-7). The mixture was then stirred at 80° C. for 2 hrs. On completion, the reaction mixture concentrated in vacuo to give a residue, which was purified by flash silica gel chromatography (ISCO®; 40 g SepaFlash® Silica Flash Column, Eluent of 0˜15% ethyl acetate/petroleum ether gradient @ 100 mL/min) to give the title compound (545 mg, 59% yield) as a white solid. ¹H NMR (400 MHz, DMSO-d₆) δ 8.32 (s, 1H), 7.62 (dd, J=2.0, 9.2 Hz, 1H), 6.67 (d, J=9.6 Hz, 1H), 5.13 (s, 1H), 1.66 (dd, J=2.0, 6.8 Hz, 6H). LC-MS (ESI⁺) m/z 240.7 (M+H)⁺.

### Step 6—3,7-Dichloro-8-fluoro-1-isopropyl-1,6-naphthyridin-2-one

To a solution of 7-chloro-8-fluoro-1-isopropyl-1,6-naphthyridin-2-one (200 mg, 831 μmol) in DMF (2.5 mL) was added NCS (666 mg, 4.99 mmol) in 2 batches. The mixture was stirred in dark at 80° C. for 16 hrs. On completion, the reaction mixture concentrated in vacuo to give a residue, which was purified by flash silica gel chromatography (ISCO®; 40 g SepaFlash® Silica Flash Column, Eluent of 0˜20% ethyl acetate/petroleum ether gradient @ 80 mL/min) to give the title compound (195 mg, 80% yield) as a white solid. LC-MS (ESI⁺) m/z 274.8 (M+H)⁺.

### Synthesis of 2-Chloro-4-(difluoromethyl)-6-(3-isopropyl-2-methyl-imidazol-4-yl)pyrimidine (Intermediate MO)

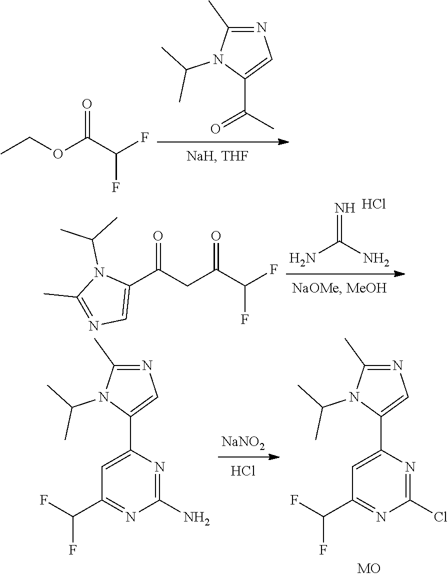

### Step 1—4,4-Difluoro-1-(3-isopropyl-2-methyl-imidazol-4-yl)butane-1,3-dione

To a solution of 1-(3-isopropyl-2-methyl-imidazol-4-yl)ethanone (3.00 g, 18.0 mmol, synthesized via Step 1 of Intermediate DY) in THF (30 mL) was added NaH (1.08 g, 27.0 mmol, 60% dispersion in mineral oil) at 0° C. and the mixture was stirred for 0.5 hrs. Then ethyl 2,2-difluoroacetate (4.48 g, 36.1 mmol, CAS #454-31-9) was added and the mixture was stirred at 25° C. for 1.5 hrs. On completion, the reaction mixture was quenched with H₂O (50 mL) at 25° C., and then extracted with EA (3×100 mL). The combined organic layers were washed with brine (2×100 mL), dried over anhydrous Na₂SO₄ filtered and concentrated in vacuo to give the title compound (4.40 g, 99% yield) as yellow oil. LCMS (ESI⁺) m/z 244.8 (M+H)⁺.

### Step 2—4-(Difluoromethyl)-6-(3-isopropyl-2-methyl-imidazol-4-yl)pyrimidin-2-amine

To a solution of 4,4-difluoro-1-(3-isopropyl-2-methyl-imidazol-4-yl)butane-1,3-dione (4.40 g, 18.0 mmol) in MeOH (50 mL) was added NaOMe (1.95 g, 36.0 mmol) and guanidine; hydrochloride (4.30 g, 45.0 mmol). The mixture was stirred at 40° C. for 16 hrs. On completion, the reaction mixture was concentrated in vacuo to give a residue. The residue was purified by prep-HPLC (column: YMC Triart C18 250*50 mm*7 um; mobile phase: [water(NH3H2O)-ACN]; B %: 23%-53%, 15 min) to give the title compound (160 mg, 3% yield) as a yellow solid. ¹H NMR (400 MHz, DMSO-d₆) δ 7.49 (s, 1H), 7.24-6.88 (m, 3H), 6.81-6.53 (m, 1H), 5.51 (td, J=7.0, 14.0 Hz, 1H), 2.47 (s, 3H), 1.48 (d, J=7.2 Hz, 6H). LCMS (ESI⁺) m/z 268.0 (M+H)⁺.

### Step 3—2-Chloro-4-(difluoromethyl)-6-(3-isopropyl-2-methyl-imidazol-4-yl)pyrimidine

To a solution of 4-(difluoromethyl)-6-(3-isopropyl-2-methyl-imidazol-4-yl)pyrimidin-2-amine (80.0 mg, 299 μmol) in HCl (4 mL) was added NaNO₂ (206 mg, 2.99 mmol) in water slowly drop by drop over a period at 0° C. The mixture was then stirred at 25° C. for 3 hrs. On completion, the reaction mixture was quenched with ION sodium hydroxide aqueous solution adjust to pH=7. Then the resulting solution was filtered to give the filter cake as the title compound (60.0 mg, 69% yield) as yellow oil. LCMS (ESI⁺) m/z 286.8 (M+H)⁺.

### Synthesis of 4-Ethylsulfonyl-2-fluoro-6-methyl-aniline (Intermediate MP)

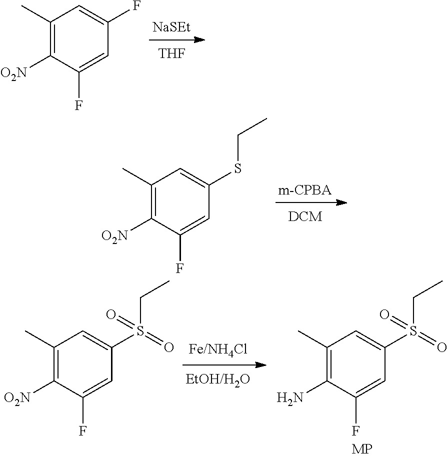

### Step 1—5-Ethylsulfanyl-1-fluoro-3-methyl-2-nitro-benzene

To a solution of 1,5-difluoro-3-methyl-2-nitro-benzene (200 mg, 1.16 mmol, CAS #1616526-80-7) in THF (1 mL) was added NaSEt (77.7 mg, 924 μmol). The mixture was stirred at 40° C. for 1 hr. On completion, the reaction mixture was partitioned between H₂O (20 mL) and DCM (20 mL). The organic phase was separated, washed with brine (20 mL), dried over anhydrous Na₂SO₄, filtered and concentrated in vacuo to give the title compound (1.10 g, 88% yield) as a yellow solid. LCMS (ESI⁺) m/z 216.0 (M+H)⁺.

### Step 2—5-Ethylsulfonyl-1-fluoro-3-methyl-2-nitro-benzene

To a solution of 5-ethylsulfanyl-1-fluoro-3-methyl-2-nitro-benzene (1.00 g, 4.65 mmol) in DCM (10 mL) was added m-CPBA (3.77 g, 18.5 mmol, 85% solution). The mixture was stirred at 25° C. for 1 hr. On completion, the reaction mixture was partitioned between Na₂SO₃ (50 mL) and DCM (50 mL×3). The organic phase was separated, washed with brine (50 mL), dried over anhydrous Na₂SO₄, filtered and concentrated in vacuo to give a residue to give the title compound (1.10 g, 95% yield) as yellow oil. LCMS (ESI⁺) m/z 248.1 (M+H)⁺.

### Step 3—4-Ethylsulfonyl-2-fluoro-6-methyl-aniline

To a solution of 5-ethylsulfonyl-1-fluoro-3-methyl-2-nitro-benzene (1.10 g, 4.45 mmol) in a mixture solution of EtOH (7 mL) and H₂O (7 mL) was added Fe (1.24 g, 22.2 mmol) and NH₄Cl (2.38 g, 44.5 mmol). The mixture was stirred at 80° C. for 2 hrs. On completion, the reaction mixture was filtered and concentrated in vacuo to give a residue. Then the residue was partitioned between EA (50 ml×3) and water (50 ml). The organic layer was collected, washed with brine (30 ml), dried over anhydrous sodium sulfate, and concentrated in vacuo to give a residue. The residue was purified by column chromatography (SiO₂, Petroleum ether/Ethyl acetate=10/1 to 1/1) to give the title compound (70 mg, 7% yield) as yellow oil. ¹H NMR (400 MHz, DMSO-d₆) δ 7.33-7.28 (m, 2H), 5.92 (s, 2H), 3.15 (q, J=7.2 Hz, 2H), 2.18 (s, 3H), 1.06 (t, J=7.2 Hz, 3H). LCMS (ESI⁺) m/z 217.8 (M+H)⁺.

### Synthesis of 3,7-dichloro-1-isopropyl-1,6-naphthyridin-2-one (Intermediate MQ)

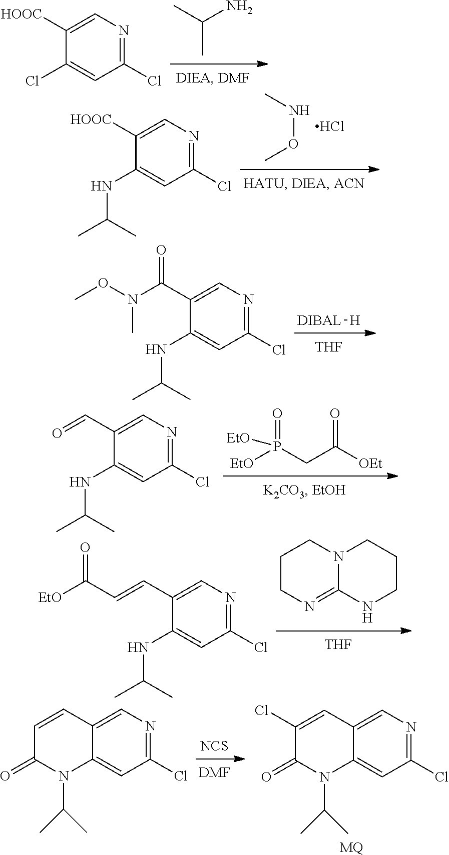

### Step 1—6-Chloro-4-(isopropylamino)pyridine-3-carboxylic acid

To a solution of 4,6-dichloropyridine-3-carboxylic acid (10 g, 52.1 mmol, CAS #73027-79-9) in DMF (100 mL) was added DIEA (6.73 g, 52.1 mmol) and propan-2-amine (3.69 g, 62.5 mmol, CAS #4432-77-3) at 0° C. The reaction mixture was stirred at 100° C. for 12 hrs. On completion, the reaction mixture concentrated in vacuo to give a residue, which was purified reverse phase (0.1% FA condition) to give the title compound (3.5 g, 31% yield) as a white solid. ¹H NMR (400 MHz, DMSO-d₆) δ 13.5 (s, 1H), 8.50 (s, 1H), 8.20-8.18 (m, 1H), 6.79 (s, 1H), 3.85-3.80 (m, 1H), 1.20 (s, 3H), 1.18 (s, 3H).

### Step 2—6-Chloro-4-(isopropylamino)-N-methoxy-N-methyl-pyridine-3-carboxamide

To a solution of 6-chloro-5-fluoro-4-(isopropylamino)pyridine-3-carboxylic acid (2 g, 9.32 mmol) and HATU (4.25 g, 11.2 mmol) in ACN (60 mL) was added N-methoxymethanamine (954 mg, 9.78 mmol) and DIEA (3.61 g, 27.9 mmol), and the mixture was stirred at 25° C. for 2 hrs. On completion, the reaction mixture was concentrated in vacuo to give a residue, which was purified by reversed-phase HPLC (0.1% FA condition) to give the title compound (1.18 g, 49% yield) as a colorless oil. LC-MS (ESI⁺) m/z 257.9 (M+H)⁺.

### Step 3—6-Chloro-4-(isopropylamino)pyridine-3-carbaldehyde

To a solution of 6-chloro-5-fluoro-4-(isopropylamino)-N-methoxy-N-methyl-pyridine-3-carboxamide (1.18 g, 4.58 mmol) in THF (15 mL) was added diisobutylalumane (1 M, 13.74 mL) at −78° C. The mixture was stirred at 0° C. for 2 hrs. The reaction mixture was with Na₂SO₄·10H₂O (5 g) before being filtered. The filtrate was diluted with water (30 mL) and extracted with EtOAc (3×30 mL). The combined organic phase was washed with brine (30 mL), dried over anhydrous Na₂SO₄, and concentrated in vacuo to give the title compound (930 mg) as a yellow oil. LC-MS (ESI⁺) m/z 198.8 (M+H)⁺.

### Step 4—Ethyl (E)-3-[6-chloro-4-(isopropylamino)-3-pyridyl]prop-2-enoate

A mixture of 6-chloro-5-fluoro-4-(isopropylamino)pyridine-3-carbaldehyde (930 mg, 4.68 mmol), ethyl 2-diethoxyphosphorylacetate (4.20 g, 18.7 mmol), and K₂CO₃ (1.62 g, 11.7 mmol) in EtOH (90 mL) was degassed and purged with N₂ three times. Then the mixture was stirred at 25° C. for 16 hrs under N₂ atmosphere. On completion, the reaction mixture was diluted with water (100 mL) and extracted with EtOAc (3×100 mL). The combined organic phase was washed with brine (100 mL), dried over anhydrous Na₂SO₄, and concentrated in vacuo to give a residue. The residue was purified by column chromatography (SiO₂, petroleum ether/ethyl acetate=I/O to 2/1) to give the title compound (350 mg, 14% yield) as a yellow oil. ¹H NMR (400 MHz, CDCl₃) δ 8.11 (s, 1H), 7.56 (d, J=16 Hz, 1H), 6.59 (s, 1H), 6.52 (s, 1H), 6.35 (d, J=16 Hz, 1H), 4.28 (q, J=7.2 Hz, 2H), 3.74-3.70 (m, 1H), 1.35 (t, J=7.2 Hz, 3H), 1.32-1.30 (m, 6H).

### Step 5—7-Chloro-1-isopropyl-1,6-naphthyridin-2-one

To a solution of ethyl (E)-3-[6-chloro-5-fluoro-4-(isopropylamino)-3-pyridyl]prop-2-enoate (350 mg, 1.30 mmol) in THF (2.5 mL) was added 3,4,6,7,8,9-hexahydro-2H-pyrimido[1,2-a]pyrimidine (906 mg, 6.51 mmol). The mixture was then stirred at 80° C. for 1 hr. On completion, the mixture was added to water (10 mL) and extracted with ethyl acetate (5 mL×3). The combined organic phase was dried over anhydrous sodium sulfate, filtered and concentrated in vacuo to give a residue, which was purified by column chromatography (SiO₂, petroleum ether/ethyl acetate=1/0 to 2/1) to give the title compound (150 mg, 51% yield) as a white solid. ¹H NMR (400 MHz, CDCl₃) δ 8.53 (s, 1H), 7.64 (d, J=9.6 Hz, 1H), 7.42 (s, 1H), 6.67 (d, J=9.6 Hz, 1H), 5.82-4.77 (m, 1H), 1.64 (d, J=7.2 Hz, 6H). LC-MS (ESI⁺) m/z 222.8 (M+H)⁺.

### Step 6—3,7-Dichloro-1-isopropyl-1,6-naphthyridin-2-one

To a solution of 7-chloro-8-fluoro-1-isopropyl-1,6-naphthyridin-2-one (300 mg, 1.34 mmol) in DMF (4 mL) was added NCS (1.08 g, 8.08 mmol). The mixture was stirred in dark at 80° C. for 24 hrs. On completion, the reaction mixture concentrated in vacuo to give a residue, which was purified by flash silica gel chromatography (ISCO®; 40 g SepaFlash® Silica Flash Column, Eluent of 0˜20% ethyl acetate/petroleum ether gradient @ 80 mL/min) to give the title compound (135 mg, 73% yield) as a white solid. LC-MS (ESI⁺) m/z 256.7 (M+H)⁺.

### Synthesis of 2-Methyl-4-(methylsulfanylmethyl)aniline (Intermediate MR)

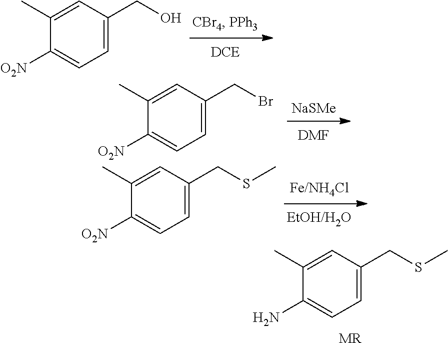

### Step 1—4-(Bromomethyl)-2-methyl-1-nitro-benzene

A mixture of (3-methyl-4-nitro-phenyl)methanol (5.00 g, 29.9 mmol, CAS #80866-75-7), PPh₃ (11.7 g, 44.8 mmol) in DCM (50 mL) was degassed and purged with N₂ three times. Then CBr₄ (14.8 g, 44.8 mmol) was added in DCM (50 mL) and then the mixture was stirred at 25° C. for 16 hrs under N₂ atmosphere. On completion, the reaction mixture was concentrated in vacuo to give a residue. The residue was purified by column chromatography (SiO₂, PE/EtOAc=50/1 to 10/1) to give the title compound (6.50 g, 94% yield) as yellow oil. ¹H NMR (400 MHz, CDCl₃) δ 7.97 (d, J=9.2 Hz, 1H), 7.39-7.35 (m, 2H), 4.47 (s, 2H), 2.61 (s, 3H).

### Step 2−2-Methyl-4-(methylsulfanylmethyl)-1-nitro-benzene

To a solution of 4-(bromomethyl)-2-methyl-1-nitro-benzene (6.50 g, 28.2 mmol) in DMF (70 mL) at 0° C. was added NaSMe (2.22 g, 31.6 mmol). The mixture was stirred at 25° C. for 16 hrs. On completion, the reaction mixture was quenched with H₂O (120 mL) at 25° C., and then extracted with EtOAc (3×100 mL). The combined organic layers were washed with brine (3×100 mL), dried over anhydrous Na₂SO₄ filtered and concentrated in vacuo to give the title compound (5.50 g, 98% yield) as yellow oil. LCMS (ESI⁺) m/z 198.2 (M+H)⁺.

### Step 3—2-Methyl-4-(methylsulfanylmethyl)aniline

To a solution of 2-methyl-4-(methylsulfanylmethyl)-1-nitro-benzene (5.10 g, 25.8 mmol) in EtOH (25 mL) and H₂O (25 mL) was added Fe (7.22 g, 129 mmol) and NH₄Cl (13.8 g, 258 mmol). The mixture was stirred at 80° C. for 2 hrs. On completion, the reaction mixture was filtered and the filtrate was partitioned between H₂O (100 mL) and EtOAc (100 mL). The organic phase was separated, washed with brine 150 mL (50 mL×3), dried over anhydrous Na₂SO₄ filtered and concentrated in vacuo to give the title compound (1.16 g 26% yield) as yellow oil. ¹H NMR (400 MHz, DMSO-d₆) δ 6.88-6.76 (m, 2H), 6.52 (d, J=8.0 Hz, 1H), 4.74 (br s, 2H), 3.50 (s, 2H), 2.04-1.89 (m, 6H).

### Synthesis of 6-Chloro-8-isopropyl-2-[2-methyl-4-(methylsulfanylmethyl)anilino]pyrido[2,3-d]pyrimidin-7-one (Intermediate MS)

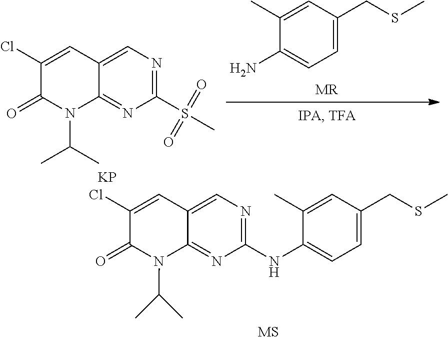

6-chloro-8-isopropyl-2-methylsulfonyl-pyrido[2,3-d]pyrimidin-7-one (400 mg, 1.33 mmol, Intermediate KP), 2-methyl-4-(methylsulfanylmethyl)aniline (554 mg, 3.31 mmol, Intermediate MR) and TFA (1.51 g, 13.2 mmol) were placed in a microwave tube in IPA (6 mL). The sealed tube was heated at 120° C. for 2 hrs under microwave. On completion, the reaction mixture was partitioned between H₂O (20 mL) and EtOAc (20 mL). The organic phase was separated, washed with brine 30 mL (10 mL×3), dried over anhydrous Na₂SO₄, filtered and concentrated in vacuo to give a residue. The residue was purified by prep-HPLC (column: Phenomenex luna C18 250*50 mm*15 um; mobile phase: [water(FA)-ACN]; B %: 55%-75%, 17 min) to give the title compound (350 mg, 67% yield) as a yellow solid. LCMS (ESI⁺) m/z 389.1 (M+H)⁺.

### Synthesis of N-(4-Benzylsulfanyl-2-methyl-phenyl)-4-chloro-5-(trifluoromethyl)pyrimidin-2-amine (Intermediate EA)

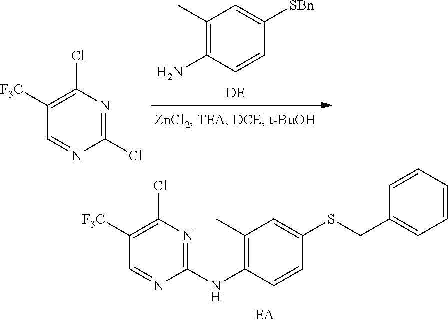

To a solution of 2,4-dichloro-5-(trifluoromethyl)pyrimidine (500 mg, 2.30 mmol, CAS #3932-97-6) in mixture solvent of DCE (6 mL) and t-BuOH (6 mL) was added ZnCl₂ (1 M, 2.77 mL) at 0° C. After 1 hour, a solution of 4-benzylsulfanyl-2-methyl-aniline (528 mg, 2.30 mmol, Intermediate DE) and TEA (256 mg, 2.5 mmol) in mixture solvent of DCE (3 mL) and t-BuOH (3 mL) was added dropwise into the above solution. The mixture was then stirred at 25° C. for 16 hrs. On completion, the mixture was diluted with H₂O (20 mL) and extracted with EA (20 mL×3). The combined organic layers were washed with saturated NaCl (10 mL), dried over anhydrous Na₂SO₄, filtered and concentrated in vacuo to give a residue. The residue was purified by column chromatography (SiO₂, PE:EA=50:1 to 20:1) to give the title compound (600 mg, 63% yield) as a yellow solid. ¹H NMR (400 MHz, DMSO-d₆) δ 10.04 (s, 1H), 8.66-8.60 (m, 1H), 7.40-7.35 (m, 2H), 7.31-7.28 (m, 2H), 7.28-7.21 (m, 3H), 7.20-7.16 (m, 1H), 4.24 (s, 2H), 2.15 (s, 3H). LC-MS (ESI⁺) m/z 410.0 (M+H)⁺.

### Synthesis of 4-[[4-[(3S)-3-hydroxy-3-methyl-1-piperidyl]-5-(trifluoromethyl)pyrimidin-2-yl]amino]-3-methyl-benzenesulfonyl chloride (Intermediate OA)

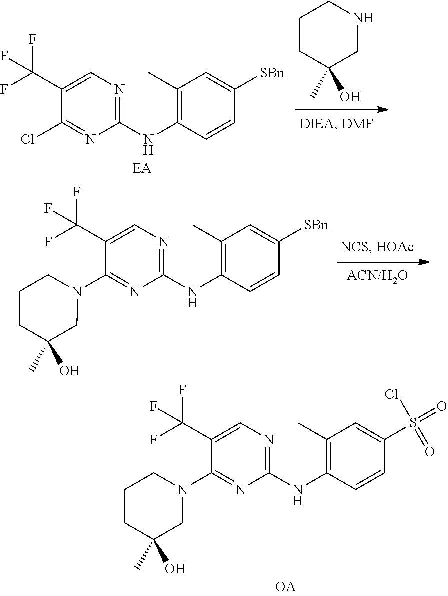

### Step 1—(3S)-1-[2-(4-benzylsulfanyl-2-methyl-anilino)-5-(trifluoromethyl)pyrimidin-4-yl]-3-methyl-piperidin-3-ol

To a mixture of N-(4-benzylsulfanyl-2-methyl-phenyl)-4-chloro-5-(trifluoromethyl)pyrimidin-2-amine (500 mg, 1.22 mmol, Intermediate EA) and (3S)-3-methylpiperidin-3-ol (140 mg, 1.22 mmol, CAS #1200132-32-6) in DMF (5 mL) was added DIEA (236 mg, 1.83 mmol). The reaction mixture was then stirred at 25° C. for 3 hrs. On completion, the residue was diluted with water (10 mL), then the residue was extracted with EA (30 mL×3). The combined organic layers were dried over Na₂SO₄, filtered and filtrate was concentrated in vacuo. The residue was purified by reverse phase (0.1% FA condition) to give the title compound (100 mg, 16% yield) as white solid. ¹H NMR (400 MHz, DMSO-d₆) δ 8.87 (s, 1H), 8.26 (s, 1H), 7.45-7.37 (m, 1H), 7.35-7.21 (m, 6H), 7.13 (d, J=8.8 Hz, 1H), 5.75 (s, 1H), 4.43 (s, 1H), 4.19 (s, 2H), 3.56-3.46 (m, 1H), 3.39-3.30 (m, 2H), 2.17 (s, 3H), 1.78-1.68 (m, 1H), 1.61-1.50 (m, 2H), 1.42 (d, J=2.4 Hz, 1H), 1.02 (s, 3H); LC-MS (ESI⁺) m/z 489.6 (M+H).

### Step 2—4-[[4-[(3S)-3-hydroxy-3-methyl-1-piperidyl]-5-(trifluoromethyl)pyrimidin-2-yl]amino]-3-methyl-benzenesulfonyl chloride

To a solution of (3S)-1-[2-(4-benzylsulfanyl-2-methyl-anilino)-5-(trifluoromethyl) pyrimidin-4-yl]-3-methyl-piperidin-3-ol (90.0 mg, 184 μmol) in ACN (1 mL), HOAc (0.1 mL) and H₂O (0.01 mL) was added NCS (73.7 mg, 552 μmol) in the dark. The reaction mixture was then stirred at 25° C. for 0.5 hr. On completion, the reaction was quenched with water (5 mL), then the mixture was extracted with EA (10 mL×3). The combined organic layers were dried over Na₂SO₄, filtered and filtrate was concentrated in vacuo to give the title compound (60.0 mg, 70% yield) as white oil. LC-MS (ESI⁺) m/z 465.2 (M+H).

### Example 2: Synthesis of Compounds of the Invention (Method 8)

### Synthesis of 2′-(4-Benzylsulfonyl-2-methyl-anilino)-7′-cyclopentyl-spiro[cyclopropane-1,5′-pyrrolo[2,3-d] pyrimidine]-6′-one (I-25)(Method 8)

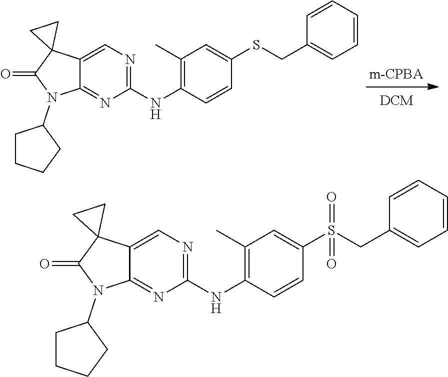 [SMILES: Cc1cc(SCc2ccccc2)ccc1Nc1ncc2c(n1)N(C1CCCC1)C(=O)C21CC1 ; Cc1cc(S(=O)(=O)Cc2ccccc2)ccc1Nc1ncc2c(n1)N(C1CCCC1)C(=O)C21CC1]

To a solution of 2′-(4-benzylsulfanyl-2-methyl-anilino)-7′-cyclopentyl-spiro[cyclopropane-1,5′-pyrrolo [2,3-d]pyrimidine]-6′-one (30.0 mg, 65.7 μmol, synthesized via Step 1 of Intermediate HC) in DCM (1 mL) was added m-CPBA (40.0 mg, 197 μmol, 85% solution). The mixture was stirred at 40° C. for 1 hr. On completion, the reaction mixture was quenched with H₂O (10 mL) and Na₂S₂O₄ aq. (10 mL) at 25° C., and then extracted with EA (3×10 mL). The combined organic layers were washed with brine (2×10 mL), dried over Na₂SO₄, filtered and concentrated in vacuo to give a residue. Then the residue was purified by prep-HPLC (column: Phenomenex C18 150*25 mm*10 um; mobile phase: [water(NH₄HCO₃)-ACN]; B %: 44%-74%, 8 min) to give the title compound (3.93 mg, 12% yield) as a white solid. LC-MS (ESI⁺) m/z 498.1 (M+1)*; ¹H NMR (400 MHz, DMSO-d₆) δ 8.83 (s, 1H), 8.00-7.91 (m, 2H), 7.54 (d, J=0.8 Hz, 1H), 7.45 (d, J=8.0 Hz, 1H), 7.31-7.29 (m, 3H), 7.19-7.18 (m, 2H), 4.72 (t, J=8.4 Hz, 1H), 4.60 (s, 2H), 2.31 (s, 3H), 2.12-2.04 (m, 2H), 1.85-1.75 (m, 4H), 1.69-1.68 (m, 2H), 1.53-1.52 (m, 4H).

TABLE 2
Compounds synthesized via Method 8, the oxidation of
the corresponding sulfanes.

| | | LCMS | |
|---|---|---|---|
| | | (ESI+) | |
| | | m/z | ¹H NMR |
| I-#^{a} | Sulfane | (M + H)⁺ | (400 MHz, DMSO-d₆) δ |
| I-24 | LL | 447.0 | 9.71 (s, 1H), 8.77 (s, 1H), 8.19 (s, 1H), |
| | | | 7.82-7.77 (m, 2H), 7.73-7.69 (m, 1H), |
| | | | 5.74 (q, J = 8.4 Hz, 1H), 3.30-3.24 (m, |
| | | | 2H), 2.36 (s, 3H), 2.16-2.06 (m, 2H), |
| | | | 1.75-1.66 (m, 4H), 1.51-1.43 (m, 2H), |
| | | | 1.12 (t, J = 7.2 Hz, 3H) |
| I-30 | LR | 421.1 | 9.69 (s, 1H), 8.76 (s, 1H), 8.18 (s, 1H), |
| | | | 7.85 (d, J = 8.4 Hz, 1H), 7.78 (d, J = 1.6 Hz, |
| | | | 1H), 7.71 (dd, J = 2.0, 8.4 Hz, 1H), |
| | | | 5.75-5.53 (m, 1H), 3.31-3.25 (m, 2H), |
| | | | 2.37 (s, 3H), 1.42 (d, J = 6.8 Hz, 6H), |
| | | | 1.14-1.05 (m, 3H) |
| I-31 | LT | 437.1 | 9.82 (s, 1H), 8.90 (s, 1H), 8.16 (s, 1H), |
| | | | 7.83 (d, J = 8.0 Hz, 1H), 7.79 (d, J = 1.6 |
| | | | Hz, 1H), 7.72 (dd, J = 2.0, 8.4 Hz, 1H), |
| | | | 7.06-6.75 (m, 1H), 5.64-5.49 (m, 1H), |
| | | | 3.29-3.26 (m, 2H), 2.37 (s, 3H), 1.40 |
| | | | (d, J = 6.4 Hz, 6H), 1.11 (t, J = 7.2 Hz, 3H) |
| I-17b | LR | 404.9 | 9.62 (s, 1 H) 8.72 (s, 1 H) 8.14 (s, 1 H) |
| | | | 7.66 (d, J = 8.4 Hz, 1 H) 7.53 (s, 1 H) |
| | | | 7.48-7.46 (m, 1 H) 5.64-5.51 (m, 1 H) |
| | | | 3.00-2.97 (m, 1 H) 2.80-2.76 (m, 1 H) 2.31 |
| | | | (s, 3 H) 1.37 (d, J = 4.4 Hz, 6 H) 1.03 |
| | | | (t, J = 7.2 Hz, 3 H) |
| I-34 | LV | 440.8 | 9.83 (s, 1H), 8.80 (s, 1H), 8.21 (s, 1H), |
| | | | 8.09 (d, J = 8.0 Hz, 1H), 8.01 (d, J = 2.0 |
| | | | Hz, 1H), 7.88 (dd, J = 2.0, 8.4 Hz, 1H), |
| | | | 5.73-5.54 (m, 1H), 3.39 (q, J = 7.2 Hz, |
| | | | 2H), 1.43 (d, J = 6.8 Hz, 6H), 1.13 |
| | | | (t, J = 7.2 Hz, 3H) |
| I-32 | LZ | 411.9 | 10.25-9.82 (m, 1H), 8.84 (s, 1H), 8.58 |
| | | | (s, 1H), 7.81 (s, 1H), 7.79-7.71 (m, 2H), |
| | | | 5.67-5.37 (m, 1H), 3.31-3.26 (m, 2H), 2.36 |
| | | | (s, 3H), 1.38 (d, J = 2.8 Hz, 6H), 1.10 |
| | | | (t, J = 7.6 Hz, 3H) |
| I-12b | LL | 430.9 | 9.64 (s, 1H), 8.73 (s, 1H), 8.15 (s, 1H), |
| | | | 7.66-7.60 (m, 1H), 7.54 (d, J = 1.6 Hz, |
| | | | 1H), 7.47 (d, J = 1.6, 8.0 Hz, 1H), 5.76-5.62 |
| | | | (m, 1H), 3.06-2.96 (m, 1H), 2.82-2.71 |
| | | | (m, 1H), 2.34-2.28 (m, 3H), 2.15-2.04 |
| | | | (m, 2H), 1.74-1.54 (m, 4H), 1.42-1.36 |
| | | | (m, 2H), 1.05 (t, J = 7.3 Hz, 3H) |
| I-14b | LT | 421.1 | .93 (s, 1H), 8.03 (s, 1H), 7.30 (s, 1H), 6.81 |
| | | | (d, J = 8.0 Hz, 1H), 6.71 (s, 1H), 6.65 (dd, |
| | | | J = 1.6, 8.0 Hz, 1H), 6.06 (t, J = 55.2 Hz, |
| | | | 1H), 4.82 - 4.54 (m, 1H), 2.29-2.09 (m, |
| | | | 1H), 2.04-1.86 (m, 1H), 1.48 (s, 3H), 0.51 |
| | | | (s, 6H), 0.19 (t, J = 7.2 Hz, 3H) |
| I-44 | MG | 468.1 | 9.55 (s, 1H), 8.70 (s, 1H), 7.95 (d, J = 8.4 |
| | | | Hz, 1H), 7.80 (s, 1H), 7.75 (s, 1H), 7.71 |
| | | | (dd, J = 2.0, 8.4 Hz, 1H), 4.51-4.37 (m, |
| | | | 1H), 3.33-3.23 (m, 2H), 2.37 (s, 6H), 1.39 |
| | | | (d, J = 6.4 Hz, 6H), 1.13-1.13 (m, 1H), |
| | | | 1.12 (t, J = 7.6 Hz, 2H) |
| I-45 | MM | 476.3 | 9.91 (s, 1H), 8.83 (s, 1H), 7.83-7.72 (m, |
| | | | 3H), 7.66 (dd, J = 2.0, 8.4 Hz, 1H), 7.44-7.13 |
| | | | (m, 1H), 4.00 (s, 3H), 3.29-3.24 (m, |
| | | | 2H), 2.35 (s, 3H), 1.11 (t, J = 7.6 Hz, 3H) |
| I-40 | MJ | 457.9 | 9.73 (s, 1H), 8.66 (s, 1H), 7.84 (d, J = 8.4 |
| | | | Hz, 1H), 7.76 (d, J = 2.0 Hz, 1H), 7.66 (dd, |
| | | | J = 2.0, 8.4 Hz, 1H), 4.39 (s, 1H), 3.29-3.23 |
| | | | (m, 2H), 2.35 (s, 3H), 1.73-1.60 (m, |
| | | | 6H), 1.49-1.37 (m, 2H), 1.08 (s, 6H) |
| I-43 | MI | 457.9 | 9.69 (b, 1H), 8.64 (s, 1H), 7.90 (d, J = 8.4 |
| | | | Hz, 1H), 7.75 (d, J = 1.6 Hz, 1H), 7.66 (dd, |
| | | | J = 2.0, 8.4 Hz, 1H), 4.02 (s, 1H), 3.29-3.24 |
| | | | (m, 2H), 2.37 (s, 3H), 2.09 (q, J = 12.0 |
| | | | Hz, 2H), 1.64 (d, J = 12.8 Hz, 2H), 1.46 (d, |
| | | | J = 11.2 Hz, 2H), 1.36 (t, J = 3.2, 13.2 Hz, |
| | | | 2H), 1.13 (s, 3H), 1.13-1.09 (m, 3H) |
| I-197n | MS | 420.8 | 9.51 (s, 1H), 8.68 (s, 1H), 8.13 (s, 1H), |
| | | | 7.45 (br d, J = 8.0 Hz, 1H), 7.31-7.24 (m, |
| | | | 2H), 5.71-5.44 (m, 1H), 4.45 (s, 2H), 2.89 |
| | | | (s, 3H), 2.23 (s, 3H), 1.37 (br d, J = 4.4 Hz,6H) |

^{a}Reaction was run from 0-40° C. for 1-3 hrs. The final compounds were purified under standard techniques including prep-HPLC and chromatography.
^{b}For the mono-oxidation, only 0.5 eq. of m-CPBA was employed and the reaction was run at 25-40° C. for 1 hr.

### Example 3: Synthesis of Compounds of the Invention (Method 9)

### Synthesis of 6-Chloro-8-cyclopentyl-2-[2-methyl-4-[(1-methyl-4-piperidyl)sulfonyl]anilino]pyrido[2,3-d]pyrimidin-7-one (I-26)

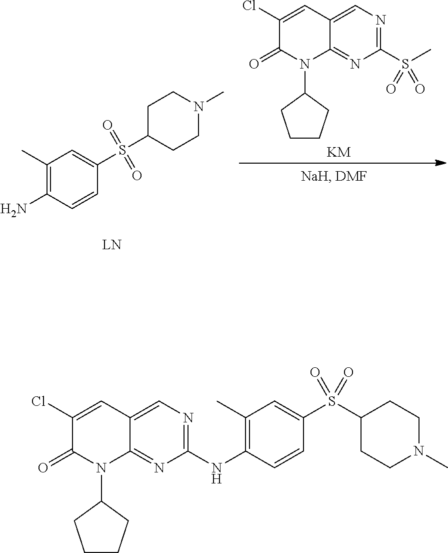

A mixture of 2-methyl-4-[(1-methyl-4-piperidyl)sulfonyl]aniline (10 mg, 37 μmol, Intermediate LN), and NaH (1.79 mg, 74 μmol) in DMF (0.25 mL) was degassed and purged with N₂ three times and the mixture was stirred at 25° C. for 0.5 hour under N₂ atmosphere. Then the 6-chloro-8-cyclopentyl-2-methylsulfonyl-pyrido[2,3-d]pyrimidin-7-one (12.2 mg, 37 μmol, Intermediate KM) was dissolved in DMF (0.25 mL) and added into the mixture and the mixture was stirred for 2 hrs at 25° C. under N₂ atmosphere. On completion, the reaction mixture was quenched with H₂O (0.01 mL) and 15% NaOH (0.01 mL) at 25° C., and then diluted with H₂O (2 mL) and extracted with EA (3×10 mL). The combined organic layers were washed with brine (2×10 mL), dried over with anhydrous Na₂SO₄, filtered and concentrated in vacuo to give a residue. The crude product was purified by prep-HPLC (column: Phenomenex luna C18 150*25 mm*10 um; mobile phase: [water(FA)-ACN]; B %: 17%-47%, 9 min) to afford the title compound (1.12 mg, 6% yield) as a yellow solid. ¹H NMR (400 MHz, DMSO-d₆) δ 9.71 (s, 1H), 8.75 (s, 1H), 8.17 (s, 1H), 7.81-7.77 (m, 1H), 7.71 (d, J=2.0 Hz, 1H), 7.64 (dd, J=2.0, 8.4 Hz, 1H), 5.78-5.66 (m, 1H), 2.78 (d, J=11.6 Hz, 3H), 2.34 (s, 3H), 2.09 (s, 3H), 1.84-1.75 (m, 5H), 1.67 (s, 5H), 1.57-1.49 (m, 2H), 1.46-1.41 (m, 2H); LC-MS (ESI⁺) m/z 516.1 (M+H)⁺.

TABLE 3
Compounds synthesized via Method 9, the coupling
of the corresponding amines and mesylates.

| | | | LCMS | |
|---|---|---|---|---|
| | | | (ESI+) m/z | ¹H NMR (400 MHz, |
| I-#^{a} | Amine | Mesylate | (M + H)⁺ | DMSO-d₆) δ |
| I-198 | LO | KP | 399.2 | 8.57 (s, 1H), 7.99 (s, 1H), |
| | | | | 7.61 (dd, J = 2.8, 4.8 Hz, |
| | | | | 1H), 5.76-5.69 (m, 1H), 3.88- |
| | | | | 3.71 (m, 1H), 3.02 (s, 1H), |
| | | | | 2.91 (s, 3H), 2.25-2.09 (m, |
| | | | | 4H), 1.58 (d, J = 3.2 Hz, |
| | | | | 1H), 1.55 (d, J = 6.8 Hz, |
| | | | | 6H), 1.53-1.38 (m, 3H) |
| I-35 | LP | HB | 427.1 | 8.91 (s, 1H), 7.99-7.92 (m, |
| | | | | 2H), 7.70 (d, J = 2.0 Hz, 1H), |
| | | | | 7.63 (d, J = 2.0, 8.4 Hz, 1H), |
| | | | | 4.76-4.67 (m, 1H), 3.24 (q, |
| | | | | J = 7.2 Hz, 2H), 2.36 (s, 3H), |
| | | | | 2.13-2.03 (m, 2H), 1.86-1.73 |
| | | | | (m, 4H), 1.68 (q, J = 4.4 Hz, |
| | | | | 2H), 1.56-1.47 (m, 4H), 1.10 |
| | | | | (t, J = 7.2 Hz, 3H) |

^{a}The reaction was run at 25 C for 2-6 hrs. Cs₂CO₃ could also be used as base. The final compounds were purified under standard techniques including prep-HPLC and chromatography.

### Example 4: Synthesis of Compounds of the Invention (Method 10)

### Synthesis of N-(4-ethylsulfonyl-2-methyl-phenyl)-4-(3-isopropyl-2-methyl-imidazol-4-yl) pyrimidin-2-amine (I-29)

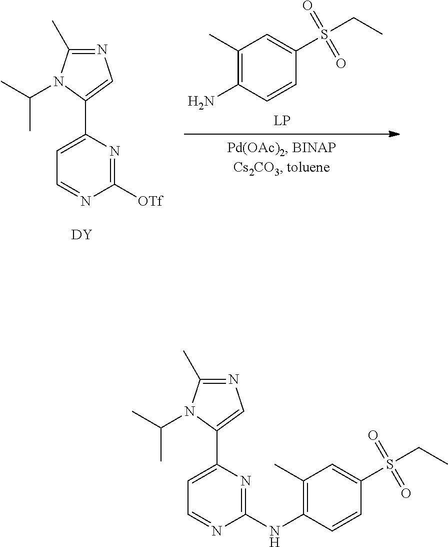

A mixture of [4-(3-isopropyl-2-methyl-imidazol-4-yl)pyrimidin-2-yl]trifluoromethanesulfonate (50.0 mg, 142 μmol, Intermediate DY), 4-ethylsulfonyl-2-methyl-aniline (29.8 mg, 149 μmol, Intermediate LP), Pd(OAc)₂ (3.20 mg, 14.2 μmol), BINAP (8.89 mg, 14.2 μmol) and Cs₂CO₃ (139 mg, 428 μmol) in toluene (5 mL) was degassed and purged with N₂ three times. Then the mixture was stirred at 100° C. for 16 hrs under N₂ atmosphere. The reaction mixture was filtered and concentrated in vacuo to give a residue. The residue was purified by prep-HPLC (column: Phenomenex Luna C18 200*40 mm*10 um; mobile phase: [water(FA)-ACN]; B %: 25%-55%, 10 min) to give the title compound (6.05 mg, 10% yield) as a yellow oil. ¹H NMR (400 MHz, DMSO-d₆) δ 9.14 (s, 1H), 8.45 (d, J=5.2 Hz, 1H), 7.79-7.74 (m, 2H), 7.68 (dd, J=2.0, 8.4 Hz, 1H), 7.64 (s, 1H), 7.12 (d, J=5.2 Hz, 1H), 5.51-5.41 (m, 1H), 3.26 (d, J=7.2 Hz, 2H), 2.50 (s, 3H), 2.34 (s, 3H), 1.27 (d, J=7.2 Hz, 6H), 1.11 (t, J=7.2 Hz, 3H). LCMS (ESI⁺) m/z 399.9 (M+H)⁺.

TABLE 4
Compounds synthesized via Method 10, the coupling of
the corresponding amines and triflates or chlorides.

| | | | LCMS | |
|---|---|---|---|---|
| | | Triflate/ | (ESI+) m/z | ¹H NMR (400 MHz, |
| I-#^{a} | Amine | Chloride | (M + H)⁺ | DMSO-d₆) δ |
| I-38 | LP | MA | 407.8 | 9.25 (s, 1H), 8.46 (s, 1H), |
| | | | | 8.14 (s, 1H), 8.08 (d, J = |
| | | | | 8.4 Hz, 1H), 7.75-7.69 (m, |
| | | | | 2H), 7.39 (s, 1H), 6.99-6.64 |
| | | | | (m, 1H), 3.91 (s, 3H), 3.29- |
| | | | | 3.23 (m, 2H), 2.39 (s, 3H), |
| | | | | 1.13 (t, J = 7.6 Hz, 3H) |
| I-36 | LP | MN | 437.9 | 8.46 (s, 1H), 8.00 (s, 1H), |
| | | | | 7.79 (d, J = 1.6 Hz, 1H), |
| | | | | 7.75-7.70 (m, 1H), 7.65 (s, |
| | | | | 1H), 7.27 (d, J = 2.0 Hz, 1H), |
| | | | | 5.24-5.01 (m, 1H), 3.27 (d, |
| | | | | J = 7.2 Hz, 2H), 2.34 (s, 3H), |
| | | | | 1.63 (dd, J = 1.2, 6.4 Hz, |
| | | | | 6H), 1.13 (t, J = 7.2 Hz, 3H) |
| I-39 | LP | MO | 450.2 | 9.52 (s, 1H), 7.78 (s, 1H), |
| | | | | 7.69 (s, 2H), 7.65 (s, 1H), |
| | | | | 7.32 (s, 1H), 6.97-6.64 (m, |
| | | | | 1H), 5.47 (td, J = 7.1, 14.0 |
| | | | | Hz, 1H), 3.29 (br s, 2H), |
| | | | | 2.43 (s, 3H), 2.33 (s, 3H), |
| | | | | 1.19 (d, J = 7.2 Hz, 6H), |
| | | | | 1.12 (t, J = 7.2 Hz, 3H) |
| I-37 | LP | MQ | 419.9 | 8.78 (s, 1H), 8.53 (s, 1H), |
| | | | | 8.26-8.15 (m, 2H), 7.70 (d, |
| | | | | J = 1.6 Hz, 1H), 7.64 (s, |
| | | | | 1H), 7.29 (s, 1H), 5.57-4.86 |
| | | | | (m, 1H), 3.26-3.20 (m, 2H), |
| | | | | 2.40 (s, 3H), 1.57 (d, J = |
| | | | | 7.2 Hz, 6H), 1.11 (s, 3H) |

^{a}Triflates or chlorides were used for the coupling with the amines. The final compounds were purified under standard techniques including prep-HPLC and chromatography.

### Example 5: Synthesis of Compounds of the Invention (Method 11)

### Synthesis of N-(4-ethylsulfonyl-2-methyl-phenyl)-4-(1-methylpyrazol-4-yl)-5-(trifluoromethyl) pyrimidin-2-amine (I-27)

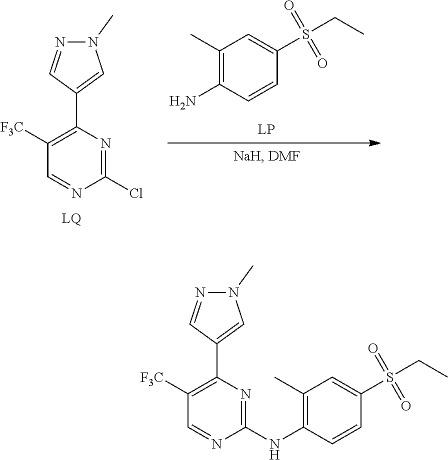 [SMILES: Cn1cc(-c2nc(Cl)ncc2C(F)(F)F)cn1 ; CCS(=O)(=O)c1ccc(N)c(C)c1 ; CCS(=O)(=O)c1ccc(Nc2ncc(C(F)(F)F)c(-c3cnn(C)c3)n2)c(C)c1]

To a solution of 4-ethylsulfonyl-2-methyl-aniline (50.0 mg, 250 μmol, Intermediate LP) in DMF (1 mL) was added NaH (30.1 mg, 752 μmol, 60% dispersion in mineral oil) at 0° C. Then 2-chloro-4-(1-methylpyrazol-4-yl)-5-(trifluoromethyl)pyrimidine (72.4 mg, 276 μmol, Intermediate LQ) was added, then the mixture was stirred at 25° C. for 1 hr under N₂ atmosphere. On completion, the mixture was quenched with water (0.5 mL) and filtered to give the residue. The residue was purified by prep-HPLC (column: Phenomenex luna C18 150*25 mm*10 um; mobile phase: [water (FA)-ACN]; B %: 37%-67%, 10 min) to give the title compound (11.4 mg, 10% yield) as a yellow solid. ¹H NMR (400 MHz, DMSO-d₆) δ 9.68 (s, 1H), 8.73 (s, 1H), 8.22 (s, 1H), 7.94-7.90 (m, 2H), 7.78-7.71 (m, 2H), 3.93 (s, 3H), 3.29-3.25 (m, 2H), 2.38 (s, 3H), 1.16-1.10 (m, 3H). LC-MS (ESI⁺) m/z 426.2 (M+H)⁺.

TABLE 5
Compounds synthesized via Method 11, the coupling of
the corresponding amines and chlorides.

| | | | LCMS | |
|---|---|---|---|---|
| | | | (ESI +) | |
| | | | m/z | |
| I-#^{a} | Amine | Chloride | (M + H)⁺ | ¹H NMR (400 MHZ, DMSO-d₆) δ |
| I-46 | LP | MB | 396.1 | 9.34 (s, 1H), 9.09 (s, 1H), 8.49 (d, |
| | | | | J = 8.4 Hz, 1H), 7.82-7.77 (m, |
| | | | | 1H), 7.76-7.72 (m, 2H), 7.70-7.68 |
| | | | | (m, 1H), 7.37 (t, J = 7.6 Hz, |
| | | | | 1H), 3.86 (q, J = 8.8 Hz, 1H), 3.26 |
| | | | | (q, J = 7.2 Hz, 2H), 2.44 (s, 3H), |
| | | | | 2.12-2.02 (m, 2H), 1.85-1.75 |
| | | | | (m, 2H), 1.73-1.61 (m, 4H), 1.12 |
| | | | | (t, J = 7.6 Hz, 3H) |
| I-28b | MD | LQ | 495.1 | 8.47 (s, 1H), 8.30 (s, 1H), 8.20 (s, |
| | | | | 1H), 7.97 (s, 1H), 7.89 (s, 1H), |
| | | | | 7.85-7.80 (m, 1H), 7.77-7.73 |
| | | | | (m, 1H), 3.95 (s, 3H), 3.30-3.24 |
| | | | | (m, 1H), 2.83 (d, J = 11.6 Hz, 2H), |
| | | | | 2.30 (s, 3H), 2.13 (s, 3H), 1.85-1.82 |
| | | | | (m, 4H), 1.61-1.51 (m, 2H) |
| I-33b | MP | KP | 438.8 | 9.80 (s, 1H), 8.72 (s, 1H), 8.15 (s, |
| | | | | 1H), 7.72 (s, 1H), 7.66 (dd, J = 1.6, |
| | | | | 9.2 Hz, 1H), 5.64-5.32 (m, |
| | | | | 1H), 3.38 (d, J = 7.2 Hz, 2H), 2.35 |
| | | | | (s, 3H), 1.36-1.23 (m, 6H), 1.12 |
| | | | | (t, J = 7.2 Hz, 3H) |

^{a}The coupling was run from 0-25° C. for 1-2 hr. The final compounds were purified under standard techniques including prep-HPLC and chromatography.
^{b}tBuOK use as the base.

### Example 6: Synthesis of Compounds of the Invention (Alternative Methods)

### Synthesis of 6-chloro-8-isopropyl-2-(((1r,4r)-4-((1-methyl-1H-pyrazol-4-yl)sulfonyl)cyclohexyl) amino)pyrido[2,3-d]pyrimidin-7(8H)-one (I-181) and 6-chloro-8-isopropyl-2-(((1s,4s)-4-((1-methyl-1H-pyrazol-4-yl)sulfonyl)cyclohexyl)amino)pyrido[2,3-d]pyrimidin-7(8H)-one (I-182)

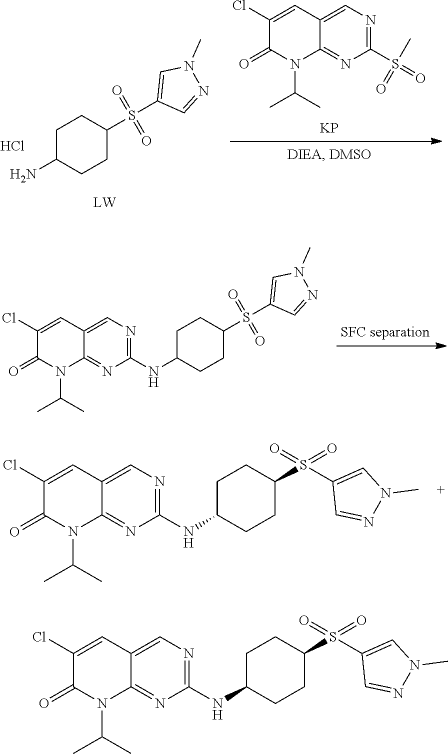

### Step 1—6-Chloro-8-isopropyl-2-[[4-(1-methylpyrazol-4-yl)sulfonylcyclohexyl]amino]pyrido[2,3-d]pyrimidin-7-one

To a mixture of 4-(1-methylpyrazol-4-yl)sulfonylcyclohexanamine (130 mg, 465 μmol, HCl salt, Intermediate LW) in DMSO (4 mL) was added DIEA (120 mg, 929 μmol, 162 uL), 4 Å molecular sieves (50.0 mg, 125 μmol) and 6-chloro-8-isopropyl-2-methylsulfonyl-pyrido[2,3-d]pyrimidin-7-one (140 mg, 465 μmol, Intermediate KP), then the mixture was stirred at 70° C. for 1 hr. On completion, the mixture was filtered to give a clear liquid. The residue was purified by prep-HPLC (column: Phenomenex luna C18 150*25 mm*10 um; mobile phase: [water(FA)-ACN]; B %: 38%-68%, 10 min) to give the title compound (25.0 mg, 11% yield) as a yellow solid. LC-MS (ESI⁺) m/z 464.9 (M+H)⁺.

### Step 2—6-chloro-8-isopropyl-2-(((1r,4r)-4-((1-methyl-1H-pyrazol-4-yl)sulfonyl)cyclohexyl)amino)pyrido[2,3-d]pyrimidin-7(8H)-one and 6-chloro-8-isopropyl-2-(((1s,4s)-4-((1-methyl-1H-pyrazol-4-yl)sulfonyl)cyclohexyl)amino)pyrido[2,3-d]pyrimidin-7(8H)-one

6-chloro-8-isopropyl-2-[[4-(1-methylpyrazol-4-yl)sulfonylcyclohexyl]amino]pyrido[2,3-d]pyrimidin-7-one (25.0 mg, 53.8 μmol) was separated by SFC [column: DAICEL CHIRALPAK AS (250 mm*50 mm, 10 um); mobile phase: [0.1% NH₃H₂O ETOH]; B %: 45%-45%, 40 min] to give 6-chloro-8-isopropyl-2-(((1r,4r)-4-((1-methyl-1H-pyrazol-4-yl)sulfonyl)cyclohexyl)amino)pyrido[2,3-d]pyrimidin-7(8H)-one (7.85 mg, 30.0% yield, FA salt) as a yellow solid (¹H NMR (400 MHz, DMSO-d₆) δ 8.60 (s, 1H), 8.34 (s, 1H), 8.06 (s, 1H), 7.78 (s, 1H), 5.95-5.55 (m, 1H), 4.53-4.33 (m, 1H), 3.90 (s, 3H), 3.61-3.50 (m, 1H), 2.18-2.07 (m, 1H), 1.98-1.88 (m, 1H), 1.76-1.60 (m, 4H), 1.51 (d, J=6.4 Hz, 7H), 1.44-1.31 (m, 2H). LC-MS (ESI⁺) m/z 464.9 (M+H)⁺) and 6-chloro-8-isopropyl-2-(((1s,4s)-4-((1-methyl-1H-pyrazol-4-yl)sulfonyl)cyclohexyl)amino)pyrido[2,3-d]pyrimidin-7(8H)-one (10.8 mg, 34.7% yield, FA salt) as a yellow solid (¹H NMR (400 MHz, DMSO-d₆) δ 8.60 (s, 1H), 8.34 (s, 1H), 8.06 (s, 1H), 7.84-7.73 (m, 1H), 5.76-5.58 (m, 1H), 4.52-4.30 (m, 1H), 3.90 (s, 3H), 3.58-3.49 (m, 1H), 2.18-2.07 (m, 1H), 1.98-1.86 (m, 1H), 1.76-1.62 (m, 4H), 1.51 (d, J=6.4 Hz, 7H), 1.43-1.35 (m, 2H). LC-MS (ESI⁺) m/z 464.9 (M+H)⁺). Absolute stereochemistry of the final products was assigned arbitrarily.

### Synthesis of N-(4-ethylsulfonyl-2-methyl-phenyl)-8-isopropyl-quinazolin-2-amine (I-47)

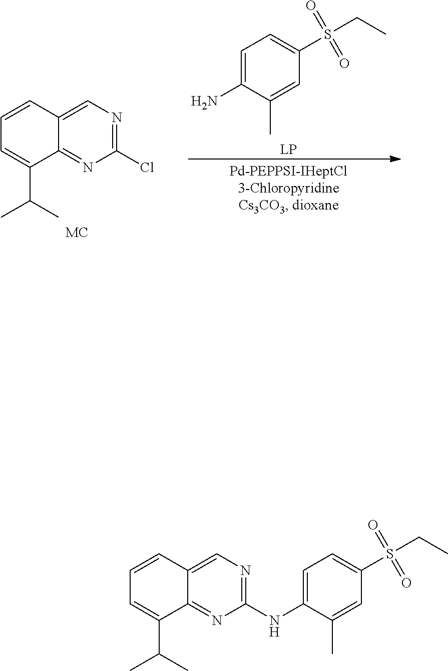 [SMILES: CC(C)c1cccc2cnc(Cl)nc12 ; CCS(=O)(=O)c1ccc(N)c(C)c1 ; CCS(=O)(=O)c1ccc(Nc2ncc3cccc(C(C)C)c3n2)c(C)c1]

To a mixture of 2-chloro-8-isopropyl-quinazoline (100 mg, 483 μmol, Intermediate MC) and 4-ethylsulfonyl-2-methyl-aniline (77.1 mg, 387 μmol, Intermediate LP) in dioxane (10 mL) was added Cs₂CO₃ (472 mg, 1.45 mmol) and 1,3-bis[2,6-bis(1-propylbutyl)phenyl]-4,5-dichloro-2H-imidazol-1-ium-2-ide; 3-chloropyridine; dichloropalladium (47.0 mg, 48.3 μmol). The reaction mixture was stirred at 90° C. for 12 hrs. On completion, the residue was diluted with water (10 mL), then the residue was extracted with EA (3×20 mL). The combined organic layers was dried over Na₂SO₄, filtered and filtrate was concentrated in vacuo. The residue was purified by prep-HPLC (column: Phenomenex luna C18 150*25 mm*10 um; mobile phase: [water(FA)-ACN]; B %: 54%-84%, 10 min) to give the title compound (41.0 mg, 20% yield, FA) as yellow solid. ¹H NMR (400 MHz, DMSO-d₆) δ 9.35 (s, 1H), 9.06 (s, 1H), 8.54 (d, J=8.4 Hz, 1H), 7.81 (dd, J=1.2, 7.6 Hz, 1H), 7.75-7.70 (m, 3H), 7.40 (t, J=7.6 Hz, 1H), 3.91 (td, J=6.8, 13.6 Hz, 1H), 3.30-3.24 (m, 2H), 2.45 (s, 3H), 1.30 (s, 3H), 1.29 (s, 3H), 1.12 (t, J=7.2 Hz, 3H). LC-MS (ESI⁺) m/z 370.0 (M+H)⁺.

### Synthesis of 4-[[4-[(3S)-3-hydroxy-3-methyl-1-piperidyl]-5-(trifluoromethyl)pyrimidin-2-yl]amino]-N,3-dimethyl-benzenesulfonamide (I-204)

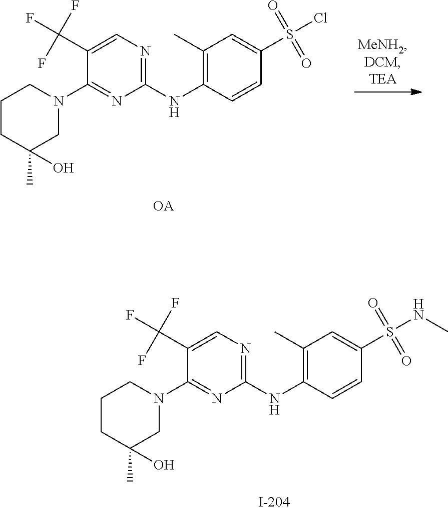 [SMILES: Cc1cc(S(=O)(=O)Cl)ccc1Nc1ncc(C(F)(F)F)c(N2CCC[C@](C)(O)C2)n1 ; CNS(=O)(=O)c1ccc(Nc2ncc(C(F)(F)F)c(N3CCC[C@](C)(O)C3)n2)c(C)c1]

To a solution of methanamine hydrochloride (37.0 mg, 548 μmol, CAS #593-51-1) in DCM (1 mL) was added TEA (127 μL, 914 μmol). Then a solution of 4-[[4-[(3S)-3-hydroxy-3-methyl-1-piperidyl]-5-(trifluoromethyl)pyrimidin-2-yl]amino]-3-methyl-benzenesulfonyl chloride (OA; 85.0 mg, 183 mol, Intermediate OA) in DCM (1 mL) was added and the mixture was stirred at 25° C. for 0.5 hr. On completion, the mixture was diluted with water (50 mL) and extracted with DCM (30 mL×3). The combined organic layers were dried over anhydrous Na₂SO₄, filtered and concentrated in vacuo to give the residue. The residue was purified by prep-HPLC (column: Waters Xbridge 150*25 mm*5 um; mobile phase: [water (NH₄HCO₃)-ACN]; gradient: 34%-64% B over 10 min) to give the title compound (30.4 mg, 36% yield) as white solid. ¹H NMR (400 MHz, DMSO-d₆) δ 9.10 (s, 1H), 8.34 (s, 1H), 7.91-7.85 (m, 1H), 7.61 (d, J=1.6 Hz, 1H), 7.57-7.54 (m, 1H), 7.32-7.29 (m, 1H), 4.46 (s, 1H), 3.65-3.56 (m, 1H), 3.44-3.37 (m, 1H), 3.31-3.29 (m, 1H), 3.28-3.23 (m, 1H), 2.44-2.38 (m, 3H), 2.33 (s, 3H), 1.83-1.72 (m, 1H), 1.60-1.52 (m, 2H), 1.46-1.44 (m, 1H), 1.03 (s, 3H). LC-MS (ESI⁺) m/z 460.0 (M+H)⁺.

### Example 7: CDK2 Inhibition Assay

Inhibition of CDK2 was measured using PhosphoSens® CSox-based Kinetic Assay Format (AssayQuant Technologies) as follows:

### Kinases:

2.5 nM CDK1/CycB1, amino acids 1-297/1-433, Carna BS (cat. #04-102, lot #20CBS-0607B)
1.5 nM CDK2/CycE1, amino acids 1-298/1-410, Carna BS (cat. #04-165, lot #20CBS-0731B)

### Reaction Set Up:

2.5 μl 10× Inhibitor in 10% DMSO (3-fold titration in 10% DMSO for a final DMSO concentration of 1%)
17.5 L Reaction Mix with ATP, DTT, & CSx Substrate
30 minute preincubation at 30° C.
5.0 L Enzyme dilution buffer (EDB) (1×) or Kinase (5× in EDB); Enzyme Dilution Buffer (EDB): 20 mM HEPES, pH 7.5, 0.01% Brij-35, 0.1 mM EGTA, 5% Glycerol, 1.0 mg/ml BSA, 1.0 mM DTT
25 μL Final reaction volume
Reaction was run at 30° C. for 240 minutes

### Reaction Conditions:

54 mM HEPES, pH 7.5
1.5× Km ATP (see ATP TAB)
1.2 mM DTT
0.012% Brij-35
0.52 mM EGTA
1% glycerol
0.2 mg/ml BSA
10 mM MgCl2
15 μM AQT0297

Reactions were run in Corning, low volume 384-well, white flat round bottom polystyrene NBS microplates (Cat. #3824) after sealing using optically-clear adhesive film (TopSealA-Plus plate seal, PerkinElmer [Cat. #6050185])

Table 4 shows the results of CDK2 inhibition. The letter codes for CDK2 inhibition include: A (<100 nM), B (≥100-500 nM), C (>500-1000 nM), and D (>1000 nM).

| | IC50 CDK2-CycE1 |
|---|---|
| | (AssayQuant) Using |
| | PhosphoSens ® CSox- |
| | based Kinetic Assay |
| I-# | Format |
| I-27 | A |
| I-29 | B |
| I-30 | B |
| I-31 | D |
| I-35 | A |
| I-40 | D |
| I-41 | A |
| I-42 | A |
| I-43 | D |
| I-44 | A |
| I-45 | C |
| I-52 | B |
| I-53 | C |
| I-67 | A |
| I-68 | D |
| I-197 | D |
| I-207 | B |
| I-208 | B |
| I-210 | D |
| I-212 | B |
| I-213 | B |
| I-215 | A |
| I-218 | B |
| I-228 | B |
| I-234 | D |
| I-235 | C |
| I-239 | D |
| I-242 | A |
| I-247 | A |
| I-252 | A |
| I-254 | A |
| I-255 | A |
| I-256 | A |
| I-265 | B |
| I-266 | A |
| I-267 | B |
| I-269 | A |
| I-271 | C |
| I-272 | A |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I-a′: 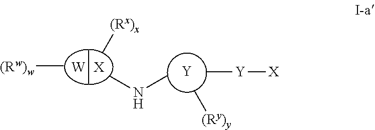
or a pharmaceutically acceptable salt thereof, wherein:
Ring W and Ring X are independently fused rings selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
Y is a covalent bond, —S(O)₂—, —S(O)—, —S(O)(NR)—, —P(O)R—, —P(O)OR—, or 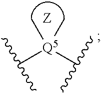
Ring Z is an optionally substituted 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur;
Q⁵ is carbon or sulfur;
X is —NR₂ or an optionally substituted group selected from C₁₋₆ aliphatic, phenyl, a 3 to 12-membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, and —NRS(O)₂R; or
two R^{w} groups attached to the same or adjacent carbon atom are optionally taken together to form a spiro fused or 1,2-fused ring selected from a 3-12 membered saturated or partially unsaturated carbocyclyl and a 3-12 membered saturated or partially unsaturated heterocyclyl having 1-3 heteroatoms independently selected from nitrogen, oxygen, and sulfur;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form a 4-7 membered saturated, partially unsaturated, or heteroaryl ring having 0-3 heteroatoms, in addition to the atom or atoms to which they are attached, independently selected from nitrogen, oxygen, and sulfur; and
w, x, and y are independently 0, 1, 2, 3, or 4.

2. The compound of claim 1, wherein said compound is a compound of any of the following formulae: 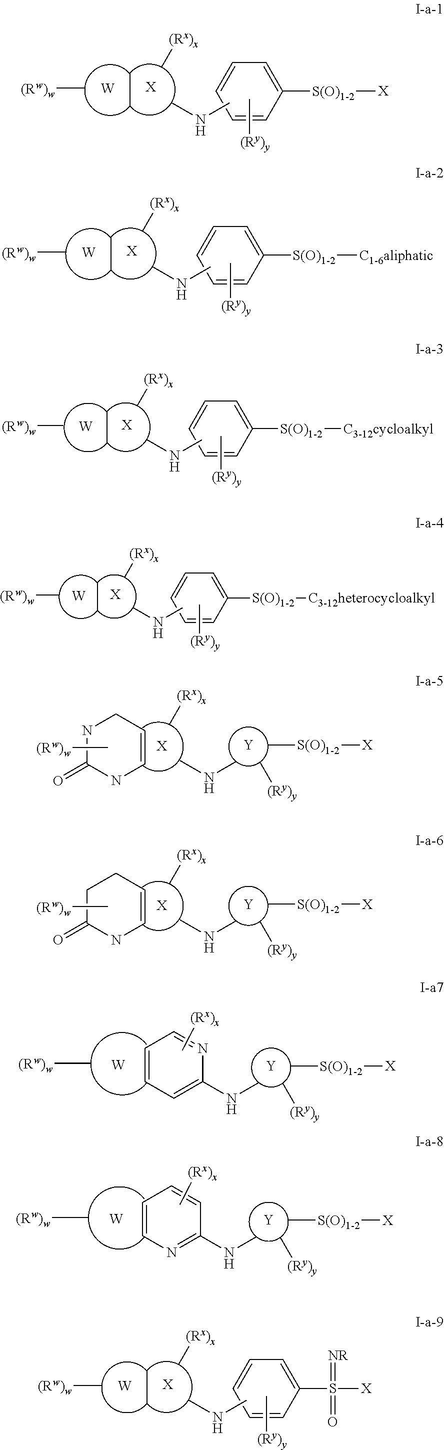 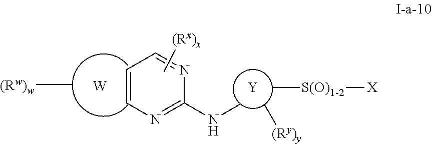
or a pharmaceutically acceptable salt thereof.

3. The compound of claim 1, wherein Ring W is a fused ring selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.

4. The compound of claim 1, wherein Ring X is a fused ring selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.

5. The compound of claim 1, wherein R^{w} is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R.

6. The compound of claim 1, wherein R^{x} is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R.

7. The compound of claim 1, wherein R^{y} is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R.

8. A compound of formula I-b′: 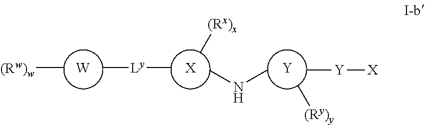
or a pharmaceutically acceptable salt thereof, wherein:
Ring W and Ring X are independently rings selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenyl, a 3 to 12-membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Y is a covalent bond, —S(O)₂—, —S(O)—, —S(O)(NR)—, —P(O)R—, —P(O)OR—, or 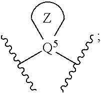
Ring Z is an optionally substituted 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur;
Q⁵ is carbon or sulfur;
X is —NR₂ or an optionally substituted group selected from C₁₋₆ aliphatic, phenyl, a 3 to 12-membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
R^{w}, R^{x}, and R^{y} are independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, and —NRS(O)₂R; or
two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form a 4-7 membered saturated, partially unsaturated, or heteroaryl ring having 0-3 heteroatoms, in addition to the atom or atoms to which they are attached, independently selected from nitrogen, oxygen, and sulfur; and
L^{y} is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CF₂—, —CRF—, —NR—, —S—, —S(O)—, —S(O)₂— or —CR═CR—, or:
L^{y} and one R^{x} are optionally taken together with their intervening atoms to form a 5-6 membered saturated, partially unsaturated or heteroaryl ring having 0-3 heteroatoms independently selected from oxygen, nitrogen or sulfur; and
w, x, and y are independently 0, 1, 2, 3, or 4.

9. The compound of claim 8, wherein said compound is a compound of any of the following formulae: 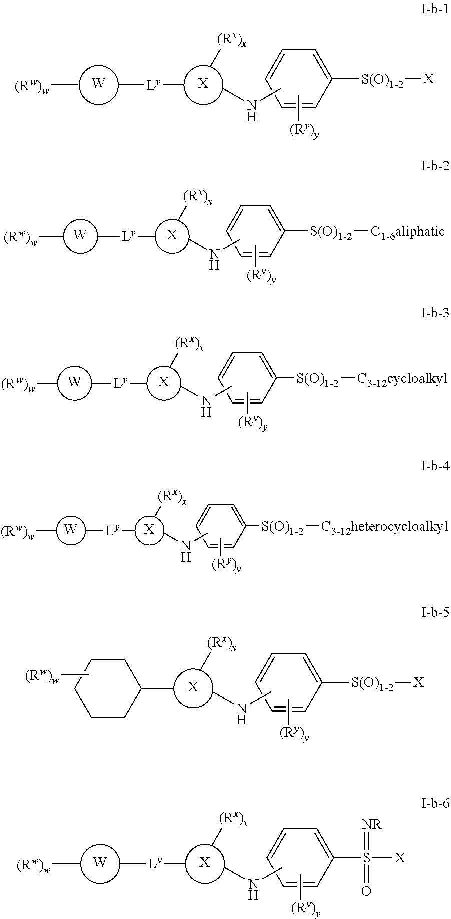
or a pharmaceutically acceptable salt thereof.

10. The compound of claim 8, wherein Ring W is a ring selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.

11. The compound of claim 8, wherein Ring X is a ring selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.

12. The compound of claim 8, wherein R^{w} is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R.

13. The compound of claim 8, wherein R^{x} is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R.

14. The compound of claim 8, wherein R^{y} is hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, or —NRS(O)₂R.

15. The compound of claim 8, wherein said compound is selected from any one of the compounds depicted in Table 1, or a pharmaceutically acceptable salt thereof.

16. A pharmaceutical composition comprising a compound of claim 8, and a pharmaceutically acceptable carrier, adjuvant, or vehicle.

17. The pharmaceutical composition according to claim 16, further comprising an additional therapeutic agent.

18. A method of inhibiting CDK2 or CDK2 and CCNE1 in a patient or biological sample comprising administering to said patient, or contacting said biological sample with a compound according to claim 8, or a pharmaceutical composition thereof.

19. A method of treating an CDK2-mediated disorder, disease, or condition in a patient comprising administering to said patient a compound according to claim 8, or a pharmaceutical composition thereof.

20. The method of claim 19, wherein CDK2-mediated disorder, disease, or condition is cancer.

21. The method of claim 20, wherein the cancer the cancer is characterized by amplification or overexpression of CCNE1.
